(19) United States
(12) Patent Application Publication
(10) Pub. No.: US 2015/0080438 A1
(43) Pub. Date: Mar. 19, 2015

(54) 6,7-Dihydro-5H-benzo[7]annulene derivatives, processes for their preparation, pharmaceutical products comprising them and their use for preparing medicaments

(71) Applicant: Bayer Intellectual Property GmbH, Monheim (DE)

(72) Inventors: Tim Wintermantel, Koln (DE); Carsten Moeller, Berlin (DE); Ulrich Bothe, Berlin (DE); Reinhard Nubbemeyer, Berlin (DE); Ludwig Zorn, Berlin (DE); Antonius Ter Laak, Berlin (DE); Rolf Bohlmann, Berlin (DE); Lars Wortmann, Berlin (DE)

(73) Assignee: BAYER INTELLECTUAL PROPERTY GMBH, Monheim (DE)

(21) Appl. No.: 14/363,811

(22) PCT Filed: Dec. 4, 2012
(86) PCT No.: PCT/EP2012/074368

§ 371 Date: Dec. 2, 2014

(30) Foreign Application Priority Data
Dec. 8, 2011 (DE) 102011087987.0

(51) Int. Cl. C07D 213/38; A61K 31/145; A61K 31/4406; C07C 317/28; C07C 317/32; C07C 323/25; A61K 45/06

(52) U.S. Cl. CPC C07D 213/38; C07C 323/25; A61K 31/145; A61K 45/06; C07C 317/28; C07C 317/32; A61K 31/4406; C07C 2102/12
USPC 514/357; 564/337; 514/657; 514/650; 546/334

(57) ABSTRACT

The invention relates to selective oestrogen receptor modulators (SERMs) and to processes for their preparation, to their use for the treatment and/or prophylaxis of diseases and to their use for preparing medicaments for the treatment and/or prophylaxis of diseases, in particular of bleeding disorders, osteoporosis, endometriosis, myomata, hormone-dependent tumours, for hormone replacement therapy and for contraception.

The invention relates to selective oestrogen receptor modulators (SERMs) and to processes for their preparation, to their use for the treatment and/or prophylaxis of diseases and to their use for preparing medicaments for the treatment and/or prophylaxis of diseases, in particular of bleeding disorders, osteoporosis, endometriosis, myomata, hormone-dependent tumours, for hormone replacement therapy and for contraception.

SERMs are compounds that have, tissue-selectively, either an anti-oestrogenic/oestrogen-inhibiting or an oestrogenic or partially oestrogenic action, for example in the case of the uterus they inhibit the action of oestrogen, but in the case of bone they have a neutral or oestrogen-like action. Tamoxifen, raloxifene and bazedoxifene may be mentioned as examples of such compounds. SERMs are to be differentiated from pure anti-oestrogens, which have a purely antagonistic action, inhibiting the action of oestrogen, in all tissues and do not display any oestrogenic or partially oestrogenic action in a tissue. SERDs (selective oestrogen receptor downregulators) belong to the anti-oestrogens and lead at the protein level to complete degradation of the oestrogen receptor in the target cells. The compound fulvestrant may be mentioned as an example of a pure anti-oestrogen or SERD.

6,7-Dihydro-5H-benzo[7]annulene derivatives as SERMs and their use in the treatment of bleeding disorders, osteoporosis, endometriosis, myomata, hormone-dependent tumours, for hormone replacement therapy and for contraception have been already described (cf. WO 00/03979).

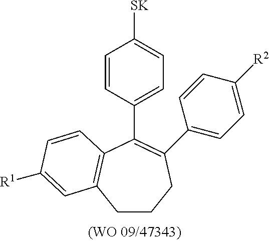

Further information on structurally more distantly related substances, SERMs or the use of particular SERMs in the treatment of specific diseases is given, for example, in EP 0584952, WO 96/21656; J. Endocrinol. 1994, 141, 335; EP 0124369; U.S. Pat. No. 6,645,951; Bioorg. Med. Chem. Lett. 2006, 14, 4803-4819; U.S. Pat. No. 6,153,768; Bioorganic & Medicinal Chemistry Letters 14 (2004) 4659-4663; DE 19521646 A1, Archiv der Pharmazie 333, (2000) 305-311; U.S. Pat. No. 6,147,105, DE 10117441, EP 138504, DE 19622457; DE 19636625, WO 98/07740, WO 99/33855, WO 00/14104, Mol. Pharmacol. 1991, 39: 421-428; J. Med. Chem. 1986, 29, 2053-2059; J. Med. Chem. 1988, 31, 1316-1326; WO 00/55137, US 20030105148, WO 2009047343, Indian Journal of Chemistry, Vol 25B, August 1986, 832-837; WO04/58682 or Bioorg. and Medicinal Chemistry 16 (2008) 9554-9573.

It is an object of the present invention to provide alternative substances acting as SERMs with improved physicochemical properties.

The present invention provides compounds of the formula (I)

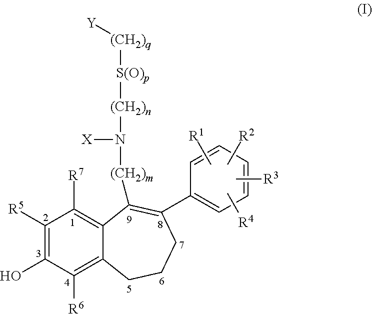

in which
R¹, R², R³ and R⁴ independently of one another represent hydrogen, hydroxy, alkoxy, nitrile, alkylsulphonyl or represent the replacement of a CH group in the aromatic ring by a nitrogen atom or
   provided one or more of the other substituents represent hydroxy, alkoxy, nitrile or alkylsulphonyl—independently of one another represent fluorine
R⁵, R⁶ and R⁷ independently of one another represent hydrogen, fluorine, chlorine, bromine, methyl, ethyl, trifluoromethyl or nitrile,
X represents hydrogen or alkyl-, cycloalkyl-, alkoxyalkyl, C₃-C₆-alkenyl or C₃-C₆-alkynyl which are optionally mono- or polysubstituted by halogen, hydroxy, —CN or deuterium,
Y represents perfluorinated or partially fluorinated C₁-C₄-alkyl or perfluorinated or partially fluorinated C₃-C₈-cycloalkyl,
m represents 4, 5, 6 or 7,
n represents 2, 3, 4, 5, 6 or 7,
p represents 0, 1 or 2 and
q represents 1, 2, 3 or 4,
   and salts, solvates or solvates of the salts thereof, including all crystal modifications.

It was found that 6,7-dihydro-5H-benzo[7]annulene derivatives (I) which have a substituted aromatic substituent as described above attached at the 8-position and which have an optionally substituted aliphatic chain attached in position 9 act as SERMs. Many of the claimed 6,7-dihydro-5H-benzo[7]annulene derivatives have a destabilizing effect on the ERα content (remaining relative ERα content less than or equal to 30%). Over the entire structural range, these compounds display a high anti-oestrogenic action in vitro (IC₅₀ values below 0.3 micromolar) and predominantly even double-digit or single-digit nanomolar IC₅₀ values for the inhibition of estradiol-induced luciferase activity.

Compounds according to the invention are the compounds of formula (I) and their salts, solvates and solvates of the salts, the compounds of the formulae given below that are covered by formula (I) and their salts, solvates and solvates of the salts and the compounds presented below as working examples, which are covered by formula (I), and their salts, solvates and solvates of the salts, provided that the compounds mentioned below that are covered by formula (I) are not already salts, solvates and solvates of the salts.

Depending on their structure, the compounds according to the invention can exist in stereoisomeric forms (enantiomers, diastereomers). In compounds of formula (I), there can be stereocentres on the sulphur atom (for p=1) and/or in the radical X. The invention therefore includes the enantiomers and/or diastereomers and respective mixtures thereof. The stereoisomerically uniform constituents can be isolated in a known way from such mixtures of enantiomers and/or diastereomers. In the context of the present invention, a compound is enantiomerically pure at an enantiomeric excess of more than 90% (>90% ee).

If the compounds according to the invention can occur in tautomeric forms, the present invention encompasses all tautomeric forms.

Preferred salts in the context of the present invention are physiologically acceptable salts of the compounds according to the invention. However, salts that for their part are not suitable for pharmaceutical uses but can be used, for example, for isolation or purification of the compounds according to the invention are also embraced.

Physiologically acceptable salts of the compounds according to the invention include acid addition salts of mineral acids, carboxylic acids and sulphonic acids, e.g. salts of hydrochloric acid, hydrobromic acid, sulphuric acid, phosphoric acid, methanesulphonic acid, ethanesulphonic acid, toluenesulphonic acid, benzenesulphonic acid, acetic acid, formic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of customary bases such as, for example and preferably, alkali metal salts (e.g. sodium salts and potassium salts), salts of alkaline earth metals (e.g. calcium salts and magnesium salts) and ammonium salts, derived from ammonia or organic amines having 1 to 16 carbon atoms such as, for example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates refers to forms of the compounds according to the invention which, in solid or liquid state, form a complex by coordination with solvent molecules. Hydrates are a special form of solvates, in which coordination takes place with water. Hydrates are preferred as solvates within the context of the present invention.

Moreover, the present invention also encompasses prodrugs of the compounds according to the invention. The term “prodrugs” includes compounds which for their part may be biologically active or inactive, however, during their residence time in the body they are converted into compounds according to the invention (for example metabolically or hydrolytically).

In the context of the present invention, the substituents have, unless stated otherwise, the following meaning:

C₃-C₆-Alkenyl represents a straight-chain or branched alkenyl radical having generally 3 to 6 carbon atoms, by way of example and preferably prop-2-en-1-yl, but-2-en-1-yl and but-3-en-1-yl.

C₃-C₆-Alkynyl represents a straight-chain or branched alkynyl radical having generally 3 to 6 carbon atoms, by way of example and preferably prop-2-yn-1-yl, but-2-yn-1-yl and but-3-yn-1-yl.

Alkyl per se and “alk” and “alkyl” in alkoxy, alkylcarbonyl, alkylamino, alkylaminocarbonyl, alkoxycarbonyl, alkoxycarbonylamino, alkylcarbonylamino and alkylsulphonyl represent a straight-chain or branched alkyl radical having generally 1 to 6, preferably 1 to 4, particularly preferably 1 to 3 carbon atoms, by way of example and preferably methyl, ethyl, n-propyl, isopropyl, tert-butyl, n-pentyl and n-hexyl.

Alkoxy by way of example and preferably represents methoxy, ethoxy, n-propoxy, isopropoxy, tert-butoxy, n-pentoxy and n-hexoxy.

Alkylsulphonyl by way of example and preferably represents methylsulphonyl, ethylsulphonyl, propylsulphonyl and isopropylsulphonyl.

Alkoxyalkyl by way of example and preferably represents methoxyethyl, ethoxyethyl, methoxypropyl and ethoxypropyl.

Cycloalkyl represents a cycloalkyl group having generally 3 to 8, preferably 5 to 7, carbon atoms, where the ring may also be partially unsaturated, by way of example and preferably cyclopropyl, cyclobutyl, cyclopentyl, cyclohexyl and cycloheptyl.

Halogen represents fluorine, chlorine, bromine and iodine.

Deuterium or D is used to describe substances where, at the position in question, the proportion of deuterium is highly elevated compared to the natural isotope ratio, i.e., for example, compounds having an isotope purity of 10-100%, in particular an isotope purity of more than 50%, more than 60%, more than 70%, more than 80% or more than 90%.

Perfluorinated—C₁-C₄Alkyl represents a fully fluorinated straight-chain or branched alkyl radical having generally 1 to 4, preferably 1 to 3, carbon atoms, by way of example and preferably represents trifluoromethyl, pentafluoroethyl, heptafluoropropyl and heptafluoroisopropyl.

Partially fluorinated—C₁-C₄-alkyl represents a partially fluorinated straight-chain or branched alkyl radical having generally 1 to 4 carbon atoms—selected from, but not limited to 1,2,2,2-tetrafluoroethyl, 1,1,2,2-tetrafluoroethyl, 2,2,2-trifluoro-1-(trifluoromethyl)ethyl, 1,1,3,3,3-pentafluoropropyl, 1,1,2,3,3,3-hexafluoropropyl, 1,1,2,2,3,3,4,4-octafluorobutyl, 1,2,2,3,3,3-hexafluoro-1-methylpropyl, 1,1,3,3,3-pentafluoro-2-(trifluoromethyl)propyl, 2,2,2-trifluoro-1-methyl-1-(trifluoromethyl)ethyl, 2-fluoro-1,1-bis(fluoromethyl)ethyl. 1,2,2,2-Tetrafluoroethyl, 1,1,3,3,3-pentafluoropropyl, 1,1,2,3,3,3-hexafluoropropyl and 2,2,2-trifluoro-1-(trifluoromethyl)ethyl are preferred. 2,2,2-Trifluoro-1-(trifluoromethyl)ethyl and 1,1,3,3,3-pentafluoropropyl are particularly preferred.

Perfluorinated—C₃-C₇-cycloalkyl represents a fully fluorinated cycloalkyl group having generally 3-7, preferably 5-6, carbon atoms, by way of example and preferably perfluorocyclopentyl and perfluorocyclohexyl.

Partially fluorinated—C₃-C₇-cycloalkyl represents a partially fluorinated cycloalkyl group having generally 3 to 7 carbon atoms—selected from, but not limited to: 2,2-difluorocycloheptyl, 2-fluorocycloheptyl, 3,3-difluorocycloheptyl, 3-fluorocycloheptyl, 4,4-difluorocycloheptyl, 4-fluorocycloheptyl, 4,4-difluorocyclohexyl, 4-fluorocyclohexyl, 3,3-difluorocyclohexyl, 3-fluorocyclohexyl, 2,2-difluorocyclohexyl, 2-difluorocyclohexyl, 3,3-difluorocyclopentyl, 3-fluorocyclopentyl, 2,2-difluorocyclopentyl, 2-fluorocyclopentyl, 3,3-difluorocyclobutyl, 3-fluorocyclobutyl, 2,2-difluorocyclobutyl, 2-fluorocyclobutyl, 2,2-difluorocyclopropyl, 2-fluorocyclopropyl. 4,4-Difluorocyclohexyl, 4-fluorocyclohexyl, 3,3-difluorocyclohexyl, 3,3-difluorocyclopentyl, 3,3-difluorocyclobutyl and 2,2-difluorocyclopropyl are preferred. 4,4-Difluorocyclohexyl is particularly preferred.

A symbol * on a bond denotes the point of attachment in the molecule.

When radicals in the compounds according to the invention are substituted, the radicals can be mono- or polysubstituted, unless indicated otherwise. In the context of the present invention, for all radicals that occur more than once, their meanings are independent of one another. Substitution by one, two or three identical or different substituents is preferred. Substitution by one substituent is very particularly preferred.

The present invention furthermore provides compounds of the formula (I) in which
R¹, R², R³ and R⁴ independently of one another represent hydrogen, hydroxy, nitrile, methylsulphonyl or represent the replacement of a CH group in the aromatic ring by a nitrogen atom or
   provided one or more of the other substituents represent hydroxy, nitrile, methylsulphonyl—independently of one another represent fluorine,
R⁵ and R⁶ independently of one another represent hydrogen, chlorine or fluorine,
R⁷ represents hydrogen,
X represents hydrogen or represents optionally hydroxy- or methoxy-substituted C₁-C₄-alkyl,
Y represents —CF₃, —C₂F₅, —CF₂CF₂CF₃ or —CF(CF₃)₂,
m represents 5 or 6,
n represents 3, 4, 5 or 6,
p represents 0, 1 or 2 and
q represents 2, 3 or 4,
   and salts, solvates or solvates of the salts thereof, including all crystal modifications.

The present invention also provides compounds of the formula (I) in which
R¹ and R² represent hydrogen and
   either
R³ represents hydrogen and
R⁴ represents hydrogen, hydroxy, nitrile, methylsulphonyl or represents the replacement of a CH group in the aromatic ring by a nitrogen atom or
R³ represents fluorine and

R⁴ represents hydroxy, nitrile or methylsulphonyl,
R⁵ and R⁶ represent hydrogen, chlorine or fluorine, but do not both represent chlorine and do not both represent fluorine
X represents optionally hydroxy- or methoxy-substituted C₁-C₄-alkyl,
Y represents —CF₃ or —C₂F₅,
m represents 5 or 6,
n represents 3, 4, 5 or 6,
p represents 0, 1 or 2 and
q represents 2 or 3,
   and salts, solvates or solvates of the salts thereof, including all crystal modifications.

The present invention likewise provides compounds of the formula (II)

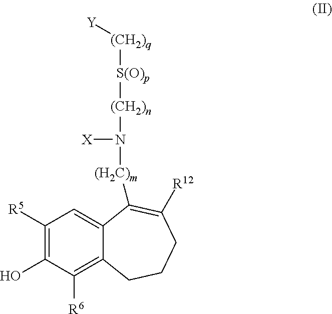

in which
R¹² represents phenyl, 3-hydroxyphenyl, 4-hydroxyphenyl, 3-fluoro-4-hydroxphenyl, 4-fluoro-3-hydroxyphenyl, 2-fluoro-5-hydroxyphenyl, 4-methylsulphonylphenyl, 3-methylsulphonylphenyl, 4-cyanophenyl or 3-pyridyl, R⁵ and R⁶ either represent hydrogen or fluorine, but do not both represent fluorine or—if

R⁵ represents hydrogen—R⁶ represents chlorine,
X represents methyl, ethyl, methoxyethyl, methoxypropyl, hydroxyethyl, 3-hydroxypropyl or 2-hydroxy-2-methylpropyl,
Y represents —CF₃ or —C₂F₅,
m represents 5 or 6,
n represents 3, 4, 5 or 6,
p represents 0, 1 or 2,
q represents 2 or 3
   and salts, solvates or solvates of the salts thereof, including all crystal modifications.

Moreover, the present invention provides the following compounds:
9-[5-(methyl{3-[(4,4,5,5,5-pentafluoropentyl)sulphanyl]propyl}amino)pentyl]-8-phenyl-6,7-dihydro-5H-benzo[7]annulen-3-ol
9-[5-(methyl{3-[(RS)-(4,4,5,5,5-pentafluoropentyl)sulphinyl]propyl}amino)pentyl]-8-phenyl-6,7-dihydro-5H-benzo[7]annulen-3-ol
9-[5-(methyl{3-[(4,4,5,5,5-pentafluoropentyl)sulphonyl]propyl}amino)pentyl]-8-phenyl-6,7-dihydro-5H-benzo[7]annulen-3-ol
8-(3-hydroxyphenyl)-9-[5-(methyl{3-[(RS)-(4,4,5,5,5-pentafluoropentyl)sulphinyl]propyl}amino)pentyl]-6,7-dihydro-5H-benzo[7]annulen-3-ol
8-(3-hydroxyphenyl)-9-[5-(methyl{3-[(4,4,5,5,5-pentafluoropentyl)sulphonyl]propyl}amino)pentyl]-6,7-dihydro-5H-benzo[7]annulen-3-ol
8-(3-hydroxyphenyl)-9-[5-(methyl{4-[(RS)-(4,4,5,5,5-pentafluoropentyl)sulphinyl]butyl}amino)pentyl]-6,7-dihydro-5H-benzo[7]annulen-3-ol
8-(3-hydroxyphenyl)-9-[5-(methyl{4-[(4,4,5,5,5-pentafluoropentyl)sulphonyl]butyl}amino)pentyl]-6,7-dihydro-5H-benzo[7]annulen-3-ol
8-(3-hydroxyphenyl)-9-[5-(methyl{5-[(RS)-(4,4,5,5,5-pentafluoropentyl)sulphinyl]pentyl}amino)pentyl]-6,7-dihydro-5H-benzo[7]annulen-3-ol
8-(3-hydroxyphenyl)-9-[5-(methyl{5-[(4,4,5,5,5-pentafluoropentyl)sulphonyl]pentyl}amino)pentyl]-6,7-dihydro-5H-benzo[7]annulen-3-ol
8-(3-hydroxyphenyl)-9-[5-(methyl{6-[(RS)-(4,4,5,5,5-pentafluoropentyl)sulphinyl]hexyl}amino)pentyl]-6,7-dihydro-5H-benzo[7]annulen-3-ol
8-(3-hydroxyphenyl)-9-[5-(methyl{6-[(4,4,5,5,5-pentafluoropentyl)sulphonyl]hexyl}amino)pentyl]-6,7-dihydro-5H-benzo[7]annulen-3-ol
8-(3-hydroxyphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol
8-(3-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol
8-(3-hydroxyphenyl)-9-[6-(methyl{4-[(RS)-(4,4,5,5,5-pentafluoropentyl)sulphinyl]butyl}amino)hexyl]-6,7-dihydro-5H-benzo[7]annulen-3-ol
8-(3-hydroxyphenyl)-9-[6-(methyl{4-[(4,4,5,5,5-pentafluoropentyl)sulphonyl]butyl}amino)hexyl]-6,7-dihydro-5H-benzo[7]annulen-3-ol
8-(3-hydroxyphenyl)-9-[6-(methyl{5-[(RS)-(4,4,5,5,5-pentafluoropentyl)sulphinyl]pentyl}amino)hexyl]-6,7-dihydro-5H-benzo[7]annulen-3-ol
8-(3-hydroxyphenyl)-9-[6-(methyl{5-[(4,4,5,5,5-pentafluoropentyl)sulphonyl]pentyl}amino)hexyl]-6,7-dihydro-5H-benzo[7]annulen-3-ol
8-(3-hydroxyphenyl)-9-[6-(methyl{6-[(RS)-(4,4,5,5,5-pentafluoropentyl)sulphinyl]hexyl}amino)hexyl]-6,7-dihydro-5H-benzo[7]annulen-3-ol
8-(3-hydroxyphenyl)-9-[6-(methyl{6-[(4,4,5,5,5-pentafluoropentyl)sulphonyl]hexyl}amino)hexyl]-6,7-dihydro-5H-benzo[7]annulen-3-ol
8-(4-hydroxyphenyl)-9-[6-(methyl{3-[(4,4,5,5,5-pentafluoropentyl)sulphanyl]propyl}amino)hexyl]-6,7-dihydro-5H-benzo[7]annulen-3-ol
8-(3-mesylphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol
4-{3-hydroxy-9-[6-(methyl{3-[(4,4,5,5,5-pentafluoropentyl)sulphonyl]propyl}amino)hexyl]-6,7-dihydro-5H-benzo[7]annulen-8-yl}benzonitrile
8-(2-fluoro-5-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol
4-chloro-8-(4-fluoro-3-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol
8-(4-fluoro-3-hydroxyphenyl)-9-[5-(methyl{4-[(4,4,5,5,5-pentafluoropentyl)sulphonyl]butyl}amino)pentyl]-6,7-dihydro-5H-benzo[7]annulen-3-ol
8-(4-hydroxyphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol
8-(4-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol
8-(4-fluoro-3-hydroxyphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol
8-(4-fluoro-3-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol
8-(4-fluoro-3-hydroxyphenyl)-9-[6-(methyl{4-[(RS)-(4,4,5,5,5-pentafluoropentyl)sulphinyl]butyl}amino)hexyl]-6,7-dihydro-5H-benzo[7]annulen-3-ol
8-(4-fluoro-3-hydroxyphenyl)-9-[6-(methyl{4-[(4,4,5,5,5-pentafluoropentyl)sulphonyl]butyl}amino)hexyl]-6,7-dihydro-5H-benzo[7]annulen-3-ol
8-(4-fluoro-3-hydroxyphenyl)-9-[6-(methyl{5-[(RS)-(4,4,5,5,5-pentafluoropentyl)sulphinyl]pentyl}amino)hexyl]-6,7-dihydro-5H-benzo[7]annulen-3-ol
8-(4-fluoro-3-hydroxyphenyl)-9-[6-(methyl{5-[(4,4,5,5,5-pentafluoropentyl)sulphonyl]pentyl}amino)hexyl]-6,7-dihydro-5H-benzo[7]annulen-3-ol
8-(4-fluoro-3-hydroxyphenyl)-9-[6-(methyl{6-[(RS)-(4,4,5,5,5-pentafluoropentyl)sulphinyl]hexyl}amino)hexyl]-6,7-dihydro-5H-benzo[7]annulen-3-ol
8-(4-fluoro-3-hydroxyphenyl)-9-[6-(methyl{6-[(4,4,5,5,5-pentafluoropentyl)sulphonyl]hexyl}amino)hexyl]-6,7-dihydro-5H-benzo[7]annulen-3-ol
8-(3-fluoro-4-hydroxyphenyl)-9-[5-(methyl{3-[(4,4,5,5,5-pentafluoropentyl)sulphanyl]propyl}amino)pentyl]-6,7-dihydro-5H-benzo[7]annulen-3-ol
8-(3-fluoro-4-hydroxyphenyl)-9-[5-(methyl{3-[(RS)-(4,4,5,5,5-pentafluoropentyl)sulphinyl]propyl}amino)pentyl]-6,7-dihydro-5H-benzo[7]annulen-3-ol
8-(3-fluoro-4-hydroxyphenyl)-9-[5-(methyl{3-[(4,4,5,5,5-pentafluoropentyl)sulphonyl]propyl}amino)pentyl]-6,7-dihydro-5H-benzo[7]annulen-3-ol
9-[5-(methyl{3-[(4,4,5,5,5-pentafluoropentyl)sulphanyl]propyl}amino)pentyl]-8-(3-pyridyl)-6,7-dihydro-5H-benzo[7]annulen-3-ol
9-[5-(methyl{3-[(RS)-(4,4,5,5,5-pentafluoropentyl)sulphinyl]propyl}amino)pentyl]-8-(3-pyridyl)-6,7-dihydro-5H-benzo[7]annulen-3-ol
9-[5-(methyl{3-[(4,4,5,5,5-pentafluoropentyl)sulphonyl]propyl}amino)pentyl]-8-(3-pyridyl)-6,7-dihydro-5H-benzo[7]annulen-3-ol
9-[6-(methyl{3-[(4,4,5,5,5-pentafluoropentyl)sulphanyl]propyl}amino)hexyl]-8-(3-pyridyl)-6,7-dihydro-5H-benzo[7]annulen-3-ol
9-[6-(methyl{3-[(RS)-(4,4,5,5,5-pentafluoropentyl)sulphinyl]propyl}amino)hexyl]-8-(3-pyridyl)-6,7-dihydro-5H-benzo[7]annulen-3-ol
9-[6-(methyl{3-[(4,4,5,5,5-pentafluoropentyl)sulphonyl]propyl}amino)hexyl]-8-(3-pyridyl)-6,7-dihydro-5H-benzo[7]annulen-3-ol
8-(4-mesylphenyl)-9-[5-(methyl{3-[(4,4,5,5,5-pentafluoropentyl)sulphanyl]propyl}amino)pentyl]-6,7-dihydro-5H-benzo[7]annulen-3-ol
8-(4-mesylphenyl)-9-[5-(methyl{3-[(RS)-(4,4,5,5,5-pentafluoropentyl)sulphinyl]propyl}amino)pentyl]-6,7-dihydro-5H-benzo[7]annulen-3-ol
8-(4-mesylphenyl)-9-[5-(methyl{3-[(4,4,5,5,5-pentafluoropentyl)sulphonyl]propyl}amino)pentyl]-6,7-dihydro-5H-benzo[7]annulen-3-ol
8-(4-mesylphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol
8-(4-mesylphenyl)-9-[6-(methyl{3-[(4,4,5,5,5-pentafluoropentyl)sulphonyl]propyl}amino)hexyl]-6,7-dihydro-5H-benzo[7]annulen-3-ol
9-[5-({3-[(RS)-(4,4,5,5,5-pentafluoropentyl)sulphinyl]propyl}amino)pentyl]-8-phenyl-6,7-dihydro-5H-benzo[7]annulen-3-ol
8-(3-hyd roxyphenyl)-9-[6-(methyl{3-[(4,4,4-trifluorobutyl)sulphonyl]propyl}amino)hexyl]-6,7-dihydro-5H-benzo[7]annulen-3-ol
8-(3-hydroxyphenyl)-9-[6-(methyl{4-[(4,4,4-trifluorobutyl)sulphonyl]butyl}amino)hexyl]-6,7-dihydro-5H-benzo[7]annulen-3-ol
8-(3-hydroxyphenyl)-9-[6-(methyl{3-[(RS)-(3,3,4,4,4-pentafluorobutyl)sulphinyl]propyl}amino)hexyl]-6,7-dihydro-5H-benzo[7]annulen-3-ol
8-(3-hydroxyphenyl)-9-[6-(methyl{3-[(3,3,4,4,4-pentafluorobutyl)sulphonyl]propyl}amino)hexyl]-6,7-dihydro-5H-benzo[7]annulen-3-ol
8-(3-hydroxyphenyl)-9-[6-(methyl{3-[(RS)-(3,3,3-trifluoropropyl)sulphinyl]propyl}amino)hexyl]-6,7-dihydro-5H-benzo[7]annulen-3-ol
8-(3-hydroxyphenyl)-9-[6-(methyl{3-[(3,3,3-trifluoropropyl)sulphonyl]propyl}amino)hexyl]-6,7-dihydro-5H-benzo[7]annulen-3-ol
9-[6-(methyl{3-[(4,4,4-trifluorobutyl)sulphonyl]propyl}amino)hexyl]-8-phenyl-6,7-dihydro-5H-benzo[7]annulen-3-ol
9-[6-(methyl{4-[(4,4,4-trifluorobutyl)sulphonyl]butyl}amino)hexyl]-8-phenyl-6,7-dihydro-5H-benzo[7]annulen-3-ol
9-[6-(methyl{3-[(RS)-(3,3,3-trifluoropropyl)sulphinyl]propyl}amino)hexyl]-8-phenyl-6,7-dihydro-5H-benzo[7]annulen-3-ol
9-[6-(methyl{3-[(3,3,3-trifluoropropyl)sulphonyl]propyl}amino)hexyl]-8-phenyl-6,7-dihydro-5H-benzo[7]annulen-3-ol
9-[6-(methyl{3-[(RS)-(3,3,4,4,4-pentafluorobutyl)sulphinyl]propyl}amino)hexyl]-8-phenyl-6,7-dihydro-5H-benzo[7]annulen-3-ol
9-[6-(methyl{3-[(3,3,4,4,4-pentafluorobutyl)sulphonyl]propyl}amino)hexyl]-8-phenyl-6,7-dihydro-5H-benzo[7]annulen-3-ol
9-[6-(methyl{3-[(4,4,5,5,5-pentafluoropentyl)sulphonyl]propyl}amino)hexyl]-8-phenyl-6,7-dihydro-5H-benzo[7]annulen-3-ol
9-[6-(methyl{3-[(RS)-(4,4,5,5,5-pentafluoropentyl)sulphinyl]propyl}amino)hexyl]-8-phenyl-6,7-dihydro-5H-benzo[7]annulen-3-ol
9-[6-(ethyl{3-[(3,3,3-trifluoropropyl)sulphonyl]propyl}amino)hexyl]-8-(3-hydroxyphenyl)-6,7-dihydro-5H-benzo[7]annulen-3-ol
9-[6-(methyl{4-[(RS)-(4,4,5,5,5-pentafluoropentyl)sulphinyl]butyl}amino)hexyl]-8-phenyl-6,7-dihydro-5H-benzo[7]annulen-3-ol
9-[6-(methyl{4-[(4,4,5,5,5-pentafluoropentyl)sulphonyl]butyl}amino)hexyl]-8-phenyl-6,7-dihydro-5H-benzo[7]annulen-3-ol
9-[6-(ethyl{3-[(RS)-(4,4,5,5,5-pentafluoropentyl)sulphinyl]propyl}amino)hexyl]-8-phenyl-6,7-dihydro-5H-benzo[7]annulen-3-ol
9-[6-(ethyl{3-[(4,4,5,5,5-pentafluoropentyl)sulphonyl]propyl}amino)hexyl]-8-phenyl-6,7-dihydro-5H-benzo[7]annulen-3-ol
9-{6-[(2-hydroxyethyl){3-[(RS)-(4,4,5,5,5-pentafluoropentyl)sulphinyl]propyl}amino]hexyl}-8-phenyl-6,7-dihydro-5H-benzo[7]annulen-3-ol
9-{6-[(2-hydroxyethyl){3-[(4,4,5,5,5-pentafluoropentyl)sulphonyl]propyl}amino]hexyl}-8-phenyl-6,7-dihydro-5H-benzo[7]annulen-3-ol
9-{6-[(3-hydroxypropyl){3-[(RS)-(4,4,5,5,5-pentafluoropentyl)sulphinyl]propyl}amino]hexyl}-8-phenyl-6,7-dihydro-5H-benzo[7]annulen-3-ol
9-{6-[(3-hydroxypropyl){3-[(4,4,5,5,5-pentafluoropentyl)sulphonyl]propyl}amino]hexyl}-8-phenyl-6,7-dihydro-5H-benzo[7]annulen-3-ol
9-{6-[(2-hydroxy-2-methylpropyl){3-[(RS)-(4,4,5,5,5-pentafluoropentyl)sulphinyl]propyl}amino]hexyl}-8-phenyl-6,7-dihydro-5H-benzo[7]annulen-3-ol
9-{6-[(2-hydroxy-2-methylpropyl){3-[(4,4,5,5,5-pentafluoropentyl)sulphonyl]propyl}amino]hexyl}-8-phenyl-6,7-dihydro-5H-benzo[7]annulen-3-ol
9-[6-(ethyl{3-[(RS)-(3,3,3-trifluoropropyl)sulphinyl]propyl}amino)hexyl]-8-phenyl-6,7-dihydro-5H-benzo[7]annulen-3-ol
9-[6-(ethyl{3-[(3,3,3-trifluoropropyl)sulphonyl]propyl}amino)hexyl]-8-phenyl-6,7-dihydro-5H-benzo[7]annulen-3-ol
9-{6-[(2-hydroxyethyl){3-[(RS)-(3,3,3-trifluoropropyl)sulphinyl]propyl}amino]hexyl}-8-phenyl-6,7-dihydro-5H-benzo[7]annulen-3-ol
9-{6-[(2-hydroxyethyl){3-[(3,3,3-trifluoropropyl)sulphonyl]propyl}amino]hexyl}-8-phenyl-6,7-dihydro-5H-benzo[7]annulen-3-ol
9-{6-[(3-hydroxypropyl){3-[(RS)-(3,3,3-trifluoropropyl)sulphinyl]propyl}amino]hexyl}-8-phenyl-6,7-dihydro-5H-benzo[7]annulen-3-ol
9-{6-[(3-hydroxypropyl){3-[(3,3,3-trifluoropropyl)sulphonyl]propyl}amino]hexyl}-8-phenyl-6,7-dihydro-5H-benzo[7]annulen-3-ol
9-{6-[(2-hydroxy-2-methylpropyl){3-[(RS)-(3,3,3-trifluoropropyl)sulphinyl]propyl}amino]hexyl}-8-phenyl-6,7-dihydro-5H-benzo[7]annulen-3-ol
9-{6-[(2-hydroxy-2-methylpropyl){3-[(3,3,3-trifluoropropyl)sulphonyl]propyl}amino]hexyl}-8-phenyl-6,7-dihydro-5H-benzo[7]annulen-3-ol
9-[5-(methyl{4-[(RS)-(4,4,5,5,5-pentafluoropentyl)sulphinyl]butyl}amino)pentyl]-8-phenyl-6,7-dihydro-5H-benzo[7]annulen-3-ol
9-[5-(methyl{4-[(4,4,5,5,5-pentafluoropentyl)sulphonyl]butyl}amino)pentyl]-8-phenyl-6,7-dihydro-5H-benzo[7]annulen-3-ol
9-[6-(ethyl{3-[(RS)-(4,4,5,5,5-pentafluoropentyl)sulphinyl]propyl}amino)hexyl]-8-(3-hydroxyphenyl)-6,7-dihydro-5H-benzo[7]annulen-3-ol
9-{6-[(2-hydroxyethyl){3-[(RS)-(4,4,5,5,5-pentafluoropentyl)sulphinyl]propyl}amino]hexyl}-8-(3-hydroxyphenyl)-6,7-dihydro-5H-benzo[7]annulen-3-ol
8-(3-hydroxyphenyl)-9-{6-[(3-hydroxypropyl){3-[(RS)-(4,4,5,5,5-pentafluoropentyl)sulphinyl]propyl}amino]hexyl}-6,7-dihydro-5H-benzo[7]annulen-3-ol
9-{6-[(2-hydroxy-2-methylpropyl){3-[(RS)-(4,4,5,5,5-pentafluoropentyl)sulphinyl]propyl}amino]hexyl}-8-(3-hydroxyphenyl)-6,7-dihydro-5H-benzo[7]annulen-3-ol
9-[6-(ethyl{3-[(4,4,5,5,5-pentafluoropentyl)sulphonyl]propyl}amino)hexyl]-8-(3-hydroxyphenyl)-6,7-dihydro-5H-benzo[7]annulen-3-ol
9-{6-[(2-hydroxyethyl){3-[(4,4,5,5,5-pentafluoropentyl)sulphonyl]propyl}amino]hexyl}-8-(3-hydroxyphenyl)-6,7-dihydro-5H-benzo[7]annulen-3-ol
8-(3-hydroxyphenyl)-9-{6-[(3-hydroxypropyl){3-[(4,4,5,5,5-pentafluoropentyl)sulphonyl]propyl}amino]hexyl}-6,7-dihydro-5H-benzo[7]annulen-3-ol
9-{6-[(2-hydroxy-2-methylpropyl){3-[(4,4,5,5,5-pentafluoropentyl)sulphonyl]propyl}amino]hexyl}-8-(3-hydroxyphenyl)-6,7-dihydro-5H-benzo[7]annulen-3-ol
9-[6-(ethyl{3-[(RS)-(3,3,3-trifluoropropyl)sulphinyl]propyl}amino)hexyl]-8-(3-hydroxyphenyl)-6,7-dihydro-5H-benzo[7]annulen-3-ol
9-{6-[(2-hydroxyethyl){3-[(RS)-(3,3,3-trifluoropropyl)sulphinyl]propyl}amino]hexyl}-8-(3-hydroxyphenyl)-6,7-dihydro-5H-benzo[7]annulen-3-ol
8-(3-hydroxyphenyl)-9-{6-[(3-hydroxypropyl){3-[(RS)-(3,3,3-trifluoropropyl)sulphinyl]propyl}amino]hexyl}-6,7-dihydro-5H-benzo[7]annulen-3-ol
9-{6-[(2-hydroxy-2-methylpropyl){3-[(RS)-(3,3,3-trifluoropropyl)sulphinyl]propyl}amino]hexyl}-8-(3-hydroxyphenyl)-6,7-dihydro-5H-benzo[7]annulen-3-ol
9-{6-[(2-hydroxyethyl){3-[(3,3,3-trifluoropropyl)sulphonyl]propyl}amino]hexyl}-8-(3-hydroxyphenyl)-6,7-dihydro-5H-benzo[7]annulen-3-ol
8-(3-hydroxyphenyl)-9-{6-[(3-hydroxypropyl){3-[(3,3,3-trifluoropropyl)sulphonyl]propyl}amino]hexyl}-6,7-dihydro-5H-benzo[7]annulen-3-ol
9-{6-[(2-hydroxy-2-methylpropyl){3-[(3,3,3-trifluoropropyl)sulphonyl]propyl}amino]hexyl}-8-(3-hydroxyphenyl)-6,7-dihydro-5H-benzo[7]annulen-3-ol
2-fluoro-8-(4-fluoro-3-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol
4-chloro-9-[6-(methyl{3-[(4,4,5,5,5-pentafluoropentyl)sulphonyl]propyl}amino)hexyl]-8-phenyl-6,7-dihydro-5H-benzo[7]annulen-3-ol
4-chloro-9-[6-(methyl{4-[(4,4,5,5,5-pentafluoropentyl)sulphonyl]butyl}amino)hexyl]-8-phenyl-6,7-dihydro-5H-benzo[7]annulen-3-ol
4-chloro-9-[6-(methyl{3-[(RS)-(4,4,5,5,5-pentafluoropentyl)sulphinyl]propyl}amino)hexyl]-8-phenyl-6,7-dihydro-5H-benzo[7]annulen-3-ol
8-(4-fluoro-3-hydroxyphenyl)-9-{6-[(2-methoxyethyl){3-[(4,4,5,5,5-pentafluoropentyl)sulphonyl]propyl}amino]hexyl}-6,7-dihydro-5H-benzo[7]annulen-3-ol
8-(3-hydroxyphenyl)-9-{6-[(2-methoxyethyl){3-[(4,4,5,5,5-pentafluoropentyl)sulphonyl]propyl}amino]hexyl}-6,7-dihydro-5H-benzo[7]annulen-3-ol
8-(3-hydroxyphenyl)-9-{6-[(3-methoxypropyl){3-[(4,4,5,5,5-pentafluoropentyl)sulphonyl]propyl}amino]hexyl}-6,7-dihydro-5H-benzo[7]annulen-3-ol
8-(3-hydroxyphenyl)-9-{6-[(3-methoxypropyl){3-[(RS)-(4,4,5,5,5-pentafluoropentyl)sulphinyl]propyl}amino]hexyl}-6,7-dihydro-5H-benzo[7]annulen-3-ol
4-chloro-9-[6-(methyl{4-[(3,3,3-trifluoropropyl)sulphonyl]butyl}amino)hexyl]-8-phenyl-6,7-dihydro-5H-benzo[7]annulen-3-ol
8-(4-fluoro-3-hydroxyphenyl)-9-[6-(methyl{4-[(3,3,3-trifluoropropyl)sulphonyl]butyl}amino)hexyl]-6,7-dihydro-5H-benzo[7]annulen-3-ol
9-[5-(methyl{4-[(3,3,3-trifluoropropyl)sulphonyl]butyl}amino)pentyl]-8-phenyl-6,7-dihydro-5H-benzo[7]annulen-3-ol
8-(3-hydroxyphenyl)-9-[5-(methyl{4-[(3,3,3-trifluoropropyl)sulphonyl]butyl}amino)pentyl]-6,7-dihydro-5H-benzo[7]annulen-3-ol
8-(3-hydroxyphenyl)-9-[6-(methyl{4-[(3,3,3-trifluoropropyl)sulphonyl]butyl}amino)hexyl]-6,7-dihydro-5H-benzo[7]annulen-3-ol
4-fluoro-8-(4-fluoro-3-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol
4-fluoro-8-(4-fluoro-3-hydroxyphenyl)-9-{6-[(2-hydroxyethyl){3-[(4,4,5,5,5-pentafluoropentyl)sulphonyl]propyl}amino]hexyl}-6,7-dihydro-5H-benzo[7]annulen-3-ol
4-fluoro-8-(4-fluoro-3-hydroxyphenyl)-9-[6-(methyl{3-[(3,3,4,4,4-pentafluorobutyl)sulphonyl]propyl}amino)hexyl]-6,7-dihydro-5H-benzo[7]annulen-3-ol
4-fluoro-8-(4-fluoro-3-hydroxyphenyl)-9-[6-(methyl{4-[(4,4,5,5,5-pentafluoropentyl)sulphonyl]butyl}amino)hexyl]-6,7-dihydro-5H-benzo[7]annulen-3-ol
4-fluoro-8-(4-fluoro-3-hydroxyphenyl)-9-[6-(methyl{4-[(4,4,4-trifluorobutyl)sulphonyl]butyl}amino)hexyl]-6,7-dihydro-5H-benzo[7]annulen-3-ol
4-fluoro-8-(4-fluoro-3-hydroxyphenyl)-9-[6-(methyl{4-[(3,3,3-trifluoropropyl)sulphonyl]butyl}amino)hexyl]-6,7-dihydro-5H-benzo[7]annulen-3-ol
4-fluoro-8-(4-fluoro-3-hydroxyphenyl)-9-[6-(methyl{4-[(RS)-(3,3,3-trifluoropropyl)sulphinyl]butyl}amino)hexyl]-6,7-dihydro-5H-benzo[7]annulen-3-ol
2-fluoro-8-(4-fluoro-3-hydroxyphenyl)-9-[6-(methyl{4-[(4,4,5,5,5-pentafluoropentyl)sulphonyl]butyl}amino)hexyl]-6,7-dihydro-5H-benzo[7]annulen-3-ol
2-fluoro-8-(4-fluoro-3-hydroxyphenyl)-9-[6-(methyl{3-[(RS)-(3,3,4,4,4-pentafluorobutyl)sulphinyl]propyl}amino)hexyl]-6,7-dihydro-5H-benzo[7]annulen-3-ol
2-fluoro-8-(4-fluoro-3-hydroxyphenyl)-9-[6-(methyl{3-[(3,3,4,4,4-pentafluorobutyl)sulphonyl]propyl}amino)hexyl]-6,7-dihydro-5H-benzo[7]annulen-3-ol
2-fluoro-8-(4-fluoro-3-hydroxyphenyl)-9-[6-(methyl{4-[(RS)-(4,4,5,5,5-pentafluoropentyl)sulphinyl]butyl}amino)hexyl]-6,7-dihydro-5H-benzo[7]annulen-3-ol
2-fluoro-8-(4-fluoro-3-hydroxyphenyl)-9-[6-(methyl{3-[(RS)-(3,3,3-trifluoropropyl)sulphinyl]propyl}amino)hexyl]-6,7-dihydro-5H-benzo[7]annulen-3-ol
2-fluoro-8-(4-fluoro-3-hydroxyphenyl)-9-[6-(methyl{3-[(3,3,3-trifluoropropyl)sulphonyl]propyl}amino)hexyl]-6,7-dihydro-5H-benzo[7]annulen-3-ol
2-fluoro-8-(4-fluoro-3-hydroxyphenyl)-9-[6-(methyl{3-[(4,4,4-trifluorobutyl)sulphonyl]propyl}amino)hexyl]-6,7-dihydro-5H-benzo[7]annulen-3-ol
2-fluoro-8-(4-fluoro-3-hydroxyphenyl)-9-[6-(methyl{4-[(4,4,4-trifluorobutyl)sulphonyl]butyl}amino)hexyl]-6,7-dihydro-5H-benzo[7]annulen-3-ol
2-fluoro-8-(4-fluoro-3-hydroxyphenyl)-9-{6-[(2-hydroxy-2-methylpropyl){3-[(RS)-(3,3,3-trifluoropropyl)sulphinyl]propyl}amino]hexyl}-6,7-dihydro-5H-benzo[7]annulen-3-ol
2-fluoro-8-(4-fluoro-3-hydroxyphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol
2-fluoro-8-(4-fluoro-3-hydroxyphenyl)-9-{6-[(2-methoxyethyl){3-[(RS)-(4,4,5,5,5-pentafluoropentyl)sulphinyl]propyl}amino]hexyl}-6,7-dihydro-5H-benzo[7]annulen-3-ol
2-fluoro-8-(4-fluoro-3-hydroxyphenyl)-9-{6-[(3-methoxypropyl){3-[(RS)-(4,4,5,5,5-pentafluoropentyl)sulphinyl]propyl}amino]hexyl}-6,7-dihydro-5H-benzo[7]annulen-3-ol
   The invention furthermore provides compounds of the formula (I) in which
R¹, R², R³ and R⁴ independently of one another represent hydrogen, hydroxy, alkoxy, nitrile, alkylsulphonyl or represent the replacement of a CH group in the aromatic ring by a nitrogen atom or
   independently of one another represent fluorine (but restricted in that at least one or more of the other substituents represents hydroxy, alkoxy, nitrile, alkylsulphonyl).
      The invention furthermore provides compounds of the formula (I) in which
R⁵, R⁶ and R⁷ independently of one another represent hydrogen, fluorine, chlorine, bromine, methyl, ethyl, trifluoromethyl or nitrile.
   The invention furthermore provides compounds of the formula (I) in which
X is selected from the group consisting of hydrogen and alkyl, cycloalkyl, alkoxyalkyl, C₂-C₆-alkenyl, C₂-C₆-alkynyl which may optionally be mono- or polysubstituted by halogen, hydroxy, —CN or deuterium.
   The invention furthermore provides compounds of the formula (I) in which
Y represents perfluorinated or partially fluorinated —C₁-C₄-alkyl or perfluorinated or partially fluorinated C₃-C₈-cycloalkyl.
   The invention furthermore provides compounds of the formula (I) in which
m represents 4, 5, 6 or 7.
   The invention furthermore provides compounds of the formula (I) in which
n represents 2, 3, 4, 5, 6 or 7.
   The invention furthermore provides compounds of the formula (I) in which
p represents 0, 1 or 2.
   The invention furthermore provides compounds of the formula (I) in which
q represents 1, 2, 3 or 4.
   The invention furthermore provides compounds of the formula (I) in which
R¹, R², R³, R⁴ independently of one another represent hydrogen, hydroxy, nitrile, methylsulphonyl or represent the replacement of a CH group in the aromatic ring by a nitrogen atom or independently of one another represent fluorine (but restricted in that at least one of the other substituents represents hydroxy, nitrile, methylsulphonyl).
   The invention furthermore provides compounds of the formula (I) in which
R⁵ and R⁶ independently of one another represent hydrogen, chlorine or fluorine.
   The invention furthermore provides compounds of the formula (I) in which
R⁷ represents hydrogen.
   The invention furthermore provides compounds of the formula (I) in which
X represents hydrogen or —C₁-C₄-alkyl, optionally substituted by hydroxy or methoxy.
   The invention furthermore provides compounds of the formula (I) in which
Y represents —CF₃, —C₂F₅, —CF₂CF₂CF₃ or —CF(CF₃)₂.
   The invention furthermore provides compounds of the formula (I) in which
m represents 5 or 6.
   The invention furthermore provides compounds of the formula (I) in which
n represents 3, 4, 5 or 6.
   The invention furthermore provides compounds of the formula (I) in which
q represents 2, 3 or 4.
   The invention furthermore provides compounds of the formula (I) in which
R¹, R², R³ represent hydrogen and
R⁴ represents hydrogen, hydroxy, nitrile, methylsulphonyl or represents the replacement of a CH group in the aromatic ring by a nitrogen atom
   or in which
R¹, R² represent hydrogen
R³ represents fluorine and
R⁴ represents hydroxy, nitrile or methylsulphonyl.
   The invention furthermore provides compounds of the formula (I) in which
R⁵ and R⁶ independently of one another represent hydrogen, chlorine or fluorine, restricted in that R⁵ and R⁶ do not both represent fluorine, do not both represent chlorine and do not both represent chlorine and fluorine.
   The invention furthermore provides compounds of the formula (I) in which
X represents optionally hydroxy- or methoxy-substituted C₁-C₄-alkyl.
   The invention furthermore provides compounds of the formula (I) in which
Y represents —CF₃, —C₂F₅.
   The invention furthermore provides compounds of the formula (I) in which
q represents 2 or 3.
   The invention furthermore provides compounds of the formula (II) in which
R¹² represents phenyl, 3-hydroxyphenyl, 4-hydroxyphenyl, 3-fluoro-4-hydroxphenyl, 4-fluoro-3-hydroxyphenyl, 2-fluoro-5-hydroxyphenyl, 4-methylsulphonylphenyl, 3-methylsulphonylphenyl, 4-cyanophenyl or 3-pyridyl.
   The invention furthermore provides compounds of the formula (II) in which
R⁵ and R⁶ independently of one another represent hydrogen or fluorine, but R⁵ and R⁶ do not both represent fluorine, or
   R⁶ represents chlorine and R⁵ represents hydrogen.
      The invention furthermore provides compounds of the formula (II) in which
X represents methyl, ethyl, methoxyethyl, methoxypropyl, hydroxyethyl, 3-hydroxypropyl or 2-hydroxy-2-methylpropyl.

The radicals given in the respective combinations or preferred combinations together form the radical definition such that, independently of the respective given combinations of radicals, any other combinations of radicals are also included.

Combinations of two or more of the preferred ranges mentioned above are very particularly preferred.

The general or preferred radical definitions given above apply both to the end product of the formula (I) and correspondingly to the starting materials or intermediates required in each case for the preparation.

The invention furthermore provides a process for preparing the compounds according to the invention. The preparation of the compounds (I) according to the invention or the compounds (II) as a subset of the formula (I) can be illustrated by the synthesis schemes below.

The synthesis of intermediates 5, prepared analogously to the patent WO 03/033461 A1, is shown in the general formula scheme below (Synthesis Scheme 1), where R¹, R², R³, R⁴, R⁵, R⁶ and R⁷ have the meaning given in formula (I).

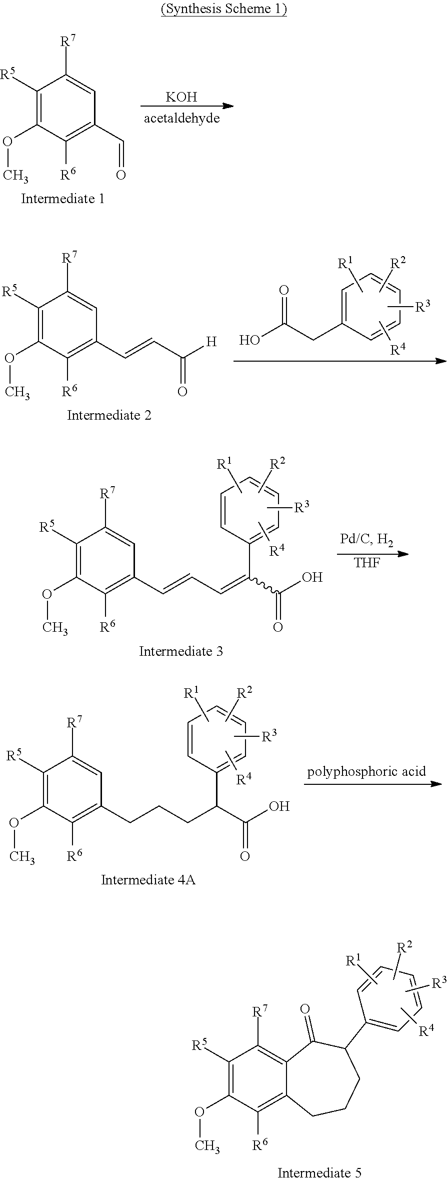

The Intermediates 2 are synthesized by condensation reactions known to the person skilled in the art of acetaldehyde with one of the Intermediates 1 (commercially available, for example, from Aldrich, ABCR) with base catalysis in water with or without addition of an organic solvent which is stable under these conditions (Organic Reactions 1968, 16, 1; Justus Liebigs Ann. Chem. 1917, 412, 322; J. Org. Chem. 1951, 16, 1519; Helv. Chim. Acta 1993, 76, 1901). Particular preference is given here to the reaction with potassium hydroxide with addition of dichloromethane at 1-30° C. The Intermediates 2 are then reacted under Knoevenagel conditions known to the person skilled in the art with an arylacetic acid (commercially available, for example, from Aldrich, ABCR) (Organic Reactions 1967, 15, 204; Tetrahedron Lett. 1998, 39, 8013). Particular preference is given to the reaction with acetic anhydride and triethylamine at reflux temperature. The Intermediates 4 are synthesized by catalytic hydrogenations known to the person skilled in the art (Houben Weyl, “Methoden der organischen Chemie” [Methods of Organic Chemistry], Vol. 4/1c part 1, p. 14 ff. (1980), Georg Thieme Verlag Stuttgart, N.Y.). The Intermediates 5 are prepared by ring closure reactions known to the person skilled in the art according to Friedel-Crafts (Chem. Rev. 1970, 70, 553; J. Org. Chem. 1958, 23, 789, J. Org. Chem. 1981, 46, 2974; J. Med. Chem. 1986, 29, 1615). The use of phosphorus pentoxide in methanesulphonic acid or trifluoromethanesulphonic acid in the temperature range of 0-30° C. may be mentioned as being particularly preferred.

Alternatively, Intermediates 5 can be prepared according to Synthesis Scheme 2 where R¹, R², R³, R⁴, R⁵, R⁶ and R⁷ have the meaning given in formula (I), but do not represent bromine.

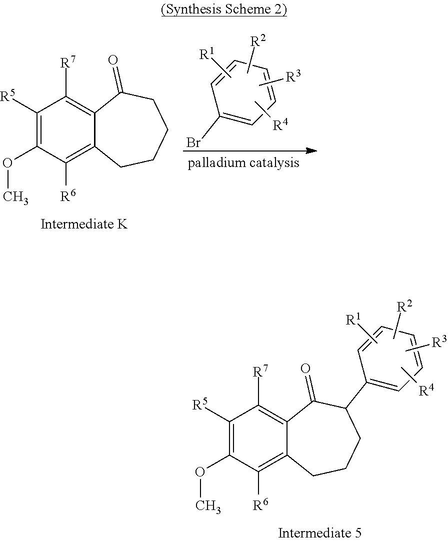

Preparation of Intermediates 5 can also be by arylation of Intermediates K, as known to the person skilled in the art (J. Am. Chem. Soc. 1997, 119, 11108; J. Am. Chem. Soc. 2002, 124, 15168; J. Am. Chem. Soc. 1997, 119, 12382; J. Am. Chem. Soc. 1999, 121, 1473; J. Am. Chem. Soc. 2000, 122, 1360; Tetrahedron 2001, 57, 5967; J. Org. Chem. 2001, 66, 3284; J. Org. Chem. 2006, 71, 3816; Org. Lett. 2002, 4, 4053; J. Organomet. Chem. 2005, 690, 5832; Org. Lett. 2003, 5, 1479; J. Org. Chem. 2006, 71, 685; Tetrahedron 2005, 61, 9716; Angew. Chem. 2005, 117, 2497; Angew. Chem. 2005, 117, 407; Angew. Chem. 2006, 118, 7789). To this end, a palladium compound (for example Pd(OAc)₂, Pd₂(dba)₃) is reacted with a ligand (for example BINAP, 2,2′-bis(diphenylphosphino)-1,1′-binaphthyl, xantphos, triphenylphosphine, DTPF, 1,1′-bis(di-o-tolylphosphino)ferrocene, 1,3-di-tert-butyl-2-chloro-1,3,2-diazaphospholidine, 2′-(dicyclohexylphosphino)-N,N-dimethylbiphenyl-2-amine) in a solvent (for example toluene, xylene, tetrahydrofuran, dioxane, dimethoxyethane, tert-butyl methyl ether) with a base (for example sodium tert-butoxide, potassium tert-butoxide, sodium hydride, potassium hydride, potassium hexamethyldisilazide, tripotassium phosphate, caesium carbonate) and an aromatic halide or triflate at a temperature of 40-160° C. The set temperature also depends on the solvent. The palladium compound used can also be connected to corresponding ligands beforehand such as, for example, allyl[1,3-bis(2,6-diisopropylphenyl)imidazol-2-ylidene]chloropalladium(II), allyl[1,3-bis(2,6-diisopropylphenyl)-2-imidazolidinylidene]chloropalladium(II), Pd(dppf)Cl, [PdBrPtBu]₂. Particularly preferred for the reactions is the use of palladium(II) acetate with BINAP or xantphos or allylchloro(1,3-bis(2,6-di-isopropylphenyl)imidazol-2-ylidene)palladium. Particular preference is given here to an alkali metal salt of an alcohol as base in THF at 60-80° C. Very particular preference is given to the reaction with palladium(II) acetate, xantphos, sodium tert-butoxide in THF under reflux. Here, the excess of aryl halide is to be kept as low as possible; preferably, only one equivalent of aryl halide and one equivalent of ketone is used.

The Intermediates 10 can be synthesized according to Synthesis Scheme 3, where R¹, R², R³, R⁴, R⁵, R⁶ and R⁷ and also m have the meaning given in formula (I).

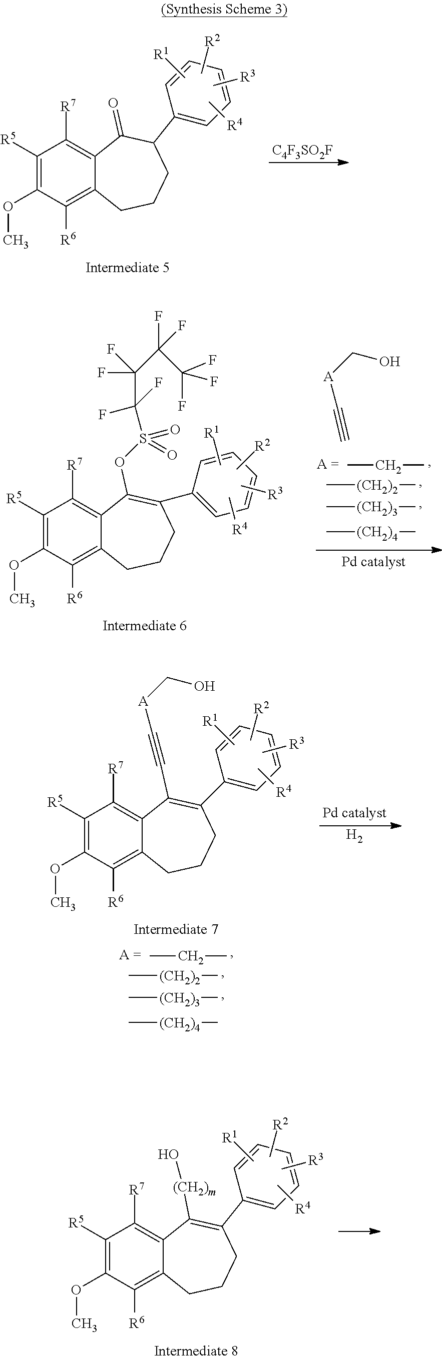

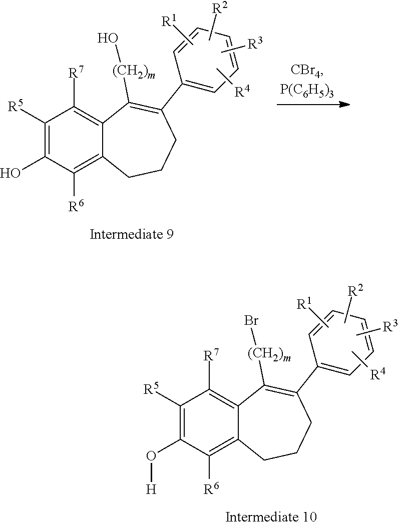

The Intermediate 6 can be prepared under conditions known to the person skilled in the art (Tetrahedron: Asymmetry 1990, 1, 97; J. Org. Chem. 1996, 61, 8536; Synthesis 2002, 2064). It is also possible to prepare analogous perfluorinated sulphonyl enol ethers where the nonafluorobutyl radical is replaced, for example, by trifluoromethyl. Particularly preferred for the preparation of Intermediate 6 is the reaction in the presence of organic amines in ethers or halogenated solvents. Very particular preference is given to the reaction with nonafluorobutylsulphonyl fluoride in tetrahydrofuran/tert-butyl methyl ether with 2,3,4,5,7,8,9,10-octahydropyrido[1,2-4][1,3]diazepine as base with cooling at 0-15° C. Intermediates 7 can be prepared according to Sonogashira using a palladium catalyst (for example tetrakis(triphenylphosphine)palladium(0), Pd(Cl)₂(PPh₃)₂ and comparable catalysts) and an amino base in an aprotic solvent (Chem. Rev. 2007, 107, 874; Synthesis 1986, 320; Angew. Chem. 1994, 106, 1568), as known to the person skilled in the art. Particular preference is given to the reaction with tetrakis(triphenylphosphine)palladium(0) and triethylamine in DMF at 60-100° C. The Intermediates 8 can be synthesized by methods known to the person skilled in the art (J. Org. Chem. 1990, 55, 3484; J. Org. Chem. 1964, 29, 3660; Chem. Ber. 1959, 92, 541) using a transition metal catalyst and hydrogen. Particular preference is given to the hydrogenation with palladium. Very particular preference is given to hydrogenation in methanol with addition of a base such as, for example, potassium hydroxide. To obtain the Intermediate 9, the methyl ether has to be cleaved by methods known to the person skilled in the art (“Protective Groups in Organic Synthesis” 3rd edition, p. 250 ff. (1999), John Wiley & Sons New York). Particular preference is given to cleavage with boron tribromide, and very particular preference is given to methyl ether cleavage with boron tribromide with addition of a pyridine derivative (for example lutidine) with cooling in an inert solvent (for example dichloromethane) at 0-10° C. Intermediate 9 is converted into Intermediate 10 by bromination of the hydroxyl group, as known to the person skilled in the art (J. Am. Chem. Soc. 1964, 86, 964; Tetrahedron Lett. 1973, 3937; Angew. Chem. Int. Ed. 1975, 14, 801; J. Org. Chem. 1969, 34, 212; J. Am. Chem. Soc. 1970, 92, 2139; J. Chem. Soc., Perkin Trans. 1, 1980, 2866; J. Org. Chem. 1986, 51, 5291; J. Org. Chem. 1962, 27, 349). Particular preference is given to using triphenylphosphine and carbon tetrabromide in an inert solvent (for example tetrahydrofuran) at 0-10° C.

Intermediates 15 can be prepared according to Synthesis Scheme 4, where Y, q, n, X have the meaning given in formula (I).

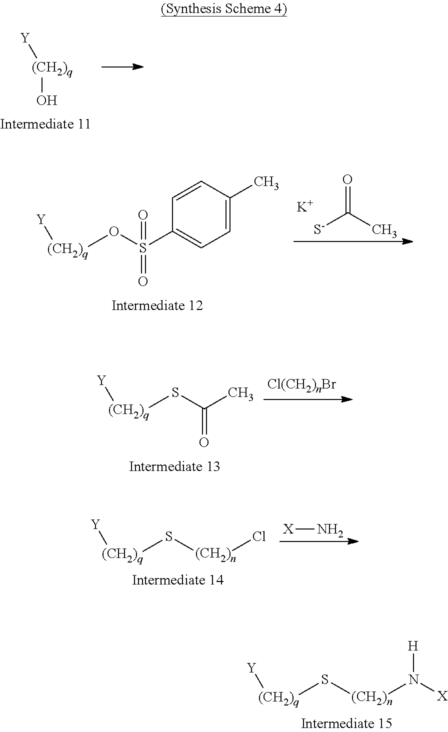

The commercially available Intermediates 11 (for example Aldrich) are converted by methods known to the person skilled in the art into Intermediates 12 (J. Chem. Soc. 1939, 1248; Synthesis 1996, 594; Helv. Chim. Acta 1946, 29, 671). Intermediates 13 can be synthesized by methods known to the person skilled in the art (J. Chem. Soc. 1950, 579; J. Am. Chem. Soc. 1953, 75, 3700). Intermediates 14 are prepared by synthesis methods known to the person skilled in the art (Pharm. Chem. J. 1989, 23, 998). Intermediates 15 are synthesized by methods known to the person skilled in the art (Org. Synth. Coll. Vol. 1, 102, 1941; Org. Synth. Coll. Vol. 2, 290, 1943; Org. Synth. Coll. Vol. 3, 256, 1953; J. Am. Chem. Soc. 1952, 74, 5105; J. Am. Chem. Soc. 1954, 76, 658).

Intermediates 17 can be prepared according to Synthesis Scheme 5, where Y, q, n and X have the meaning given in formula (I).

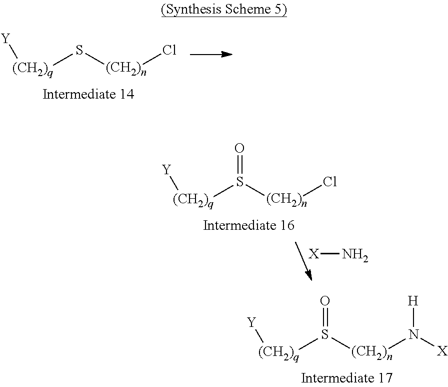

Intermediates 16 can be prepared by methods known to the person skilled in the art (Org. Prep. Proced. Int. 1982, 14, 45; J. Org. Chem. 1962, 27, 282). Particular preference is given here to oxidation with metaperiodate. Very particular preference is given to oxidation with sodium metaperiodate. The Intermediates 17 can be prepared as described for the Intermediates 15.

The Intermediates 19 can be prepared according to Synthesis Scheme 6, where Y, q, n and X have the meaning given in formula (I).

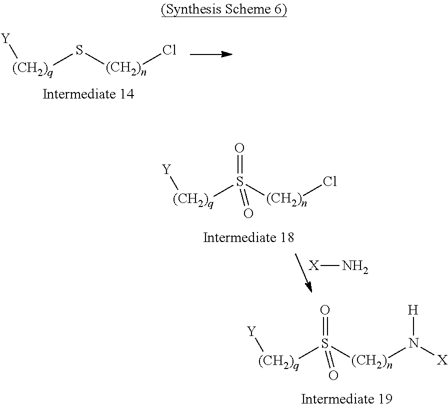

The Intermediates 18 can be prepared by methods known to the person skilled in the art (J. Org. Chem. 1957, 22, 241; J. Org. Chem. 2004, 69, 3824; J. Am. Chem. Soc. 1941, 63, 2939; Org. Lett. 1999, 1, 189). Particular preference is given here to oxidation with peracids. The Intermediates 19 can be prepared as described for the Intermediates 15.

Intermediates 13 can also be prepared according to Synthesis Scheme 7, where Y and q have the meaning given in formula (I).

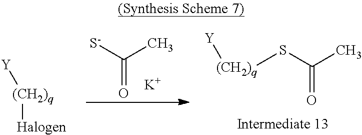

The Intermediates 13 can also be prepared from the corresponding halogen compounds by methods known to the person skilled in the art (J. Am. Chem. Soc. 1953, 75, 3700; J. Org. Chem. 1984, 49, 3231).

Intermediate 23 can be prepared according to Synthesis Scheme 8.

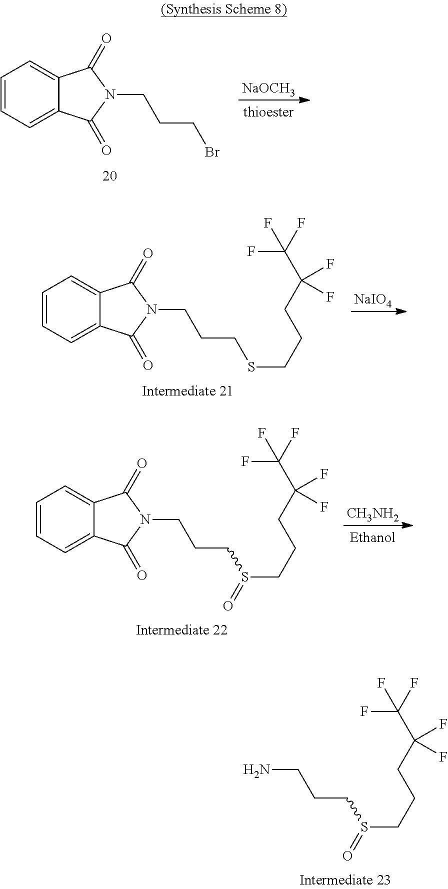

The Intermediate 21 can be synthesized as described for the Intermediates 14. The Intermediate 22 is prepared analogously to Intermediates 16. Deprotection of the amino function in the Intermediate 23 can be carried out by methods known to the person skilled in the art (for example “Protective Groups in Organic Synthesis” 3rd. edition, p. 565 f. (1999), John Wiley & Sons New York).

The Example compounds can be synthesized according to Synthesis Scheme 9 by reacting the Intermediates 15, 17, 19 or 23 with the Intermediate 10, where R¹, R², R³, R⁴, R⁵, R⁶, R⁷, m, n, p, q, X, Y have the meaning given in formula (I).

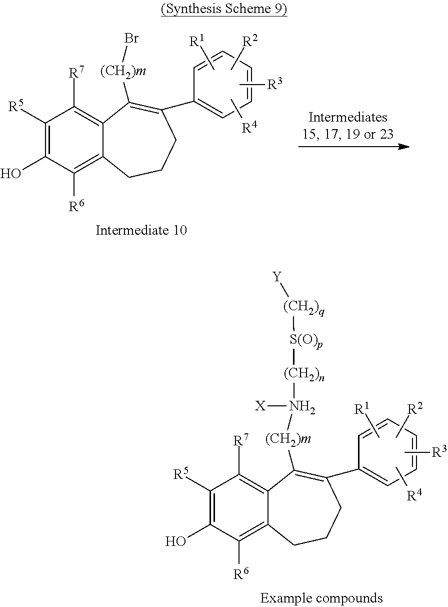

The reactions can be carried out according to methods known to the person skilled in the art as described for the conversion of Intermediate 14 into Intermediate 15. Particular preference is given to the reaction in the presence of an alkali metal iodide and a carbonate of the alkali metals in an aprotic solvent such as, for example, DMF or NMP.

The compounds according to the invention have an unforeseeable, useful pharmacological and pharmacokinetic spectrum of action. They are therefore suitable for use as medicaments for the treatment and/or prophylaxis of diseases in humans and animals. In the context of the present invention, the term “treatment” includes prophylaxis. The pharmaceutical efficacy of the compounds according to the invention can be explained by their action as SERMs.

The present invention further relates to the use of the compounds according to the invention for the treatment and/or prophylaxis of diseases, preferably of gynaecological diseases, in particular for alleviating the symptoms of the andropause and menopause, i.e. for male and female hormone replacement therapy (HRT), namely both for prevention and for treatment; for the treatment of problems accompanying dysmenorrhoea; treatment of dysfunctional uterine bleeding; treatment of acne; prevention and treatment of cardiovascular diseases; treatment of hypercholesterolaemia and hyperlipidaemia; prevention and treatment of atherosclerosis; for inhibiting proliferation of arterial smooth muscle cells; for the treatment of respiratory distress syndrome of the newborn; treatment of primary pulmonary hypertension; for prevention and treatment of osteoporosis (Black, L. J., Sato, M., Rowley, E. R., Magee, D. E., Bekele, A., Williams, D. C., Cullinan, G. J., Bendele, R., Kauffman, R. F., Bensch, W. R., Frolik, C. A., Termine, J. D. and Bryant, H. U.: Raloxifene [LY 139481 HCl] prevents bone loss and reduces serum cholesterol without causing uterine hypertrophy in ovariectomized rats; J. Clin. Invest. 93: 63-69, 1994); for preventing bone loss in postmenopausal women, in hysterectomized women or in women who have been treated with LHRH agonists or antagonists; inhibition of sperm maturation; treatment of rheumatoid arthritis; for the prevention of Alzheimer's disease; treatment of endometriosis; treatment of myomata; treatment of myomata and endometriosis in combination with LHRH analogues; treatment of hormone-dependent tumours, e.g. of breast cancer or e.g. of endometrial carcinoma, treatment of prostatic diseases, treatment of benign diseases of the breast e.g. mastopathy. Moreover, based on their pharmacological profile, the compounds according to the invention are suitable both for male and for female contraception.

The present invention furthermore provides the use of the compounds according to the invention for the treatment of infertility and for induction of ovulation.

The present invention furthermore provides the use of the compounds according to the invention for the treatment and prophylaxis of stroke and Alzheimer's and other diseases of the central nervous system, which is accompanied by cellular death of neurons.

The present invention furthermore provides the use of the compounds according to the invention for the production of a medicinal product for the treatment and/or prophylaxis of diseases, in particular the diseases mentioned above.

The present invention furthermore provides a method of treatment and/or prophylaxis of diseases, in particular the aforementioned diseases, using an effective amount of the compounds according to the invention.

The present invention furthermore provides the use of the compounds according to the invention for the treatment and/or prophylaxis of diseases, in particular the diseases mentioned above.

The present invention furthermore provides the compounds according to the invention for use in a method of treatment and/or prophylaxis of the diseases mentioned above.

The present invention furthermore provides medicaments comprising at least one compound according to the invention and at least one or more further active compounds, in particular for the treatment and/or prophylaxis of the diseases mentioned above. By way of example, the following may be mentioned as suitable active compounds for combinations: gestagens, oestrogens (for example in the context of an add-back therapy) and progesterone receptor antagonists.

Oestrogens are compounds (naturally occurring or synthetic, steroidal and non-steroidal compounds) that display oestrogenic efficacy. Such compounds are, for example: ethynyl estradiol, estradiol, estradiol sulphamate, estradiol valerate, estradiol benzoate, estrone, mestranol, estriol, estriol succinate and conjugated oestrogens, including conjugated oestrogens such as estrone sulphate, 17β-estradiol sulphate, 17α-estradiol sulphate, equilin sulphate, 17β-dihydroequilin sulphate, 17α-dihydroequilin sulphate, equilenin sulphate, 17β-dihydroequilenin sulphate and 17α-dihydroequilenin sulphate. Especially interesting oestrogens are ethynyl estradiol, estradiol, estradiol sulphamate, estradiol valerate, estradiol 15-benzoate, estrone, mestranol and estrone sulphate. Ethynyl estradiol, estradiol and mestranol are preferred as oestrogens, and ethynyl estradiol is especially preferred.

Gestagens are understood in the sense of the present invention either as natural progesterone itself or synthetic (steroidal and non-steroidal) derivatives, which like progesterone itself bind to the progesterone receptor and, in dosages that are above the ovulation inhibiting dose, inhibit ovulation. The following may be mentioned as examples of gestagens: levonorgestrel, norgestimate, norethisterone, dydrogesterone, drospirenone, 3β-hydroxydesogestrel, 3-ketodesogestrel (=etonogestrel), 17-deacetylnorgestimate, 19-norprogesterone, acetoxypregnenolone, allylestrenol, amgestone, chloromadinone, cyproterone, demegestone, desogestrel, dienogest, dihydrogesterone, dimethisterone, ethisterone, ethynodiol diacetate, flurogestone acetate, gestrinone, gestodene, gestrinone, hydroxymethylprogesterone, hydroxyprogesterone, lynestrenol (=lynoestrenol), mecirogestone, medroxyprogesterone, megestrol, melengestrol, nomegestrol, norethindrone (=norethisterone), norethynodrel, norgestrel (including d-norgestrel and dl-norgestrel), norgestrienone, normethisterone, progesterone, quingestanol, (17α)-17-hydroxy-11-methylene-19-norpregna-4,15-dien-20-yn-3-one, tibolone, trimegestone, algestone acetophenide, nestorone, promegestone, 17-hydroxyprogesterone ester, 19-nor-17-hydroxyprogesterone, 17α-ethynyl-testosterone, 17α-ethynyl-19-nortestosterone, d-17β-acetoxy-13β-ethyl-17α-ethynyl-gon-4-en-3-one oxime or the compounds disclosed in WO 00/66570, in particular tanaproget. Levonorgestrel, norgestimate, norethisterone, drospirenone, dydrogesterone and dienogest are preferred. Drospirenone and dienogest are especially preferred.

Progesterone receptor antagonists are compounds which inhibit the action of progesterone on its receptor. As examples we may mention RU 486, onapristone, lonaprisan (11β-(4-acetylphenyl)-17β-hydroxy-17α-(1,1,2,2,2-pentafluoroethyl)estra-4,9-dien-3-one cf. WO 98/34947) and the compounds claimed in WO 08/58767.

However, combinations with one or more further active compounds are also feasible, in particular combinations with aromatase inhibitors, 17beta HSD1 inhibitors, steroid sulphatase (STS) inhibitors, LHRH analogues, LHRH antagonists, GnRH agonists and antagonists, kisspeptin receptor (KISSR) antagonists, selective androgen receptor modulators (SARMs), androgens, selective progesterone receptor modulators (SPRMs), gestagens, antigestagens (progesterone receptor antagonists), oral contraceptives, oestrogens, inhibitors of mitogen activated protein (MAP) kinases and inhibitors of MAP kinases kinases (Mkk3/6, Mek1/2, Erk1/2) inhibitors of protein kinases B (PKBα/β/γ; Akt1/2/3), inhibitors of phosphoinositide 3-kinases (PI3K), inhibitors of cyclin-dependent kinase (CDK1/2), inhibitors of the hypoxia-induced signal pathway (HIF1alpha inhibitors, activators of prolyl hydroxylases), histone deacetylase (HDAC) inhibitors, prostaglandin F receptor (FP) (PTGFR) antagonists and non-steroidal antiinflammatory drugs (NSAIDs) for the treatment of endometriosis.

The invention also relates to pharmaceutical preparations comprising at least one compound of general formula I (or physiologically acceptable addition salt thereof with organic and inorganic acids) and to the use of these compounds for the preparation of medicaments, in particular for the indications mentioned above.

The compounds can be used for the indications mentioned above, both by oral and parenteral administration.

The compounds can also be used in combination with the natural vitamin D3 or with calcitriol analogues for osteogenesis or as supporting therapy for therapies that cause loss of bone mass (for example therapy with glucocorticoids, chemotherapy).

The compounds of general formula I can also be used in combination with progesterone receptor antagonists or in combination with pure oestrogens, and in particular for use in hormone replacement therapy and for the treatment of gynaecological disorders and for controlling female fertility. A therapeutic product, comprising an oestrogen and a pure anti-oestrogen for simultaneous, sequential or separate use for selective oestrogen therapy of perimenopausal or postmenopausal states has already been described in EP-A 0 346 014.

The compounds of general formula I can also be given in combination with gestagens and substances with gestagenic action, in particular for use in premenopausal women for the treatment of gynaecological diseases such as endometriosis, myomata or disturbances of menstruation e.g. dysmenorrhoea or hypermenorrhoea, or for the treatment of hormone-dependent tumours, e.g. breast cancer.

The compounds of general formula I can be administered either continuously (for example once daily) or in intermittent regimens. Treatment regimes such as once weekly, once monthly, daily for a period of several days, on particular days of the female menstrual cycle (e.g. on 14 consecutive days of the secretory phase or several days in the middle of the menstrual cycle) may be mentioned by way of example (but not exclusively). The compounds of general formula I can also be administered continuously over a longer treatment period (e.g. 14-168 successive days) followed by a treatment pause, which is either fixed (e.g. 14-84 days) or is flexible and lasts until the next menstruation. In these intermittent treatment regimens the compounds of general formula I can be administered alone or in combination with the combination therapies mentioned above, and these for their part can be administered continuously or also intermittently.

The compounds according to the invention can have systemic and/or local action. For this purpose, they can be administered in a suitable way, for example orally, parenterally, pulmonarily, nasally, sublingually, lingually, buccally, rectally, dermally, transdermally, conjunctivally, otically or as an implant or stent.

For these routes of application, the compounds according to the invention can be administered in suitable dosage forms.

Dosage forms that function according to the prior art, with rapid and/or modified release of the compounds according to the invention, containing the compounds according to the invention in crystalline and/or amorphisized and/or dissolved form, are suitable for oral administration, for example tablets (uncoated or coated tablets, for example with enteric coatings or coatings with delayed dissolution or insoluble coatings, which control the release of the compound according to the invention), tablets that disintegrate rapidly in the oral cavity or films/wafers, films/lyophilisates, capsules (for example hard-gelatin or soft-gelatin capsules), sugar-coated tablets, granules, pellets, powders, emulsions, suspensions, aerosols or solutions.

Parenteral administration can take place with avoidance of an absorption step (for example intravenously, intra-arterially, intracardially, intraspinally or intralumbally) or with inclusion of absorption (for example intramuscularly, subcutaneously, intracutaneously, percutaneously or intraperitoneally). Suitable dosage forms for parenteral administration are inter alia injection and infusion preparations in the form of solutions, suspensions, emulsions, lyophilisates or sterile powders.

Suitable dosage forms for the other routes of administration are, for example, pharmaceutical forms for inhalation (inter alia powder inhalers, nebulizers), nasal drops, solutions, and sprays; tablets for lingual, sublingual or buccal administration, films/wafers or capsules, suppositories, ear or eye preparations, vaginal capsules, aqueous suspensions (lotions, shaking mixtures), lipophilic suspensions, ointments, creams, transdermal therapeutic systems (such as, for example, patches), milk, pastes, foams, dusting powders, implants, intrauterine systems, vaginal rings or stents.

The compounds according to the invention can be converted into the stated dosage forms. This can take place in a manner that is known per se, by mixing with inert, non-toxic, pharmaceutically suitable auxiliaries. These auxiliaries include inter alia vehicles (for example microcrystalline cellulose, lactose, mannitol), solvents (for example liquid polyethylene glycols), emulsifiers and dispersants or wetting agents (for example sodium dodecylsulphate, polyoxysorbitan oleate), binders (for example polyvinylpyrrolidone), synthetic and natural polymers (for example albumin), stabilizers (for example antioxidants such as, for example, ascorbic acid), colorants (for example inorganic pigments such as, for example, iron oxides) and taste and/or odour correctants.

The present invention further relates to medicinal products comprising at least one compound according to the invention, usually together with one or more inert, non-toxic, pharmaceutically suitable auxiliaries, and their use for the purposes stated above.

In the case of oral administration, the amount per day is from about 0.01 to 100 mg/kg of body weight. The amount of a compound of the general formula I to be administered varies over a wide range and can cover every effective amount. Depending on the condition to be treated and the method of administration, the amount of the compound administered can be 0.01-100 mg/kg of body weight per day.

Nevertheless, it may optionally be necessary to deviate from the stated amounts, mainly depending on body weight, route of administration, individual response to the active substance, type of preparation and time point or interval in which administration takes place. Thus, in some cases it may be sufficient to use less than the aforementioned minimum amount, whereas in other cases the stated upper limit must be exceeded. In the case of administration of relatively large amounts, it may be advisable to divide these into several individual doses throughout the day.

The percentages in the following tests and examples are, unless stated otherwise, percentages by weight; parts are parts by weight. Proportions of solvents, dilution ratios and information about concentration for liquid/liquid solutions relate in each case to volume.

### List of Abbreviations, Chemistry

### Abbreviations and Acronyms:

CI chemical ionization (in MS)
TLC thin layer chromatography
DMF dimethylformamide
DMSO dimethyl sulphoxide
ESI electrospray ionization (in MS)
GC-MS gas chromatography-coupled mass spectrometry
h hour(s)
HPLC high pressure, high performance liquid chromatography
LC-MS liquid chromatography-coupled mass spectrometry
Mass found mass found in the mass spectrum
min minute(s)
MS mass spectrometry
NMR nuclear magnetic resonance spectroscopy
R_{f} retention index (in TLC)
Rₜ retention time (in HPLC)
RT room temperature
TFA trifluoroacetic acid
THF tetrahydrofuran

### Purification of the Compounds According to the Invention

In some cases the compounds according to the invention could be purified by preparative HPLC for example using an autopurifier apparatus from the company Waters (detection of the compounds by UV-detection and electrospray ionization) in combination with commercially available, prepacked HPLC columns (for example XBridge column (from Waters), C18, 5 μm, 30×100 mm). Acetonitrile/water with additions of ammonia, ammonium acetate, trifluoroacetic acid or formic acid was used as the solvent system. Instead of acetonitrile, methanol for example could also be used.

The flow during purification was 50 ml/min.

In some cases the compounds according to the invention were purified by the following methods:

Waters HPLC autopurification system pump 2525, sample manager 2767, CFO, DAD 2996, ELSD 2424, ZQ 4000, column: XBridge C18, 5 μm, 100×30 mm, 50 ml/min, mobile phase:

Method X (see enumeration below), detection by DAD scan range 210-400 nm, ELSD, MS ESI (+), ESI (−), scan range 160-1000 m/z.

Method 1: mobile phase: water with 0.2% ammonia-acetonitrile 50:50, 0-1 minute; 50:50->20:80, 1-7.5 minutes; 20:80->1:99, 7.5-7.52 minutes; 1:99, 7.52-10 minutes

Method 2: mobile phase: water with 0.2% ammonia-acetonitrile 99:1, 0-1 minute; 99:1->1:99, 1-7.5 minutes; 1:99, 7.5-10 minutes

Method 3: mobile phase: water with 0.2% ammonia-acetonitrile 99:1, 0-1 minute; 99:1->1:99, 1-7 minutes; 1:99, 7-10 minutes

Method 4: mobile phase: water with 0.2% ammonia-acetonitrile 30:70, 0-1 minute; 30:70->1:99, 1-7.5 minutes; 1:99, 7.5-10 minutes

Method 5: mobile phase: water with 0.1% ammonia-acetonitrile 30:70, 0-1 minute; 30:70->1:99, 1-7.5 minutes; 1:99, 7.5-10 minutes

Method 6: mobile phase: water with 0.1% ammonium acetate-acetonitrile 30:70, 0-1 minute; 30:70->1:99, 1-7.5 minutes; 1:99, 7.5-10 minutes

Method 7: mobile phase: water with 0.1% ammonium acetate-acetonitrile 99:1, 0-1 minute; 99:1->1:99, 1-7.5 minutes; 1:99, 7.5-10 minutes

Method 8: mobile phase: water with 0.1% ammonium acetate-acetonitrile 70:30, 0-1 minute; 70:30->40:60, 1-7.5 minutes; 40:60, 7.5-10 minutes

Method 9: mobile phase: water with 0.1% ammonium acetate-acetonitrile 50:50, 0-1 minute; 50:50->1:99, 1-7.5 minutes; 1:99, 7.5-10 minutes

Method 10: mobile phase: water with 0.1% ammonium acetate-acetonitrile 50:50, 0-1 minute; 50:50->20:80, 1-7.5 minutes; 20:80->1:99, 7.5-7.52 minutes; 1:99, 7.52-10 minutes

Method 11: mobile phase: water with 0.1% formic acid-methanol 70:30, 0-1 minute; 70:30->1:99, 1-7.5 minutes; 1:99, 7.5-10 minutes

Method 12: mobile phase: water with 0.1% formic acid-acetonitrile 99:1, 0-1 minute; 99:1->1:99, 1-7.5 minutes; 1:99, 7.5-10 minutes

Freeze-drying or vacuum centrifugation was used for removing the HPLC solvent mixture. The compounds thus obtained could be in the form of TFA salts or formate salts and could be transformed into the respective free bases by standard laboratory procedures known to the person skilled in the art.

In some cases the compounds according to the invention could be purified by silica gel chromatography. Suitable for this purpose are, for example, prepacked silica gel cartridges (for example from Separtis, Isolute® Flash silica gel) in combination with the Flashmaster II chromatograph (Argonaut/Biotage) and chromatography solvents or solvent mixtures such as, for example, hexane, ethyl acetate and also dichloromethane and methanol, and additions of aqueous ammonia solution could also be used.

### Structural Analysis of the Compounds According to the Invention:

In some cases the compounds according to the invention were analysed by LC-MS:

One method of analysis used was based on the following parameters:

System Waters Acquity UPLC-MS: Binary Solvent Manager, Sample Manager/Organizer, Column Manager, PDA, ELSD, SQD 3001, column: Acquity BEH C18, 1.7 μm, 50×2.1 mm. Water with 0.1% TFA or with 0.1% formic acid was used as solvent A. Solvent B consisted of acetonitrile. Gradient 0-1.6 min 1-99% B, 1.6-2.0 min 99% B, flow 0.8 mL/min, temperature 60° C., sample solution 1.0 mg/mL in acetonitrile/water 7:3, injection 2.0 μl, detection per DAD scan range 210-400 nm, ELSD, MS ESI (+), ESI (−), scan range 160-1000 m/z.

In some cases the compounds according to the invention were analysed by LC-MS: retention times Rₜ from LC-MS analysis: detection: UV=200-400 nm (Acquity HPLC system from the company Waters)/MS 100-800 dalton; 20 V (Micromass/Waters ZQ 4000) in ESIpos mode (for production of positively charged molecular ions); HPLC column: X Bridge (Waters), 2.1×50 mm, BEH 1.7 μm; solvent: A: water/0.05% formic acid, B: acetonitrile. Gradient: 10-90% B in 1.7 min, 90% B for 0.2 min, 98-2% B in 0.6 min; flow rate: 1.3 mL/min.

In some cases a Waters ZQ4000 instrument or a Single Quadrupol API (atomic pressure ionization) mass detector (Waters) was used for recording a mass spectrum.

The following symbols are used in the NMR data of the compounds according to the invention:

| | |
|---|---|
| s | singlet |
| d | doublet |
| t | triplet |
| q | quartet |
| quin | quintet |
| m | multiplet |
| br | broad |
| mc | centred multiplet |

Substances which have not been described in the patent Ser. No. 03/033,461 A1 but which can be synthesized in an analogous manner are described below.

### Intermediate 1-2

### (E)-3-(2-Chloro-3-methoxyphenyl)acrylaldehyde

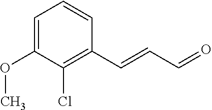

52 ml of 20% strength potassium hydroxide solution were added to 20 g (0.117 mol) of 2-chloro-3-methoxybenzaldehyde in 130 ml of dichloromethane and 80 ml of water. 26 ml of acetaldehyde in 77 ml of water were then added dropwise over 3 hours, the temperature being kept below 30° C. The mixture was stirred at room temperature overnight. The reaction mixture was extracted three times with dichloromethane. The combined organic phases were adjusted to pH 5-6 with acetic acid-water 1:3, washed with water, dried over magnesium sulphate and concentrated. The product was chromatographed on silica gel 60 (mobile phase: hexane, hexane-ethyl acetate 9:1 and 4:1). 9.2 g (40% of theory) of product were obtained.

¹H NMR (400 MHz, chloroform-d₁): δ=3.94 (s, 3H), 6.70 (dd, 1H), 6.98-7.04 (m, 1H), 7.25-7.32 (m, 2H), 8.00 (d, 1H), 9.77 (d, 1H).

### Intermediate 2-2

### (E)-3-(2-Fluoro-3-methoxyphenyl)acrylaldehyde

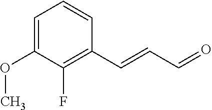

50 g of potassium hydroxide were dissolved in 250 ml of water, and 50 g (0.324 mol) of 2-fluoro-3-methoxybenzaldehyde in 200 ml of dichloromethane were added. 57.16 g of acetaldehyde in 250 ml of water were added dropwise over 3 hours. Stirring was then continued overnight and for another day at room temperature. 15 g of acetaldehyde in 60 ml of water were added dropwise. The mixture was stirred at room temperature for another 24 hours. The mixture was extracted three times with dichloromethane. The combined organic phases were adjusted to pH 5-6 with acetic acid-water 1:4, washed with water, dried over magnesium sulphate and concentrated. The product was purified on silica gel 60 (mobile phase: hexane, hexane-ethyl acetate 95:5 and 90:10). 38 g (65% of theory) of product were obtained.

¹H NMR (400 MHz, chloroform-d₁): δ=3.92 (s, 3H), 6.77 (dd, 1H), 7.02-7.07 (m, 1H), 7.10-7.18 (m, 2H), 7.69 (d, 1H), 9.73 (d, 1H).

### Intermediate 3-2

### (E)-3-(4-Fluoro-3-methoxyphenyl)acrylaldehyde

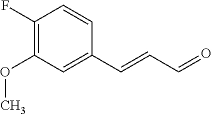

142 ml of 20% strength potassium hydroxide solution were added to 50 g (0.324 mol) of 4-fluoro-3-methoxybenzaldehyde in 250 ml of dichloromethane. Over 2 hours, 73 ml (1.298 mol) of acetaldehyde in 210 ml of water were added dropwise at below 30° C. The mixture was stirred at room temperature overnight. On four days, in each case 1 molar equivalent of acetaldehyde was added dropwise in 3 portions of 6 ml, and the mixture was stirred overnight or over the weekend. The reaction mixture was extracted three times with dichloromethane. The combined organic phases were adjusted to pH 5-6 with acetic acid-water 1:3, washed with water, dried over magnesium sulphate and concentrated. The product was purified on silica gel 60 (mobile phase: hexane, hexane-ethyl acetate 95:5, 90:10, 85:15, 80:20 and 70:30). 17.56 g (30% of theory) of product were obtained.

¹H NMR (400 MHz, chloroform-d₁): δ=3.93 (s, 3H), 6.64 (dd, 1H), 7.11-7.17 (m, 3H), 7.42 (d, 1H), 9.69 (d, 1H).

### Intermediate 1-3

### (4E)-5-(2-Chloro-3-methoxyphenyl)-2-phenylpenta-2,4-dienoic acid

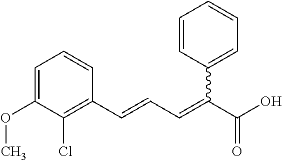 [SMILES: COc1cccc(/C=C/C=C(C(=O)O)c2ccccc2)c1Cl]

6.232 g (0.046 mol) of phenylacetic acid and 12.69 ml (0.091 mol) of triethylamine were added to 9.0 g (0.046 mol) of (E)-3-(2-chloro-3-methoxyphenyl)acrylaldehyde and 8.64 ml (0.091 mol) of acetic anhydride. The mixture was stirred at 100° C. for 4 hours. The reaction was poured onto ice/water with 5% by volume of concentrated hydrochloric acid and extracted three times with chloroform. The combined organic phases were washed twice with water, dried over magnesium sulphate and concentrated. Diisopropyl ether was added to the residue, and the product was filtered off with suction and dried in a drying cabinet. 6.5 g (45% of theory) of product were isolated.

¹H NMR (300 MHz, chloroform-d₁): δ=3.90 (s, 3H), 6.76-6.88 (m, 2H), 7.03 (dd, 1H), 7.13 (t, 1H), 7.29-7.34 (m, 2H), 7.37-7.53 (m, 4H), 7.80 (d, 1H).

### Intermediate 2-3

### (4E)-5-(2-Fluoro-3-methoxyphenyl)-2-(4-fluoro-3-methoxyphenyl)penta-2,4-dienoic acid

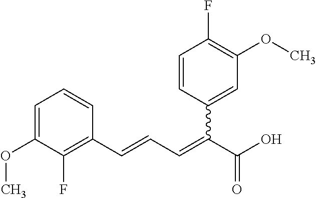 [SMILES: COc1cc(C(=C/C=C/c2cccc(OC)c2F)C(=O)O)ccc1F]

2.811 g (0.015 mol) of (4-fluoro-3-methoxyphenyl)acetic acid and 4.23 ml (0.031 mol) of triethylamine were added to 2.5 g (0.014 mol) of (E)-3-(2-fluoro-3-methoxyphenyl)acrylaldehyde and 2.88 ml (0.031 mol) of acetic anhydride. The mixture was stirred at 100° C. for 10 hours and at room temperature overnight. The reaction was poured onto ice/water with 5% by volume of concentrated hydrochloric acid and extracted three times with chloroform. The combined organic phases were washed twice with water, dried over magnesium sulphate and concentrated. Diisopropyl ether was added to the residue, and the product was filtered off with suction and dried in a drying cabinet. 2.2 g (46% of theory) of product were isolated.

¹H NMR (300 MHz, DMSO-d₆): δ=3.78 (s, 3H), 3.79 (s, 3H), 6.73-6.84 (m, 2H), 6.95-7.02 (m, 2H), 7.03-7.09 (m, 2H), 7.14-7.25 (m, 2H), 7.55 (d, 1H).

### Intermediate 3-3

### (2E/Z,4E)-2,5-Bis(4-fluoro-3-methoxyphenyl)penta-2,4-dienoic acid

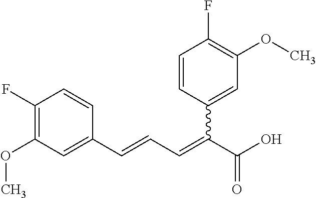 [SMILES: COc1cc(/C=C/C=C(C(=O)O)c2ccc(F)c(OC)c2)ccc1F]

30.05 g (0.163 mol) of (4-fluoro-3-methoxyphenyl)acetic acid and 47.4 ml (0.342 mol) of triethylamine were added to 28.00 g (0.155 mol) of (E)-3-(4-fluoro-3-methoxyphenyl)acrylaldehyde and 32.3 ml (0.342 mol) of acetic anhydride. The mixture was stirred at 100° C. for 18 hours. The reaction was poured onto ice/water with 5% by volume of concentrated hydrochloric acid, stirred for 1 hour and extracted three times with chloroform. The combined organic phases were washed four times with water, dried over magnesium sulphate and concentrated. 200 ml of diisopropyl ether were added to the residue, and the mixture was stirred at 60° C. for 2 hours, filtered off with suction, washed with cold diisopropyl ether and dried at 40° C. in a drying cabinet. 31.3 g (55% of theory) of product were isolated.

¹H NMR (400 MHz, chloroform-d₁): main isomer: δ=3.88 (s, 3H), 3.90 (s, 3H), 6.70 (dd, 1H), 6.84 (ddd, 1H), 6.88-7.10 (m, 5H), 7.14 (dd, 1H), 7.72 (d, 1H).

### Intermediate 4-3

### (4E)-5-(2-Chloro-3-methoxyphenyl)-2-(4-fluoro-3-methoxyphenyl)penta-2,4-dienoic acid

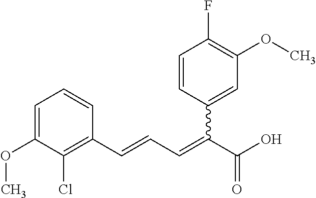 [SMILES: COc1cc(C(=C/C=C/c2cccc(OC)c2Cl)C(=O)O)ccc1F]

A mixture of 12.2 g of 3-(2-chloro-3-methoxyphenyl)acrylaldehyde and 12.6 g (4-fluoro-3-methoxyphenyl)acetic acid in 12.9 ml of acetic anhydride and 18.9 ml of triethylamine was stirred at 100° C. for 7.5 h. The mixture was poured onto ice-water (acidified with 5% strength hydrochloric acid solution), extracted with dichloromethane, washed with saturated sodium chloride solution, dried over sodium sulphate and concentrated. The residue was precipitated with ice-cold ether. This gave 3.6 g of a solid. C₁₉H₁₆ClFO₄ (362.79). MS (ESIpos) mass found: 362.00.

### Intermediates 4

General Procedure 4 for the preparation of 4: 1 g of diencarboxylic acid were dissolved in 20-25 ml of tetrahydrofuran and hydrogenated with 0.1-0.2 g of 10% by weight palladium on activated carbon at standard pressure until the uptake of hydrogen was complete. The catalyst was filtered off through kieselguhr and washed with tetrahydrofuran. The filtrate was concentrated to dryness. The product was obtained in quantitative yield.

### Intermediate 1-4

### 5-(2-Chloro-3-methoxyphenyl)-2-phenylpentanoic acid

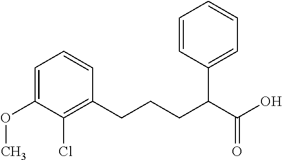 [SMILES: COc1cccc(CCCC(C(=O)O)c2ccccc2)c1Cl]

10.0 g (31.8 mmol) of (4E)-5-(2-chloro-3-methoxyphenyl)-2-phenylpenta-2,4-dienoic acid were reacted according to General Procedure 4.

¹H NMR (300 MHz, chloroform-d₁): δ=1.47-1.72 (m, 2H), 1.78-1.93 (m, 1H), 2.07-2.22 (m, 1H), 2.65-2.88 (m, 2H), 3.58 (t, 1H), 3.88 (s, 3H), 6.74-6.81 (m, 2H), 7.11 (t, 1H), 7.21-7.35 m, 5H).

### Intermediate 2-4

### 5-(2-Fluoro-3-methoxyphenyl)-2-(4-fluoro-3-methoxyphenyl)pentanoic acid

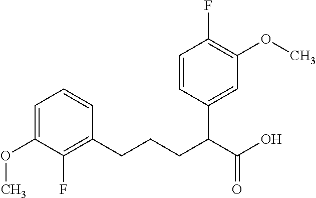 [SMILES: COc1cc(C(CCCc2cccc(OC)c2F)C(=O)O)ccc1F]

36.0 g (103.9 mmol) of (4E)-5-(2-fluoro-3-methoxyphenyl)-2-(4-fluoro-3-methoxyphenyl)penta-2,4-dienoic acid were reacted according to General Procedure 4.

¹H NMR (300 MHz, chloroform-d₁): δ=1.46-1.70 (m, 2H), 1.73-1.91 (m, 1H), 2.02-2.16 (m, 1H), 2.56-2.74 (m, 2H), 3.52 (t, 1H), 3.86 (s, 3H), 3.87 (s, 3H), 6.62-7.07 (m, 6H).

### Intermediate 3-4

### 2,5-Bis(4-fluoro-3-methoxyphenyl)pentanoic acid

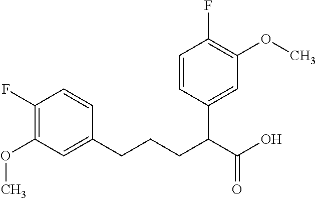 [SMILES: COc1cc(CCCC(C(=O)O)c2ccc(F)c(OC)c2)ccc1F]

33.3 g (96.2 mmol) of (4E)-2,5-bis(4-fluoro-3-methoxyphenyl)penta-2,4-dienoic acid were reacted according to General Procedure 4.

¹H NMR (300 MHz, chloroform-d₁): δ=1.44-1.69 (m, 2H), 1.70-1.89 (m, 1H), 1.99-2.14 (m, 1H), 2.48-2.66 (m, 2H), 3.50 (t, 1H), 3.85 (s, 3H), 3.86 (s, 3H), 6.63 (ddd, 1H), 6.70 (dd, 1H), 6.80 (ddd, 1H), 6.87 (dd, 1H), 6.91-7.05 (m, 2H).

### Intermediate 4-4

### 5-(2-Chloro-3-methoxyphenyl)-2-(4-fluoro-3-methoxyphenyl)pentanoic acid

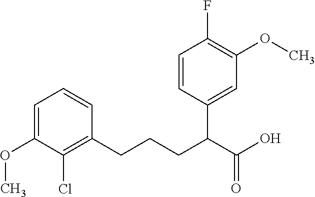 [SMILES: COc1cc(C(CCCc2cccc(OC)c2Cl)C(=O)O)ccc1F]

3.6 g of (2E,4E)-5-(2-chloro-3-methoxyphenyl)-2-(4-fluoro-3-methoxyphenyl)penta-2,4-dienoic acid were reacted according to General Procedure 4. This gave 3.6 g of the title compound. C₁₉H₂₀ClFO₄ (366.82). 1H NMR (400 MHz, chloroform-d): δ 1.48-1.70 (m, 2H), 1.81-1.90 (m, 2H), 2.04-2.19 (m, 1H), 2.71-2.80 (m, 1H), 3.54 (t, 1H), 6.75-6.86 (3H), 6.90 (dd, 1H), 7.12 (t, 1H).

### Intermediates 5

General Procedure 5 for the preparation of 5 under exclusion of atmospheric moisture: 1 g of carboxylic acid was dissolved in 5-7.2 ml of methanesulphonic acid and a little at a time 2.7-2.8 equivalents of phosphorus pentoxide were added with cooling. The mixture was stirred at room temperature for 3-16 hours. The reaction mixture was poured into ice/water and extracted three times with ethyl acetate. The combined organic phases were adjusted to pH 7-8 with 2 M aqueous sodium hydroxide solution, washed with saturated sodium chloride solution, dried over sodium sulphate and concentrated.

General Procedure 5-A for the preparation of 5 under exclusion of atmospheric moisture: 1 g of carboxylic acid was dissolved in about 5-10 ml of trifluoromethanesulphonic acid. At 5-20° C., 2.8 equivalents of phosphorus pentoxide were added in 3 portions. The mixture was stirred overnight. The reaction was poured into ice/water and stirred for another half an hour. The aqueous phase was extracted three times with ethyl acetate. The combined organic phases were adjusted to pH 7-8 with water, saturated sodium chloride solution and sodium carbonate solution, dried over magnesium sulphate and concentrated.

### Preparation of the Intermediates 5 via Palladium Catalysis

General Procedure 5-vPd for the preparation of 5 via palladium catalysis under an atmosphere of argon: 1.3 equivalents of sodium tert-butoxide, 0.05 equivalents of palladium(II) acetate and 0.024 equivalents of xantphos were initially charged in tetrahydrofuran (20 ml/1 g of ketone) under argon. 1 equivalent of 2-methoxy-6,7,8,9-tetrahydro-5H-benzo[7]annulen-5-one (ketone) dissolved in tetrahydrofuran (5 ml/1 g of ketone) were added dropwise. The mixture was stirred for 10 minutes, and 1 equivalent of aryl bromide in tetrahydrofuran (5 ml/1 g of aryl bromide) was then added dropwise. The mixture was stirred under reflux for 10-25 hours. The reaction was cooled and poured into potassium phosphate buffer pH 7. The mixture was extracted four times with ethyl acetate. The combined organic phases were dried over magnesium sulphate or sodium sulphate and concentrated. The residue was purified on silica gel 60.

### Intermediate 1-5

### 1-Chloro-2-methoxy-6-phenyl-6,7,8,9-tetrahydro-5H-benzo[7]annulen-5-one

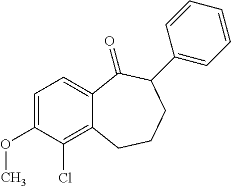 [SMILES: COc1ccc2c(c1Cl)CCCC(c1ccccc1)C2=O]

11.00 g (34.5 mmol) of 5-(2-chloro-3-methoxyphenyl)-2-phenylpentanoic acid were reacted according to General Procedure 5-A. The residue and the residues of an 8 g and a 4.5 g reaction were chromatographed on silica gel 60 (mobile phase: hexane-dichloromethane 1:1, hexane-dichloromethane-acetone 10:10:1). This gave 12.75 g (58% of theory) of product.

¹H NMR (300 MHz, chloroform-d₁): δ=1.76-1.94 (m, 1H), 2.04-2.26 (m, 3H), 2.84-2.97 (m, 1H), 3.51-3.62 (m, 1H), 3.96 (s, 3H), 4.01 (dd, 1H), 6.88 (d, 1H), 7.02-7.38 (m, 5H), 7.53 (d, 1H).

### Intermediate 2-5

### 1-Fluoro-6-(4-fluoro-3-methoxyphenyl)-2-methoxy-6,7,8,9-tetrahydro-5H-benzo[7]annulen-5-one

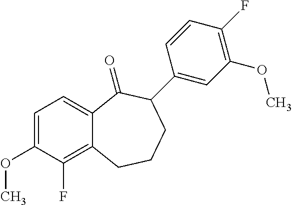 [SMILES: COc1cc(C2CCCc3c(ccc(OC)c3F)C2=O)ccc1F]

35 g (99.90 mmol) of 5-(2-fluoro-3-methoxyphenyl)-2-(4-fluoro-3-methoxyphenyl)pentanoic acid were reacted according to General Procedure 5. The reaction mixture was poured into ice/water and stirred for half an hour. The precipitated product was filtered off with suction, washed seven times with water and dried in a drying cabinet at 40° C. The material was chromatographed on silica gel 60 (mobile phase: hexane, hexane-acetone 95:5 and 9:1). This gave 5.8 g (17% of theory) of product.

¹H NMR (300 MHz, chloroform-d₁): δ=1.71-1.87 (m, 1H), 2.07-2.30 (m, 3H), 2.74-2.88 (m, 1H), 3.36-3.48 (m, 1H), 3.89 (s, 3H), 3.94 (s, 3H), 4.05 (dd, 1H), 6.77 (ddd, 1H), 6.86-6.95 (m, 2H), 7.03 (dd, 1H), 7.49 (dd, 1H).

### Intermediate 3-5

### 3-Fluoro-6-(4-fluoro-3-methoxyphenyl)-2-methoxy-6,7,8,9-tetrahydro-5H-benzo[7]annulen-5-one

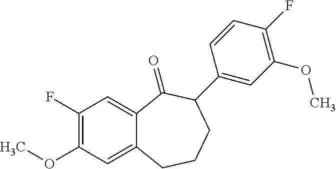

33.0 g (94.2 mmol) of 2,5-bis(4-fluoro-3-methoxyphenyl)pentanoic acid were reacted according to General Procedure 5. After 3 hours of stirring at room temperature, the mixture was poured into ice/water and stirred for half an hour. The mixture was extracted three times with chloroform. The combined organic phases were washed three times with water, dried over magnesium sulphate and concentrated. The product was recrystallized from isopropanol and a little acetone and dried at 35° C. in a drying cabinet. 21.06 g (67% of theory) of product were obtained.

¹H NMR (300 MHz, chloroform-d₁): δ=1.73-1.89 (m, 1H), 2.03-2.28 (m, 3H), 2.91-3.02 (m, 1H), 3.05-3.19 (m, 1H), 3.89 (s, 3H), 3.95 (s, 3H), 4.01 (dd, 1H), 6.75 (ddd, 1H), 6.81 (d, 1H), 6.89 (dd, 1H), 7.03 (dd, 1H), 7.49 (d, 1H).

### Intermediate 4-5

### 1-Chloro-6-(4-fluoro-3-methoxyphenyl)-2-methoxy-6,7,8,9-tetrahydro-5H-benzo[7]annulen-5-one

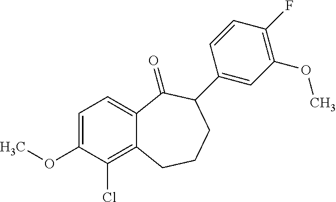

3.6 g of 5-(2-chloro-3-methoxyphenyl)-2-(4-fluoro-3-methoxyphenyl)pentanoic acid were dissolved in 19 ml of trifluoromethanesulphonic acid, 3.9 g of diphosphorus pentoxide were added a little at a time at 10-20° C. and the mixture was stirred at RT overnight. The reaction mixture was poured onto ice/water, and sodium bicarbonate as added carefully a little at a time until a pH of 7 had been reached. Ethyl acetate was added and the phases were separated after brief stirring. The aqueous phase was extracted twice with ethyl acetate, and the combined organic phases were then washed with saturated sodium chloride solution, dried over sodium sulphate and concentrated. Purification by column chromatography on silica gel (hexane/ethyl acetate) gave 1.08 g of the title compound. C₁₆H₁₈ClFO₃ (348.80) MS (ESIpos) mass found: 348.00. 1H NMR (400 MHz, DMSO-d₆): δ 1.54-1.66 (m, 1H), 1.89-2.16 (m, 3H), 2.89-2.99 (m, 1H), 3.34-3.42 (m, 1H), 3.76 (s, 3H), 3.88 (s, 3H), 4.18 (dd, 1H), 6.74-6.79 (m, 1H). 7.02 (dd, 1H), 7.05-7.12 (2H), 7.45 (d, 1H).

### Intermediate 1-5-vPd

### 2-Methoxy-6-(4-methoxyphenyl)-6,7,8,9-tetrahydro-5H-benzo[7]annulen-5-one

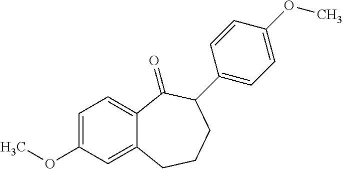

According to General Procedure 5-vPd, 20 g (105.13 mmol) of 2-methoxy-6,7,8,9-tetrahydro-5H-benzo[7]annulen-5-one were stirred under reflux with 13.16 ml (105.13 mmol) of 1-bromo-4-methoxybenzene for 16 hours. The residue was purified on silica gel 60 (mobile phase: hexane, hexane-acetone 9:1, 8:2 and 6:4). 20.00 g (64% of theory) of product were isolated.

¹H NMR (300 MHz, methanol-d₄): δ=1.63-1.81 (m, 1H), 1.93-2.21 (m, 3H), 2.93 (mc, 1H), 3.12 (mc, 1H), 3.75 (s, 3H), 3.82 (s, 3H), 4.01 (dd, 1H), 6.79-8.87 (m, 4H), 7.11 (mc, 2H), 7.57 (d, 1H).

### Intermediate 2-5-vPd

### 2-Methoxy-6-(3-methoxyphenyl)-6,7,8,9-tetrahydro-5H-benzo[7]annulen-5-one

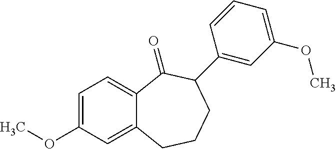

According to General Procedure 5-vPd, 20 g (105.13 mmol) of 2-methoxy-6,7,8,9-tetrahydro-5H-benzo[7]annulen-5-one were stirred under reflux with 19.66 g (105.13 mmol) of 1-bromo-3-methoxybenzene for 16 hours. The residue was purified on silica gel 60 (mobile phase: hexane, hexane-acetone 9:1). 20.00 g (64% of theory) were isolated.

¹H NMR (300 MHz, DMSO-d₆): δ=1.52-1.70 (m, 1H), 1.79-2.18 (m, 3H), 2.88 (dt, 1H), 3.11 (mc, 1H), 3.69 (s, 3H), 3.78 (s, 3H), 4.09 (dd, 1H), 6.73-6.89 (m, 5H), 7.17 (mc, 1H), 7.51 (d, 1H).

### Intermediate 3-5-vPd

### 6-(3-Fluoro-4-methoxyphenyl)-2-methoxy-6,7,8,9-tetrahydro-5H-benzo[7]annulen-5-one

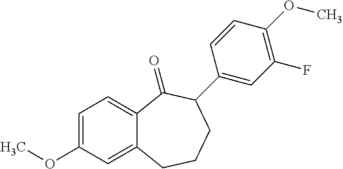

According to General Procedure 5-vPd, 20 g (105.13 mmol) of 2-methoxy-6,7,8,9-tetrahydro-5H-benzo[7]annulen-5-one were stirred under reflux with 21.55 g (105.13 mmol) of 4-bromo-2-fluoro-1-methoxybenzene, 0.1 equivalent of palladium(II) acetate and 0.048 equivalent of xantphos for 7 hours. The residue was purified on silica gel 60 (mobile phase: hexane, hexane-acetone 9:1, 8:2). This gave 17.92 g (54% of theory) of product.

¹H NMR (400 MHz, chloroform-d₁): δ=1.75-1.88 (m, 1H), 2.02-2.23 (m, 3H), 2.95 (ddd, 1H), 3.11 (mc, 1H), 3.86 (s, 3H), 3.88 (s, 3H), 3.99 (dd, 1H), 6.76 (d, 1H), 6.82 (dd, 1H), 6.90-6.98 (m, 2H), 7.03 (dd, 1H), 7.71 (d, 1H).

### Intermediate 4-5-vPd

### 6-(4-Fluoro-3-methoxyphenyl)-2-methoxy-6,7,8,9-tetrahydro-5H-benzo[7]annulen-5-one

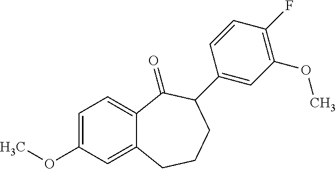

According to General Procedure 5-vPd, 20 g (105.13 mmol) of 2-methoxy-6,7,8,9-tetrahydro-5H-benzo[7]annulen-5-one were stirred under reflux with 21.56 g (105.13 mmol) of 4-bromo-1-fluoro-2-methoxybenzene for 24 hours. The residue was purified on silica gel 60 (mobile phase: hexane, hexane-acetone 95:5, 90:10). This gave 17.6 g (53% of theory) of product.

¹H NMR (300 MHz, chloroform-d₁): δ=1.74-1.90 (m, 1H), 2.02-2.26 (m, 3H), 2.96 (mc, 1H), 3.12 (mc, 1H), 3.86 (s, 3H), 3.89 (s, 3H), 4.02 (dd, 1H), 6.73-6.79 (m, 2H), 6.82 (dd, 1H), 6.90 (dd, 1H), 7.03 (dd, 1H), 7.71 (d, 1H).

### Intermediate 5-5-vPd

### 2-Methoxy-6-(3-pyridyl)-6,7,8,9-tetrahydro-5H-benzo[7]annulen-5-one

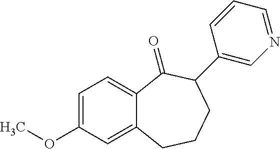 [SMILES: COc1ccc2c(c1)CCCC(c1cccnc1)C2=O]

According to General Procedure 5-vPd, 25 g (131.41 mmol) of 2-methoxy-6,7,8,9-tetrahydro-5H-benzo[7]annulen-5-one were stirred under reflux with 20.76 g (131.39 mmol) of 3-bromopyridine for 10 hours. Dichloromethane was used for the extraction. The residue was purified on silica gel 60 (mobile phase: dichloromethane, dichloromethane-acetone 95:5, 90:10). This gave 25 g (71% of theory) of product.

¹H NMR (400 MHz, chloroform-d₁): δ=1.78-1.91 (m, 1H), 2.05-2.27 (m, 3H), 2.98 (mc, 1H), 3.17 (mc, 1H), 3.86 (s, 3H), 4.09 (dd, 1H), 6.78 (d, 1H), 6.83 (dd, 1H), 7.29 (dd, 1H), 7.68 (dt, 1H), 7.72 (d, 1H), 8.47 (d, 1H), 8.52 (dd, 1H).

### Intermediate 6-5-vPd

### 6-(4-Mesylphenyl)-2-methoxy-6,7,8,9-tetrahydro-5H-benzo[7]annulen-5-one

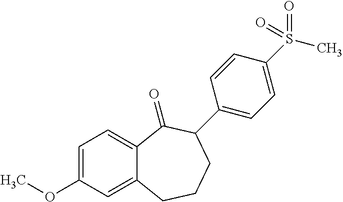

According to General Procedure 5-vPd, 5 g (26.28 mmol) of 2-methoxy-6,7,8,9-tetrahydro-5H-benzo[7]annulen-5-one were stirred under reflux with 6.18 g (26.28 mmol) of 1-bromo-4-mesylbenzene for 16 hours. The residue was purified on silica gel 60 (mobile phase: hexane, hexane-acetone 9:1, 8:2). 3.65 g (39% of theory) of the product were isolated.

¹H NMR (400 MHz, chloroform-d₁): δ=1.79-1.92 (m, 1H), 2.05-2.27 (m, 3H), 3.06 (s, 3H), 2.98 (mc, 1H), 3.15 (mc, 1H), 3.87 (s, 3H), 4.15 (dd, 1H), 6.78 (d, 1H), 6.83 (dd, 1H), 7.47 (d, 2H), 7.73 (d, 1H), 7.91 (d, 2H).

### Intermediate 7-5-vPd

### 6-(3-Mesylphenyl)-2-methoxy-6,7,8,9-tetrahydro-5H-benzo[7]annulen-5-one

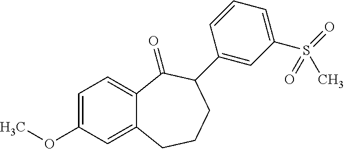 [SMILES: COc1ccc2c(c1)CCCC(c1cccc(S(C)(=O)=O)c1)C2=O]

2-Methoxy-6,7,8,9-tetrahydro-5H-benzo[7]annulen-5-one (2.7 g) and 1-bromo-3-mesylbenzene (4.0 g, 1.2 equivalents) were initially charged in THF (50 ml), sodium tert-butoxide (5 g) and allylchloro[1,3-bis(2,6-diisopropyl)imidazol-2-yliden]palladium(II) (CAS 478980-03-9) (500 mg) were added and the mixture was heated under reflux for 115 h. Water was added and most of the THF was removed. Ethyl acetate and water were then added, the organic phase was removed and the aqueous phase was extracted three times with ethyl acetate. The combined organic phases were washed with saturated sodium chloride solution and dried over sodium sulphate. Column chromatography on silica gel (hexane/acetone) gave the title compound (crude product) as a solid. C₁₉H₂₀O₄S (344.43). 1H NMR (300 MHz, chloroform-d): δ 1.81-1.98 (m, 1H), 2.1-2.35 (m, 3H), 2.97-3.08 (m, 1H), 3.12 (s, 3H), 3.14-3.27 (m, 1H), 3.91 (s, 3H), 4.17-4.26 (m, 1H), 6.81-6.91 (m, 2H), 7.57-7.68 (m, 2H), 7.77 (d, 1H), 7.85-7.93 (m, 2H).

### Intermediate 8-5-vPd

### 4-(2-Methoxy-5-oxo-6,7,8,9-tetrahydro-5H-benzo[7]annulen-6-yl)benzonitrile

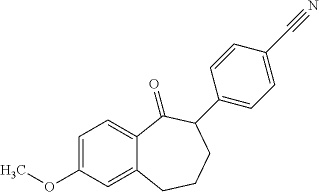 [SMILES: COc1ccc2c(c1)CCCC(c1ccc(C#N)cc1)C2=O]

A solution of 5.74 g of 4-bromobenzonitrile in 25 ml of THF was added dropwise to a mixture of 5 g (26 mmol) of 2-methoxy-6,7,8,9-tetrahydro-5H-benzo[7]annulen-5-one, 12.8 g of potassium phosphate, 456 mg of 9,9-dimethyl-4,5-bis-(diphenylphosphino)-xanthene and 361 mg of tris(dibenzylideneacetone)dipalladium(0) in 100 ml of THF. The mixture was heated under reflux for 20 h, another 228 mg of 9,9-dimethyl-4,5-bis-(diphenylphosphino)-xanthene and 180 mg of tris(dibenzylideneacetone)dipalladium(0) were then added and the mixture was heated under reflux for 1 h. The reaction mixture was concentrated, and water and ethyl acetate were added. The organic phase was separated off, the aqueous phase was extracted three times with ethyl acetate and the combined organic phases were washed with sodium chloride solution, dried over sodium sulphate and concentrated. Purification by column chromatography on silica gel gave 7.45 g of a crude product. MS (ESIpos) mass found: 291.00.

### Intermediate 9-5-vPd

### 6-(2-Fluoro-5-hydroxyphenyl)-2-methoxy-6,7,8,9-tetrahydro-5H-benzo[7]annulen-5-one

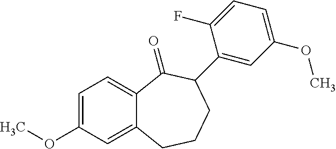

1.5 g of 2-methoxy-6,7,8,9-tetrahydro-5H-benzo[7]annulen-5-one were initially charged in 12 ml of THF, 2.8 g of sodium tert-butoxide, 1.20 ml of 2-chloro-1-fluoro-4-methoxybenzene and 135 mg of allylchloro[1,3-bis(2,6-diisopropylphenyl)imidazol-2-yliden]palladium(II) were added and the mixture was heated in a microwave vessel (pressure vessel) at 120° C. for 90 min. Two analogous reactions were carried out, and the batches were combined, Water was added, and the THF was removed under reduced pressure. The residue was diluted with ethyl acetate and water, the phases were separated and the aqueous phase was extracted with ethyl acetate. The combined organic phases were washed with sodium chloride solution, dried over sodium sulphate and concentrated. Purification by column chromatography (hexane/acetone) gave 2.29 g of the title compound. C₁₉H₁₉FO₃ (314.36). MS (ESIpos) mass found: 314.00.

### Intermediates 6

General Procedure 6-1 for the preparation of 6 under an atmosphere of argon: 1 g of ketone was dissolved in 4.5-12.5 ml of anhydrous tetrahydrofuran, and 1.2 equivalents of 2,3,4,6,7,8,9,10-octahydropyrimido[1,2-a]azepine were added at 3° C. At this temperature, 1.2 equivalents of 1,1,2,2,3,3,4,4,4-nonafluorobutane-1-sulphonyl fluoride in anhydrous tetrahydrofuran (1 g in 0.6-4.5 ml) were added dropwise. The mixture was stirred at 3° C. for 2 hours and at room temperature overnight. The mixture was then poured into saturated sodium bicarbonate solution (10-20 ml of solution per 1 g of ketone) and extracted three times with methyl tert-butyl ether (about 10-20 ml per 1 g of ketone). The combined organic phases were washed twice with saturated sodium chloride solution (about 5-20 ml per 1 g of ketone), dried over magnesium sulphate and concentrated to dryness. Pentane was added to the residue, and the mixture was stirred at room temperature for one hour. The mixture was then filtered off with suction, washed with pentane and dried in a drying cabinet at room temperature.

General Procedure 6-2 for the preparation of 6 under an atmosphere of argon: 1 g of ketone was dissolved in 5-7.5 ml of anhydrous tetrahydrofuran/methyl tert-butyl ether (1:1 to 4:3), and 2.4 equivalents of 2,3,4,6,7,8,9,10-octahydropyrimido[1,2-a]azepine were added at 3° C.

At this temperature, 2.4 equivalents of 1,1,2,2,3,3,4,4,4-nonafluorobutane-1-sulphonyl fluoride in anhydrous tetrahydrofuran (1 g in 1 ml) were added dropwise. The mixture was stirred at 3° C. for 3 hours. The reaction was allowed to warm to room temperature, a saturated potassium carbonate solution was added, the phases were separated and the aqueous phase was extracted twice with methyl tert-butyl ether. The combined organic phases were dried over sodium sulphate and concentrated to dryness.

### Intermediate 1-6

### 3-Methoxy-8-phenyl-6,7-dihydro-5H-benzo[7]annulen-9-yl-1,1,2,2,3,3,4,4,4-nonafluorobutane-1-sulphonate

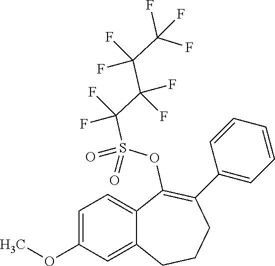 [SMILES: COc1ccc2c(c1)CCCC(c1ccccc1)=C2OS(=O)(=O)C(F)(F)C(F)(F)C(F)(F)C(F)(F)F]

10.2 g (38.3 mmol) of 2-methoxy-6-phenyl-6,7,8,9-tetrahydro-5H-benzo[7]annulen-5-one were reacted according to General Procedure 6-1. 19.7 g (94% of theory) of product were obtained.

¹H NMR (400 MHz, chloroform-d₁): δ=2.26 (t, 2H), 2.40 (mc, 2H), 2.86 (t, 2H), 3.86 (s, 3H), 6.83 (d, 1H), 6.88 (dd, 1H), 7.30-7.36 (m, 1H), 7.38-7.49 (m, 5H).

### Intermediate 2-6

### 3-Methoxy-8-(3-methoxyphenyl)-6,7-dihydro-5H-benzo[7]annulen-9-yl-1,1,2,2,3,3,4,4,4-nonafluorobutane-1-sulphonate

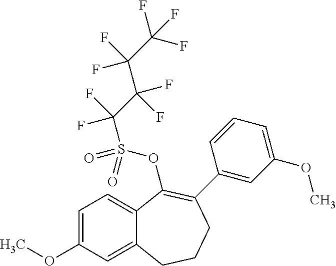

9 g (30.37 mmol) of 2-methoxy-6-(3-methoxyphenyl)-6,7,8,9-tetrahydro-5H-benzo[7]annulen-5-one were reacted according to General Procedure 6-1. 17.57 g (100% of theory) of product were isolated.

¹H NMR (300 MHz, chloroform-d₁): δ=2.20-2.29 (m, 2H), 2.39 (mc, 2H), 2.85 (t, 2H), 3.85 (s, 3H), 3.86 (s, 3H), 6.82 (d, 1H), 6.84-6.90 (m, 2H), 6.97-7.04 (m, 2H), 7.32 (t, 1H), 7.44 (d, 1H).

### Intermediate 3-6

### 3-Methoxy-8-(4-methoxyphenyl)-6,7-dihydro-5H-benzo[7]annulen-9-yl-1,1,2,2,3,3,4,4,4-nonafluorobutane-1-sulphonate

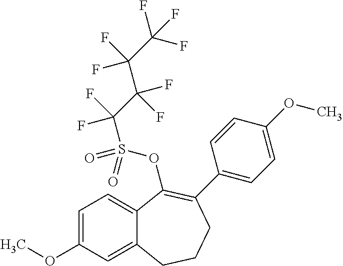

20 g (67.48 mmol) of 2-methoxy-6-(4-methoxyphenyl)-6,7,8,9-tetrahydro-5H-benzo[7]annulen-5-one were reacted according to General Procedure 6-2. This gave 64.12 g (164% of theory) of residue.

¹H NMR (400 MHz, chloroform-d₁): δ=2.24 (t, 2H), 2.38 (mc, 2H), 2.84 (t, 2H), 3.83 (s, 3H), 3.85 (s, 3H), 6.81 (d, 1H), 6.87 (dd, 1H), 6.93 (d, 2H), 7.36-7.45 (m, 3H).

### Intermediate 4-6

### 8-(4-Fluoro-3-methoxyphenyl)-3-methoxy-6,7-dihydro-5H-benzo[7]annulen-9-yl-1,1,2,2,3,3,4,4,4-nonafluorobutane-1-sulphonate

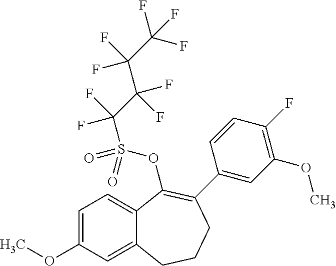

g (28.63 mmol) of 6-(4-fluoro-3-methoxyphenyl)-2-methoxy-6,7,8,9-tetrahydro-5H-benzo[7]annulen-5-one were reacted according to General Procedure 6-1. This gave 17.0 g (100% of theory) of product.

MS (pos): m/z=596 m+

### Intermediate 5-6

### 8-(3-Fluoro-4-methoxyphenyl)-3-methoxy-6,7-dihydro-5H-benzo[7]annulen-9-yl-1,1,2,2,3,3,4,4,4-nonafluorobutane-1-sulphonate

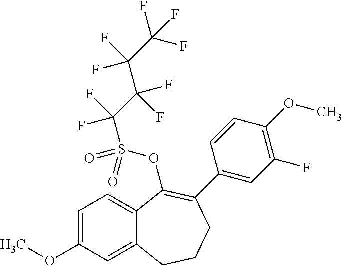

6.3 g (20.04 mmol) of 6-(3-fluoro-4-methoxyphenyl)-2-methoxy-6,7,8,9-tetrahydro-5H-benzo[7]annulen-5-one were reacted according to General Procedure 6-2. 24.7 g (207% of theory) of crude product were isolated.

¹H NMR (300 MHz, chloroform-d₁): δ=2.18-2.26 (m, 2H), 2.37 (mc, 2H), 2.83 (t, 2H), 3.85 (s, 3H), 3.92 (s, 3H), 6.81 (d, 1H), 6.87 (dd, 1H), 6.97 (t, 1H), 7.15-7.22 (m, 2H), 7.42 (d, 1H).

### Intermediate 6-6

### 3-Methoxy-8-(3-pyridyl)-6,7-dihydro-5H-benzo[7]annulen-9-yl-1,1,2,2,3,3,4,4,4-nonafluorobutane-1-sulphonate

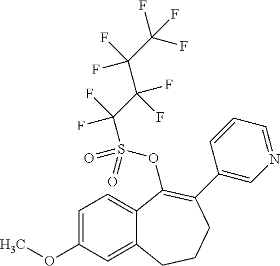 [SMILES: COc1ccc2c(c1)CCCC(c1cccnc1)=C2OS(=O)(=O)C(F)(F)C(F)(F)C(F)(F)C(F)(F)F]

10 g (37.41 mmol) of 2-methoxy-6-(3-pyridyl)-6,7,8,9-tetrahydro-5H-benzo[7]annulen-5-one were dissolved in 45 ml of anhydrous tetrahydrofuran, and 6.71 ml (44.88 mmol) of 2,3,4,6,7,8,9,10-octahydropyrimido[1,2-a]azepine were added on an ice bath. At this temperature, 8.1 ml (45.02 mmol) of 1,1,2,2,3,3,4,4,4-nonafluorobutane-1-sulphonyl fluoride in 20 ml of anhydrous tetrahydrofuran were added dropwise. The mixture was then stirred at 0-5° C. for 2 hours and poured into 150 ml of saturated sodium bicarbonate solution and extracted three times with 100 ml of ethyl acetate. The combined organic phases were washed twice with 70 ml of water, dried over magnesium sulphate and concentrated to dryness. This gave 20.55 g (100% of theory).

¹H NMR (300 MHz, chloroform-d₁): δ=2.21-2.31 (m, 2H), 2.42 (mc, 2H), 2.87 (t, 2H), 3.86 (s, 3H), 6.84 (d, 1H), 6.89 (dd, 1H), 7.34 (ddd, 1H), 7.47 (d, 1H), 7.76 (ddd, 1H), 8.58 (dd, 1H), 8.68 (dd, 1H).

### Intermediate 7-6

### 8-(4-Mesylphenyl)-3-methoxy-6,7-dihydro-5H-benzo[7]annulen-9-yl-1,1,2,2,3,3,4,4,4-nonafluorobutane-1-sulphonate

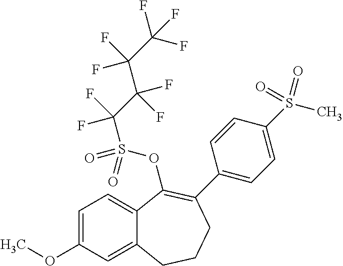

4.17 g (12.11 mmol) of 6-(4-mesylphenyl)-2-methoxy-6,7,8,9-tetrahydro-5H-benzo[7]annulen-5-one were reacted according to General Procedure 6-2. 15.77 g (208% of theory) of crude product were isolated.

¹H NMR (400 MHz, chloroform-d₁): δ=2.27 (t, 2H), 2.43 (mc, 2H), 2.87 (t, 2H), 3.07 (s, 3H), 3.87 (s, 3H), 6.84 (d, 1H), 6.86 (dd, 1H), 7.45 (d, 1H), 7.63 (mc, 2H), 7.98 (mc, 2H).

### Intermediate 8-6

### 4-Chloro-3-methoxy-8-phenyl-6,7-dihydro-5H-benzo[7]annulen-9-yl-1,1,2,2,3,3,4,4,4-nonafluorobutane-1-sulphonate

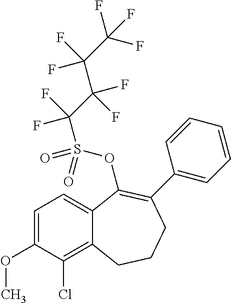 [SMILES: COc1ccc2c(c1Cl)CCCC(c1ccccc1)=C2OS(=O)(=O)C(F)(F)C(F)(F)C(F)(F)C(F)(F)F]

12.50 g (41.6 mmol) of 1-chloro-2-methoxy-6-phenyl-6,7,8,9-tetrahydro-5H-benzo[7]annulen-5-one were reacted according to General Procedure 6-1. 24.22 g (100% of theory) of crude product were isolated.

¹H NMR (300 MHz, chloroform-d₁): δ=2.23 (t, 2H), 2.41 (quin, 2H), 3.13 (t, 2H), 3.96 (s, 3H), 6.93 (d, 1H), 7.30-7.46 (m, 6H).

### Intermediate 9-6

### 4-Fluoro-8-(4-fluoro-3-methoxyphenyl)-3-methoxy-6,7-dihydro-5H-benzo[7]annulen-9-yl-1,1,2,2,3,3,4,4,4-nonafluorobutane-1-sulphonate

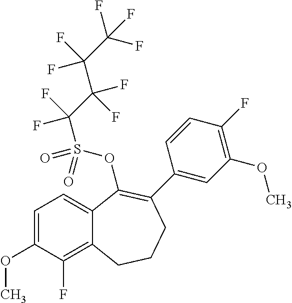 [SMILES: COc1cc(C2=C(OS(=O)(=O)C(F)(F)C(F)(F)C(F)(F)C(F)(F)F)c3ccc(OC)c(F)c3CCC2)ccc1F]

5.80 g (17.5 mmol) of 1-fluoro-6-(4-fluoro-3-methoxyphenyl)-2-methoxy-6,7,8,9-tetrahydro-5H-benzo[7]annulen-5-one were reacted according to General Procedure 6-1, without treatment with pentane. 10.7 g (100% of theory) of crude product were isolated.

### Intermediate 10-6

### 2-Fluoro-8-(4-fluoro-3-methoxyphenyl)-3-methoxy-6,7-dihydro-5H-benzo[7]annulen-9-yl-1,1,2,2,3,3,4,4,4-nonafluorobutane-1-sulphonate

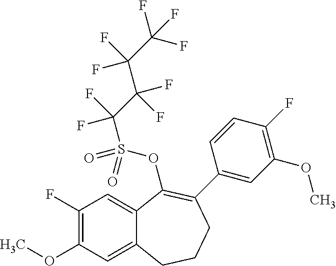

21.00 g (63.2 mmol) of 3-fluoro-6-(4-fluoro-3-methoxyphenyl)-2-methoxy-6,7,8,9-tetrahydro-5H-benzo[7]annulen-5-one were reacted according to General Procedure 6-2. 40.68 g (105% of theory) of crude product were isolated.

¹H NMR (400 MHz, chloroform-d₁): δ=2.26 (t, 2H), 2.40 (quin, 2H), 2.83 (t, 2H), 3.94 (s, 3H), 3.95 (s, 3H), 6.87 (d, 1H), 6.93 (ddd, 1H), 7.06-7.14 (m, 2H), 7.22 (d, 1H).

### Intermediate 11-6

### 8-(2-Fluoro-5-methoxyphenyl)-3-methoxy-6,7-dihydro-5H-benzo[7]annulen-9-yl-1,1,2,2,3,3,4,4,4-nonafluorobutane-1-sulphonate

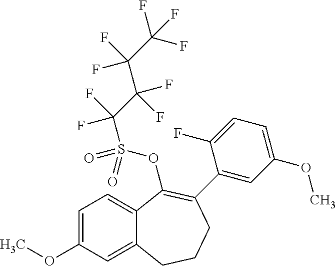

Analogously to General Procedure 6-1, 2.20 g of 6-(2-fluoro-5-hydroxyphenyl)-2-methoxy-6,7,8,9-tetrahydro-5H-benzo[7]annulen-5-one were converted into 4.8 g of crude product. C₂₃H₁₈F₁₀O₅S (596.44). MS (chemical ionization, NH₃) mass found 614.

### Intermediate 12-6

### 4-Chloro-8-(4-fluoro-3-methoxyphenyl)-3-methoxy-6,7-dihydro-5H-benzo[7]annulen-9-yl-1,1,2,2,3,3,4,4,4-nonafluorobutane-1-sulphonate

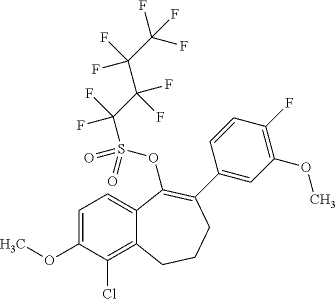

Analogously to General Procedure 6-1, 1.00 g of 1-chloro-6-(4-fluoro-3-methoxyphenyl)-2-m methoxy-6,7,8,9-tetrahydro-5H-benzo[7]annulen-5-one was converted into 2.8 g of the title compound (crude product). C₂₃H₁₇ClF₁₀O₅S (630.89). MS (ESIpos) mass found: 630.00.

### Intermediate 13-6

### 8-(3-Mesylphenyl)-3-methoxy-6,7-dihydro-5H-benzo[7]annulen-9-yl-1,1,2,2,3,3,4,4,4-nonafluorobutane-1-sulphonate

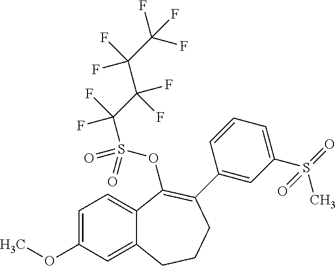 [SMILES: COc1ccc2c(c1)CCCC(c1cccc(S(C)(=O)=O)c1)=C2OS(=O)(=O)C(F)(F)C(F)(F)C(F)(F)C(F)(F)F]

Analogously to General Procedure 6-1, 1.5 g of 6-(3-mesylphenyl)-2-methoxy-6,7,8,9-tetrahydro-5H-benzo[7]annulen-5-one were converted into 3.3 g of the title compound. C₂₃H₁₉F₉O₆S₂ (626.52). MS (ESIpos) mass found: 626.00.

### Intermediate 14-6

### 8-(4-Cyanophenyl)-3-methoxy-6,7-dihydro-5H-benzo[7]annulen-9-yl-1,1,2,2,3,3,4,4,4-nonafluorobutane-1-sulphonate

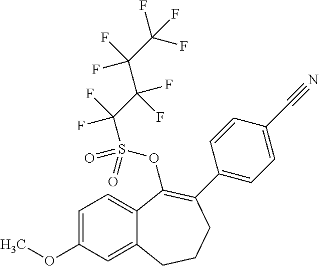 [SMILES: COc1ccc2c(c1)CCCC(c1ccc(C#N)cc1)=C2OS(=O)(=O)C(F)(F)C(F)(F)C(F)(F)C(F)(F)F]

Analogously to General Procedure 6-1, 7.45 g of 4-(2-methoxy-5-oxo-6,7,8,9-tetrahydro-5H-benzo[7]annulen-6-yl)benzonitrile were converted into 18.7 g of the title compound as crude product.

### Intermediate 7

General Procedure 7 for the preparation of 7 under an atmosphere of argon and with exclusion of moisture: 1 g of nonaflate enol ether was dissolved in about 6-13 ml of anhydrous N,N-dimethylformamide. 2.5-2.6 equivalents of alkynol, 4.1 equivalents of triethylamine and 0.033 equivalents of tetrakis(triphenylphosphine)palladium(0) were added. The mixture was stirred at 80° C. for 0.5-1.5 hours. The reaction was cooled and the volatile components were removed under oil pump vacuum on a rotary evaporator. The residue was taken up in ethyl acetate and washed three times with water. The mixture was dried over magnesium sulphate or sodium sulphate and concentrated to dryness. The residue was purified on silica gel 60.

### Intermediate 1-7

### 5-(3-Methoxy-8-phenyl-6,7-dihydro-5H-benzo[7]annulen-9-yl)pent-4-yn-1-ol

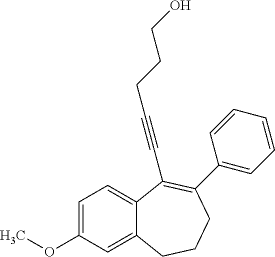 [SMILES: COc1ccc2c(c1)CCCC(c1ccccc1)=C2C#CCCCO]

5.53 g (10.08 mmol) of 3-methoxy-8-phenyl-6,7-dihydro-5H-benzo[7]annulen-9-yl-1,1,2,2,3,3,4,4,4-nonafluorobutane-1-sulphonate and 2.18 g (30.55 mmol) of pent-4-yn-1-ol were reacted according to General Procedure 7. 39 g (71.11 mmol) of 3-methoxy-8-phenyl-6,7-dihydro-5H-benzo[7]annulen-9-yl-1,1,2,2,3,3,4,4,4-nonafluorobutane-1-sulphonate and 15.37 g (182.72 mmol) of pent-4-yn-1-ol were reacted according to General Procedure 7. Both reactions were combined and purified on silica gel 60 (mobile phase: hexane, hexane-ethyl acetate 9:1, 8:2, 3:1 and 1:1). Hexane was added to the residue, and the mixture was filtered off with suction. The product was dried under reduced pressure at room temperature. This gave 15 g (63% of theory).

¹H NMR (300 MHz, chloroform-d₁): δ=1.67 (quin, 2H), 2.16-2.29 (m, 2H), 2.29-2.42 (m, 4H), 2.69 (t, 2H), 3.57 (t, 2H), 3.84 (s, 3H), 6.77 (d, 1H), 6.84 (dd, 1H), 7.25-7.32 (m, 1H), 7.33-7.42 (m, 2H), 7.50 (d, 1H), 7.58-7.65 (m, 2H).

### Intermediate 2-7

### 5-[3-Methoxy-8-(3-methoxyphenyl)-6,7-dihydro-5H-benzo[7]annulen-9-yl]pent-4-yn-1-ol

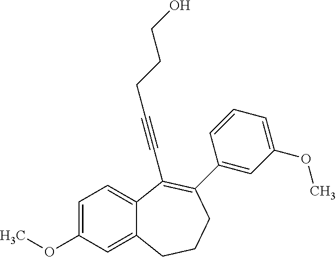

17.5 g (30.25 mmol) of 3-methoxy-8-(3-methoxyphenyl)-6,7-dihydro-5H-benzo[7]annulen-9-yl-1,1,2,2,3,3,4,4,4-nonafluorobutane-1-sulphonate and 6.54 g (77.75 mmol) of pent-4-yn-1-ol were reacted according to General Procedure 7. The product was purified on silica gel 60 (mobile phase: hexane, hexane-ethyl acetate 8:2, 6:4 and 1:1). 6.6 g (60% of theory) of product were isolated.

¹H NMR (300 MHz, chloroform-d₁): δ=1.49 (t-broad, 1H), 1.68 (quin, 2H), 2.15-2.36 (m, 4H), 2.39 (t, 2H), 2.68 (t, 2H), 3.60 (q, 2H), 3.83 (s, 3H), 3.85 (s, 3H), 6.76 (d, 1H), 6.81-6.87 (m, 2H), 7.16 (dt, 1H), 7.24-7.33 (m, 2H), 7.50 (d, 1H).

### Intermediate 3-7

### 6-[3-Methoxy-8-(3-methoxyphenyl)-6,7-dihydro-5H-benzo[7]annulen-9-yl]hex-5-yn-1-ol

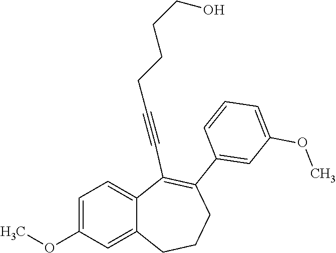

17.5 g (30.25 mmol) of 3-methoxy-8-(3-methoxyphenyl)-6,7-dihydro-5H-benzo[7]annulen-9-yl-1,1,2,2,3,3,4,4,4-nonafluorobutane-1-sulphonate and 8.57 ml (77.72 mmol) of hex-5-yn-1-ol were reacted according to General Procedure 7. The crude product was purified on silica gel 60 (mobile phase: hexane, hexane-ethyl acetate 8:2, 2:1 and 1:1). 8 g (70% of theory) of product were isolated.

¹H NMR (400 MHz, chloroform-d₁): δ=1.30 (s-broad, 1H), 1.48-1.56 (m, 4H), 2.17-2.27 (m, 2H), 2.28-2.36 (m, 4H), 2.68 (t, 2H), 3.57 (mc, 2H), 3.84 (s, 3H), 3.85 (s, 3H), 6.76 (d, 1H), 6.81-6.86 (m, 2H), 7.16 (d, 1H), 7.25 (mc, 1H), 7.29 (t, 1H), 7.51 (d, 1H).

### Intermediate 4-7

### 5-[3-Methoxy-8-(4-methoxyphenyl)-6,7-dihydro-5H-benzo[7]annulen-9-yl]pent-4-yn-1-ol

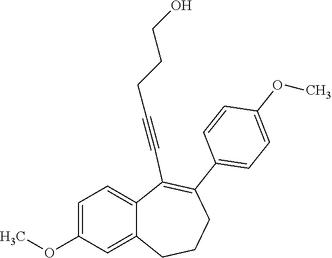

10.7 g (18.43 mmol) of 3-methoxy-8-(4-methoxyphenyl)-6,7-dihydro-5H-benzo[7]annulen-9-yl-1,1,2,2,3,3,4,4,4-nonafluorobutane-1-sulphonate and 3.88 g (46.08 mmol) of pent-4-yn-1-ol were reacted according to General Procedure 7. The crude product was purified on silica gel 60 (mobile phase: hexane, hexane-ethyl acetate 9:1, 4:1, 3:1 and 1:1). This gave 3.56 g (53% of theory) of product.

¹H NMR (400 MHz, chloroform-d₁): δ=1.37 (s-broad, 1H), 1.76 (quin, 2H), 2.18-2.40 (m, 4H), 2.44 (t, 2H), 2.71 (t, 2H), 3.67 (t, 2H), 3.88 (s, 3H), 3.89 (s, 3H), 6.80 (d, 1H), 6.88 (dd, 1H), 6.95 (mc, 2H), 7.54 (d, 1H), 7.64 (mc, 2H).

### Intermediate 5-7

### 6-[3-Methoxy-8-(4-methoxyphenyl)-6,7-dihydro-5H-benzo[7]annulen-9-yl]hex-5-yn-1-ol

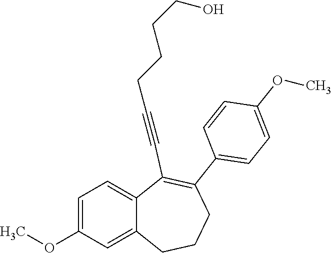

19.5 g (33.71 mmol) of 3-methoxy-8-(4-methoxyphenyl)-6,7-dihydro-5H-benzo[7]annulen-9-yl-1,1,2,2,3,3,4,4,4-nonafluorobutane-1-sulphonate and 9.29 ml (84.28 mmol) of hex-5-yn-1-ol were reacted according to General Procedure 7. The crude product was purified on silica gel 60 (mobile phase: hexane, hexane-ethyl acetate 9:1, 4:1, 3:1 and 1:1 and ethyl acetate). This gave 4.18 g (33% of theory) of product.

¹H NMR (400 MHz, chloroform-d₁): δ=1.18 (t-broad, 1H), 1.48-1.61 (m, 4H), 2.14-2.26 (m, 2H), 2.27-2.35 (m, 4H), 2.67 (t, 2H), 3.59 (mc, 2H), 3.83 (s, 3H), 3.84 (s, 3H), 6.75 (d, 1H), 6.83 (dd, 1H), 6.90 (mc, 2H), 7.50 (d, 1H), 7.60 (mc, 2H).

### Intermediate 6-7

### 6-[8-(4-Fluoro-3-methoxyphenyl)-3-methoxy-6,7-dihydro-5H-benzo[7]annulen-9-yl]hex-5-yn-1-ol

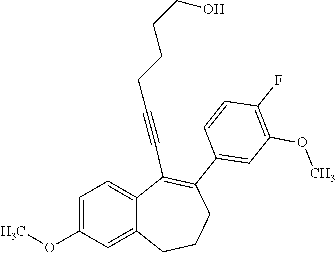

17 g (28.50 mmol) of 8-(4-fluoro-3-methoxyphenyl)-3-methoxy-6,7-dihydro-5H-benzo[7]annulen-9-yl-1,1,2,2,3,3,4,4,4-nonafluorobutane-1-sulphonate and 7.19 g (73.26 mmol) of hex-5-yn-1-ol were reacted according to General Procedure 7. The crude product was purified on silica gel 60 (mobile phase: hexane, hexane-ethyl acetate 8:2, 6:4 and 1:1). This gave 6.9 g (61% of theory) of product.

¹H NMR (300 MHz, chloroform-d₁): δ=1.58 (mc, 4H), 2.20-2.42 (m, 6H), 2.73 (t, 2H), 3.63 (mc, 2H), 3.88 (s, 3H), 3.97 (s, 3H), 6.80 (d, 1H), 6.88 (dd, 1H), 7.05-7.18 (m, 2H), 7.37 (dd, 1H), 7.54 (d, 1H).

### Intermediate 7-7

### 5-[8-(4-Fluoro-3-methoxyphenyl)-3-methoxy-6,7-dihydro-5H-benzo[7]annulen-9-yl]pent-4-yn-1-ol

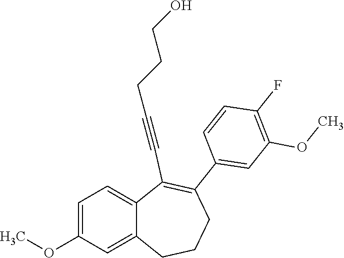

70.3 g of 8-(4-fluoro-3-methoxyphenyl)-3-methoxy-6,7-dihydro-5H-benzo[7]annulen-9-yl-1,1,2,2,3,3,4,4,4-nonafluorobutane-1-sulphonate were reacted analogously with 13.0 ml of 4-pentyn-1-ol in the presence of 1 g of copper iodide and 5.4 g of Pd(PPh₃)₄. Purification by column chromatography on silica gel (hexane/ethyl acetate) gave 20.8 g (46% of theory) of the title compound.

C₂₄H₂₅FO₃ (380.46). 1H NMR (300 MHz, chloroform-d): δ 1.89 (quint., 2H), 2.14-2.34 (m), 2.40 (t, 2H), 2.67 (t, 2H), 3.61 (t, 2H), 3.83 (s, 3H), 3.93 (s, 3H), 6.76 (d, 1H), 6.84 (dd, 1H), 7.01-7.14 (m, 2H), 7.29-7.37 (m, 1H), 7.49 (d, 1H).

### Intermediate 8-7

### 5-[8-(3-Fluoro-4-methoxyphenyl)-3-methoxy-6,7-dihydro-5H-benzo[7]annulen-9-yl]pent-4-yn-1-ol

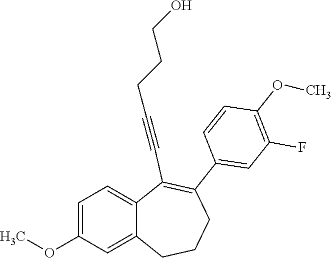

9.28 g (15.56 mmol) of 8-(3-fluoro-4-methoxyphenyl)-3-methoxy-6,7-dihydro-5H-benzo[7]annulen-9-yl-1,1,2,2,3,3,4,4,4-nonafluorobutane-1-sulphonate and 3.27 g (38.87 mmol) of pent-4-yn-1-ol were reacted according to General Procedure 7. The crude product was purified on silica gel 60 (mobile phase: hexane, hexane-ethyl acetate 9:1, 4:1, 3:1 and 1:1). 4.56 g (78% of theory) of product were isolated.

¹H NMR (400 MHz, chloroform-d₁): δ=1.34 (t, 1H), 1.74 (quin., 2H), 2.16-2.33 (m, 4H), 2.42 (t, 2H), 2.66 (t, 2H), 3.66 (q, 2H), 3.83 (s, 3H), 3.92 (s, 3H), 6.75 (d, 1H), 6.83 (dd, 1H), 6.95 (t, 1H), 7.33 (mc, 1H), 7.46-7.53 (m, 2H).

### Intermediate 9-7

### 5-[3-Methoxy-8-(3-pyridyl)-6,7-dihydro-5H-benzo[7]annulen-9-yl]pent-4-yn-1-ol

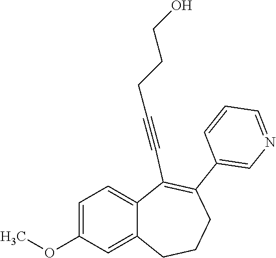 [SMILES: COc1ccc2c(c1)CCCC(c1cccnc1)=C2C#CCCCO]

7.8 g (14.20 mmol) of 3-methoxy-8-(3-pyridyl)-6,7-dihydro-5H-benzo[7]annulen-9-yl-1,1,2,2,3,3,4,4,4-nonafluorobutane-1-sulphonate and 3.07 g (36.50 mmol) of pent-4-yn-1-ol were reacted according to General Procedure 7. The residue was purified on silica gel 60 (mobile phase: hexane-acetone 9:1, 7:3 and 1:1). The combined fractions were concentrated to dryness, diisopropyl ether was added and the material was filtered off with suction and dried at room temperature in a drying cabinet. 4 g (85% of theory) of product were isolated.

¹H NMR (600 MHz, DMSO-d₆): δ=1.51 (quin., 2H), 2.15-2.26 (m, 4H), 2.28 (t, 2H), 2.63 (t-broad, 2H), 3.35 (q, 2H), 3.79 (s, 3H), 4.41 (t, 1H), 6.85-6.90 (m, 2H), 7.38-7.43 (m, 2H), 7.97 (dt, 1H), 8.48 (dd, 1H), 8.81 (d, 1H).

### Intermediate 10-7

### 6-[3-Methoxy-8-(3-pyridyl)-6,7-dihydro-5H-benzo[7]annulen-9-yl]hex-5-yn-1-ol

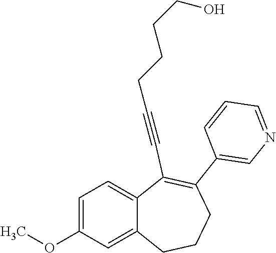 [SMILES: COc1ccc2c(c1)CCCC(c1cccnc1)=C2C#CCCCCO]

10.2 g (18.57 mmol) of 3-methoxy-8-(3-pyridyl)-6,7-dihydro-5H-benzo[7]annulen-9-yl-1,1,2,2,3,3,4,4,4-nonafluorobutane-1-sulphonate and 5.26 ml (47.71 mmol) of hex-5-yn-1-ol were reacted according to General Procedure 7. The residue was purified on silica gel 60 (mobile phase: dichloromethane, dichloromethane-acetone 9:1, 8:2, 7:3, 6:4, 1:1 and 1:3). The mixture was concentrated and the residue was taken up in ethyl acetate, washed once with phosphate buffer pH 7 and twice with water, dried over magnesium sulphate and concentrated to dryness. 5.6 g (87% of theory) of product were obtained.

¹H NMR (300 MHz, DMSO-d₆): δ=1.27-1.43 (m, 4H), 2.07-2.26 (m, 6H), 2.58 (t-broad, 2H), 3.74 (s, 3H), 4.33 (t, 1H), 6.82-6.87 (m, 2H), 7.33-7.40 (m, 2H), 7.93 (d, 1H), 8.44 (dd, 1H), 8.68 (d, 1H).

### Intermediate 11-7

### 5-[8-(4-Mesylphenyl)-3-methoxy-6,7-dihydro-5H-benzo[7]annulen-9-yl]pent-4-yn-1-ol

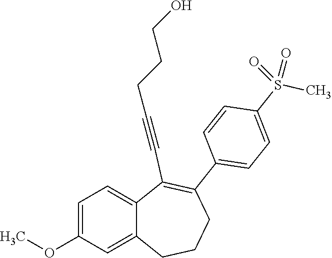

7.58 g (12.10 mmol) of 8-(4-mesylphenyl)-3-methoxy-6,7-dihydro-5H-benzo[7]annulen-9-yl-1,1,2,2,3,3,4,4,4-nonafluorobutane-1-sulphonate and 4.17 g (12.11 mmol) of pent-4-yn-1-ol were reacted according to General Procedure 7. The residue was purified on silica gel 60 (mobile phase: hexane, hexane-ethyl acetate 9:1, 4:1, 3:1 and 1:1). 4.44 g (89% of theory) of product were isolated.

¹H NMR (600 MHz, DMSO-d₆): δ=1.52 (quin., 2H), 2.16-2.27 (m, 4H), 2.30 (t, 2H), 2.63 (t, 2H), 3.24 (s, 3H), 3.25-3.35 (m, 2H), 3.79 (s, 3H), 4.42 (t, 1H), 6.85-6.90 (m, 2H), 7.41 (d, 1H), 7.85 (d, 2H), 7.92 (d, 2H).

### Intermediate 12-7

### 6-[8-(4-Mesylphenyl)-3-methoxy-6,7-dihydro-5H-benzo[7]annulen-9-yl]hex-5-yn-1-ol

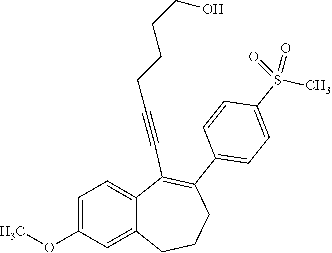

6.4 g (10.22 mmol) of 8-(4-mesyl phenyl)-3-methoxy-6,7-dihydro-5H-benzo[7]annulen-9-yl-1,1,2,2,3,3,4,4,4-nonafluorobutane-1-sulphonate and 2.82 ml (25.54 mmol) of hex-5-yn-1-ol were reacted according to General Procedure 7. The residue was purified on silica gel 60 (mobile phase: hexane, hexane-ethyl acetate 9:1, 4:1, 3:1 and 1:1). 3.64 g (84% of theory) of product were isolated.

¹H NMR (300 MHz, methanol-d₄): δ=1.47 (mc, 4H), 2.15-2.34 (m, 6H), 2.66 (t, 2H), 3.13 (s, 3H), 3.43-3.50 (m, 2H), 3.80 (s, 3H), 6.79 (d, 1H), 6.82 (dd, 1H), 7.43 (d, 1H), 7.79-7.84 (m, 2H), 7.89-7.94 (m, 2H).

### Intermediate 13-7

### 6-(3-Methoxy-8-phenyl-6,7-dihydro-5H-benzo[7]annulen-9-yl)hex-5-yn-1-ol

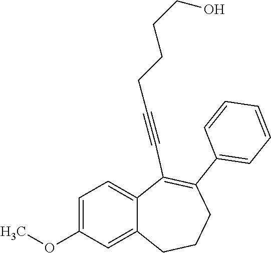 [SMILES: COc1ccc2c(c1)CCCC(c1ccccc1)=C2C#CCCCCO]

25.79 g (48.86 mmol) of 3-methoxy-8-phenyl-6,7-dihydro-5H-benzo[7]annulen-9-yl-1,1,2,2,3,3,4,4,4-nonafluorobutane-1-sulphonate and 13.28 ml (120.44 mmol) of hex-5-yn-1-ol were reacted according to General Procedure 7. The residue was purified on silica gel 60 (mobile phase: hexane, hexane-ethyl acetate 8:2 and 1:1). 11.78 g (73% of theory) of product were isolated.

¹H NMR (300 MHz, chloroform-d₁): δ=1.14 (mc, 1H), 1.45-1.57 (m, 4H), 2.14-2.40 (m, 6H), 2.69 (t, 2H), 3.50-3.63 (m, 2H), 3.84 (s, 3H), 6.76 (d, 1H), 6.84 (dd, 1H), 7.29 (mc, 1H), 7.33-7.41 (m, 2H), 7.51 (d, 1H), 7.59-7.66 (m, 2H).

### Intermediate 14-7

### 6-(4-Chloro-3-methoxy-8-phenyl-6,7-dihydro-5H-benzo[7]annulen-9-yl)hex-5-yn-1-ol

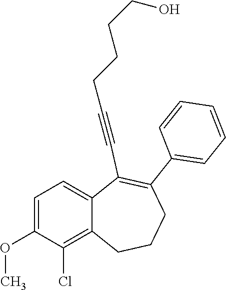 [SMILES: COc1ccc2c(c1Cl)CCCC(c1ccccc1)=C2C#CCCCCO]

24.0 g (41.2 mmol) of 4-chloro-3-methoxy-8-phenyl-6,7-dihydro-5H-benzo[7]annulen-9-yl-1,1,2,2,3,3,4,4,4-nonafluorobutane-1-sulphonate and 10.39 g (105.8 mmol) of hex-5-yn-1-ol were reacted according to General Procedure 7. The residue was purified on silica gel 60 (mobile phase: hexane, hexane-ethyl acetate 3:1 and 1:1). 14.8 g (94% of theory) of product were isolated.

¹H NMR (400 MHz, chloroform-d₁): δ=1.52 (mc, 4H), 2.18-2.34 (m, 6H), 2.96 (t, 2H), 3.57 (mc, 2H), 3.93 (s, 3H), 6.88 (d, 1H), 7.29 (tt, 1H), 7.34-7.40 (m, 2H), 7.47 (d, 1H), 7.60-7.64 (m, 2H).

### Intermediate 15-7

### 6-[4-Fluoro-8-(4-fluoro-3-methoxyphenyl)-3-methoxy-6,7-dihydro-5H-benzo[7]annulen-9-yl]hex-5-yn-1-ol

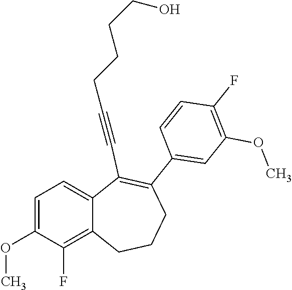 [SMILES: COc1cc(C2=C(C#CCCCCO)c3ccc(OC)c(F)c3CCC2)ccc1F]

10.7 g (17.4 mmol) of 4-fluoro-3-methoxy-8-phenyl-6,7-dihydro-5H-benzo[7]annulen-9-yl-1,1,2,2,3,3,4,4,4-nonafluorobutane-1-sulphonate and 4.39 g (44.7 mmol) of hex-5-yn-1-ol were reacted according to General Procedure 7. The residue was purified on silica gel 60 (mobile phase: hexane, hexane-ethyl acetate 8:2, 6:4 and 1:1). 5.7 g (79% of theory) of product were isolated.

¹H NMR (400 MHz, chloroform-d₁): δ=1.53 (mc, 4H), 2.15-2.24 (m, 2H), 2.27-2.36 (m, 4H), 2.78 (dt, 2H), 3.59 (mc, 2H), 3.92 (s, 3H), 3.93 (s, 3H), 6.88 (t, 1H), 7.03-7.12 (m, 2H), 7.29 (d, 1H), 7.31 (d, 1H).

### Intermediate 16-7

### 6-[2-Fluoro-8-(4-fluoro-3-methoxyphenyl)-3-methoxy-6,7-dihydro-5H-benzo[7]annulen-9-yl]hex-5-yn-1-ol

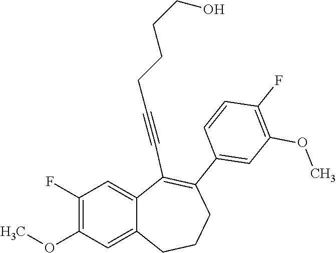

40.60 g (66.1 mmol) of 2-fluoro-8-(4-fluoro-3-methoxyphenyl)-3-methoxy-6,7-dihydro-5H-benzo[7]annulen-9-yl-1,1,2,2,3,3,4,4,4-nonafluorobutane-1-sulphonate and 16.7 g (170.1 mmol) of hex-5-yn-1-ol were reacted according to General Procedure 7, but using only 0.003 equivalents of catalyst. The residue was purified twice on silica gel 60 (mobile phase: hexane, hexane-ethyl acetate 9:1, 8:2, 7:3, 6:4 and 1:1). 7.37 g (27% of theory) of product were isolated.

¹H NMR (300 MHz, chloroform-d₁): δ=1.46-1.60 (m, 4H), 2.17-2.37 (m, 6H), 2.65 (t, 2H), 3.59 (mc, 2H), 3.92 (s, 3H), 3.93 (s, 3H), 6.79 (d, 1H), 7.02-7.11 (m, 2H), 7.27-7.34 (m, 2H).

### Intermediate 17-7

### 6-[8-(2-Fluoro-5-methoxyphenyl)-3-methoxy-6,7-dihydro-5H-benzo[7]annulen-9-yl]hex-5-yn-1-ol

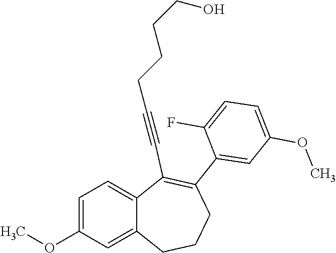

4.80 g of 8-(2-fluoro-5-methoxyphenyl)-3-methoxy-6,7-dihydro-5H-benzo[7]annulen-9-yl-1,1,2,2,3,3,4,4,4-nonafluorobutane-1-sulphonate were reacted analogously with 5-hexyn-1-ol. Purification by column chromatography on silica gel (hexane/ethyl acetate) gave 2.50 g of the title compound. C₂₅H₂₇FO₃ (394.5). MS (ESIpos) mass found: 394.00. 1H NMR (selected signals, 400 MHz, chloroform-d): δ 1.41-1.48 (m, 4H), 2.19-2.31 (m, 6H), 2.67-2.74 (m, 2H), 3.50-3.57 (m, 2H), 6.75-6.86 (m, 3H), 6.97-7.07 (d, 2H), 7.49 (d, 1H).

### Intermediate 18-7

### 6-[4-Chloro-8-(4-fluoro-3-methoxyphenyl)-3-methoxy-6,7-dihydro-5H-benzo[7]annulen-9-yl]hex-5-yn-1-ol

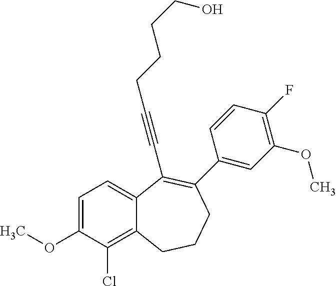

2.75 g of 4-chloro-8-(4-fluoro-3-methoxyphenyl)-3-methoxy-6,7-dihydro-5H-benzo[7]annulen-9-yl-1,1,2,2,3,3,4,4,4-nonafluorobutane-1-sulphonate were reacted analogously with 856 mg of 5-hexyn-1-ol. Purification by column chromatography on silica gel (hexane/ethyl acetate) gave 1.00 g of the title compound. C₂₅H₂₆ClFO₃ (428.94). MS (ESIpos) mass found: 428.00

### Intermediate 19-7

### 6-[8-(3-Mesylphenyl)-3-methoxy-6,7-dihydro-5H-benzo[7]annulen-9-yl]hex-5-yn-1-ol

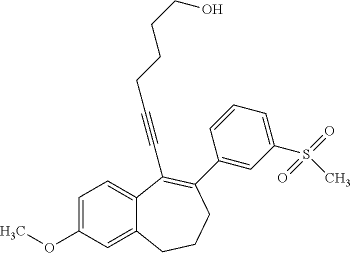 [SMILES: COc1ccc2c(c1)CCCC(c1cccc(S(C)(=O)=O)c1)=C2C#CCCCCO]

3.3 g of 8-(3-mesylphenyl)-3-methoxy-6,7-dihydro-5H-benzo[7]annulen-9-yl-1,1,2,2,3,3,4,4,4-nonafluorobutane-1-sulphonate were dissolved in 20 ml of DMF, 1.45 ml of 5-hexyn-1-ol, 3.0 ml of triethylamine and 0.30 g of Pd(PPh₃)₄ were added and the mixture was stirred at 85° C. under protective gas for 105 min. The DMF was removed under reduced pressure and the residue was dissolved in ethyl acetate, washed with water and sodium chloride solution and dried over sodium sulphate. Purification by column chromatography (silica gel, hexane/ethyl acetate) gave 1.16 g of a yellowish oil. C₂₅H₂₈O₄₅ (424.6). 1H NMR (400 MHz, chloroform-d): δ 1.47-1.55 (m, 4H), 2.22-2.37 (m, 6H), 2.66-2.73 (m, 2H), 3.10 (s, 3H), 3.55-3.61 (m, 2H), 3.84 (s, 3H), 6.76-6.78 (1H), 6.83-6.87 (1H), 7.50 (d, 1H), 7.57 (t, 1H), 7.81-7.86 (m, 2H), 8.31 (m, 1H).

### Intermediate 20-7

### 4-[9-(6-Hydroxyhex-1-yn-1-yl)-3-methoxy-6,7-dihydro-5H-benzo[7]annulen-8-yl]benzonitrile

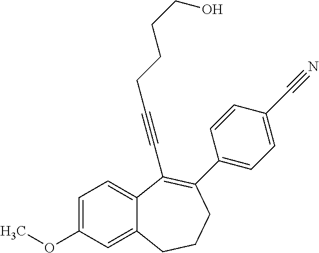 [SMILES: COc1ccc2c(c1)CCCC(c1ccc(C#N)cc1)=C2C#CCCCCO]

18.7 g of 8-(4-cyanophenyl)-3-methoxy-6,7-dihydro-5H-benzo[7]annulen-9-yl-1,1,2,2,3,3,4,4,4-nonafluorobutane-1-sulphonate were reacted analogously with 5-hexyn-1-ol. Purification by column chromatography on silica gel (hexane/ethyl acetate) and trituration with diethyl ether gave 2.01 g of 4-[9-(6-hydroxyhex-1-yn-1-yl)-3-methoxy-6,7-dihydro-5H-benzo[7]annulen-8-yl]benzonitrile as crude product. C₂₃H₁₆F₉NO₄S (573.44). 1H NMR (selected signals, 300 MHz, DMSO-d₆): δ 3.75 (s, 3H), 4.35 (t, 1H), 6.81-6.88 (m, 2H), 7.34-7.39 (m, 1H), 7.72-7.83 (m, 4H). MS (ESIpos) mass found: 371.00.

### Intermediates 8

### Intermediate 1-8

### 5-(3-Methoxy-8-phenyl-6,7-dihydro-5H-benzo[7]annulen-9-yl)pentan-1-ol

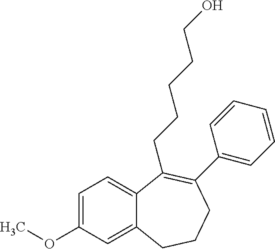 [SMILES: COc1ccc2c(c1)CCCC(c1ccccc1)=C2CCCCCO]

7.55 g (22.71 mmol) of 5-(3-methoxy-8-phenyl-6,7-dihydro-5H-benzo[7]annulen-9-yl)pent-4-yn-1-ol and 755 mg of 10% by weight palladium on activated carbon in 80 ml of ethyl acetate and 80 ml of tetrahydrofuran were hydrogenated at room temperature and standard pressure until 2 molar equivalents of hydrogen gas had been taken up. The mixture was filtered off with suction through Celite, the filter cake was washed with ethyl acetate and the filtrate was concentrated to dryness. This gave 7.64 g (100% of theory) of product.

¹H NMR (300 MHz, chloroform-d₁): δ=1.10-1.48 (m, 6H), 2.07-2.19 (m, 4H), 2.41 (mc, 2H), 2.67 (t, 2H), 3.49 (t, 2H), 3.84 (s, 3H), 6.78 (d, 1H), 6.82 (dd, 1H), 7.21-7.30 (m, 4H), 7.32-7.40 (m, 2H).

### Intermediate 2-8

### 5-[3-Methoxy-8-(3-methoxyphenyl)-6,7-dihydro-5H-benzo[7]annulen-9-yl]pentan-1-ol

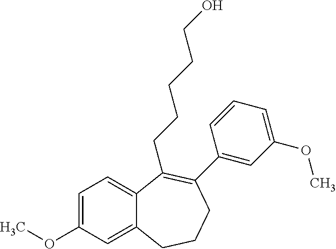

6.6 g (18.21 mmol) of 5-[3-methoxy-8-(3-methoxyphenyl)-6,7-dihydro-5H-benzo[7]annulen-9-yl]pent-4-yn-1-ol and 660 mg of 10% by weight palladium on calcium carbonate in 250 ml of tetrahydrofuran were hydrogenated at room temperature and standard pressure. The mixture was filtered off with suction through Celite, the filter cake was washed with tetrahydrofuran and the filtrate was concentrated to dryness. This gave 6.67 g (100% of theory) of product.

¹H NMR (400 MHz, chloroform-d₁): δ=1.07-1.29 (m, 4H), 1.38 (mc, 2H), 2.05-2.19 (m, 4H), 2.42 (mc, 2H), 2.66 (t, 2H), 3.50 (t, 2H), 3.84 (s, 3H), 3.84 (s, 3H), 6.76-6.87 (m, 5H), 7.21-7.30 (m, 2H).

### Intermediate 3-8

### 6-[3-Methoxy-8-(3-methoxyphenyl)-6,7-dihydro-5H-benzo[7]annulen-9-yl]hexan-1-ol

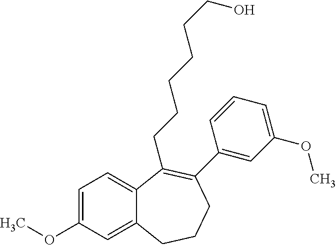

7 g (18.59 mmol) of 6-[3-methoxy-8-(3-methoxyphenyl)-6,7-dihydro-5H-benzo[7]annulen-9-yl]hex-5-yn-1-ol and 700 mg of 10% by weight palladium on calcium carbonate in 210 ml of tetrahydrofuran were hydrogenated at room temperature and standard pressure. The mixture was filtered off with suction through Celite, the filter cake was washed with tetrahydrofuran and the filtrate was concentrated to dryness. This gave 7.1 g (100% of theory) of product.

¹H NMR (400 MHz, chloroform-d₁): δ=1.10-1.27 (m, 6H), 1.37-1.46 (m, 2H), 2.06-2.17 (m, 4H), 2.40 (mc, 2H), 2.66 (t-broad, 2H), 3.53 (mc, 2H), 3.84 (s, 3H), 3.84 (s, 3H), 6.76-6.87 (m, 5H), 7.21-7.30 (m, 2H).

### Intermediate 4-8

### 5-[3-Methoxy-8-(4-methoxyphenyl)-6,7-dihydro-5H-benzo[7]annulen-9-yl]pentan-1-ol

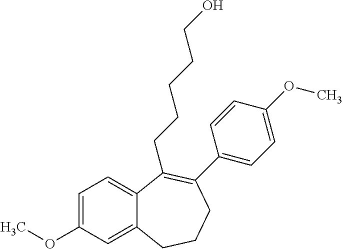

3.56 g (9.82 mmol) of 5-[3-methoxy-8-(4-methoxyphenyl)-6,7-dihydro-5H-benzo[7]annulen-9-yl]pent-4-yn-1-ol and 356 mg of 10% by weight palladium on activated carbon in 40 ml of tetrahydrofuran and 40 ml of ethyl acetate were hydrogenated at room temperature and standard pressure. The mixture was filtered off with suction through Celite, the filter cake was washed with ethyl acetate and the filtrate was concentrated to dryness. This gave 3.42 g (95% of theory) of product.

¹H NMR (400 MHz, chloroform-d₁): δ=1.12-1.46 (m, 6H), 2.06-2.18 (m, 4H), 2.42 (mc, 2H), 2.65 (t-broad, 2H), 3.50 (t, 2H), 3.84 (s, 6H), 6.77 (d, 1H), 6.81 (dd, 1H), 6.90 (mc, 2H), 7.18 (mc, 2H), 7.23 (d, 1H).

### Intermediate 5-8

### 6-[3-Methoxy-8-(4-methoxyphenyl)-6,7-dihydro-5H-benzo[7]annulen-9-yl]hexan-1-ol

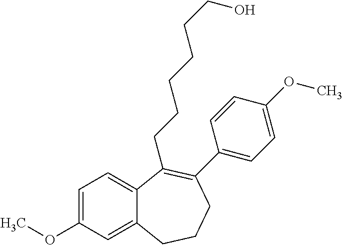

4.16 g (11.05 mmol) of 6-[3-methoxy-8-(4-methoxyphenyl)-6,7-dihydro-5H-benzo[7]annulen-9-yl]hex-5-yn-1-ol and 420 mg of 10% by weight palladium on activated carbon in 45 ml of tetrahydrofuran and 70 ml of ethyl acetate were hydrogenated at room temperature and standard pressure. The mixture was filtered off with suction through Celite, the filter cake was washed with ethyl acetate and the filtrate was concentrated to dryness. This gave 4.2 g (100% of theory) of product.

¹H NMR (300 MHz, chloroform-d₁): δ=1.09-1.30 (m, 6H), 1.36-1.49 (m, 2H), 1.59 (s-broad, 1H), 2.06-2.17 (m, 4H), 2.40 (mc, 2H), 2.65 (mc, 2H), 3.53 (t, 2H), 3.84 (s, 6H), 6.77 (d, 1H), 6.81 (dd, 1H), 6.90 (mc, 2H), 7.14-7.25 (m, 3H).

### Intermediate 6-8

### 6-[8-(4-Fluoro-3-methoxyphenyl)-3-methoxy-6,7-dihydro-5H-benzo[7]annulen-9-yl]hexan-1-ol

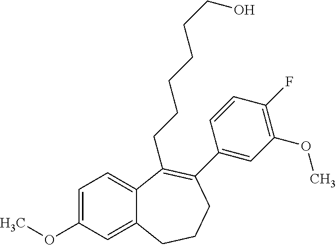

6.5 g (16.48 mmol) of 6-[8-(4-fluoro-3-methoxyphenyl)-3-methoxy-6,7-dihydro-5H-benzo[7]annulen-9-yl]hex-5-yn-1-ol and 650 mg of 10% by weight palladium on calcium carbonate in 250 ml of tetrahydrofuran were hydrogenated at room temperature and standard pressure. The mixture was filtered off with suction through Celite, the filter cake was washed with tetrahydrofuran and the filtrate was concentrated to dryness. This gave 6.57 g (100% of theory) of product.

¹H NMR (400 MHz, chloroform-d₁): δ=1.10-1.30 (m, 6H), 1.37-1.49 (m, 2H), 2.05-2.17 (m, 4H), 2.38 (mc, 2H), 2.65 (t, 2H), 3.54 (mc, 2H), 3.84 (s, 3H), 3.91 (s, 3H), 6.75-6.86 (m, 4H), 7.05 (dd, 1H), 7.23 (d, 1H).

### Intermediate 7-8

### 5-[8-(3-Fluoro-4-methoxyphenyl)-3-methoxy-6,7-dihydro-5H-benzo[7]annulen-9-yl]pentan-1-ol

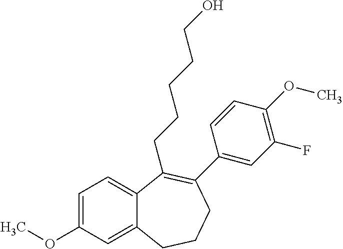

4.56 g (11.99 mmol) of 5-[8-(3-fluoro-4-methoxyphenyl)-3-methoxy-6,7-dihydro-5H-benzo[7]annulen-9-yl]pent-4-yn-1-ol and 533.6 mg of 10% by weight palladium on activated carbon in 50 ml of ethyl acetate and 50 ml of tetrahydrofuran were hydrogenated at room temperature and standard pressure. The mixture was filtered off with suction through Celite, the filter cake was washed with ethyl acetate and the filtrate was concentrated to dryness. This gave 4.58 g (99% of theory) of product.

¹H NMR (400 MHz, chloroform-d₁): δ=1.09-1.28 (m, 4H), 1.39 (mc., 2H), 2.03-2.16 (m, 4H), 2.41 (mc, 2H), 2.64 (t, 2H), 3.51 (t, 2H), 3.84 (s, 3H), 3.92 (s, 3H), 6.77 (d, 1H), 6.81 (dd, 1H), 6.90-7.02 (m, 3H), 7.22 (d, 1H).

### Intermediate 8-8

### 5-[3-Methoxy-8-(3-pyridyl)-6,7-dihydro-5H-benzo[7]annulen-9-yl]pentan-1-ol

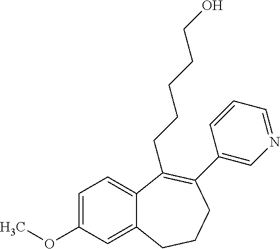 [SMILES: COc1ccc2c(c1)CCCC(c1cccnc1)=C2CCCCCO]

5.4 g (16.20 mmol) of 5-[3-methoxy-8-(3-pyridyl)-6,7-dihydro-5H-benzo[7]annulen-9-yl]pent-4-yn-1-ol and 800 mg of 10% by weight palladium on activated carbon were hydrogenated at room temperature and standard pressure. The mixture was filtered off with suction through Celite, the filter cake was washed with tetrahydrofuran and the filtrate was concentrated and dried at 50° C. under oil pump vacuum. 5.4 g (99% of theory) of product were isolated.

¹H NMR (300 MHz, DMSO-d₆): δ=0.97-1.26 (m, 6H), 1.91-2.11 (m, 4H), 2.30 (mc, 2H), 2.60 (t, 2H), 3.19 (t, 2H), 3.74 (s, 3H), 4.20 (s-broad, 1H), 6.78-6.85 (m, 2H), 7.25 (mc, 1H), 7.38 (ddd, 1H), 7.64 (dt, 1H), 8.42-8.49 (m, 2H).

### Intermediate 9-8

### 6-[3-Methoxy-8-(3-pyridyl)-6,7-dihydro-5H-benzo[7]annulen-9-yl]hexan-1-ol

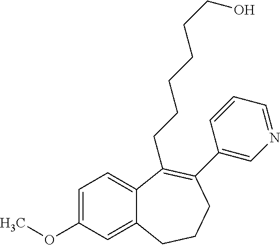 [SMILES: COc1ccc2c(c1)CCCC(c1cccnc1)=C2CCCCCCO]

5.1 g (14.68 mmol) of 6-[3-methoxy-8-(3-pyridyl)-6,7-dihydro-5H-benzo[7]annulen-9-yl]hex-5-yn-1-ol and 714 mg of 10% by weight palladium on activated carbon in 100 ml of tetrahydrofuran were hydrogenated at room temperature and standard pressure. The mixture was filtered off with suction through Celite, the filter cake was washed with tetrahydrofuran and the filtrate was concentrated and dried at 50° C. under oil pump vacuum. 5.1 g (99% of theory) of product were obtained.

¹H NMR (400 MHz, DMSO-d₆): δ=0.95-1.28 (m, 8H), 1.93-2.10 (m, 4H), 2.30 (mc, 2H), 2.60 (t, 2H), 3.22 (t, 2H), 3.74 (s, 3H), 4.20 (s-broad, 1H), 6.78-6.86 (m, 2H), 7.25 (mc, 1H), 7.41 (dd, 1H), 7.67 (dt, 1H), 8.44-8.50 (m, 2H).

### Intermediate 10-8

### 5-[8-(4-Mesylphenyl)-3-methoxy-6,7-dihydro-5H-benzo[7]annulen-9-yl]pentan-1-ol

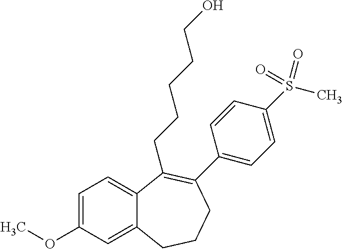

4.4 g (10.72 mmol) of 5-[8-(4-mesylphenyl)-3-methoxy-6,7-dihydro-5H-benzo[7]annulen-9-yl]pent-4-yn-1-ol and 388.5 mg of 10% by weight palladium on activated carbon in 45 ml of ethyl acetate and 45 ml of tetrahydrofuran were hydrogenated at room temperature and standard pressure. The mixture was filtered off with suction through Celite, the filter cake was washed with ethyl acetate and the filtrate was concentrated. The residue was purified on silica gel 60 (mobile phase: hexane, hexane-ethyl acetate 1:1). 3.77 g (85% of theory) of product were isolated.

¹H NMR (600 MHz, DMSO-d₆): δ=1.05-1.23 (m, 6H), 2.00-2.12 (m, 4H), 2.35 (mc, 2H), 2.64 (t, 2H), 3.22 (mc, 2H), 3.25 (s, 3H), 3.78 (s, 3H), 4.23 (t, 1H), 6.84-6.87 (m, 2H), 7.29 (mc, 1H), 7.52 (mc, 2H), 7.93 (mc, 2H).

### Intermediate 11-8

### 6-[8-(4-Mesylphenyl)-3-methoxy-6,7-dihydro-5H-benzo[7]annulen-9-yl]hexan-1-ol

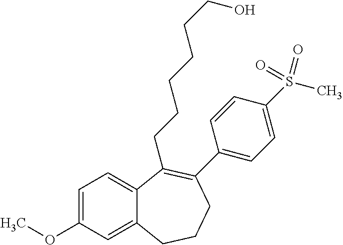

3.64 g (8.57 mmol) of 6-[8-(4-mesylphenyl)-3-methoxy-6,7-dihydro-5H-benzo[7]annulen-9-yl]hex-5-yn-1-ol and 337.6 mg of 10% by weight palladium on calcium carbonate in 35 ml of to ethyl acetate and 35 ml of tetrahydrofuran were hydrogenated at room temperature and standard pressure. The mixture was filtered off with suction through Celite, the filter cake was washed with ethyl acetate and the filtrate was concentrated. The residue was purified on silica gel 60 (mobile phase: hexane, hexane-ethyl acetate 3:1 and 1:1). This gave 2.8 g (76% of theory) of product.

¹H NMR (400 MHz, chloroform-d₁): δ=1.07-1.47 (m, 8H), 2.03-2.23 (m, 4H), 2.36 (mc, 2H), 2.67 (t, 2H), 3.10 (s, 3H), 3.53 (t, 2H), 3.84 (s, 3H), 6.79 (d, 1H), 6.83 (dd, 1H), 7.23 (d, 1H), 7.45 (mc, 2H), 7.93 (mc, 2H).

### Intermediate 12-8

### 6-(3-Methoxy-8-phenyl-6,7-dihydro-5H-benzo[7]annulen-9-yl)hexan-1-ol

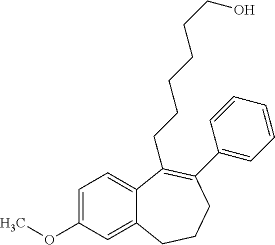 [SMILES: COc1ccc2c(c1)CCCC(c1ccccc1)=C2CCCCCCO]

8.45 g (24.39 mmol) of 6-(3-methoxy-8-phenyl-6,7-dihydro-5H-benzo[7]annulen-9-yl)hex-5-yn-1-ol and 422.5 mg of 5% by weight palladium on activated carbon in 340 ml of 0.02 M methanolic KOH were hydrogenated at room temperature and standard pressure. The resulting precipitate was dissolved in tetrahydrofuran. The mixture was filtered off with suction through Celite, the filter cake was washed with tetrahydrofuran and the filtrate was concentrated. The residue was taken up in ethyl acetate and washed three times with water, dried over magnesium sulphate and concentrated. The crude product was dissolved in 60 ml of methanol, and 5.84 g of potassium carbonate were added. The mixture was stirred at room temperature for 2 hours. 150 ml of water were added, and the mixture was extracted three times with methyl tert-butyl ether. The combined organic phases were washed once with water and once with saturated sodium chloride solution, dried over magnesium sulphate and concentrated. This gave 8.0 g (94% of theory) of a white solid.

¹H NMR (300 MHz, chloroform-d₁): δ=0.89 (t, 1H), 1.05-1.33 (m, 6H), 1.35-1.48 (m, 2H), 2.06-2.20 (m, 4H), 2.39 (mc, 2H), 2.67 (t, 2H), 3.53 (mc, 2H), 3.84 (s, 3H), 6.78 (d, 1H), 6.82 (dd, 1H), 7.21-7.29 (m, 4H), 7.32-7.40 (m, 2H).

### Intermediate 13-8

### 6-(4-Chloro-3-methoxy-8-phenyl-6,7-dihydro-5H-benzo[7]annulen-9-yl)hexan-1-ol

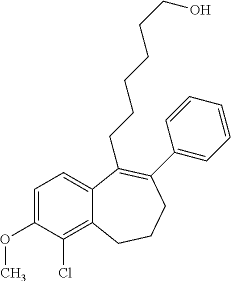 [SMILES: COc1ccc2c(c1Cl)CCCC(c1ccccc1)=C2CCCCCCO]

12.7 g (33.3 mmol) of 6-(4-chloro-3-methoxy-8-phenyl-6,7-dihydro-5H-benzo[7]annulen-9-yl)hex-5-yn-1-ol were dissolved in 200 ml of methanol with 2 g of potassium hydroxide. 1.52 g of 5% by weight palladium on activated carbon were added, and the mixture was hydrogenated at room temperature and standard pressure. The mixture was filtered off with suction through Celite, the filter cake was washed with methanol and the filtrate was concentrated. The residue was taken up in dichloromethane and washed three times with water, dried over magnesium sulphate and concentrated. The material was chromatographed on silica gel 60 (mobile phase: hexane, hexane-ethyl acetate 3:1 and 1:1). This gave 12.5 g (100% of theory) of product.

¹H NMR (300 MHz, chloroform-d₁): δ=1.10-1.27 (m, 6H), 1.42 (mc, 2H), 2.02-2.18 (m, 4H), 2.40 (mc, 2H), 2.95 (mc, 2H), 3.53 (t, 2H), 3.93 (s, 3H), 6.85 (d, 1H), 7.19 (d, 1H), 7.21-7.30 (m, 3H), 7.33-7.40 (m, 2H).

### Intermediate 14-8

### 6-[4-Fluoro-8-(4-fluoro-3-methoxyphenyl)-3-methoxy-6,7-dihydro-5H-benzo[7]annulen-9-yl]hexan-1-ol

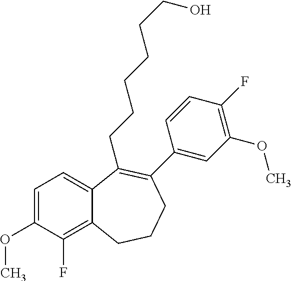 [SMILES: COc1cc(C2=C(CCCCCCO)c3ccc(OC)c(F)c3CCC2)ccc1F]

5.7 g (13.8 mmol) of 6-[4-fluoro-8-(4-fluoro-3-methoxyphenyl)-3-methoxy-6,7-dihydro-5H-benzo[7]annulen-9-yl]hex-5-yn-1-ol and 0.632 g of 5% by weight palladium on activated carbon in 100 ml of 0.2% strength methanolic potassium hydroxide solution were hydrogenated at room temperature and standard pressure. The mixture was filtered off with suction through Celite, the filter cake was washed with methanol and the filtrate was concentrated. The residue was taken up in dichloromethane and washed three times with water, dried over magnesium sulphate and concentrated. This gave 5.4 g (84% of theory) of product.

¹H NMR (300 MHz, chloroform-d₁): δ=1.10-1.29 (m, 6H), 1.37-1.49 (m, 2H), 2.02-2.16 (m, 4H), 2.32-2.42 (m, 2H), 2.70-2.80 (m, 2H), 3.49-3.60 (m, 2H), 3.91 (s, 3H), 3.92 (s, 3H), 6.75 (ddd, 1H), 6.79-6.90 (m, 2H), 7.00-7.09 (m, 2H).

### Intermediate 15-8

### 6-[2-Fluoro-8-(4-fluoro-3-methoxyphenyl)-3-methoxy-6,7-dihydro-5H-benzo[7]annulen-9-yl]hexan-1-ol

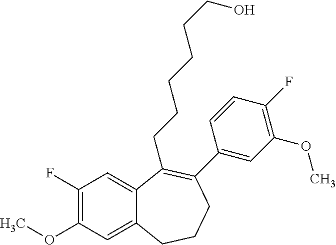

7.35 g (17.8 mmol) of 6-[2-fluoro-8-(4-fluoro-3-methoxyphenyl)-3-methoxy-6,7-dihydro-5H-benzo[7]annulen-9-yl]hex-5-yn-1-ol and 0.796 g of 5% by weight palladium on activated carbon in 200 ml of 0.2% strength methanolic potassium hydroxide solution were hydrogenated at room temperature and standard pressure. The mixture was filtered off with suction through Celite, the filter cake was washed with methanol and the filtrate was concentrated. The residue was taken up in dichloromethane and washed three times with water, dried over magnesium sulphate and concentrated. This gave 6.82 g (92% of theory) of product.

¹H NMR (300 MHz, chloroform-d₁): δ=1.09-1.29 (m, 6H), 1.37-1.49 (m, 2H), 2.03-2.19 (m, 4H), 2.28-2.37 (m, 2H), 2.62 (t, 2H), 3.54 (mc, 2H), 3.91 (s, 3H), 3.92 (s, 3H), 6.75 (ddd, 1H), 6.79-6.85 (m, 2H), 7.00-7.09 (m, 2H).

### Intermediate 16-8

### 6-[8-(3-Mesylphenyl)-3-methoxy-6,7-dihydro-5H-benzo[7]annulen-9-yl]hexan-1-ol

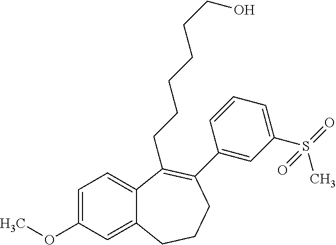 [SMILES: COc1ccc2c(c1)CCCC(c1cccc(S(C)(=O)=O)c1)=C2CCCCCCO]

Starting with 1.1 g of 6-[8-(3-mesylphenyl)-3-methoxy-6,7-dihydro-5H-benzo[7]annulen-9-yl]hex-5-yn-1-ol (crude product), 1.10 g of the title compound were prepared analogously as crude product. C₂₅H₃₂O4 S (428.6). MS (ESIpos) mass found: 428.00.

### Intermediate 17-8

### 4-[9-(6-Hydroxyhexyl)-3-methoxy-6,7-dihydro-5H-benzo[7]annulen-8-yl]benzonitrile

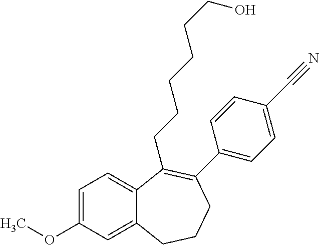 [SMILES: COc1ccc2c(c1)CCCC(c1ccc(C#N)cc1)=C2CCCCCCO]

Starting with 2.00 g of 4-[9-(6-hydroxyhex-1-yn-1-yl)-3-methoxy-6,7-dihydro-5H-benzo[7]annulen-8-yl]benzonitrile (crude product), 2.35 g of 4-[9-(6-hydroxyhexyl)-3-methoxy-6,7-dihydro-5H-benzo[7]annulen-8-yl]benzonitrile were obtained analogously as crude product.

### Intermediate 18-8

### 6-[8-(2-Fluoro-5-methoxyphenyl)-3-methoxy-6,7-dihydro-5H-benzo[7]annulen-9-yl]hexan-1-ol

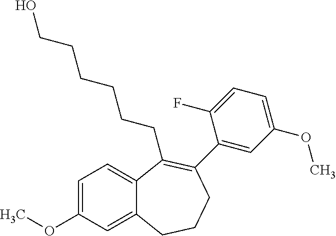

2.50 g of 6-[8-(2-fluoro-5-methoxyphenyl)-3-methoxy-6,7-dihydro-5H-benzo[7]annulen-9-yl]hex-5-yn-1-ol were reacted analogously in the presence of 10% palladium on calcium carbonate to give 2.48 g (98% of theory) of the title compound. C₂₅H₃₁FO₃ (398.52). MS (ES+) mass found 399.

### Intermediate 19-8

### 6-[4-Chloro-8-(4-fluoro-3-methoxyphenyl)-3-methoxy-6,7-dihydro-5H-benzo[7]annulen-9-yl]hexan-1-ol

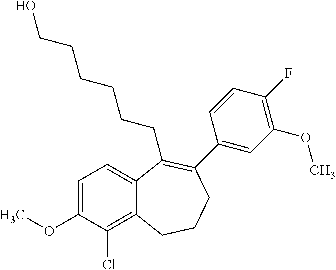

5% palladium on carbon was added to a mixture of 1.00 g of 6-[4-chloro-8-(4-fluoro-3-methoxyphenyl)-3-methoxy-6,7-dihydro-5H-benzo[7]annulen-9-yl]hex-5-yn-1-ol and 200 mg of potassium hydroxide in 20 ml of methanol, and the mixture was stirred under an atmosphere of hydrogen. The mixture was filtered and concentrated, giving 1.30 g of a crude product which was reacted further without purification. C₂₅H₃₀ClFO₃ (432.97). MS (ESIpos) mass found: 432.00.

### Intermediate 20-8

### 5-[8-(4-Fluoro-3-methoxyphenyl)-3-methoxy-6,7-dihydro-5H-benzo[7]annulen-9-yl]pentan-1-ol

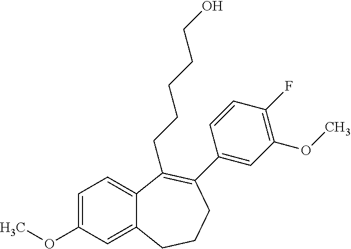

20.8 g of 5-[8-(4-fluoro-3-methoxyphenyl)-3-methoxy-6,7-dihydro-5H-benzo[7]annulen-9-yl]pent-4-yn-1-ol were reacted analogously in the presence of palladium on calcium carbonate. This gave 20.1 g (96% of theory) of a crude product.

### Intermediates 9

General Procedure 9-1 for the preparation of 9 under an atmosphere of protective gas and with exclusion of moisture: 1 g of methyl ether were dissolved in about 11.1-20.3 ml of anhydrous N,N-dimethylformamide. 2.7-5.5 equivalents of sodium thiomethoxide were added, and the mixture was stirred at 140° C. for 4-9 hours. The mixture was cooled to room temperature, poured into water and extracted three or four times with ethyl acetate, and the extracts were washed with water, semisaturated sodium chloride solution and saturated sodium chloride solution, dried over magnesium sulphate or sodium sulphate and concentrated to dryness.

General Procedure 9-2 for the preparation of 9 under an atmosphere of protective gas and with exclusion of moisture: At 3-5° C., 3.5 equivalents of 2,6-dimethylpyridine in dichloromethane (about 4.4-5.5 ml/g) were added to 3.5 equivalents of boron tribromide (1 mmol of boron tribromide in 1.5-4 ml of dichloromethane). At 3-5° C., 1 equivalent of methyl ether, dissolved in dichloromethane (4.3-6.1 ml/g), was added dropwise, and the mixture was stirred at room temperature overnight. The mixture was poured onto ice-water, the phases were separated and the aqueous phase was extracted three times with dichloromethane.

The combined organic phases were washed with water, dried over magnesium sulphate and concentrated.

### Intermediate 1-9

### 9-(5-Hydroxypentyl)-8-phenyl-6,7-dihydro-5H-benzo[7]annulen-3-ol

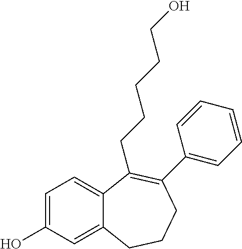 [SMILES: OCCCCCC1=C(c2ccccc2)CCCc2cc(O)ccc21]

7.4 g (21.99 mmol) of 5-(3-methoxy-8-phenyl-6,7-dihydro-5H-benzo[7]annulen-9-yl)pentan-1-ol and 4.14 g of sodium thiomethoxide were reacted according to General Procedure 9-1. The residue was chromatographed on silica gel 60 (mobile phase: hexane, hexane-ethyl acetate 9:1, 8:2, 7:3 and 6:4). The solid was triturated with diisopropyl ether, filtered off with suction and dried. This gave 3.42 g (48% of theory) of product.

¹H NMR (300 MHz, methanol-d₄): δ=1.06-1.36 (m, 6H), 1.99-2.14 (m, 4H), 2.37 (mc, 2H), 2.61 (mc, 2H), 3.36 (t, 2H), 6.62-6.71 (m, 2H), 7.12 (d, 1H), 7.16-7.24 (m, 3H), 7.28-7.36 (m, 2H).

### Intermediate 2-9

### 9-(5-Hydroxypentyl)-8-(3-hydroxyphenyl)-6,7-dihydro-5H-benzo[7]annulen-3-ol

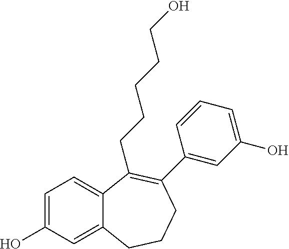 [SMILES: OCCCCCC1=C(c2cccc(O)c2)CCCc2cc(O)ccc21]

6.6 g (18.01 mmol) of 5-[3-methoxy-8-(3-methoxyphenyl)-6,7-dihydro-5H-benzo[7]annulen-9-yl]pentan-1-ol and 6.82 g of sodium thiomethoxide were reacted according to General Procedure 9-1. The residue was purified on silica gel 60 (mobile phase: hexane, hexane-ethyl acetate 8:2, 6:4 and 4:6). This gave 4 g (66% of theory) of product.

¹H NMR (300 MHz, methanol-d₄): δ=1.07-1.40 (m, 6H), 1.98-2.13 (m, 4H), 2.38 (mc, 2H), 2.60 (t, 2H), 3.38 (t, 2H), 6.61-6.70 (m, 5H), 7.08-7.17 (m, 2H).

### Intermediate 3-9

### 9-(6-Hydroxyhexyl)-8-(3-hydroxyphenyl)-6,7-dihydro-5H-benzo[7]annulen-3-ol

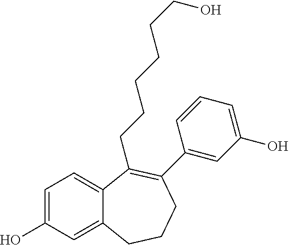 [SMILES: OCCCCCCC1=C(c2cccc(O)c2)CCCc2cc(O)ccc21]

7.6 g (19.97 mmol) of 6-[3-methoxy-8-(3-methoxyphenyl)-6,7-dihydro-5H-benzo[7]annulen-9-yl]hexan-1-ol and 7.56 g of sodium thiomethoxide were reacted according to Procedure 9-1. 40 ml of diisopropyl ether were added to the residue, and the solid residue was filtered off with suction and dried. This gave 3.52 g (50% of theory) of product. The mother liquor was chromatographed on silica gel 60 (mobile phase: hexane, hexane-ethyl acetate 8:2, 6:4 and 4:6). 0.82 g (12% of theory) of product were obtained.

¹H NMR (600 MHz, DMSO-d₆): δ=1.02-1.16 (m, 6H), 1.25 (mc, 2H), 1.93-1.98 (m, 2H), 1.98-2.06 (m, 2H), 2.33 (mc, 2H), 2.53 (t, 2H), 3.26 (mc, 2H), 4.24 (t, 1H), 6.61-6.68 (m, 5H), 7.09-7.16 (m, 2H), 9.28 (s-broad, 1H), 9.32 (s-broad, 1H).

### Intermediate 4-9

### 9-(5-Hydroxypentyl)-8-(4-hydroxyphenyl)-6,7-dihydro-5H-benzo[7]annulen-3-ol

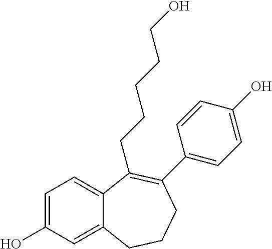 [SMILES: OCCCCCC1=C(c2ccc(O)cc2)CCCc2cc(O)ccc21]

3.4 g (9.28 mmol) of 5-[3-methoxy-8-(4-methoxyphenyl)-6,7-dihydro-5H-benzo[7]annulen-9-yl]pentan-1-ol and 3.51 g of sodium thiomethoxide were reacted according to General Procedure 9-1. The residue was chromatographed on silica gel 60 (mobile phase: hexane, hexane-ethyl acetate 9:1 and 8:2). 2.12 g (68% of theory) of product were obtained.

¹H NMR (300 MHz, methanol-d₄): δ=1.08-1.26 (m, 4H), 1.32 (mc, 2H), 1.97-2.11 (m, 4H), 2.38 (mc, 2H), 2.58 (mc, 2H), 3.38 (t, 2H), 6.60-6.69 (m, 2H), 6.74 (mc, 2H), 7.03 (mc, 2H), 7.10 (d, 1H).

### Intermediate 5-9

### 9-(6-Hydroxyhexyl)-8-(4-hydroxyphenyl)-6,7-dihydro-5H-benzo[7]annulen-3-ol

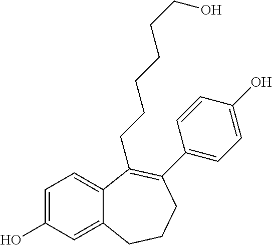 [SMILES: OCCCCCCC1=C(c2ccc(O)cc2)CCCc2cc(O)ccc21]

4.19 g (11.01 mmol) of 6-[3-methoxy-8-(4-methoxyphenyl)-6,7-dihydro-5H-benzo[7]annulen-9-yl]hexan-1-ol and 4.17 g of sodium thiomethoxide were reacted according to General Procedure 9-1. The crude product was purified on silica gel 60 (mobile phase: hexane, hexane-ethyl acetate 9:1, 8:2, 7:3, 6:4, 1:1 and 2:3). This gave 3.48 g (90% of theory) of product.

¹H NMR (300 MHz, methanol-d₄): δ=1.05-1.25 (m, 6H), 1.28-1.43 (m, 2H), 1.96-2.12 (m, 4H), 2.38 (mc, 2H), 2.58 (t, 2H), 3.41 (t, 2H), 6.59-6.70 (m, 2H), 6.70-6.77 (m, 2H), 6.99-7.06 (m, 2H), 7.10 (d, 1H).

### Intermediate 6-9

### 8-(4-Fluoro-3-hydroxyphenyl)-9-(6-hydroxyhexyl)-6,7-dihydro-5H-benzo[7]annulen-3-ol

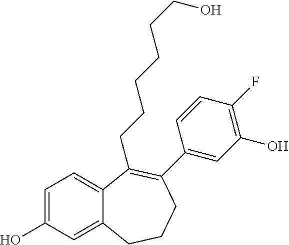 [SMILES: OCCCCCCC1=C(c2ccc(F)c(O)c2)CCCc2cc(O)ccc21]

6.5 g (16.31 mmol) of 6-[8-(4-fluoro-3-methoxyphenyl)-3-methoxy-6,7-dihydro-5H-benzo[7]annulen-9-yl]hexan-1-ol and 6.17 g of sodium thiomethoxide were reacted according to General Procedure 9-1. 50 ml of diisopropyl ether were added to the residue and the mixture was stored overnight in a freezer, filtered off with suction and dried in a drying cabinet overnight. 1.55 g (25% of theory) of white crystals were obtained. The mother liquor was chromatographed on silica gel 60 (mobile phase: hexane, hexane-ethyl acetate 8:2, 6:4 and 4:6). 4.35 g (72% of theory) of a white product were obtained.

¹H NMR (300 MHz, methanol-d₄): δ=1.05-1.26 (m, 6H), 1.28-1.43 (m, 2H), 1.95-2.15 (m, 4H), 2.37 (mc, 2H), 2.59 (t, 2H), 3.41 (t, 2H), 6.58-6.70 (m, 3H), 6.76 (dd, 1H), 6.98 (dd, 1H), 7.10 (d, 1H).

### Intermediate 7-9

### 8-(3-Fluoro-4-hydroxyphenyl)-9-(5-hydroxypentyl)-6,7-dihydro-5H-benzo[7]annulen-3-ol

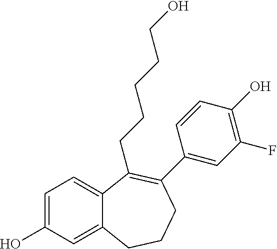 [SMILES: OCCCCCC1=C(c2ccc(O)c(F)c2)CCCc2cc(O)ccc21]

4.58 g (11.91 mmol) of 5-[8-(3-fluoro-4-methoxyphenyl)-3-methoxy-6,7-dihydro-5H-benzo[7]annulen-9-yl]pentan-1-ol and 4.62 g of sodium thiomethoxide were reacted according to General Procedure 9-1. The residue was purified on silica gel 60 (mobile phase: hexane, hexane-ethyl acetate 9:1, 8:2, 7:3, 6:4 and 1:1). This gave 2.9 g (68% of theory) of product.

¹H NMR (300 MHz, methanol-d₄): δ=1.07-1.40 (m, 6H), 1.96-2.12 (m, 4H), 2.34-2.44 (m, 2H), 2.58 (t, 2H), 3.38 (t, 2H), 6.60-6.69 (m, 2H), 6.83-6.93 (m, 3H), 7.11 (d, 1H).

### Intermediate 8-9

### 9-(5-Hydroxypentyl)-8-(3-pyridyl)-6,7-dihydro-5H-benzo[7]annulen-3-ol

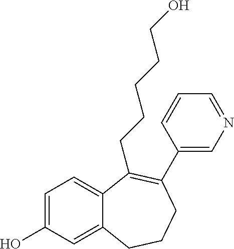 [SMILES: OCCCCCC1=C(c2cccnc2)CCCc2cc(O)ccc21]

5.4 g (16.00 mmol) of 5-[3-methoxy-8-(3-pyridyl)-6,7-dihydro-5H-benzo[7]annulen-9-yl]pentan-1-ol and 3.02 g of sodium thiomethoxide were reacted according to General Procedure 9-1. Diisopropyl ether was added to the residue, the mixture was filtered off with suction and the material was dried in a drying cabinet overnight. This gave 3.35 g (68% of theory) of product.

¹H NMR (300 MHz, DMSO-d₆): δ=0.94-1.23 (m, 6H), 1.88-2.08 (m, 4H), 2.27 (mc, 2H), 2.52 (mc, 2H), 3.19 (mc, 2H), 4.21 (t, 1H), 6.58-6.69 (m, 2H), 7.12 (d, 1H), 7.36 (ddd, 1H), 7.61 (dt, 1H), 8.39-8.51 (m, 2H), 9.32 (s, 1H).

### Intermediate 9-9

### 9-(6-Hydroxyhexyl)-8-(3-pyridyl)-6,7-dihydro-5H-benzo[7]annulen-3-ol

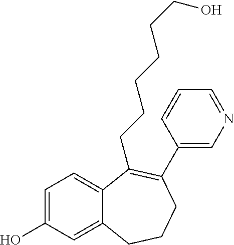 [SMILES: OCCCCCCC1=C(c2cccnc2)CCCc2cc(O)ccc21]

5.1 g (14.51 mmol) of 6-[3-methoxy-8-(3-pyridyl)-6,7-dihydro-5H-benzo[7]annulen-9-yl]hexan-1-ol and 2.74 g of sodium thiomethoxide were reacted according to General Procedure 9-1. The residue was stirred in diisopropyl ether and diethyl ether, filtered off with suction and dried. This gave 1.85 g (38% of theory) of product.

¹H NMR (300 MHz, DMSO-d₆): δ=0.93-1.27 (m, 8H), 1.88-2.07 (m, 4H), 2.27 (mc, 2H), 2.52 (t, 2H), 3.22 (mc, 2H), 4.22 (t, 1H), 6.59-6.67 (m, 2H), 7.11 (d, 1H), 7.36 (dd, 1H), 7.61 (dt, 1H), 8.39-8.47 (m, 2H), 9.32 (s, 1H).

### Intermediate 10-9

### 9-(5-Hydroxypentyl)-8-(4-mesylphenyl)-6,7-dihydro-5H-benzo[7]annulen-3-ol

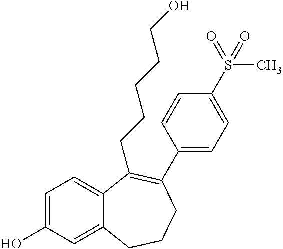 [SMILES: CS(=O)(=O)c1ccc(C2=C(CCCCCO)c3ccc(O)cc3CCC2)cc1]

3.7 g (8.93 mmol) of 5-[8-(4-mesylphenyl)-3-methoxy-6,7-dihydro-5H-benzo[7]annulen-9-yl]pentan-1-ol and 3.38 g of sodium thiomethoxide were reacted according to General Procedure 9-1. The residue was purified on silica gel 60 (mobile phase: hexane, hexane-ethyl acetate 1:1 and 2:3). This gave 1.18 g (33% of theory) of product.

¹H NMR (400 MHz, DMSO-d₆): δ=1.03-1.26 (m, 6H), 1.98-2.11 (m, 4H), 2.33 (mc, 2H), 2.57 (mc, 2H), 3.18-3.27 (m, 5H), 4.21 (t, 1H), 6.65-6.72 (m, 2H), 7.16 (d, 1H), 7.51 (d, 2H), 7.92 (d, 2H), 9.34 (s, 1H).

### Intermediate 11-9

### 9-(6-Hydroxyhexyl)-8-(4-mesylphenyl)-6,7-dihydro-5H-benzo[7]annulen-3-ol

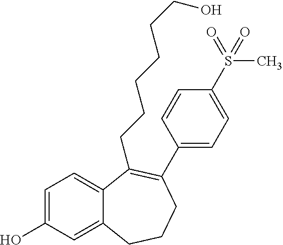 [SMILES: CS(=O)(=O)c1ccc(C2=C(CCCCCCO)c3ccc(O)cc3CCC2)cc1]

2.8 g (6.53 mmol) of 6-[8-(4-mesylphenyl)-3-methoxy-6,7-dihydro-5H-benzo[7]annulen-9-yl]hexan-1-ol and 2.47 g of sodium thiomethoxide were reacted according to General Procedure 9-1. The residue was purified on silica gel 60 (mobile phase: hexane, hexane-ethyl acetate 1:1 and 1:4). This gave 0.63 g (23% of theory) of product.

¹H NMR (400 MHz, chloroform-d₁): δ=1.06-1.29 (m, 6H), 1.40 (mc, 2H), 1.76 (s-broad, 1H), 2.00-2.17 (m, 4H), 2.34 (mc, 2H), 2.62 (t, 2H), 3.11 (s, 3H), 3.53 (t, 2H), 6.32 (s-broad, 1H), 6.72-6.81 (m, 2H), 7.16 (d, 1H), 7.44 (d, 2H), 7.92 (d, 2H).

### Intermediate 12-9

### 9-(6-Hydroxyhexyl)-8-phenyl-6,7-dihydro-5H-benzo[7]annulen-3-ol

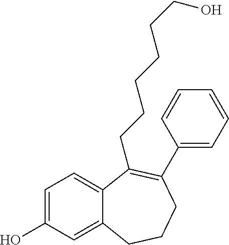 [SMILES: OCCCCCCC1=C(c2ccccc2)CCCc2cc(O)ccc21]

8.75 g (24.96 mmol) of 6-(3-methoxy-8-phenyl-6,7-dihydro-5H-benzo[7]annulen-9-yl)hexan-1-ol and 9.45 g of sodium thiomethoxide were reacted according to General Procedure 9-1. The residue was recrystallized from ethyl acetate and hexane and dried in a vacuum drying cabinet at 40° C. 5.6 g (67% of theory) of product were obtained.

¹H NMR (300 MHz, DMSO-d₆): δ=0.95-1.27 (m, 8H), 1.90-2.06 (m, 4H), 2.24-2.32 (m, 2H), 2.49-2.56 (m, 2H), 3.22 (mc, 2H), 4.20 (t, 1H), 6.59-6.66 (m, 2H), 7.10 (d, 1H), 7.16-7.25 (m, 3H), 7.29-7.37 (m, 2H), 9.26 (s, 1H).

### Intermediate 13-9

### 4-Chloro-9-(6-hydroxyhexyl)-8-phenyl-6,7-dihydro-5H-benzo[7]annulen-3-ol

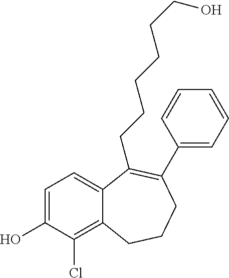 [SMILES: OCCCCCCC1=C(c2ccccc2)CCCc2c1ccc(O)c2Cl]

12.5 g (32.47 mmol) of 6-(4-chloro-3-methoxy-8-phenyl-6,7-dihydro-5H-benzo[7]annulen-9-yl)hexan-1-ol and 6.145 g of sodium thiomethoxide were reacted according to General Procedure 9-1. The residue was purified on silica gel 60 (dichloromethane, dichloromethane-methanol 97:3 and 95:5). 8.2 g (68% of theory) of product were obtained.

¹H NMR (400 MHz, chloroform-d₁): δ=1.10-1.28 (m, 6H), 1.42 (mc, 2H), 2.04-2.19 (m, 4H), 2.39 (t, 2H), 2.89 (t, 2H), 3.54 (q, 2H), 5.67 (s, 1H), 6.95 (d, 1H), 7.16 (d, 1H), 7.21-7.29 (m, 3H), 7.33-7.39 (m, 2H).

### Intermediate 14-9

### 4-Fluoro-8-(4-fluoro-3-hydroxyphenyl)-9-(6-hydroxyhexyl)-6,7-dihydro-5H-benzo[7]annulen-3-ol

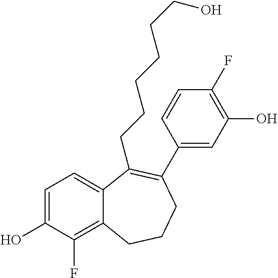 [SMILES: OCCCCCCC1=C(c2ccc(F)c(O)c2)CCCc2c1ccc(O)c2F]

5.4 g (12.96 mmol) of 6-[4-fluoro-8-(4-fluoro-3-methoxyphenyl)-3-methoxy-6,7-dihydro-5H-benzo[7]annulen-9-yl]hexan-1-ol were reacted according to General Procedure 9-2, but using 4 equivalents each of boron tribromide and 2,6-dimethylpyridine. The mixture was poured into ice/water and the precipitate was then filtered off with suction, washed with water and dried in a drying cabinet at 40° C. 3.8 g (75% of theory) of product were obtained.

¹H NMR (400 MHz, DMSO-d₆): δ=0.98-1.14 (m, 6H), 1.17-1.26 (m, 2H), 1.87-2.03 (m, 4H), 2.24-2.33 (m, 2H), 2.58 (mc, 2H), 3.23 (t, 2H), 6.59 (ddd, 1H), 6.74-6.82 (m, 2H), 6.93 (d, 1H), 7.07 (dd, 1H), 9.71 (s, 1H), 9.81 (s, 1H).

### Intermediate 15-9

### 2-Fluoro-8-(4-fluoro-3-hydroxyphenyl)-9-(6-hydroxyhexyl)-6,7-dihydro-5H-benzo[7]annulen-3-ol

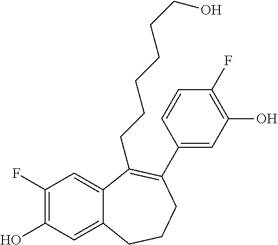 [SMILES: OCCCCCCC1=C(c2ccc(F)c(O)c2)CCCc2cc(O)c(F)cc21]

6.3 g (15.13 mmol) of 6-[2-fluoro-8-(4-fluoro-3-methoxyphenyl)-3-methoxy-6,7-dihydro-5H-benzo[7]annulen-9-yl]hexan-1-ol were reacted according to General Procedure 9-2, but using 4 equivalents each of boron tribromide and 2,6-dimethylpyridine. The mixture was poured into ice/water and the precipitate was then filtered off with suction, washed with water, taken up in ethyl acetate, dried over magnesium sulphate and concentrated. 3.46 g (59% of theory) of product were obtained. The organic phase of the filtrate was separated off and discarded. The aqueous phase was extracted three times with chloroform. The combined organic phases were washed twice with water, dried over magnesium sulphate and concentrated. 1.25 g (21% of theory) of product were isolated.

¹H NMR (300 MHz, DMSO-d₆): δ=0.95-1.15 (m, 6H), 1.16-1.28 (m, 2H), 1.85-2.03 (m, 4H), 2.21-2.32 (m, 2H), 3.23 (t, 2H), 6.57 (ddd, 1H), 6.72-6.79 (m, 2H), 7.01-7.10 (m, 2H), 9.68 (s, 1H), 9.77 (s, 1H).

### Intermediate 16-9

### 9-(6-Hydroxyhexyl)-8-(3-mesylphenyl)-6,7-dihydro-5H-benzo[7]annulen-3-ol

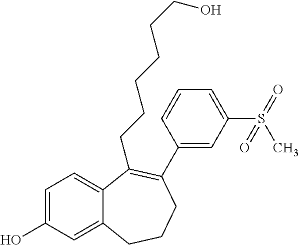 [SMILES: CS(=O)(=O)c1cccc(C2=C(CCCCCCO)c3ccc(O)cc3CCC2)c1]

A mixture of 521 mg (1.22 mmol) of 6-[8-(4-mesylphenyl)-3-methoxy-6,7-dihydro-5H-benzo[7]annulen-9-yl]hexan-1-ol in 6 ml of dichloromethane was cooled in an ice bath, and 4.25 ml of a 1 M solution of boron tribromide in dichloromethane were added. At a temperature of below 5° C., a solution of 496 microlitres of 2,6-lutidine in 2 ml of dichloromethane was then added. The mixture was stirred with ice bath cooling for 2 h and at room temperature for 18 h 45 min. The mixture was poured into ice water and extracted three times with dichloromethane, and the extracts were washed with sodium chloride solution, dried over sodium sulphate and concentrated. Purification of the residue by column chromatography (mobile phase dichloromethane/methanol) gave 266 mg (53% of theory) of the title compound. C₂₄H₃₀O₄S (414.6). MS (ESIpos) mass found: 414.00.

### Intermediate 17-9

### 4-[3-Hydroxy-9-(6-hydroxyhexyl)-6,7-dihydro-5H-benzo[7]annulen-8-yl]benzonitrile

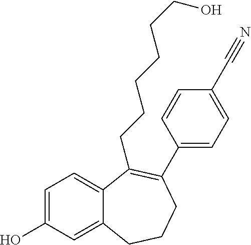 [SMILES: N#Cc1ccc(C2=C(CCCCCCO)c3ccc(O)cc3CCC2)cc1]

2.35 g of 4-[9-(6-hydroxyhexyl)-3-methoxy-6,7-dihydro-5H-benzo[7]annulen-8-yl]benzonitrile were reacted analogously. Purification by column chromatography on silica gel (hexane/ethyl acetate) gave 818 mg of 4-[3-hydroxy-9-(6-hydroxyhexyl)-6,7-dihydro-5H-benzo[7]annulen-8-yl]benzonitrile as crude product (yellowish foam). C₂₄H₂₇NO₂ (361.49). 1H NMR (400 MHz, chloroform-d): δ 1.08-1.26 (m), 1.38-1.47 (m), 2.06-2.16 (m, 4H), 2.31-2.38 (m, 2H), 2.60-2.65 (m, 2H), 3.55 (t, 2H), 6.72 (d, 1H), 6.75 (dd, 1H), 7.18 (d, 1H), 7.32-7.39 (m, 2H), 7.62-7.69 (m, 2H).

### Intermediate 18-9

### 8-(2-Fluoro-5-hydroxyphenyl)-9-(6-hydroxyhexyl)-6,7-dihydro-5H-benzo[7]annulen-3-ol

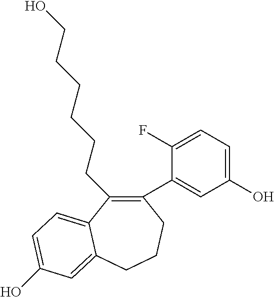 [SMILES: OCCCCCCC1=C(c2cc(O)ccc2F)CCCc2cc(O)ccc21]

2.4 g of 6-[8-(2-fluoro-5-methoxyphenyl)-3-methoxy-6,7-dihydro-5H-benzo[7]annulen-9-yl]hexan-1-ol were reacted analogously. Purification by column chromatography (dichloromethane/methanol) gave 1.73 g (78% of theory) of the title compound. C₂₃H₂₇FO₃ (370.47). MS (ESIpos) mass found: 370.00.

### Intermediate 19-9

### 4-Chloro-8-(4-fluoro-3-hydroxyphenyl)-9-(6-hydroxyhexyl)-6,7-dihydro-5H-benzo[7]annulen-3-ol

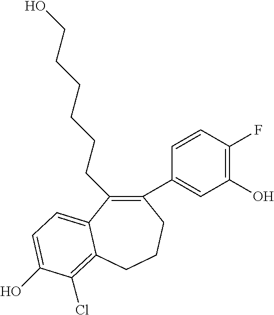 [SMILES: OCCCCCCC1=C(c2ccc(F)c(O)c2)CCCc2c1ccc(O)c2Cl]

1.00 g of 6-[4-chloro-8-(4-fluoro-3-methoxyphenyl)-3-methoxy-6,7-dihydro-5H-benzo[7]annulen-9-yl]hexan-1-ol were reacted analogously. Purification by column chromatography on silica gel (hexane/ethyl acetate) and subsequent purification by preparative HPLC (XBridge column from Waters, solvent acetonitrile/water with 0.1% formic acid added) gave 316 mg of the title compound. C₂₃H₂₆ClFO₃ (404.91). MS (ESIpos) mass found: 404.00. 1H NMR (300 MHz, DMSO d₆): δ 0.96-1.29 (m, 8H), 1.82-2.04 (m, 4H), 2.26-2.37 (m, 2H), 2.70-2.82 (m, 2H), 3.23 (t, 2H), 4.0-4.4 (broad s.), 6.55-6.63 (m, 1H), 6.77 (dd, 1H), 6.84 (d, 1H), 7.02-7.12 (2H), 9.7-10.1 (broad s.).

### Intermediate 20-9

### 8-(4-Fluoro-3-hydroxyphenyl)-9-(5-hydroxypentyl)-6,7-dihydro-5H-benzo[7]annulen-3-ol

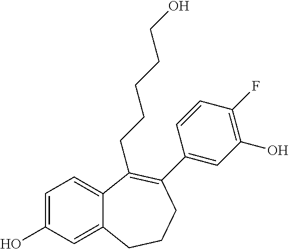 [SMILES: OCCCCCC1=C(c2ccc(F)c(O)c2)CCCc2cc(O)ccc21]

10.6 g of 5-[8-(4-fluoro-3-methoxyphenyl)-3-methoxy-6,7-dihydro-5H-benzo[7]annulen-9-yl]pentan-1-ol (crude product) were reacted analogously with boron tribromide in the presence of lutidine. Purification by column chromatography on silica gel (hexane/ethyl acetate) gave 2.17 g of the title compound. C₂₂H₂₅FO₃ (356.44). MS (EI+): mass found 356 [100]. 1H NMR (600 MHz, DMSO d₆): δ 1.05-1.66 (m, 4H), 1.21 (quint. 2H), 1.92-1.97 (m, 2H), 1.99-2.05 (m, 2H), 2.31 (t, 2H), 2.51-2.55 (m, obscured by water signal), 3.24 (t, 2H), 4.17-4.31 (broad s., 1H), 6.60-6.68 (m, 3H), 6.80 (dd, 1H), 7.09 (dd, 1H), 7.12 (d, 1H), 9.22-9.35 (broad s., 1H), 9.69-9.83 (broad s., 1H).

### Intermediates 10

General Procedure 10 for the preparation of 10 under an atmosphere of protective gas and with exclusion of moisture: 1 g of alcohol was dissolved in about 13-33 ml of dichloromethane, a mixture of dichloromethane and tetrahydrofuran or pure tetrahydrofuran. At 0-5° C., 1.5-1.6 equivalents of triphenylphosphine and 1.5-1.6 equivalents of carbon tetrabromide were added a little at a time. The mixture was stirred for another 2-3 hours at 3-5° C., unless described otherwise. The reaction mixture was diluted with dichloromethane or methyl tert-butyl ether, washed with saturated sodium bicarbonate solution and saturated sodium chloride solution, dried over magnesium sulphate or sodium sulphate and concentrated. The residue was then chromatographed on silica gel 60.

### Intermediate 1-10

### 9-(5-Bromopentyl)-8-phenyl-6,7-dihydro-5H-benzo[7]annulen-3-ol

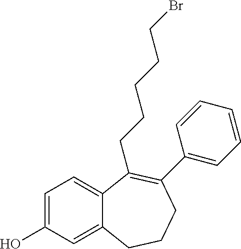 [SMILES: Oc1ccc2c(c1)CCCC(c1ccccc1)=C2CCCCCBr]

3.3 g (10.23 mmol) of 9-(5-hydroxypentyl)-8-phenyl-6,7-dihydro-5H-benzo[7]annulen-3-ol were reacted with 4.11 g of triphenylphosphine and 5.19 g of carbon tetrabromide according to General Procedure 10. The product was purified on silica gel 60 (mobile phase: hexane, hexane-ethyl acetate 9:1, 8:2 and 7:3). Pentane was added to the residue, and the mixture was filtered off with suction. This gave 3.50 g (89% of theory) of product.

¹H NMR (300 MHz, DMSO-d₆): δ=1.04-1.21 (m, 4H), 1.52 (mc, 2H), 1.90-2.07 (m, 4H), 2.29 (mc, 2H), 2.52 (mc, 2H), 3.28-3.35 (m, 2H and water), 6.58-6.67 (m, 2H), 7.10 (d, 1H), 7.16-7.26 (m, 3H), 7.28-7.38 (m, 2H), 9.27 (s, 1H).

### Intermediate 2-10

### 9-(5-Bromopentyl)-8-(3-hydroxyphenyl)-6,7-dihydro-5H-benzo[7]annulen-3-ol

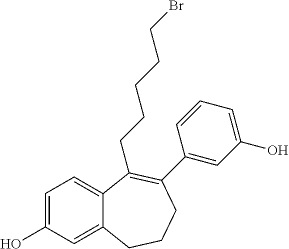 [SMILES: Oc1cccc(C2=C(CCCCCBr)c3ccc(O)cc3CCC2)c1]

4 g (11.82 mmol) of 9-(5-hydroxypentyl)-8-(3-hydroxyphenyl)-6,7-dihydro-5H-benzo[7]annulen-3-ol were reacted with 4.74 g of triphenylphosphine and 6 g of carbon tetrabromide according to General Procedure 10. The product was purified on silica gel 60 (mobile phase: hexane, hexane-ethyl acetate 9:1, 8:2 and 7:3). 3.95 g (83% of theory) of product were obtained.

¹H NMR (400 MHz, DMSO-d₆): δ=1.09-1.24 (m, 4H), 1.58 (mc, 2H), 1.92-2.08 (m, 4H), 2.34 (mc, 2H), 2.54 (mc, 2H), 3.36 (t, 2H), 6.61-6.68 (m, 5H), 7.10-7.18 (m, 2H), 9.26 (s, 1H), 9.31 (s, 1H).

### Intermediate 3-10

### 9-(6-Bromohexyl)-8-(3-hydroxyphenyl)-6,7-dihydro-5H-benzo[7]annulen-3-ol

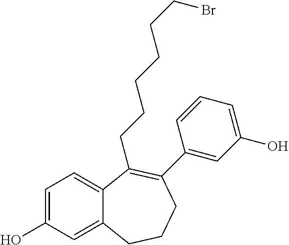 [SMILES: Oc1cccc(C2=C(CCCCCCBr)c3ccc(O)cc3CCC2)c1]

4 g (11.35 mmol) of 9-(6-hydroxyhexyl)-8-(3-hydroxyphenyl)-6,7-dihydro-5H-benzo[7]annulen-3-ol were reacted with 4.55 g of triphenylphosphine and 5.76 g of carbon tetrabromide according to General Procedure 10. The residue was purified on silica gel 60 (mobile phase: hexane, hexane-ethyl acetate 9:1, 8:2 and 7:3). 3.12 g (66% of theory) of product were obtained.

¹H NMR (400 MHz, DMSO-d₆): δ=1.02-1.21 (m, 6H), 1.63 (mc, 2H), 1.91-2.07 (m, 4H), 2.30-2.38 (m, 2H), 2.54 (t, 2H), 3.39 (t, 2H), 6.60-6.68 (m, 5H), 7.09-7.18 (m, 2H), 9.26 (s-broad, 1H), 9.31 (s-broad, 1H).

### Intermediate 4-10

### 9-(5-Bromopentyl)-8-(4-hydroxyphenyl)-6,7-dihydro-5H-benzo[7]annulen-3-ol

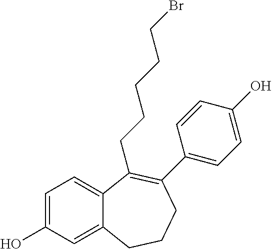 [SMILES: Oc1ccc(C2=C(CCCCCBr)c3ccc(O)cc3CCC2)cc1]

2.0 g (5.91 mmol) of 9-(5-hydroxypentyl)-8-(4-hydroxyphenyl)-6,7-dihydro-5H-benzo[7]annulen-3-ol were reacted with 2.371 g of triphenylphosphine and 2.998 g of carbon tetrabromide according to General Procedure 10. The mixture was stirred at room temperature overnight, and another 2.7 g of triphenylphosphine and 3 g of carbon tetrabromide were added. After a further 24 hours, the reaction was worked up. The residue was purified on silica gel 60. 1.20 g (51% of theory) of product were obtained.

¹H NMR (400 MHz, DMSO-d₆): δ=1.06-1.20 (m, 4H), 1.55 (m, 2H), 1.89-2.02 (m, 4H), 2.30 (m, 2H), 2.49 (m, 2H), 3.34 (t, 2H), 6.57-6.63 (m, 2H), 6.71 (m, 2H), 7.00 (m, 2H), 7.08 (d, 1H), 9.21 (s, 1H), 9.28 (s, 1H).

### Intermediate 5-10

### 9-(6-Bromohexyl)-8-(4-hydroxyphenyl)-6,7-dihydro-5H-benzo[7]annulen-3-ol

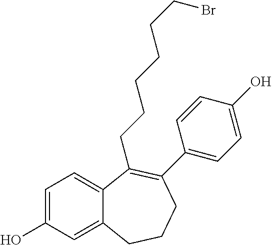 [SMILES: Oc1ccc(C2=C(CCCCCCBr)c3ccc(O)cc3CCC2)cc1]

3.45 g (9.79 mmol) of 9-(6-hydroxyhexyl)-8-(4-hydroxyphenyl)-6,7-dihydro-5H-benzo[7]annulen-3-ol were reacted with 3.93 g of triphenylphosphine and 4.97 g of carbon tetrabromide according to General Procedure 10. The residue was purified on silica gel 60 (mobile phase: hexane, hexane-ethyl acetate 8:2 and 1:1). This gave 2.462 g (61% of theory) of product.

¹H NMR (300 MHz, methanol-d₄): δ=1.04-1.26 (m, 6H), 1.65 (mc, 2H), 1.97-2.13 (m, 4H), 2.38 (mc, 2H), 2.58 (t, 2H), 3.25-3.32 (m, 2H and methanol signal), 6.61-6.69 (m, 2H), 6.72-6.78 (m, 2H), 7.00-7.06 (m, 2H), 7.10 (d, 1H).

### Intermediate 6-10

### 9-(5-Bromopentyl)-8-(3-fluoro-4-hydroxyphenyl)-6,7-dihydro-5H-benzo[7]annulen-3-ol

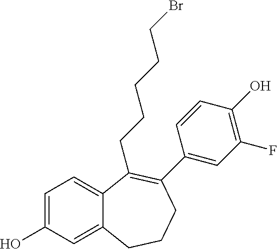 [SMILES: Oc1ccc2c(c1)CCCC(c1ccc(O)c(F)c1)=C2CCCCCBr]

420 mg (1.18 mmol) of 8-(3-fluoro-4-hydroxyphenyl)-9-(5-hydroxypentyl)-6,7-dihydro-5H-benzo[7]annulen-3-ol were reacted with 598 mg of triphenylphosphine and 473 mg of carbon tetrabromide according to General Procedure 10. After 3 hours, the same amounts of reagents were added, and the reaction was worked up the next morning. The crude product was purified on silica gel 60 (mobile phase: hexane, hexane-ethyl acetate 1:1). This gave 177 mg (36% of theory) of product.

¹H NMR (400 MHz, methanol-d₄): δ=1.12-1.31 (m, 4H), 1.62 (mc, 2H), 1.97-2.15 (m, 4H), 2.35-2.44 (m, 2H), 2.59 (t, 2H), 3.26 (t, 2H), 6.62-6.69 (m, 2H), 6.83-6.93 (m, 3H), 7.11 (d, 1H).

### Intermediate 7-10

### 9-(6-Bromohexyl)-8-(4-fluoro-3-hydroxyphenyl)-6,7-dihydro-5H-benzo[7]annulen-3-ol

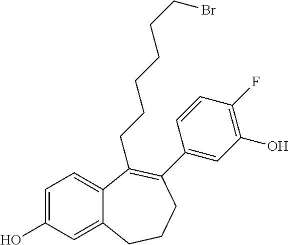 [SMILES: Oc1ccc2c(c1)CCCC(c1ccc(F)c(O)c1)=C2CCCCCCBr]

4.35 g (11.74 mmol) of 8-(4-fluoro-3-hydroxyphenyl)-9-(6-hydroxyhexyl)-6,7-dihydro-5H-benzo[7]annulen-3-ol were reacted with 4.71 g of triphenylphosphine and 5.96 g of carbon tetrabromide according to General Procedure 10. The residue was chromatographed on silica gel 60 (mobile phase: hexane, hexane-ethyl acetate 9:1, 8:2 and 7:3). The residue was triturated with hexane-diisopropyl ether, filtered off with suction and dried in a drying cabinet at 50° C. 3.85 g (81% of theory) of white product were obtained.

¹H NMR (600 MHz, DMSO-d₆): δ=1.05-1.20 (m, 6H), 1.63 (mc, 2H), 1.95 (t, 2H), 2.02 (mc, 2H), 2.33 (mc, 2H), 2.53 (t, 2H), 3.40 (t, 2H), 6.60-6.67 (m, 3H), 6.80 (dd, 1H), 7.09 (dd, 1H), 7.12 (d, 1H), 9.30 (s, 1H), 9.78 (s, 1H).

### Intermediate 8-10

### 9-(5-Bromopentyl)-8-(4-fluoro-3-hydroxyphenyl)-6,7-dihydro-5H-benzo[7]annulen-3-ol

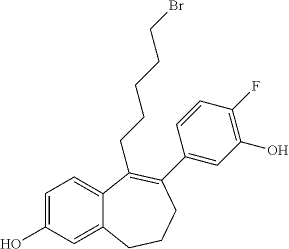 [SMILES: Oc1ccc2c(c1)CCCC(c1ccc(F)c(O)c1)=C2CCCCCBr]

6.85 g of 8-(4-fluoro-3-hydroxyphenyl)-9-(5-hydroxypentyl)-6,7-dihydro-5H-benzo[7]annulen-3-ol were reacted according to General Procedure 10 in tetrahydrofuran. Purification by column chromatography on silica gel (hexane/ethyl acetate) gave 6.25 g (78% of theory) of the title compound. C₂₂H₂₄BrFO₂ (419.34). 1H NMR (selected signals, 300 MHz, DMSO-d₆): δ 1.04-1.22 (m, 4H), 1.54 (quint., 2H), 1.86-2.02 (m, 4H), 2.25-2.34 (m, 2H), 3.23-3.37 (m, partially obscured by water signal), 6.55-6.65 (m, 3H), 6.73-6.80 (m 1H), 7.01-7.13 (m, 2H), 9.27 (s, 1H), 9.76 (s, 1H).

### Intermediate 9-10

### 9-(5-Bromopentyl)-8-(3-pyridyl)-6,7-dihydro-5H-benzo[7]annulen-3-ol

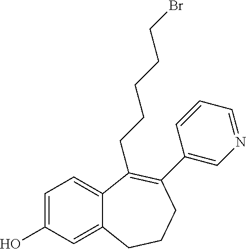 [SMILES: Oc1ccc2c(c1)CCCC(c1cccnc1)=C2CCCCCBr]

3 g (9.28 mmol) of 9-(5-hydroxypentyl)-8-(3-pyridyl)-6,7-dihydro-5H-benzo[7]annulen-3-ol were reacted with 3.72 g of triphenylphosphine and 4.71 g of carbon tetrabromide according to General Procedure 10, the reaction being stirred at room temperature for another 2 hours. The residue was purified on silica gel 60 (mobile phase: hexane, hexane-acetone 9:1 and 8:2). 2.0 g (56% of theory) of product were isolated.

¹H NMR (300 MHz, DMSO-d₆): δ=1.05-1.27 (m, 4H), 1.52-1.63 (m, 2H), 1.95-2.13 (m, 4H), 2.33 (mc, 2H), 2.54-2.63 (m, 2H), 3.33-3.40 (m, 2H and water), 6.65-6.71 (m, 2H), 7.17 (d, 1H), 7.41 (dd, 1H), 7.67 (dt, 1H), 8.45-8.52 (m, 2H), 9.37 (s, 1H).

### Intermediate 10-10

### 9-(6-Bromohexyl)-8-(3-pyridyl)-6,7-dihydro-5H-benzo[7]annulen-3-ol

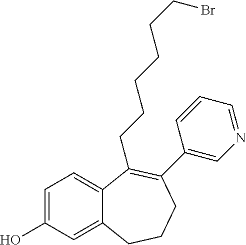 [SMILES: Oc1ccc2c(c1)CCCC(c1cccnc1)=C2CCCCCCBr]

1.85 g (5.48 mmol) of 9-(6-hydroxyhexyl)-8-(3-pyridyl)-6,7-dihydro-5H-benzo[7]annulen-3-ol were reacted with 2.2 g of triphenylphosphine and 2.78 g of carbon tetrabromide according to General Procedure 10, the reaction being stirred at room temperature for another 2 hours. The residue was purified on silica gel 60 (mobile phase: hexane, hexane-acetone 90:10 and 85:15). 1.05 g (48% of theory) of product were isolated.

¹H NMR (400 MHz, DMSO-d₆): δ=0.97-1.17 (m, 6H), 1.54-1.63 (m, 2H), 1.91-2.08 (m, 4H), 2.27 (mc, 2H), 2.52 (t, 2H), 3.36 (t, 2H), 6.60-6.67 (m, 2H), 7.12 (d, 1H), 7.36 (dd, 1H), 7.62 (dt, 1H), 8.41-8.48 (m, 2H), 9.33 (s, 1H).

### Intermediate 11-10

### 9-(5-Bromopentyl)-8-(4-mesylphenyl)-6,7-dihydro-5H-benzo[7]annulen-3-ol

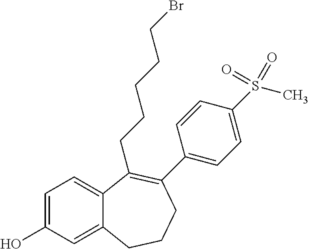 [SMILES: CS(=O)(=O)c1ccc(C2=C(CCCCCBr)c3ccc(O)cc3CCC2)cc1]

1.1 g (2.75 mmol) of 9-(5-hydroxypentyl)-8-(4-mesylphenyl)-6,7-dihydro-5H-benzo[7]annulen-3-ol were reacted with 1.1 g of triphenylphosphine and 1.39 g of carbon tetrabromide according to General Procedure 10. The residue was chromatographed on silica gel 60 (mobile phase: hexane, hexane-ethyl acetate 1:1). This gave 1.14 g (90% of theory) of product.

¹H NMR (600 MHz, chloroform-d₁): δ=1.17-1.29 (m, 4H), 1.66 (mc, 2H), 2.08-2.17 (m, 4H), 2.37 (mc, 2H), 2.65 (t, 2H), 3.11 (s, 3H), 3.26 (t, 2H), 4.86 (s, 1H), 6.74 (d, 1H), 6.77 (dd, 1H), 7.18 (d, 1H), 7.42-7.46 (m, 2H), 7.92-7.95 (m, 2H).

### Intermediate 12-10

### 9-(6-Bromohexyl)-8-(4-mesylphenyl)-6,7-dihydro-5H-benzo[7]annulen-3-ol

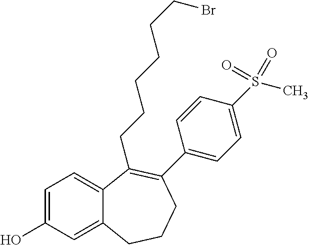 [SMILES: CS(=O)(=O)c1ccc(C2=C(CCCCCCBr)c3ccc(O)cc3CCC2)cc1]

0.6 g (1.45 mmol) of 9-(6-hydroxyhexyl)-8-(4-mesylphenyl)-6,7-dihydro-5H-benzo[7]annulen-3-ol were reacted with 0.58 g of triphenylphosphine and 0.73 g of carbon tetrabromide according to General Procedure 10. The residue was chromatographed on silica gel KP-SIL (mobile phase: hexane, hexane-ethyl acetate 9:1, 8:2 and 7:3). This gave 0.285 g (41% of theory) of product.

¹H NMR (400 MHz, chloroform-d₁): δ=1.08-1.35 (m, 6H), 1.71 (mc, 2H), 2.07-2.18 (m, 4H), 2.35 (mc, 2H), 2.64 (mc, 2H), 3.11 (s, 3H), 3.30 (t, 2H), 4.94 (s-broad, 1H), 6.73 (d, 1H), 6.77 (dd, 1H), 7.18 (d, 1H), 7.44 (dt, 2H), 7.93 (mc, 2H).

### Intermediate 13-10

### 9-(6-Bromohexyl)-8-phenyl-6,7-dihydro-5H-benzo[7]annulen-3-ol

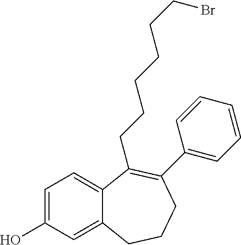 [SMILES: Oc1ccc2c(c1)CCCC(c1ccccc1)=C2CCCCCCBr]

5.6 g (16.64 mmol) of 9-(6-hydroxyhexyl)-8-phenyl-6,7-dihydro-5H-benzo[7]annulen-3-ol were reacted with 6.68 g of triphenylphosphine and 8.44 g of carbon tetrabromide according to General Procedure 10. The residue was chromatographed on silica gel 60 (mobile phase: hexane, hexane-ethyl acetate 9:1 and 8:2). This gave 5.79 g (87% of theory) of product.

¹H NMR (300 MHz, chloroform-d₁): δ=1.06-1.30 (m, 6H), 1.64-1.76 (m, 2H), 2.05-2.19 (m, 4H), 2.38 (mc, 2H), 2.64 (mc, 2H), 3.29 (t, 2H), 4.69 (s, 1H), 6.70-6.78 (m, 2H), 7.19 (d, 1H), 7.22-7.29 (m, 3H), 7.32-7.40 (m, 2H).

### Intermediate 14-10

### 9-(6-Bromohexyl)-4-chloro-8-phenyl-6,7-dihydro-5H-benzo[7]annulen-3-ol

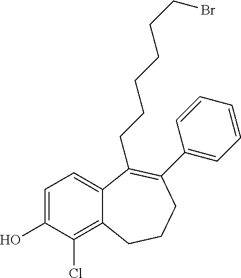 [SMILES: Oc1ccc2c(c1Cl)CCCC(c1ccccc1)=C2CCCCCCBr]

4.80 g (12.94 mmol) of 4-chloro-9-(6-hydroxyhexyl)-8-phenyl-6,7-dihydro-5H-benzo[7]annulen-3-ol were reacted with 5.19 g of triphenylphosphine and 6.57 g of carbon tetrabromide according to General Procedure 10. The residue was chromatographed on silica gel 60 (mobile phase: hexane, hexane-ethyl acetate 95:5, 9:1 and 8:2). This gave 4.61 g (82% of theory) of product.

¹H NMR (300 MHz, chloroform-d₁): δ=1.07-1.30 (m, 6H), 1.71 (quip, 2H), 2.03-2.20 (m, 4H), 2.39 (t, 2H), 2.89 (t, 2H), 3.29 (t, 2H), 5.63 (s, 1H), 6.95 (d, 1H), 7.16 (d, 1H), 7.20-7.30 (m, 3H), 7.32-7.41 (m, 2H).

### Intermediate 15-10

### 9-(6-Bromohexyl)-4-fluoro-8-(4-fluoro-3-hydroxyphenyl)-6,7-dihydro-5H-benzo[7]annulen-3-ol

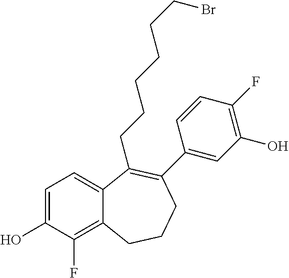 [SMILES: Oc1cc(C2=C(CCCCCCBr)c3ccc(O)c(F)c3CCC2)ccc1F]

3.6 g (9.27 mmol) of 4-fluoro-8-(4-fluoro-3-hydroxyphenyl)-9-(6-hydroxyhexyl)-6,7-dihydro-5H-benzo[7]annulen-3-ol were reacted with 3.72 g of triphenylphosphine and 4.70 g of carbon tetrabromide according to General Procedure 10. The residue was chromatographed on silica gel 60 (mobile phase: hexane, hexane-ethyl acetate 9:1 and 8:2). This gave 3.2 g (77% of theory) of product.

¹H NMR (400 MHz, chloroform-d₁): δ=1.09-1.30 (m, 6H), 1.72 (quin, 2H), 2.03-2.15 (m, 4H), 2.37 (t, 2H), 2.69-2.75 (m, 2H), 3.31 (t, 2H), 5.09-5.13 (m, 2H), 6.71 (ddd, 1H), 6.86-6.92 (m, 2H), 6.98 (dd, 1H), 7.06 (dd, 1H).

### Intermediate 16-10

### 9-(6-Bromohexyl)-2-fluoro-8-(4-fluoro-3-hydroxyphenyl)-6,7-dihydro-5H-benzo[7]annulen-3-ol

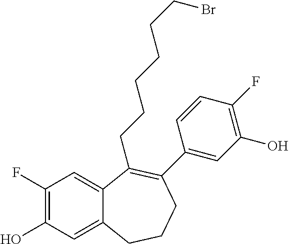 [SMILES: Oc1cc(C2=C(CCCCCCBr)c3cc(F)c(O)cc3CCC2)ccc1F]

5.1 g (13.13 mmol) of 2-fluoro-8-(4-fluoro-3-hydroxyphenyl)-9-(6-hydroxyhexyl)-6,7-dihydro-5H-benzo[7]annulen-3-ol were reacted with 5.61 g of triphenylphosphine and 7.1 g of carbon tetrabromide according to General Procedure 10; however, the mixture was diluted with ethyl acetate. The residue was chromatographed on silica gel 60 (mobile phase: hexane, hexane-ethyl acetate 9:1, 8:2 and 7:3). 4.8 g (81% of theory) of product were obtained.

¹H NMR (400 MHz, chloroform-d₁): δ=1.10-1.31 (m, 6H), 1.72 (quin, 2H), 2.01-2.14 (m, 4H), 2.33 (t, 2H), 2.58 (t, 2H), 3.31 (t, 2H), 5.05 (d, 1H), 5.12 (d, 1H), 6.70 (ddd, 1H), 6.83-6.89 (m, 2H), 6.98-7.09 (m, 2H).

### Intermediate 17-10

### 4-[9-(6-Bromohexyl)-3-hydroxy-6,7-dihydro-5H-benzo[7]annulen-8-yl]benzonitrile

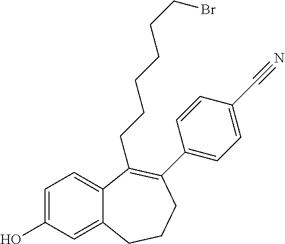 [SMILES: N#Cc1ccc(C2=C(CCCCCCBr)c3ccc(O)cc3CCC2)cc1]

814 mg of 4-[3-hydroxy-9-(6-hydroxyhexyl)-6,7-dihydro-5H-benzo[7]annulen-8-yl]benzonitrile (crude product) gave analogously, after purification by column chromatography on silica gel (hexane/ethyl acetate) and preparative HPLC (column Chiralpak AD-H 5 μm 250×20 mm, hexane/2-propanol 80:20+0.1% diethylamine), 525 mg of 4-[9-(6-bromohexyl)-3-hydroxy-6,7-dihydro-5H-benzo[7]annulen-8-yl]benzonitrile (crude product). 1H NMR (selected signals, 400 MHz, DMSO-d₆): δ 1.53-1.63 (m, 2H), 2.24-2.31 (m, 2H), 2.49-2.55 (m, 2H), 3.36 (t, 2H), 6.61-6.66 (m, 2H), 7.12 (d, 1H), 7.41 (d, 2H), 7.80 (d, 2H), 9.34 (s, 1H).

### Intermediate 18-10

### 9-(6-Bromohexyl)-8-(2-fluoro-5-hydroxyphenyl)-6,7-dihydro-5H-benzo[7]annulen-3-ol

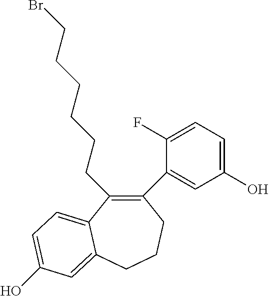 [SMILES: Oc1ccc2c(c1)CCCC(c1cc(O)ccc1F)=C2CCCCCCBr]

1.7 g of 4-fluoro-3-[9-(6-hydroxyhexyl)-3-methoxy-6,7-dihydro-5H-benzo[7]annulen-8-yl]phenol were reacted analogously to General Procedure 10 with triphenylphosphine and carbon tetrabromide in a mixture of THF (2 ml) and dichloromethane (50 ml). This gave, after purification by column chromatography on silica gel (hexane/ethyl acetate), 1.75 g of the title compound contaminated with 9-(6-chlorohexyl)-8-(2-fluoro-5-hydroxyphenyl)-6,7-dihydro-5H-benzo[7]annulen-3-ol. C₂₃H₂₆BrFO₂ (433.37). MS (ESIpos) mass found: 433.00. 1H NMR (400 MHz, DMSO-d₆): δ 1.95-2.05 (m, 2H), 2.18-2.27 (m, 2H), 2.49-2.55 (m, 2H), 3.36 (t), 6.53-6.57 (m, 1H), 6.59-6.66 (m, 3H), 6.96 (t, 1H), 7.10 (d, 1H), 9.28-9.31 (m, 1.6H).

### Intermediate 19-10

### 9-(6-Bromohexyl)-4-chloro-8-(4-fluoro-3-hydroxyphenyl)-6,7-dihydro-5H-benzo[7]annulen-3-ol

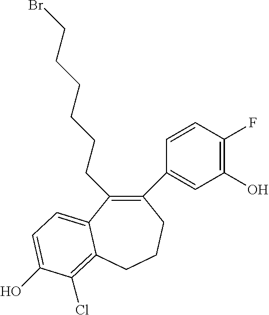 [SMILES: Oc1cc(C2=C(CCCCCCBr)c3ccc(O)c(Cl)c3CCC2)ccc1F]

300 mg of 4-chloro-8-(4-fluoro-3-hydroxyphenyl)-9-(6-hydroxyhexyl)-6,7-dihydro-5H-benzo[7]annulen-3-ol were reacted with carbon tetrabromide and triphenylphosphine in THF analogously to General Procedure 10. This gave, after purification by column chromatography on silica gel (hexane/ethyl acetate), 350 mg of the title compound. C₂₃H₂₅BrClFO₂ (467.81). MS (ESIpos) mass found: 667.00. 1H NMR (300 MHz, DMSO-d₆): δ 0.97-1.27 (m), 1.41-1.68 (m), 1.74-1.92 (m), 1.92-2.06 (m), 2.25-2.38 (m, 2H), 2.67-2.87 (m, 2H), 3.50 (t, 2H), 6.56-6.64 (m, 1H), 6.77 (dd, 1H), 6.84 (d, 1H), 7.02-7.13 (2H), 9.78 (s, 1H), 10.0 (s, 1H).

### Intermediate 20-10

### 9-(6-Bromohexyl)-8-(3-mesylphenyl)-6,7-dihydro-5H-benzo[7]annulen-3-ol

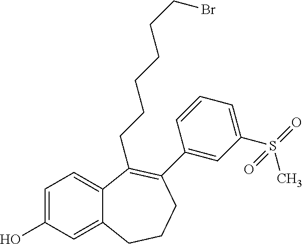 [SMILES: CS(=O)(=O)c1cccc(C2=C(CCCCCCBr)c3ccc(O)cc3CCC2)c1]

266 mg of 9-(6-hydroxyhexyl)-8-(3-mesylphenyl)-6,7-dihydro-5H-benzo[7]annulen-3-ol in 5 ml of dichloromethane were reacted analogously to General Procedure 10. Purification by column chromatography (hexane/ethyl acetate) gave 276 mg of the title compound as crude product. C₂₄H₂₉BrO₃S (477.5); MS (ESIpos) mass found: 477.00. 1H NMR (selected signals, 300 MHz, chloroform d₁): δ 2.07-2.17 (m, 4H), 2.29-2.40 (m, 2H), 2.69-2.71 (m, 2H), 3.20 (t), 6.71-6.81 (m, 2H), 7.18 (d, 1H), 7.53-7.59 (m, 2H), 7.81-7.87 (m, 2H).

Intermediates 12

General Procedure 12 for the preparation of 12 with exclusion of moisture: 1 molar equivalent of alcohol was dissolved in 5 molar equivalents of pyridine, and 1.1 molar equivalents of tosyl chloride were added at 0-5° C. The mixture was then stirred at 0° C. for 2.5 hours and at room temperature for 1-2 hours or overnight. The reaction mixture was stirred into a mixture of ice-water and concentrated sulphuric acid (10 ml: 1 ml). Here, per 10 ml of pyridine 29-53 ml of water were used as base. The mixture was extracted three times with diethyl ether, and the combined organic phases were washed once with water and with saturated sodium chloride solution, dried over sodium sulphate or magnesium sulphate and concentrated.

### Intermediate 1-12

### 4,4,5,5,5-Pentafluoropentyl 4-methylbenzenesulphonate

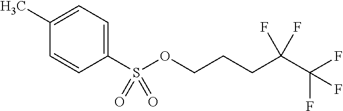 [SMILES: Cc1ccc(S(=O)(=O)OCCCC(F)(F)C(F)(F)F)cc1]

40 g (224.6 mmol) of 4,4,5,5,5-pentafluorpentan-1-ol were reacted with 47.04 g of tosyl chloride according to General Procedure 12. 39.5 g (53% of theory) of product were obtained.

¹H NMR (400 MHz, chloroform-d₁): δ=1.90-2.00 (m, 2H), 2.01-2.17 (m, 2H), 2.46 (s, 3H), 4.10 (t, 2H), 7.37 (d, 2H), 7.80 (d, 2H).

### Intermediate 2-12

### 3,3,4,4,4-Pentafluorobutyl 4-methylbenzenesulphonate

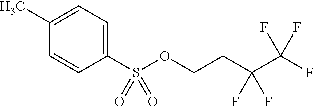 [SMILES: Cc1ccc(S(=O)(=O)OCCC(F)(F)C(F)(F)F)cc1]

19.82 g (120.8 mmol) of 3,3,4,4,4-pentafluorobutan-1-ol were reacted with 25.33 g of tosyl chloride according to General Procedure 12. 27.5 g (72% of theory) of product were obtained.

¹H NMR (400 MHz, chloroform-d₁): δ=2.40-2.54 (m, 5H), 4.28 (t, 2H), 7.38 (d, 2H), 7.80 (dt, 2H).

### Intermediate 3-12

### 3,3,3-Trifluoropropyl 4-methylbenzenesulphonate

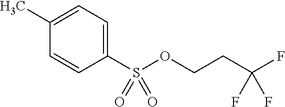 [SMILES: Cc1ccc(S(=O)(=O)OCCC(F)(F)F)cc1]

25.5 g (223.5 mmol) of 3,3,3-trifluoropropan-1-ol were reacted with 45.93 g of tosyl chloride according to General Procedure 12. 47.26 g (80% of theory) of product were obtained.

¹H NMR (300 MHz, chloroform-d₁): δ=2.43-2.59 (m, 5H), 4.22 (t, 2H), 7.37 (d, 2H), 7.80 (dt, 2H).

### Intermediates 13

General Procedure 13 for the preparation of 13: 1 molar equivalent of tosylate/iodide/chloride was stirred under reflux with 1.63 molar equivalents of potassium thioacetate in acetone (5.1-8.1 ml of acetone per g of substance) for 3-3.5 hours. After cooling, the solvent was removed under reduced pressure and the residue was added to water. The mixture was extracted three times with diethyl ether. The combined organic phases were washed once with water and once or twice with saturated sodium chloride solution, dried over sodium sulphate or magnesium sulphate and concentrated.

General Procedure 13a for the preparation of 13: 1 molar equivalent of halide and 1.63 molar equivalents of potassium thioacetate in acetone (5.1-8.1 ml of acetone per g of substance) were stirred under reflux for 3-3.5 hours. After cooling, the mixture was filtered off with suction and the filtrate was concentrated. Water was added, and the mixture was extracted three times with diethyl ether. The combined organic phases were dried over magnesium sulphate and concentrated.

### Intermediate 1-13

### S-(4,4,5,5,5-Pentafluoropentyl) thioacetate

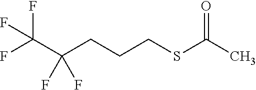

155 g (466.5 mmol) of 4,4,5,5,5-pentafluoropentyl 4-methylbenzenesulphonate were reacted with 86.92 g of potassium thioacetate according to General Procedure 13. The residue was distilled over a short Vigreux column (10 cm) under atmospheric pressure. At 170° C., 84.3 g (77% of theory) of product were obtained.

¹H NMR (300 MHz, chloroform-d₁): δ=1.82-1.95 (m, 2H), 2.00-2.20 (m, 2H), 2.35 (s, 3H), 2.95 (t, 2H).

### Intermediate 2-13

### S-(3,3,4,4,4-Pentafluorobutyl) thioacetate

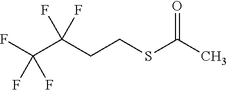

35.6 g (111.9 mmol) of 3,3,4,4,4-pentafluorobutyl 4-methylbenzenesulphonate were reacted with 20.82 g of potassium thioacetate according to General Procedure 13. The residue was distilled over a short Vigreux column (10 cm) under atmospheric pressure. At 70° C., 16.6 g (67% of theory) of product were obtained.

¹H NMR (300 MHz, chloroform-d₁): δ=2.24-2.44 (m, 5H), 3.07 (mc, 2H).

### Intermediate 3-13

### S-(3,3,3-Trifluoropropyl) thioacetate

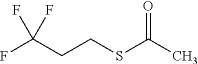

44.88 g (167.3 mmol) of 3,3,3-trifluoropropyl 4-methylbenzenesulphonate were reacted with 31.18 g of potassium thioacetate according to General Procedure 13. The residue was distilled over a short Vigreux column (10 cm) under atmospheric pressure. At 135-137° C., 20.71 g (72% of theory) of product were obtained.

¹H NMR (400 MHz, chloroform-d₁): δ=2.33-2.45 (m, 5H), 3.03 (mc, 2H).

### Intermediate 4-13

### S-(4,4,4-Trifluorobutyl) thioacetate

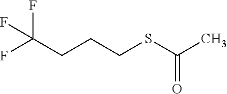

125 g (0.525 mol) of 1,1,1-trifluoro-4-iodobutane were reacted with 97.8 g of potassium thioacetate according to General Procedure 13a. The reaction was distilled at 95 mbar. The first fraction contained 36.57 g (37% of theory; 35-95° C.) and the second fraction 48.02 g (49% of theory; 95-98° C.).

¹H NMR (400 MHz, chloroform-d₁): δ=1.81-1.90 (m, 2H), 2.09-2.23 (m, 2H), 2.35 (s, 3H), 2.93 (t, 2H).

### Intermediates 14

General Procedure 14 for the preparation of 14: 1 molar equivalent of thioacetate was added dropwise with ice cooling to 1.1-2.0 molar equivalents of a 30% strength solution of sodium methoxide in methanol. The mixture was stirred at room temperature for 30 minutes. At room temperature, this solution was added dropwise to 1.3-2 molar equivalents of 1-bromo-ω-chloroalkane in methanol (1.2-1.7 ml per g of halide). The mixture was stirred at room temperature for 2-4 hours. Diethyl ether or methyl tert-butyl ether was added, the phases were separated and the organic phase was washed with water, if required with saturated sodium chloride solution, dried over sodium sulphate or magnesium sulphate and concentrated. The residue was subjected to fractional distillation over a short Vigreux column (10 cm).

### Intermediate 1-14

### 3-Chloropropyl 4,4,5,5,5-pentafluoropentyl sulphide

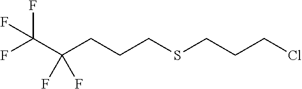 [SMILES: FC(F)(F)C(F)(F)CCCSCCCCl]

132 g (558.54 mmol) of S-(4,4,5,5,5-pentafluoropentyl) thioacetate were reacted with 131.97 g (558.84 mmol) of 1-bromo-3-chloropropane according to General Procedure 14. 126 g (83% of theory) of product were obtained. B.p._{18 mbar}=117° C.

¹H NMR (400 MHz, chloroform-d₁): δ=1.85-1.94 (m, 2H), 2.04 (quin, 2H), 2.10-2.25 (m, 2H), 2.61 (t, 2H), 2.68 (t, 2H), 3.66 (t, 2H).

### Intermediate 2-14

### 4-Chlorobutyl 4,4,5,5,5-pentafluoropentyl sulphide

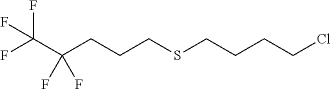 [SMILES: FC(F)(F)C(F)(F)CCCSCCCCCl]

30 g (127.01 mmol) of S-(4,4,5,5,5-pentafluoropentyl) thioacetate were reacted with 32.67 g (190.51 mmol) of 1-bromo-4-chlorobutane according to General Procedure 14. 32.28 g (89% of theory) of product were obtained. B.p._{3.6 mbar}=110-112° C.

¹H NMR (300 MHz, chloroform-d₁): δ=1.74-1.86 (m, 2H), 1.88-2.00 (m, 4H), 2.12-2.32 (m, 2H), 2.55-2.68 (m, 4H), 3.61 (t, 2H).

### Intermediate 3-14

### 3-Chloropropyl 3,3,4,4,4-pentafluorobutyl sulphide

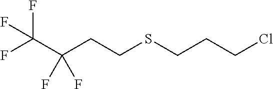 [SMILES: FC(F)(F)C(F)(F)CCSCCCCl]

16.6 g (74.72 mmol) of S-(3,3,4,4,4-pentafluorobutyl) thioacetate in 10 ml of methanol were reacted with 14.7 ml (149.43 mmol) of 1-bromo-3-chloropropane according to General Procedure 14. 17.6 g (92% of theory) of product were obtained. B.p._{55 mbar}=70° C.

¹H NMR (300 MHz, chloroform-d₁): δ=2.05 (quip, 2H), 2.24-2.44 (m, 2H), 2.69-2.77 (m, 4H), 3.66 (t, 2H).

### Intermediate 4-14

### 3-Chloropropyl 3,3,3-trifluoropropyl sulphide

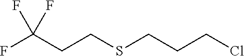 [SMILES: FC(F)(F)CCSCCCCl]

40 g (232.33 mmol) of S-(3,3,3-trifluoropropyl) thioacetate in 60 ml of methanol were reacted with 47.55 g (302.03 mmol) of 1-bromo-3-chloropropane according to General Procedure 14. The crude product was subjected to fractional distillation over a Vigreux column under reduced pressure. 36.5 g (76% of theory) of product were obtained. B.p._{10 mbar}=75° C.

¹H NMR (400 MHz, chloroform-d₁): δ=2.05 (quip, 2H), 2.32-2.46 (m, 2H), 2.67-2.75 (m, 4H), 3.66 (t, 2H).

### Intermediate 5-14

### 3-Chloropropyl 4,4,4-trifluorobutyl sulphide

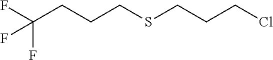 [SMILES: FC(F)(F)CCCSCCCCl]

3.0 g (16.11 mmol) of S-(4,4,4-trifluorobutyl) thioacetate in 10 ml of methanol were reacted with 5.07 g (32.22 mmol) of 1-bromo-3-chloropropane according to General Procedure 14. All volatile components were removed at the pump. 3.7 g (104% of theory) of product were obtained.

¹H NMR (400 MHz, chloroform-d₁): δ=1.82-1.91 (m, 2H), 2.04 (quip, 2H), 2.16-2.33 (m, 2H), 2.59 (t, 2H), 2.68 (t, 2H), 3.66 (t, 2H).

### Intermediate 6-14

### 4-Chlorobutyl 3,3,3-trifluoropropyl sulphide

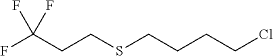 [SMILES: FC(F)(F)CCSCCCCCl]

19.3 g (0.112 mol) of S-(3,3,3-trifluoropropyl) thioacetate in 30 ml of methanol were reacted with 24.99 g (0.146 mol) of 1-bromo-4-chlorobutanane according to General Procedure 14. The solvent was removed at 150 mbar and 40° C. The crude product was subjected to fractional distillation over a Vigreux column. 18.5 g (75% of theory) of product were obtained. B.p._{3mbar}=85° C.

¹H NMR (400 MHz, chloroform-d₁): δ=1.72-1.82 (m, 2H), 1.85-1.94 (m, 2H), 2.31-2.45 (m, 2H), 2.59 (t, 2H), 2.66-2.72 (m, 2H), 3.57 (t, 2H).

### Intermediate 7-14

### 4-Chlorobutyl 4,4,4-trifluorobutyl sulphide

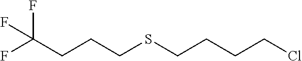 [SMILES: FC(F)(F)CCCSCCCCCl]

6.0 g (32.2 mmol) of S-(4,4,4-trifluorobutyl) thioacetate in 20 ml of methanol were reacted with 6.08 g (35.4 mmol) of 1-bromo-4-chlorobutane in 20 ml of methanol according to General Procedure 14, the mixture being stirred at room temperature overnight. All volatile components were removed at the pump. 7.0 g (93% of theory) of product remained.

¹H NMR (400 MHz, chloroform-d₁): δ=1.71-1.80 (m, 2H), 1.81-1.93 (m, 4H), 2.16-2.29 (m, 2H), 2.52-2.61 (m, 4H), 3.56 (t, 2H).

### Intermediate 8-14

### 5-Chloropentyl 4,4,5,5,5-pentafluoropentyl sulphide

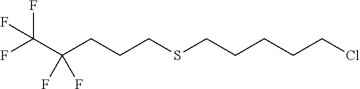 [SMILES: FC(F)(F)C(F)(F)CCCSCCCCCCl]

30 g (127.01 mmol) of S-(4,4,5,5,5-pentafluoropentyl) thioacetate were reacted with 25.1 ml (190.51 mmol) of 1-bromo-5-chloropentane according to General Procedure 14. 32.78 g (86% of theory) of product were obtained. B.p._{3.8 mbar}=124-125° C.

¹H NMR (300 MHz, chloroform-d₁): δ=1.48-1.68 (m, 4H), 1.74-1.94 (m, 4H), 2.07-2.27 (m, 2H), 2.53 (t, 2H), 2.59 (t, 2H), 3.54 (t, 2H).

### Intermediate 9-14

### 6-Chlorohexyl 4,4,5,5,5-pentafluoropentyl sulphide

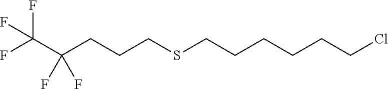 [SMILES: FC(F)(F)C(F)(F)CCCSCCCCCCCl]

30 g (127.01 mmol) of S-(4,4,5,5,5-pentafluoropentyl) thioacetate were reacted with 38.01 g (190.51 mmol) of 1-bromo-6-chlorohexane according to General Procedure 14. 34.71 g (87% of theory) of product were obtained. B.p._{3.2 mbar}=134-136° C.

¹H NMR (300 MHz, chloroform-d₁): δ=1.40-1.57 (m, 4H), 1.65 (mc, 2H), 1.77-1.99 (m, 4H), 2.11-2.32 (m, 2H), 2.57 (t, 2H), 2.64 (t, 2H), 3.58 (t, 2H).

### Intermediates 16

General Procedure 16 for the preparation of 16: 1 molar equivalent of thioether was to dissolved in acetone (1 g of substance in 7.3-11.2 ml), methanol (1 g of substance in 4.3-6.7 ml) and water (2 ml of water per 1 g of sodium metaperiodate), and 1.1 molar equivalents of sodium metaperiodate were added. The mixture was stirred at room temperature for 24-60 hours. The precipitate was filtered off with suction and washed thoroughly with acetone. The filtrate was concentrated to dryness, and the residue was dissolved in methyl tert-butyl ether, washed with water, dried over sodium sulphate or magnesium sulphate and concentrated.

### Intermediate 1-16

### (RS)-3-Chloropropyl 4,4,5,5,5-pentafluoropentyl sulphoxide

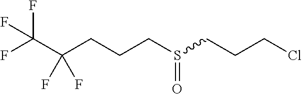 [SMILES: O=S(CCCCl)CCCC(F)(F)C(F)(F)F]

18 g (66.5 mmol) of 3-chloropropyl 4,4,5,5,5-pentafluoropentyl sulphide were reacted according to General Procedure 16. The crude product was digested in hot hexane, filtered off with suction and dried. 17.3 g (91% of theory) of white crystals were obtained.

¹H NMR (300 MHz, chloroform-d₁): δ=2.15-2.41 (m, 6H), 2.75-3.01 (m, 4H), 3.69-3.83 (m, 2H).

### Intermediate 2-16

### (RS)-4-Chlorobutyl 4,4,5,5,5-pentafluoropentyl sulphoxide

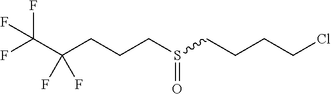 [SMILES: O=S(CCCCCl)CCCC(F)(F)C(F)(F)F]

13 g (45.66 mmol) of 4-chlorobutyl 4,4,5,5,5-pentafluoropentyl sulphide were reacted according to General Procedure 16. The crude product was digested in hot hexane, filtered off with suction and dried. 12.77 g (93% of theory) of white crystals were obtained.

¹H NMR (300 MHz, chloroform-d₁): δ=1.90-2.12 (m, 4H), 2.15-2.41 (m, 4H), 2.68-2.90 (m, 4H), 3.62 (t, 2H).

### Intermediate 3-16

### (RS)-3-Chloropropyl 3,3,4,4,4-pentafluorobutyl sulphoxide

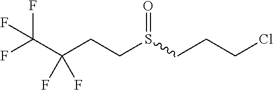 [SMILES: O=S(CCCCl)CCC(F)(F)C(F)(F)F]

5.02 g (19.56 mmol) of 3-chloropropyl 3,3,4,4,4-pentafluorobutyl sulphide were reacted according to General Procedure 16. 4.8 g (90% of theory) of product were obtained.

¹H NMR (400 MHz, chloroform-d₁): δ=2.31 (quin, 2H), 2.50-2.66 (m, 2H), 2.83-3.01 (m, 4H), 3.66-3.78 (m, 2H).

### Intermediate 4-16

### (RS)-3-Chloropropyl 3,3,3-trifluoropropyl sulphoxide

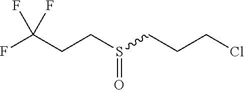 [SMILES: O=S(CCCCl)CCC(F)(F)F]

18 g (87.1 mmol) of 3-chloropropyl 3,3,3-trifluoropropyl sulphide were reacted according to General Procedure 16. 17.5 g (90% of theory) of product were obtained.

¹H NMR (300 MHz, chloroform-d₁): δ=2.25-2.36 (m, 2H), 2.54-2.71 (m, 2H), 2.80-2.99 (m, 4H), 3.64-3.78 (m, 2H).

### Intermediate 5-16

### (RS)-5-Chloropentyl 4,4,5,5,5-pentafluoropentyl sulphoxide

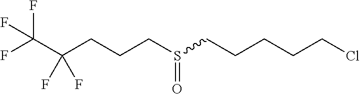 [SMILES: O=S(CCCCCCl)CCCC(F)(F)C(F)(F)F]

14 g (46.86 mmol) of 5-chloropentyl 4,4,5,5,5-pentafluoropentyl sulphide were reacted according to General Procedure 16. The crude product was digested in hot hexane, filtered off with suction and dried. 14.31 g (97% of theory) of white crystals were obtained.

¹H NMR (300 MHz, chloroform-d₁): δ=1.54-1.74 (m, 2H), 1.77-1.90 (m, 4H), 2.09-2.35 (m, 4H), 2.60-2.82 (m, 4H), 3.56 (t, 2H).

### Intermediate 6-16

### (RS)-6-Chlorohexyl 4,4,5,5,5-pentafluoropentyl sulphoxide

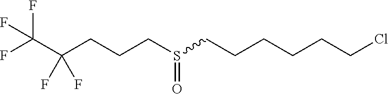 [SMILES: O=S(CCCCCCCl)CCCC(F)(F)C(F)(F)F]

15 g (47.96 mmol) of 6-chlorohexyl 4,4,5,5,5-pentafluoropentyl sulphide were reacted according to General Procedure 16. The crude product was digested in hot hexane, filtered off with suction and dried. 14.5 g (92% of theory) of white crystals were obtained.

¹H NMR (300 MHz, chloroform-d₁): δ=1.47-1.67 (m, 4H), 1.75-1.97 (m, 4H), 2.12-2.40 (m, 4H), 2.65-2.85 (m, 4H), 3.59 (t, 2H).

### Intermediate 7-16

### (RS)-4-Chlorobutyl 3,3,3-trifluoropropyl sulphoxide

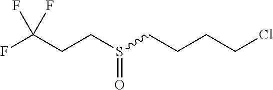 [SMILES: O=S(CCCCCl)CCC(F)(F)F]

20.0 g (0.091 mol) of 4-chlorobutyl 3,3,3-trifluoropropyl sulphide were reacted according to General Procedure 16. 19 g (95% of theory) of product were obtained.

¹H NMR (400 MHz, chloroform-d₁): δ=1.88-2.05 (m, 4H), 2.55-2.95 (m, 6H), 3.55-3.62 (m, 2H).

### Intermediates 18

General Procedure 18 for the preparation of 18: 1 molar equivalent of thioether was dissolved in chloroform. On an ice bath, meta-chloroperbenzoic acid (about 80-90% strength) was added a little at a time such that the temperature did not rise above 10° C. The mixture was stirred at room temperature for 1.5-3 hours and then diluted with dichloromethane. Excess peracid was reduced by washing with 39% strength sodium bisulphite solution. The organic phase was washed with saturated sodium bicarbonate solution and/or with saturated sodium carbonate solution and/or with 2 M NaOH and optionally with water, dried over sodium sulphate or magnesium sulphate and concentrated.

### Intermediate 1-18

### 3-Chloropropyl 4,4,5,5,5-pentafluoropentyl sulphone

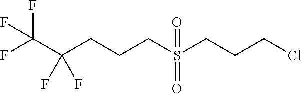 [SMILES: O=S(=O)(CCCCl)CCCC(F)(F)C(F)(F)F]

2.7 g (9.97 mmol) of 3-chloropropyl 4,4,5,5,5-pentafluoropentyl sulphide in 27 ml of chloroform were reacted with 3.44 g (19.95 mmol) of meta-chloroperbenzoic acid according to General Procedure 18. 2.81 g (93% of theory) of product were obtained.

¹H NMR (300 MHz, chloroform-d₁): δ=2.15-2.40 (m, 6H), 3.09 (t, 2H), 3.19 (mc, 2H), 3.71 (t, 2H).

### Intermediate 2-18

### 4-Chlorobutyl 4,4,5,5,5-pentafluoropentyl sulphone

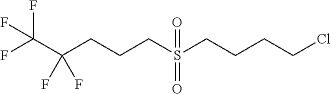 [SMILES: O=S(=O)(CCCCCl)CCCC(F)(F)C(F)(F)F]

15 g (52.68 mmol) of 4-chlorobutyl 4,4,5,5,5-pentafluoropentyl sulphide in 143 ml of chloroform were reacted with 27.27 g (158.05 mmol) according to General Procedure 18. 16.25 g (97% of theory) of product were obtained.

¹H NMR (300 MHz, chloroform-d₁): δ=1.91-2.12 (m, 4H), 2.14-2.38 (m, 4H), 2.99-3.11 (m, 4H), 3.59 (t, 2H).

### Intermediate 3-18

### 3-Chloropropyl 3,3,4,4,4-pentafluorobutyl sulphone

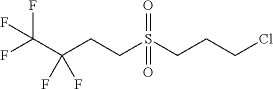 [SMILES: O=S(=O)(CCCCl)CCC(F)(F)C(F)(F)F]

7 g (27.27 mmol) of 3-chloropropyl 3,3,4,4,4-pentafluorobutyl sulphide in 75 ml of chloroform were reacted with 15.06 g (87.27 mmol) of meta-chloroperbenzoic acid according to General Procedure 18. 7.28 g (92% of theory) of product were obtained.

¹H NMR (300 MHz, chloroform-d₁): δ=2.38 (mc, 2H), 2.54-2.75 (m, 2H), 3.21-3.31 (m, 4H), 3.72 (t, 2H).

### Intermediate 4-18

### 3-Chloropropyl 3,3,3-trifluoropropyl sulphone

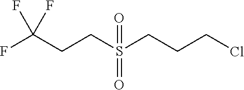 [SMILES: O=S(=O)(CCCCl)CCC(F)(F)F]

18.2 g (88.07 mmol) of 3-chloropropyl 3,3,3-trifluoropropyl sulphide in 300 ml of chloroform were reacted with 45.59 g (264.2 mmol) of meta-chloroperbenzoic acid according to General Procedure 18. The crude product was stirred with hexane, filtered off with suction and dried in a drying cabinet. 20.6 g (98% of theory) of product were obtained.

¹H NMR (400 MHz, chloroform-d₁): δ=2.32-2.40 (m, 2H), 2.63-2.76 (m, 2H), 3.19-3.27 (m, 4H), 3.72 (t, 2H).

### Intermediate 5-18

### 4-Chlorobutyl 3,3,3-trifluoropropyl sulphone

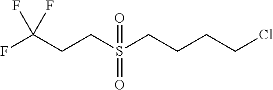 [SMILES: O=S(=O)(CCCCCl)CCC(F)(F)F]

20.0 g (0.091 mol) of 4-chlorobutyl 3,3,3-trifluoropropyl sulphide in 200 ml of chloroform were reacted with 46.92 g (0.272 mol) of meta-chloroperbenzoic acid according to General Procedure 18. The crude product was triturated with pentane, filtered off with suction and dried in a drying cabinet. 22.5 g (98% of theory) of product were obtained.

¹H NMR (300 MHz, chloroform-d₁): δ=1.91-2.14 (m, 4H), 2.60-2.78 (m, 2H), 3.08 (t, 2H), 3.15-3.24 (mc, 2H), 3.60 (t, 2H).

### Intermediate 6-18

### 4-Chlorobutyl 4,4,4-trifluorobutyl sulphone

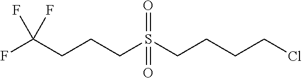 [SMILES: O=S(=O)(CCCCCl)CCCC(F)(F)F]

1 g (4.26 mmol) of 4-chlorobutyl 4,4,4-trifluorobutyl sulphide in 10 ml of chloroform was reacted with 3 g (17.38 mmol) of meta-chloroperbenzoic acid according to General Procedure 18. 1.1 g (97% of theory) of product were obtained.

¹H NMR (300 MHz, chloroform-d₁): δ=1.90-2.22 (m, 6H), 2.25-2.43 (m, 2H), 2.98-3.10 (m, 4H), 3.59 (t, 2H).

### Intermediate 7-18

### 3-Chloropropyl 4,4,4-trifluorobutyl sulphone

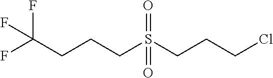 [SMILES: O=S(=O)(CCCCl)CCCC(F)(F)F]

5 g (22.7 mmol) of 3-chloropropyl 4,4,4-trifluorobutyl sulphide in 53 ml of chloroform were reacted with 14.66 g (85.0 mmol) of meta-chloroperbenzoic acid according to General Procedure 18; however, the mixture was stirred at room temperature overnight. Pentane was added to the residue, and the mixture was filtered off with suction. 4.9 g (86% of theory) of product were obtained.

¹H NMR (300 MHz, chloroform-d₁): δ=2.11-2.24 (m, 2H), 2.26-2.43 (m, 4H), 3.08 (mc, 2H), 3.16 (mc, 2H), 3.71 (t, 2H).

### Intermediate 8-18

### 5-Chloropentyl 4,4,5,5,5-pentafluoropentyl sulphone

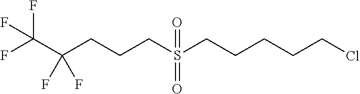 [SMILES: O=S(=O)(CCCCCCl)CCCC(F)(F)C(F)(F)F]

15 g (50.21 mmol) of 5-chloropentyl 4,4,5,5,5-pentafluoropentyl sulphide in 135 ml of chloroform were reacted with 26 g (150.64 mmol) of meta-chloroperbenzoic acid according to General Procedure 18. 16.11 g (97% of theory) of product were obtained.

¹H NMR (400 MHz, chloroform-d₁): δ=1.59-1.68 (m, 2H), 1.80-1.94 (m, 4H), 2.15-2.36 (m, 4H), 3.01 (mc, 2H), 3.06 (t, 2H), 3.56 (t, 2H).

### Intermediate 9-18

### 6-Chlorohexyl 4,4,5,5,5-pentafluoropentyl sulphone

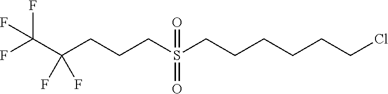 [SMILES: O=S(=O)(CCCCCCCl)CCCC(F)(F)C(F)(F)F]

15 g (47.96 mmol) of 6-chlorohexyl 4,4,5,5,5-pentafluoropentyl sulphide in 130 ml of chloroform were reacted with 24.83 g (143.87 mmol) of meta-chloroperbenzoic acid according to General Procedure 18. 16.44 g (99% of theory) of product were obtained.

¹H NMR (400 MHz, chloroform-d₁): δ=1.45-1.55 (m, 4H), 1.75-1.93 (m, 4H), 2.15-2.36 (m, 4H), 3.00 (mc, 2H), 3.05 (t, 2H), 3.54 (t, 2H).

### Intermediates 15, 17, 19

General Procedure 15-17-19-A for the preparation of 15-17-19: 1 molar equivalent of chloride was dissolved in ethanol (1.7-5.5 ml per g of chloride), and a 40% strength aqueous methylamine solution (12-18 ml per g of chloride) was added. The mixture was stirred in an autoclave at 40° C. for 4 hours. After cooling, the mixture was extracted three times with methyl tert-butyl ether. The combined organic phases were washed with 1 M NaOH, dried over sodium sulphate and concentrated

General Procedure 15-17-19-B for the preparation of 15-17-19: 1 g of chloride was dissolved in 10-25 ml of 33% strength ethanolic methylamine solution and stirred in an autoclave at 40° C. After cooling, the mixture was concentrated.

General Procedure 15-17-19-C for the preparation of 15-17-19: 1 g of chloride was dissolved in 7-14 ml of methanol and stirred with 1.05 molar equivalents of triethylamine and 2-5 molar equivalents of amine at 60° C. Alternatively, the mixture could also be stirred in a microwave. The reaction mixture was concentrated on a rotary evaporator, saturated sodium carbonate solution or water and 2 M aqueous sodium hydroxide solution were added and the mixture was extracted three or four times with dichloromethane or chloroform. The combined organic phases were washed with water if required, dried over magnesium sulphate and concentrated.

General Procedure 15-17-19-D for the preparation of 15-17-19: 1 g of chloride was dissolved in 10-67 ml of 33% strength ethanolic methylamine solution and stirred at 40° C. in an autoclave. After cooling, the mixture was concentrated. The residue was taken up in water and extracted twice with dichloromethane. The aqueous phase was adjusted to pH>10 with 2 M aqueous sodium hydroxide solution and extracted three times with dichloromethane. The combined organic phases were dried over magnesium sulphate and concentrated.

### Intermediate 1-15

### Step A

### 3-Iodopropyl 4,4,5,5,5-pentafluoropentyl sulphide

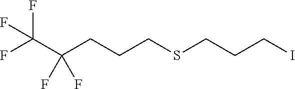 [SMILES: FC(F)(F)C(F)(F)CCCSCCCI]

10 g (36.94 mmol) of 3-chloropropyl 4,4,5,5,5-pentafluoropentyl sulphide were dissolved in 220 ml of methyl ethyl ketone, and 17.6 g (117.4 mmol) of sodium iodide were added. The mixture was stirred at a bath temperature of 100° C. for 5 hours. After cooling, water was added, the mixture was extracted with ethyl acetate and the extract was dried over sodium sulphate and concentrated. This gave 13.32 g (99% of theory) of product.

¹H NMR (300 MHz, chloroform-d₁): δ=1.84-1.96 (m, 2H), 2.01-2.31 (m, 4H), 2.57-2.67 (m, 4H), 3.29 (t, 2H).

### Step B

### N-Methyl-3-[(4,4,5,5,5-pentafluoropentyl)sulphanyl]propane-1-amine

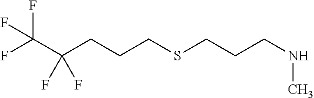 [SMILES: CNCCCSCCCC(F)(F)C(F)(F)F]

13.2 g (36.45 mmol) of 3-iodopropyl 4,4,5,5,5-pentafluoropentyl sulphide were dissolved in 20 ml of ethanol and 140 ml of 40% strength aqueous methylamine solution. The mixture was stirred in an autoclave at 40° C. for 4 hours. After cooling, the mixture was extracted three times with methyl tert-butyl ether. The combined organic phases were washed once with 1 M sodium hydroxide, dried over sodium sulphate and concentrated. The product was purified on silica gel 60 (mobile phase: dichloromethane, dichloromethane-methanol 4:1, 3:1, 2:1, 1:1 and methanol). This gave 5.15 g (53% of theory) of product.

¹H NMR (300 MHz, chloroform-d₁): δ=1.78-1.93 (m, 4H), 2.05-2.26 (m, 2H), 2.47 (s, 3H), 2.58 (t, 2H), 2.59 (t, 2H), 2.74 (t, 2H).

### Intermediate 1-17

### N-Methyl-3-[(RS)-(4,4,5,5,5-pentafluoropentyl)sulphinyl]propane-1-amine

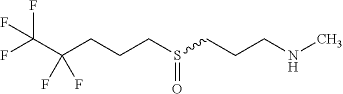 [SMILES: CNCCCS(=O)CCCC(F)(F)C(F)(F)F]

30 g (104.6 mmol) of 3-chloropropyl 4,4,5,5,5-pentafluoropentyl sulphoxide were reacted according to General Procedure 15-17-19-A at 40° C. for 24 hours. The product was purified on silica gel 60 (mobile phase: dichloromethane, dichloromethane-methanol 1:1 and methanol). This gave 12.84 g (44% of theory) of product.

¹H NMR (300 MHz, chloroform-d₁): δ=1.12 (s-br, 1H), 1.90-2.05 (m, 2H), 2.08-2.34 (m, 4H), 2.43 (s, 3H), 2.70-2.81 (m, 6H).

### Intermediate 2-17

### N-Methyl-4-[(RS)-(4,4,5,5,5-pentafluoropentyl)sulphinyl]butane-1-amine

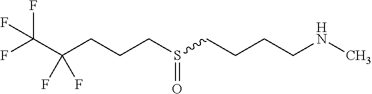 [SMILES: CNCCCCS(=O)CCCC(F)(F)C(F)(F)F]

14 g (46.56 mmol) of 4-chlorobutyl 4,4,5,5,5-pentafluoropentyl sulphoxide were reacted according to General Procedure 15-17-19-A. The product was purified on silica gel 60 (mobile phase: dichloromethane, dichloromethane-methanol 4:1, 3:1, 2:1, 1:1 and methanol with 1% by volume and 10% by volume of 33% strength ammonia solution). This gave 12.09 g (88% of theory) of product.

¹H NMR (300 MHz, chloroform-d₁): δ=1.56-1.93 (m, 4H), 1.96-2.36 (m, 5H), 2.44 (s, 3H), 2.60-2.83 (m, 6H).

### Intermediate 3-17

### N-Methyl-3-[(RS)-(3,3,3-trifluoropropyl)sulphinyl]propane-1-amine

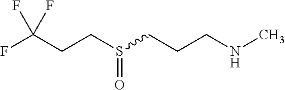

4.2 g (18.86 mmol) of 3-chloropropyl 3,3,3-trifluoropropyl sulphoxide were reacted according to General Procedure 15-17-19-B for 20 hours. The product was purified on silica gel 60 (mobile phase: dichloromethane, dichloromethane-methanol 2:1, 1:1 and methanol with 2% by volume and 5% by volume of 33% strength ammonia solution). This gave 1.86 g (45% of theory) of product.

¹H NMR (400 MHz, DMSO-d₆): δ=1.72-1.88 (m, 2H), 2.25-2.33 (m, 3H), 2.54-2.92 (m, 7H), 2.96-3.06 (m, 1H).

### Intermediate 4-17

### 2-Methyl-1-({3-[(RS)-(3,3,3-trifluoropropyl)sulphinyl]propyl}amino)propan-2-ol

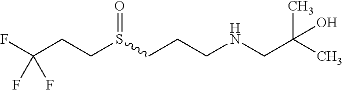 [SMILES: CC(C)(O)CNCCCS(=O)CCC(F)(F)F]

4 g (17.96 mmol) of 3-chloropropyl 3,3,3-trifluoropropyl sulphoxide and 5.61 ml of 1-amino-2-methylpropan-2-ol were stirred for 30 hours according to General Procedure 15-17-19-C and worked up. The product was purified on silica gel 60 (mobile phase: dichloromethane, dichloromethane-methanol 4:1, 1:1 and methanol). This gave 2.2 g (44% of theory) of product.

¹H NMR (300 MHz, methanol-d₄): δ=1.23 (s, 6H), 2.09 (quin, 2H), 2.58-2.78 (m, 4H), 2.84-3.06 (m, 5H), 3.12 (ddd, 1H).

### Intermediate 5-17

### 2-Methyl-1-({3-[(RS)-(4,4,5,5,5-pentafluoropentyl)sulphinyl]propyl}amino)propan-2-ol

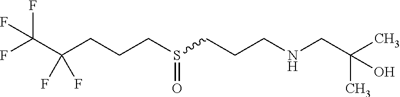 [SMILES: CC(C)(O)CNCCCS(=O)CCCC(F)(F)C(F)(F)F]

6.126 g (21.4 mmol) of 3-chloropropyl 4,4,5,5,5-pentafluoropentyl sulphoxide and 4.84 g (54.3 mmol) of 1-amino-2-methylpropan-2-ol were stirred according to General Procedure 15-17-19-C at 60° C. for 5 days and worked up. The product was purified on silica gel 60 (mobile phase: dichloromethane, dichloromethane-methanol 2:1, 1:1 and methanol with 5% by volume and 10% by volume of 33% strength ammonia solution). This gave 2.3 g (31% of theory) of product.

¹H NMR (400 MHz, chloroform-d₁): δ=1.18 (s, 6H), 1.95-2.06 (m, 2H), 2.11-2.32 (m, 4H), 2.56 (AB, 2H), 2.69-2.88 (m, 6H).

### Intermediate 6-17

### N-Methyl-3-[(RS)-(3,3,4,4,4-pentafluorobutyl)sulphinyl]propane-1-amine

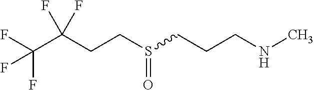 [SMILES: CNCCCS(=O)CCC(F)(F)C(F)(F)F]

4.75 g (17.4 mmol) of 3-chloropropyl 3,3,4,4,4-pentafluorobutyl sulphoxide were stirred in 100 ml of 33% strength ethanolic methylamine solution for 20 hours and worked up. The product was purified on silica gel 60 (mobile phase: dichloromethane, dichloromethane-methanol 2:1, 1:1 and methanol with 5% by volume and 10% by volume of 33% strength ammonia solution). This gave 4.45 g (96% of theory) of product.

¹H NMR (300 MHz, methanol-d₄): δ=1.74 (mc, 2H), 2.25 (s, 3H), 2.44-2.91 (m, 7H), 3.06 (ddd, 1H).

### Intermediate 7-17

### N-Methyl-5-[(RS)-(4,4,5,5,5-pentafluoropentyl)sulphinyl]pentane-1-amine

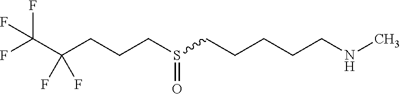 [SMILES: CNCCCCCS(=O)CCCC(F)(F)C(F)(F)F]

14 g (44.48 mmol) of 5-chloropentyl 4,4,5,5,5-pentafluoropentyl sulphoxide were reacted according to General Procedure 15-17-19-A. The product was purified on silica gel 60 (mobile phase: dichloromethane, dichloromethane-methanol 2:1, 1:1 and methanol with 1% by volume and 10% by volume of 33% strength ammonia solution). This gave 11.4 g (83% of theory) of product.

¹H NMR (400 MHz, chloroform-d₁): δ=1.43-1.62 (m, 2H), 1.72-1.92 (m, 4H), 2.04-2.32 (m, 4H), 2.67 (s, 3H), 2.71-2.84 (m, 2H), 2.91 (t, 2H).

### Intermediate 8-17

### N-Methyl-6-[(RS)-(4,4,5,5,5-pentafluoropentyl)sulphinyl]hexane-1-amine

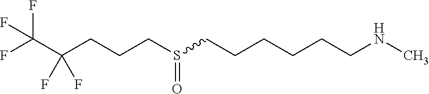 [SMILES: CNCCCCCCS(=O)CCCC(F)(F)C(F)(F)F]

14 g (42.58 mmol) of 6-chlorohexyl 4,4,5,5,5-pentafluoropentyl sulphoxide were reacted according to General Procedure 15-17-19-A. The product was purified on silica gel 60 (mobile phase: dichloromethane, dichloromethane-methanol 2:1, 1:1 and methanol with 1% by volume and 10% by volume of 33% strength ammonia solution). This gave 12.92 g (94% of theory) of product.

¹H NMR (600 MHz, chloroform-d₁): δ=1.37-1.56 (m, 6H), 1.74-1.84 (m, 2H), 2.12-2.30 (m, 4H), 2.44 (s, 3H), 2.59 (t, 2H), 2.62-2.78 (m, 4H).

### Intermediate 9-17

### N-Ethyl-3-[(RS)-(3,3,3-trifluoropropyl)sulphinyl]propane-1-amine

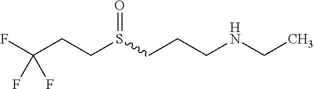

4 g (17.96 mmol) of 3-chloropropyl 3,3,3-trifluoropropyl sulphoxide were stirred with 25 ml of 30-40% strength methanolic ethylamine solution at 60° C. for 30 hours. The cold reaction solution was concentrated, saturated sodium carbonate solution was added and the mixture was extracted three times with dichloromethane. The combined organic phases were washed once with water, dried over magnesium sulphate and concentrated. 3.4 g (82% of theory) of product were isolated.

¹H NMR (300 MHz, chloroform-d₁): δ=1.10 (t, 3H), 1.28 (s-br, 1H), 1.87-2.04 (m, 2H), 2.51-2.70 (m, 4H), 2.73-2.97 (m, 6H).

### Intermediate 10-17

### 2-({3-[(RS)-(3,3,3-Trifluoropropyl)sulphinyl]propyl}amino)ethanol

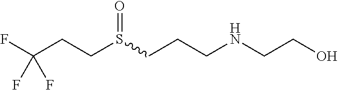 [SMILES: O=S(CCCNCCO)CCC(F)(F)F]

4 g (17.96 mmol) of 3-chloropropyl 3,3,3-trifluoropropyl sulphoxide and 6.41 ml of 2-aminoethanol were stirred for 30 hours according to General Procedure 15-17-19-C and worked up. The product was purified on silica gel 60 (mobile phase: dichloromethane, dichloromethane-methanol 4:1, 1:1 and methanol). This gave 1.67 g (38% of theory) of product.

¹H NMR (400 MHz, chloroform-d₁): δ=1.98 (quin, 2H), 2.54-2.68 (m, 2H), 2.74-2.94 (m, 8H), 3.63 (t, 2H).

### Intermediate 11-17

### 3-({3-[(RS)-(3,3,3-Trifluoropropyl)sulphinyl]propyl}amino)propan-1-ol

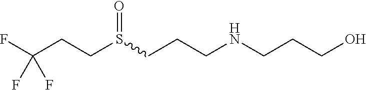 [SMILES: O=S(CCCNCCCO)CCC(F)(F)F]

4 g (17.96 mmol) of 3-chloropropyl 3,3,3-trifluoropropyl sulphoxide and 6.41 ml of 3-aminopropan-1-ol were stirred for 30 hours according to General Procedure 15-17-19-C and worked up. The product was purified on silica gel 60 (mobile phase: dichloromethane, dichloromethane-methanol 4:1, 1:1 and methanol). This gave 2.1 g (45% of theory) of product.

¹H NMR (400 MHz, chloroform-d₁): δ=1.70 (quin, 2H), 1.99 (quin, 2H), 2.54-2.69 (m, 2H), 2.72-2.94 (m, 8H), 3.79 (t, 2H).

### Intermediate 12-17

### N-Ethyl-3-[(RS)-(4,4,5,5,5-pentafluoropentyl)sulphinyl]propane-1-amine

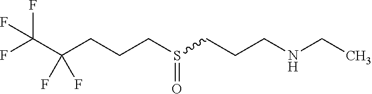 [SMILES: CCNCCCS(=O)CCCC(F)(F)C(F)(F)F]

At −5° C., 35 ml of 30-40% strength methanolic ethylamine solution were added to 6.5 g (22.67 mmol) of 3-chloropropyl 4,4,5,5,5-pentafluoropentyl sulphoxide. The mixture was stirred in an autoclave at 40° C. for 120 hours. The mixture was concentrated, saturated sodium carbonate solution was added to the residue and the mixture was extracted three times with dichloromethane. The combined organic phases were dried over magnesium sulphate and concentrated. The product was purified on silica gel 60 (mobile phase: dichloromethane, dichloromethane-methanol 4:1 and 2.1). This gave 3.08 g (46% of theory) of product.

¹H NMR (300 MHz, chloroform-d₁): δ=1.44 (t, 3H), 2.03-2.61 (m, 6H), 2.82-3.28 (m, 8H).

### Intermediate 13-17

### 2-({3-[(RS)-(4,4,5,5,5-Pentafluoropentyl)sulphinyl]propyl}amino)ethanol

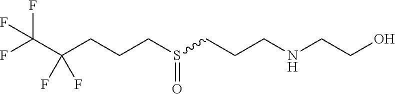 [SMILES: O=S(CCCNCCO)CCCC(F)(F)C(F)(F)F]

7 g (24.4 mmol) of 3-chloropropyl 4,4,5,5,5-pentafluoropentyl sulphoxide and 5.85 ml (97.7 mmol) of 2-aminoethanol were stirred in a microwave at 120 Watt for 30 minutes according to General Procedure 15-17-19-C and worked up. The product was purified on silica gel 60 (mobile phase: dichloromethane, dichloromethane-methanol 4:1, 2:1, 1:1 and methanol mit 5% by volume of 33% strength ammonia solution). This gave 2.85 g (37% of theory) of product.

¹H NMR (300 MHz, chloroform-d₁): δ=1.98 (quin, 2H), 2.05-2.33 (m, 6H), 2.67-2.87 (m, 8H), 3.64 (mc, 2H).

### Intermediate 14-17

### 3-({3-[(RS)-(4,4,5,5,5-Pentafluoropentyl)sulphinyl]propyl}amino)propan-1-ol

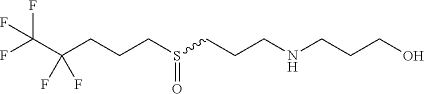 [SMILES: O=S(CCCNCCCO)CCCC(F)(F)C(F)(F)F]

7 g (24.4 mmol) of 3-chloropropyl 4,4,5,5,5-pentafluoropentyl sulphoxide and 7.45 ml (97.7 mmol) of 3-aminopropan-1-ol were stirred in a microwave at 120 Watt for 30 minutes according to General Procedure 15-17-19-C and worked up. The product was purified on silica gel 60 (mobile phase: dichloromethane, dichloromethane-methanol 4:1, 1:1 and methanol with 5% by volume of 33% strength ammonia solution). This gave 3.54 g (45% of theory) of product.

¹H NMR (400 MHz, chloroform-d₁): δ=1.71 (quin, 2H), 1.99 (quin, 2H), 2.10-2.32 (m, 4H), 2.70-2.83 (m, 6H), 2.89 (t, 2H), 3.80 (t, 2H).

### Intermediate 15-17

### N-(2-Methoxyethyl)-3-[(RS)-(4,4,5,5,5-pentafluoropentyl)sulphinyl]propane-1-amine

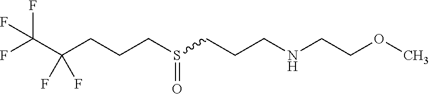 [SMILES: COCCNCCCS(=O)CCCC(F)(F)C(F)(F)F]

6.2 g (21.6 mmol) of 3-chloropropyl 4,4,5,5,5-pentafluoropentyl sulphoxide and 6.50 g (86.5 mmol) of 2-methoxyethane-1-amine were reacted according to General Procedure 15-17-19-C at 60° C. for 5 days. The mixture was purified twice on silica gel 60 (mobile phase: dichloromethane, dichloromethane-methanol 90:10, 80:20, 67:33 and methanol with 5% by volume of 33% strength ammonia solution, and dichloromethane, dichloromethane-methanol 9:1, 4:1, 2:1 and methanol with 5% by volume of 33% strength ammonia solution). This gave 2.30 g (33% of theory) of product.

¹H NMR (300 MHz, chloroform-d₁): δ=1.96 (quin, 2H), 2.07-2.33 (m, 4H), 2.68-2.85 (m, 8H), 3.35 (s, 3H), 3.47 (t, 2H).

### Intermediate 16-17

### 3-Methoxy-N-{3-[(RS)-(4,4,5,5-pentafluoropentyl)sulphinyl]propyl}propane-1-amine

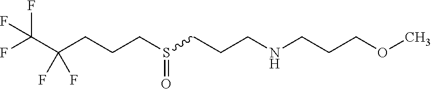 [SMILES: COCCCNCCCS(=O)CCCC(F)(F)C(F)(F)F]

6.3 g (22.0 mmol) of 3-chloropropyl 4,4,5,5,5-pentafluoropentyl sulphoxide and 7.84 g (88.0 mmol) of 3-methoxypropane-1-amine were reacted according to General Procedure 15-17-19-C at 60° C. for 5 days. The product was purified on silica gel 60 (mobile phase: dichloromethane, dichloromethane-methanol 90:10, 50:50 and methanol with 5% by volume of 33% strength ammonia solution). This gave 3.33 g (42% of theory) of product.

¹H NMR (300 MHz, chloroform-d₁): δ=1.74 (quin, 2H), 1.94 (quin, 2H), 2.08-2.35 (m, 4H), 2.61-2.84 (m, 8H), 3.32 (s, 3H), 3.43 (t, 2H).

### Intermediate 17-17

### N-Methyl-4-[(RS)-(3,3,3-trifluoropropyl)sulphinyl]butane-1-amine

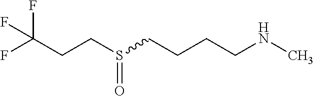

4.40 g (18.6 mmol) of 4-chlorobutyl 3,3,3-trifluoropropyl sulphoxide were stirred in 75 ml of 33% strength ethanolic methylamine solution at 40° C. for 24 hours. The volatile components were removed under reduced pressure, 50 ml of water were added and the mixture was washed twice with dichloromethane. The pH was adjusted to 14 using 2 M sodium hydroxide solution, and the mixture was extracted three times with dichloromethane. These combined organic phases were dried over magnesium sulphate and concentrated. 2.5 g (58% of theory) of product were obtained.

¹H NMR (400 MHz, chloroform-d₁): δ=1.58-1.74 (m, 2H), 1.77-1.92 (m, 2H), 2.43 (s, 3H), 2.55-2.93 (m, 8H).

### Intermediate 1-19

### N-Methyl-3-[(4,4,5,5,5-pentafluoropentyl)sulphonyl]propane-1-amine

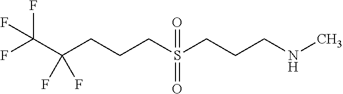 [SMILES: CNCCCS(=O)(=O)CCCC(F)(F)C(F)(F)F]

30 g (99.1 mmol) of 3-chloropropyl 4,4,5,5,5-pentafluoropentyl sulphone were reacted at 40° C. for 24 hours according to General Procedure 15-17-19-A and worked up. This gave 27.8 g (94% of theory) of product.

¹H NMR (400 MHz, chloroform-d₁): δ=1.22 (s-br, 1H), 2.00 (mc, 2H), 2.13-2.34 (m, 4H), 2.42 (s, 3H), 2.73 (t, 2H), 3.06 (t, 2H) 3.11 (mc, 2H).

### Intermediate 2-19

### N-Methyl-4-[(4,4,5,5,5-pentafluoropentyl)sulphonyl]butane-1-amine

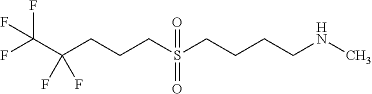 [SMILES: CNCCCCS(=O)(=O)CCCC(F)(F)C(F)(F)F]

16.2 g (51.15 mmol) of 4-chlorobutyl 4,4,5,5,5-pentafluoropentyl sulphone were reacted at 40° C. for 20 hours according to General Procedure 15-17-19-B and worked up. The product was purified on silica gel 60 (mobile phase: dichloromethane, dichloromethane-methanol 2:1, 1:1 and methanol with 1% by volume and 10% by volume of 33% strength ammonia solution). This gave 14.2 g (89% of theory) of product.

¹H NMR (600 MHz, chloroform-d₁): δ=1.49 (s-br, 1H), 1.66 (quin, 2H), 1.92 (mc, 2H), 2.16-2.34 (m, 4H), 2.44 (s, 3H), 2.64 (t, 2H), 3.01-3.08 (m, 4H).

### Intermediate 3-19

### N-Methyl-3-[(3,3,3-trifluoropropyl)sulphonyl]propane-1-amine

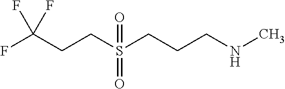

5.8 g (24.2 mmol) of 3-chloropropyl 3,3,3-trifluoropropyl sulphone were stirred for 20 hours according to General Procedure 15-17-19-B and worked up. The product was purified on silica gel 60 (mobile phase: dichloromethane, dichloromethane-methanol 2:1, 1:1 and methanol with 1.5% by volume of 33% strength ammonia solution). This gave 3.92 g (69% of theory) of product.

¹H NMR (400 MHz, DMSO-d₆): δ=2.03 (quin, 2H), 2.49 (s, 3H), 2.66-2.81 (m, 2H), 2.94 (t, 2H), 3.33-3.45 (m, 4H).

### Intermediate 4-19

### N-Ethyl-3-[(3,3,3-trifluoropropyl)sulphonyl]propane-1-amine

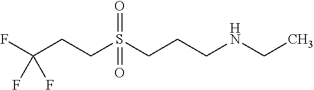

4 g (16.76 mmol) of 3-chloropropyl 3,3,3-trifluoropropyl sulphone and 25 ml of 30-40% strength methanolic ethylamine solution were stirred at 60° C. for 30 hours. The cold reaction solution was concentrated, saturated sodium carbonate solution was added and the mixture was extracted three times with dichloromethane. The combined organic phases were washed once with water, dried over magnesium sulphate and concentrated. 3.6 g (87% of theory) of product were isolated.

¹H NMR (300 MHz, chloroform-d₁): δ=1.05 (s-br, 1H), 1.09 (t, 3H), 1.96-2.07 (m, 2H), 2.59-2.81 (m, 6H), 3.13-3.25 (m, 4H).

### Intermediate 5-19

### 2-({3-[(3,3,3-Trifluoropropyl)sulphonyl]propyl}amino)ethanol

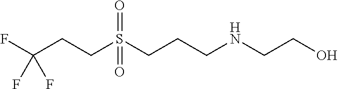 [SMILES: O=S(=O)(CCCNCCO)CCC(F)(F)F]

4 g (16.76 mmol) of 3-chloropropyl 3,3,3-trifluoropropyl sulphone and 5.98 ml of 2-aminoethan-1-ol were stirred for 30 hours according to General Procedure 15-17-19-C and worked up. The product was purified on silica gel 60 (mobile phase: dichloromethane, dichloromethane-methanol 4:1, 1:1 and methanol). This gave 2.3 g (52% of theory) of product.

¹H NMR (400 MHz, chloroform-d₁): δ=1.82 (s-br, 2H), 2.04 (mc, 2H), 2.62-2.74 (m, 2H), 2.75-2.84 (m, 4H), 3.14-3.23 (m, 4H), 3.66 (t, 2H).

### Intermediate 6-19

### 3-({3-[(3,3,3-Trifluoropropyl)sulphonyl]propyl}amino)propan-1-ol

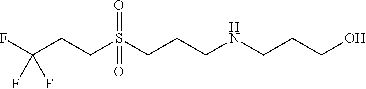 [SMILES: O=S(=O)(CCCNCCCO)CCC(F)(F)F]

4 g (16.76 mmol) of 3-chloropropyl 3,3,3-trifluoropropyl sulphone and 5.88 ml of 3-aminopropan-1-ol were stirred for 30 hours according to General Procedure 15-17-19-C and worked up. The product was purified on silica gel 60 (mobile phase: dichloromethane, dichloromethane-methanol 4:1, 1:1 and methanol). This gave 2.7 g (58% of theory) of product.

¹H NMR (400 MHz, chloroform-d₁): δ=1.70 (quin, 2H), 2.04 (mc, 2H), 2.61-2.74 (m, 2H), 2.79 (t, 2H), 2.86 (t, 2H), 3.13 (mc, 2H), 3.19 (mc, 2H), 3.79 (t, 2H).

### Intermediate 7-19

### 2-Methyl-1-({3-[(3,3,3-trifluoropropyl)sulphonyl]propyl}amino)propan-2-ol

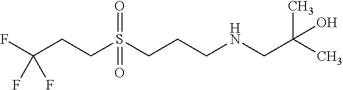 [SMILES: CC(C)(O)CNCCCS(=O)(=O)CCC(F)(F)F]

4 g (16.76 mmol) of 3-chloropropyl 3,3,3-trifluoropropyl sulphone and 5.24 ml of 1-amino-2-methylpropan-2-ol were stirred for 30 hours according to General Procedure 15-17-19-C and worked up. The product was purified on silica gel 60 (mobile phase: dichloromethane, dichloromethane-methanol 4:1 and 1:1). This gave 2.1 g (43% of theory) of product.

¹H NMR (300 MHz, methanol-d₄): δ=1.19 (s, 6H), 1.93-2.05 (m, 2H), 2.53 (s, 2H), 2.62-2.79 (m, 4H), 3.24 (mc, 2H), 3.30-3.42 (m, 2H).

### Intermediate 8-19

### N-Methyl-3-[(3,3,4,4,4-pentafluorobutyl)sulphonyl]propane-1-amine

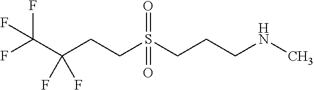 [SMILES: CNCCCS(=O)(=O)CCC(F)(F)C(F)(F)F]

7.7 g (26.67 mmol) of 3-chloropropyl 3,3,4,4,4-pentafluorobutyl sulphone were stirred for 20 hours according to General Procedure 15-17-19-B and worked up. The product was purified on silica gel 60 (mobile phase: dichloromethane, dichloromethane-methanol 2:1, 1:1 and methanol with 1.5% by volume of 33% strength ammonia solution). This gave 5.21 g (69% of theory) of product.

¹H NMR (400 MHz, DMSO-d₆): δ=2.03 (quin, 2H), 2.50 (s, 3H), 2.57-2.77 (m, 2H), 2.94 (t, 2H), 3.39 (t, 2H), 3.45 (mc, 2H).

### Intermediate 9-19

### 2-({3-[(4,4,5,5,5-Pentafluoropentyl)sulphonyl]propyl}amino)ethanol

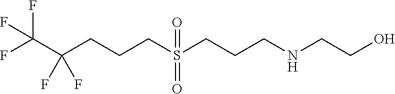 [SMILES: O=S(=O)(CCCNCCO)CCCC(F)(F)C(F)(F)F]

7.39 g (24.4 mmol) of 3-chloropropyl 4,4,5,5,5-pentafluoropentyl sulphone and 5.97 g (97.7 mmol) of 3-aminopropan-1-ol were stirred in a microwave at 120 Watt for 30 minutes and extracted four times with chloroform according to General Procedure 15-17-19-C. After the extraction, a white precipitate was filtered off with suction from the combined organic phases and dried. This gave 385 mg (5% of theory) of product. A precipitate was filtered off with suction from the aqueous phase, this precipitate was taken up in chloroform, washed once with water, dried over magnesium sulphate and concentrated. 0.92 g (12% of theory) of white product was obtained. The combined organic phases were dried over magnesium sulphate and concentrated. The product was purified on silica gel 60 (mobile phase: dichloromethane, dichloromethane-methanol 4:1, 1:1 and methanol with 4% by volume of 33% strength ammonia solution). This gave 1.36 g (17% of theory) of product.

¹H NMR (300 MHz, chloroform-d₁): δ=1.98-2.09 (m, 2H), 2.14-2.38 (m, 4H), 2.75-2.85 (m, 4H), 3.03-3.16 (m, 4H) 3.66 (mc, 2H).

### Intermediate 10-19

### 3-({3-[(4,4,5,5,5-Pentafluoropentyl)sulphonyl]propyl}amino)propan-1-ol

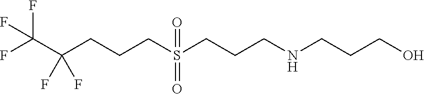 [SMILES: O=S(=O)(CCCNCCCO)CCCC(F)(F)C(F)(F)F]

7 g (23.1 mmol) of 3-chloropropyl 4,4,5,5,5-pentafluoropentyl sulphone and 6.95 g (92.5 mmol) of 3-aminopropan-1-ol were stirred at 60° C. for 7 days according to General Procedure 15-17-19-C and worked up. The product was purified on silica gel 60 (mobile phase: dichloromethane, dichloromethane-methanol 4:1, 1:1 and methanol with 2% by volume and 5% by volume of 33% strength ammonia solution). This gave 4.18 g (53% of theory) of product.

¹H NMR (400 MHz, chloroform-d₁): δ=1.71 (quin, 2H), 1.98-2.08 (m, 2H), 2.14-2.35 (m, 4H), 2.71 (br s, 2H), 2.79 (t, 2H), 2.87 (t, 2H), 3.03-3.11 (m, 4H) 3.79 (t, 2H).

### Intermediate 11-19

### 2-Methyl-1-({3-[(4,4,5,5,5-pentafluoropentyl)sulphonyl]propyl}amino)propan-2-ol

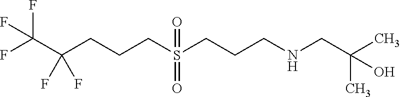 [SMILES: CC(C)(O)CNCCCS(=O)(=O)CCCC(F)(F)C(F)(F)F]

6.5 g (21.5 mmol) of 3-chloropropyl 4,4,5,5,5-pentafluoropentyl sulphone and 4.86 g (54.6 mmol) of 1-amino-2-methylpropan-2-ol were stirred at 60° C. for 8 days according to General Procedure 15-17-19-C and worked up. The product was purified on silica gel 60 (mobile phase:dichloromethane, dichloromethane-methanol 4:1, 1:1 and methanol with 4% by volume and 5% by volume of 33% strength ammonia solution). This gave 1.45 g (19% of theory) of product.

¹H NMR (400 MHz, chloroform-d₁): δ=1.19 (s, 6H), 2.03 (mc, 2H), 2.15-2.38 (m, 4H), 2.55 (s, 2H), 2.84 (t, 2H), 3.07 (t, 2H) 3.12 (mc, 2H).

### Intermediate 12-19

### N-(2-Methoxyethyl)-3-[(4,4,5,5,5-pentafluoropentyl)sulphonyl]propane-1-amine

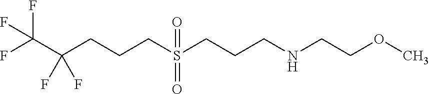 [SMILES: COCCNCCCS(=O)(=O)CCCC(F)(F)C(F)(F)F]

8.00 g (26.4 mmol) of 3-chloropropyl 4,4,5,5,5-pentafluoropentyl sulphone and 5.96 g (79.3 mmol) of 2-methoxyethylamine were reacted according to General Procedure 15-17-19-C at 60° C. for 7 days. The product was purified on silica gel 60 (mobile phase: dichloromethane, dichloromethane-methanol 95:5, 90:10, 80:20, 50:50 and methanol with 4% by volume of 33% strength ammonia solution). This gave 3.36 g (37% of theory) of product.

¹H NMR (300 MHz, chloroform-d₁): δ=2.02 (mc, 2H), 2.12-2.38 (m, 4H), 2.75-2.83 (m, 4H), 3.06 (t, 2H), 3.13 (mc, 2H), 3.36 (s, 3H), 3.48 (t, 2H).

### Intermediate 13-19

### 3-Methoxy-N-{3-[(4,4,5,5,5-pentafluoropentyl)sulphonyl]propyl}propane-1-amine

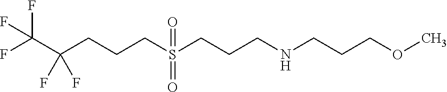 [SMILES: COCCCNCCCS(=O)(=O)CCCC(F)(F)C(F)(F)F]

8.00 g (26.4 mmol) of 3-chloropropyl 4,4,5,5,5-pentafluoropentyl sulphone and 5.89 g (66.1 mmol) of 3-methoxypropylamine were reacted at 60° C. for 7 days according to General Procedure 15-17-19-C. The product was purified on silica gel 60 (mobile phase: dichloromethane, dichloromethane-methanol 95:5, 90:10, 70:30, 50:50 and methanol with 4% by volume of 33% strength ammonia solution). This gave 3.99 g (42% of theory) of product.

¹H NMR (300 MHz, chloroform-d₁): δ=1.74 (quin, 2H), 2.00 (mc, 2H), 2.12-2.37 (m, 4H), 2.68 (t, 2H), 2.76 (t, 2H), 3.06 (t, 2H), 3.12 (mc, 2H), 3.32 (s, 3H), 3.44 (t, 2H).

### Intermediate 14-19

### N-Methyl-4-[(3,3,3-trifluoropropyl)sulphonyl]butane-1-amine

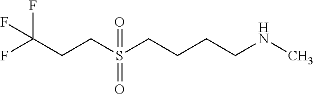

5.0 g (19.8 mmol) of 4-chlorobutyl 3,3,3-trifluoropropyl sulphone were stirred in 80 ml of 33% strength ethanolic methylamine solution at 40° C. for 24 hours. The volatile components were removed under reduced pressure, 50 ml of water were added and the mixture was washed twice with dichloromethane. The pH was adjusted to 14 using 2 M sodium hydroxide solution and the mixture was extracted three times with dichloromethane. These combined organic phases were dried over magnesium sulphate and concentrated. 4.4 g (90% of theory) of product were obtained.

¹H NMR (400 MHz, chloroform-d₁): δ=1.65 (quin, 2H), 1.88-1.98 (m, 2H), 2.43 (s, 3H), 2.66-2.75 (m, 4H), 3.08 (mc, 2H), 3.15-3.21 (m, 2H).

### Intermediate 15-19

### N-Methyl-3-[(4,4,4-trifluorobutyl)sulphonyl]propane-1-amine

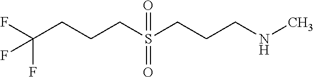

1.0 g (3.96 mmol) of 3-chloropropyl 4,4,4-trifluorobutyl sulphone was stirred in 50 ml of 33% strength solution of methylamine in ethanol for 24 hours and worked up according to General Procedure 15-17-19-D. This gave 0.56 g (57% of theory) of product.

¹H NMR (400 MHz, chloroform-d₁): δ=2.00 (mc, 2H), 2.10-2.19 (m, 2H), 2.25-2.38 (m, 2H), 2.42 (s, 3H), 2.73 (t, 2H), 3.04 (mc, 2H), 3.10 (mc, 2H).

### Intermediate 16-19

### N-Methyl-4-[(4,4,4-trifluorobutyl)sulphonyl]butane-1-amine

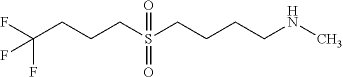 [SMILES: CNCCCCS(=O)(=O)CCCC(F)(F)F]

15.0 g (56.2 mmol) of 4-chlorobutyl 4,4,4-trifluorobutyl sulphone were stirred in 300 ml of 33% strength solution of methylamine in ethanol for 36 hours according to General Procedure 15-17-19-D and worked up. This gave 12.8 g (87% of theory) of product.

¹H NMR (400 MHz, chloroform-d₁): δ=1.65 (quin, 2H), 1.87-1.97 (m, 2H), 2.10-2.20 (m, 2H), 2.26-2.41 (m, 2H), 2.43 (s, 3H), 2.64 (t, 2H), 3.00-3.07 (mc, 4H).

### Intermediate 17-19

### N-Methyl-5-[(4,4,5,5,5-pentafluoropentyl)sulphonyl]pentane-1-amine

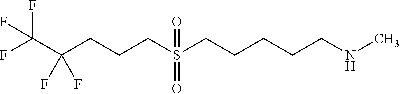 [SMILES: CNCCCCCS(=O)(=O)CCCC(F)(F)C(F)(F)F]

16 g (48.36 mmol) of 5-chloropentyl 4,4,5,5,5-pentafluoropentyl sulphone were reacted according to General Procedure 15-17-19-B at 40° C. for 20 hours. The product was purified on silica gel 60 (mobile phase: dichloromethane, dichloromethane-methanol 2:1, 1:1 and methanol with 1% by volume and 10% by volume of 33% strength ammonia solution). This gave 12.02 g (76% of theory) of product.

¹H NMR (300 MHz, methanol-d₄): δ=1.49-1.62 (m, 2H), 1.66-1.78 (m, 2H), 1.86 (mc, 2H), 2.02-2.14 (m, 2H), 2.25-2.46 (m, 2H), 2.68 (s, 3H), 2.99 (mc, 2H), 3.14 (mc, 2H), 3.21 (t, 2H).

### Intermediate 18-19

### N-Methyl-6-[(4,4,5,5,5-pentafluoropentyl)sulphonyl]hexane-1-amine

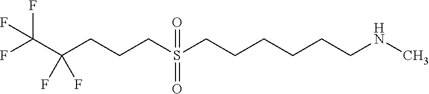 [SMILES: CNCCCCCCS(=O)(=O)CCCC(F)(F)C(F)(F)F]

16.4 g (47.57 mmol) of 6-chlorohexyl 4,4,5,5,5-pentafluoropentyl sulphone were reacted according to General Procedure 15-17-19-B at 40° C. for 16 hours. The product was purified on silica gel 60 (mobile phase: dichloromethane, dichloromethane-methanol 2:1, 1:1 and methanol with 1% by volume and 10% by volume of 33% strength ammonia solution). This gave 10.07 g (62% of theory) of product.

¹H NMR (600 MHz, DMSO-d₆): δ=1.30-1.36 (m, 2H), 1.37-1.43 (m, 2H), 1.53 (quin, 2H), 1.64-1.71 (m, 2H), 1.90-1.98 (m, 2H), 2.34-2.48 (m, 5H), 2.75 (t, 2H), 3.12 (mc, 2H), 3.22 (t, 2H).

### Intermediate 19-19

### N-Ethyl-3-[(4,4,5,5,5-pentafluoropentyl)sulphonyl]propane-1-amine

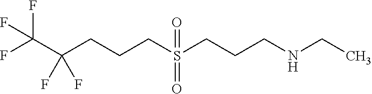 [SMILES: CCNCCCS(=O)(=O)CCCC(F)(F)C(F)(F)F]

6.5 g (21.5 mmol) of 3-chloropropyl 4,4,5,5,5-pentafluoropentyl sulphone were stirred with 35 ml of 30-40% strength methanolic ethylamine solution in an autoclave at 40° C. for 2 days. The cold reaction solution was concentrated, saturated sodium carbonate solution was added and the mixture was extracted three times with dichloromethane. The combined organic phases were dried over magnesium sulphate and concentrated. The product was purified on silica gel 60 (mobile phase:dichloromethane, dichloromethane-methanol 4:1, 1:1 and methanol with 5% by volume of 33% strength ammonia solution). 2.48 g (37% of theory) of product were isolated.

¹H NMR (400 MHz, chloroform-d₁): δ=1.10 (t, 3H), 1.97-2.06 (m, 2H), 2.14-2.34 (m, 4H), 2.65 (q, 2H), 2.78 (t, 2H), 3.06 (t, 2H) 3.12 (mc, 2H).

### Intermediate 21

### 2-{3-[(4,4,5,5,5-Pentafluoropentyl)sulphanyl]propyl}-2,3-dihydro-1H-isoindole-1,3-dione

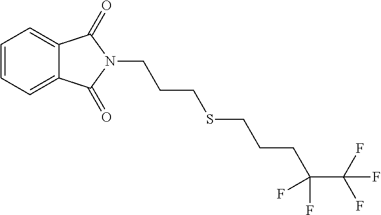 [SMILES: O=C1c2ccccc2C(=O)N1CCCSCCCC(F)(F)C(F)(F)F]

On an ice bath, 1 molar equivalent of a 30% strength solution of sodium methoxide in methanol was added to 5 g (21.17 mmol) of S-(4,4,5,5,5-pentafluoropentyl) thioacetate in 25 ml of methanol, and the mixture was stirred on the ice bath for 30 minutes. 5.68 g (21.18 mmol) of 2-(3-bromopropyl)-2,3-dihydro-1H-isoindole-1,3-dione were added a little at a time, and the mixture was stirred at room temperature overnight. The reaction was concentrated, taken up in water and extracted three times with ether. The combined organic phases were twice washed with water, dried over magnesium sulphate and concentrated. 8 g (99% of theory) of product were isolated.

¹H NMR (400 MHz, chloroform-d₁): δ=1.82-1.91 (m, 2H), 1.97 (quin, 2H), 2.07-2.22 (m, 2H), 2.56 (t, 2H), 2.60 (t, 2H), 3.80 (t, 2H), 7.69-7.75 (m, 2H), 7.82-8.87 (m, 2H).

### Intermediate 22

### 2-{3-[(RS)-(4,4,5,5,5-Pentafluoropentyl)sulphinyl]propyl}-2,3-dihydro-1H-isoindole-1,3-dione

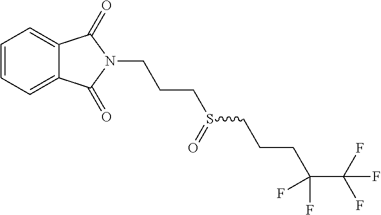 [SMILES: O=C1c2ccccc2C(=O)N1CCCS(=O)CCCC(F)(F)C(F)(F)F]

8 g (20.98 mmol) of 2-{3-[(4,4,5,5,5-pentafluoropentyl)sulphanyl]propyl}-2,3-dihydro-1H-isoindole-1,3-dione were reacted according to General Procedure 17. The residue was digested with hexane, filtered off with suction and dried. 7 g (84% of theory) of white crystals were isolated.

¹H NMR (300 MHz, chloroform-d₁): δ=2.07-2.33 (m, 6H), 2.66-2.83 (m, 4H), 3.80-3.94 (m, 2H), 7.74 (dd, 2H), 7.85 (dd, 2H).

### Intermediate 23

### 3-[(RS)-(4,4,5,5,5-Pentafluoropentyl)sulphinyl]propane-1-amine

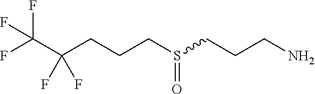 [SMILES: NCCCS(=O)CCCC(F)(F)C(F)(F)F]

3.5 g (8.81 mmol) of 2-{3-[(RS)-(4,4,5,5,5-pentafluoropentyl)sulphinyl]propyl}-2,3-dihydro-1H-isoindole-1,3-dione in 30 ml of a 33% strength solution of methylamine in ethanol were stirred under reflux for 4 hours. The mixture was concentrated to dryness. The residue was purified on silica gel 60 (mobile phase: methanol and methanol with 1.5% by volume of 25% strength ammonia solution). The collected fractions were concentrated to dryness, taken up in dichloromethane, dried over magnesium sulphate and concentrated. Hexane was added to the residue, the material was filtered off with suction and dried in a drying cabinet over potassium hydroxide. 400 mg (17% of theory) of product were isolated.

¹H NMR (300 MHz, DMSO-d₆): δ=1.59-1.70 (m, 2H), 1.87 (quin, 2H), 2.24-2.46 (m, 2H), 2.56-2.88 (m, 6H).

### EXAMPLES

General Procedure 11 for the preparation of the examples under an atmosphere of protective gas and with exclusion of moisture: 1 g of bromide was dissolved in about 30-55 ml of DMF. 1.2-1.4 equivalents of amine (based on the bromide), 0.5 equivalents of sodium iodide (based on the bromide) and 1.0 equivalent of sodium carbonate (based on the bromide) were added. The mixture was stirred at a bath temperature of 80-85° C. for 10-24 hours. After cooling to room temperature, the solution was concentrated under oil pump vacuum on a rotary evaporator. The residue was taken up in ethyl acetate or dichloromethane, washed twice or three times (water, optionally saturated sodium chloride solution), dried over magnesium sulphate and concentrated. The product was then chromatographed on silica gel 60 or by HPLC.

### Example 1

### 9-[5-(Methyl{3-[(4,4,5,5,5-pentafluoropentyl)sulphanyl]propyl}amino)pentyl]-8-phenyl-6,7-dihydro-5H-benzo[7]annulen-3-ol

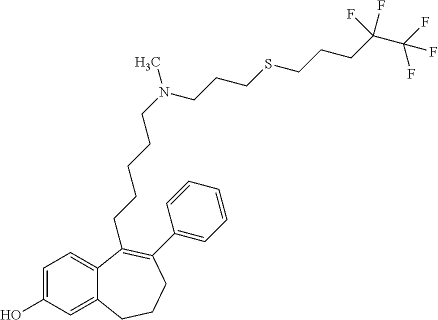 [SMILES: CN(CCCCCC1=C(c2ccccc2)CCCc2cc(O)ccc21)CCCSCCCC(F)(F)C(F)(F)F]

300 mg (0.78 mmol) of 9-(5-bromopentyl)-8-phenyl-6,7-dihydro-5H-benzo[7]annulen-3-ol were reacted with 289.2 mg (1.09 mmol) of N-methyl-3-[(4,4,5,5,5-pentafluoropentyl)sulphanyl]propane-1-amine according to Procedure 11. The product was purified on silica gel 60 (mobile phase: dichloromethane, dichloromethane-methanol 95:5). This gave 240 mg (54% of theory) of product.

¹H NMR (300 MHz, chloroform-d₁): δ=1.08-1.46 (m, 6H), 1.74-1.97 (m, 4H), 2.08-2.71 (m, 21H), 6.70-6.78 (m, 2H), 7.20 (mc, 1H), 7.25-7.33 (m, 3H), 7.36-7.44 (m, 2H).

### Example 2

### 9-[5-(Methyl{3-[(RS)-(4,4,5,5,5-pentafluoropentyl)sulphinyl]propyl}amino)pentyl]-8-phenyl-6,7-dihydro-5H-benzo[7]annulen-3-ol

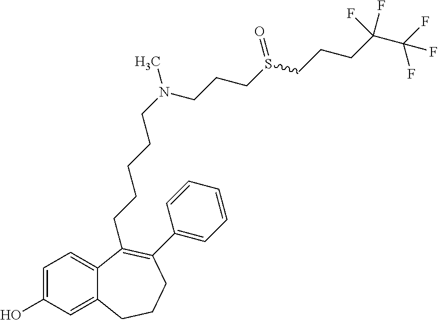 [SMILES: CN(CCCCCC1=C(c2ccccc2)CCCc2cc(O)ccc21)CCCS(=O)CCCC(F)(F)C(F)(F)F]

300 mg (0.78 mmol) of 9-(5-bromopentyl)-8-phenyl-6,7-dihydro-5H-benzo[7]annulen-3-ol were reacted with 306.6 mg (1.09 mmol) of N-methyl-3-[(RS)-(4,4,5,5,5-pentafluoropentyl)sulphinyl]propane-1-amine according to Procedure 11. The product was purified on silica gel 60 (mobile phase: dichloromethane, dichloromethane-methanol 95:5 and 90:10). This gave 330 mg (72% of theory) of product.

¹H NMR (300 MHz, chloroform-d₁): δ=1.01-1.15 (m, 2H), 1.16-1.35 (m, 4H), 1.90 (mc, 2H), 2.03-2.47 (m, 17H), 2.57-2.85 (m, 6H), 6.70-6.79 (m, 2H), 7.16 (d, 1H), 7.20-7.29 (m, 3H), 7.30-7.39 (m, 2H).

### Example 3

### 9-[5-(Methyl{3-[(4,4,5,5,5-pentafluoropentyl)sulphonyl]propyl}amino)pentyl]-8-phenyl-6,7-dihydro-5H-benzo[7]annulen-3-ol

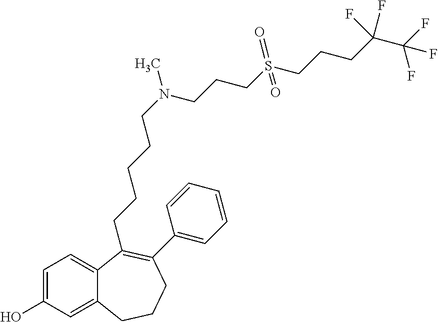 [SMILES: CN(CCCCCC1=C(c2ccccc2)CCCc2cc(O)ccc21)CCCS(=O)(=O)CCCC(F)(F)C(F)(F)F]

300 mg (0.78 mmol) of 9-(5-bromopentyl)-8-phenyl-6,7-dihydro-5H-benzo[7]annulen-3-ol were reacted with 324 mg (1.09 mmol) of N-methyl-3-[(4,4,5,5,5-pentafluoropentyl)sulphonyl]propane-1-amine according to Procedure 11. The product was purified on silica gel 60 (mobile phase: dichloromethane, dichloromethane-methanol 95:5). Hexane was added to the residue, and the product was filtered off with suction and dried in a drying cabinet at 50° C. This gave 280 mg (60% of theory) of product. M.p.: 111° C.

¹H NMR (300 MHz, chloroform-d₁): δ=1.01-1.32 (m, 6H), 1.94 (mc, 2H), 2.04-2.44 (m, 17H), 2.63 (mc, 2H), 2.96-3.08 (m, 4H), 6.68-6.76 (m, 2H), 7.17 (d, 1H), 7.21-7.29 (m, 3H), 7.31-7.40 (m, 2H).

### Example 4

### 8-(3-Hydroxyphenyl)-9-[5-(methyl{3-[(RS)-(4,4,5,5,5-pentafluoropentyl)sulphinyl]propyl}amino)pentyl]-6,7-dihydro-5H-benzo[7]annulen-3-ol

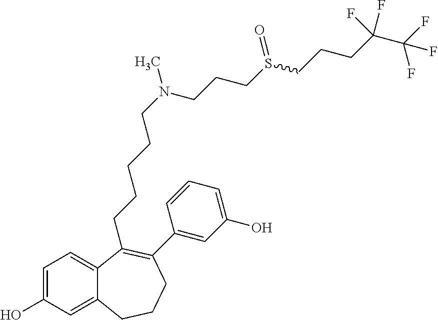 [SMILES: CN(CCCCCC1=C(c2cccc(O)c2)CCCc2cc(O)ccc21)CCCS(=O)CCCC(F)(F)C(F)(F)F]

250 mg (0.62 mmol) of 9-(5-bromopentyl)-8-(3-hydroxyphenyl)-6,7-dihydro-5H-benzo[7]annulen-3-ol were reacted with 245 mg (0.87 mmol) of N-methyl-3-[(RS)-(4,4,5,5,5-pentafluoropentyl)sulphinyl]propane-1-amine according to Procedure 11. The material was purified by HPLC method 2. This gave 369 mg (98% of theory) of product.

¹H NMR (400 MHz, chloroform-d₁): δ=1.13-1.23 (m, 2H), 1.23-1.32 (m, 2H), 1.40 (mc, 2H), 1.95-2.34 (m, 15H), 2.43 (t, 2H), 2.51 (mc, 2H), 2.60 (t, 2H), 2.69-2.86 (m, 4H), 6.65-6.76 (m, 5H), 7.15 (d, 1H), 7.20 (t, 1H).

### Example 5

### 8-(3-Hydroxyphenyl)-9-[5-(methyl{3-[(4,4,5,5,5-pentafluoropentyl)sulphonyl]propyl}amino)pentyl]-6,7-dihydro-5H-benzo[7]annulen-3-ol

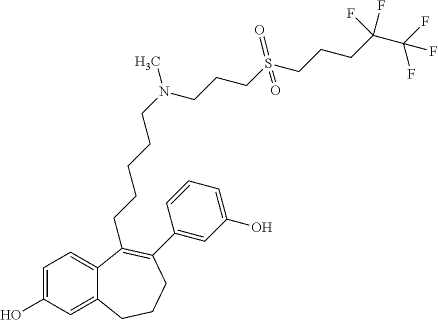 [SMILES: CN(CCCCCC1=C(c2cccc(O)c2)CCCc2cc(O)ccc21)CCCS(=O)(=O)CCCC(F)(F)C(F)(F)F]

250 mg (0.62 mmol) of 9-(5-bromopentyl)-8-(3-hydroxyphenyl)-6,7-dihydro-5H-benzo[7]annulen-3-ol were reacted with 259 mg (0.87 mmol) of N-methyl-3-[(4,4,5,5,5-pentafluoropentyl)sulphonyl]propane-1-amine according to Procedure 11. The material was purified by HPLC method 2. This gave 285 mg (74% of theory) of product.

¹H NMR (400 MHz, chloroform-d₁): δ=1.16-1.34 (m, 4H), 1.46 (mc, 2H), 2.03-2.33 (m, 13H), 2.37 (mc, 2H), 2.46 (t, 2H), 2.54 (t, 2H), 2.62 (t, 2H), 3.02-3.11 (m, 4H), 6.61-6.64 (m, 1H), 6.69-6.76 (m, 4H), 7.17 (d, 1H), 7.22 (t, 1H).

### Example 6

### 8-(3-Hydroxyphenyl)-9-[5-(methyl{4-[(RS)-(4,4,5,5,5-pentafluoropentyl)sulphinyl]butyl}amino)pentyl]-6,7-dihydro-5H-benzo[7]annulen-3-ol

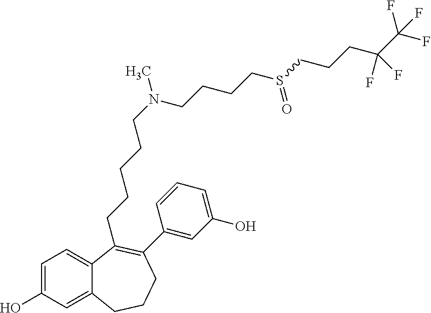 [SMILES: CN(CCCCCC1=C(c2cccc(O)c2)CCCc2cc(O)ccc21)CCCCS(=O)CCCC(F)(F)C(F)(F)F]

250 mg (0.62 mmol) of 9-(5-bromopentyl)-8-(3-hydroxyphenyl)-6,7-dihydro-5H-benzo[7]annulen-3-ol were reacted with 258 mg (0.87 mmol) of N-methyl-4-[(RS)-(4,4,5,5,5-pentafluoropentyl)sulphinyl]butane-1-amine according to Procedure 11. The material was purified by HPLC method 3. This gave 339 mg (88% of theory) of product.

¹H NMR (400 MHz, chloroform-d₁): δ=1.16-1.35 (m, 4H), 1.45 (mc, 2H), 1.60-1.87 (m, 4H), 2.02-2.37 (m, 13H), 2.38-2.50 (m, 4H), 2.60 (t, 2H), 2.65-2.85 (m, 4H), 6.61-6.64 (m, 1H), 6.68-6.76 (m, 4H), 7.16 (d, 1H), 7.21 (t, 1H).

### Example 7

### 8-(3-Hydroxyphenyl)-9-[5-(methyl{4-[(4,4,5,5,5-pentafluoropentyl)sulphonyl]butyl}amino)pentyl]-6,7-dihydro-5H-benzo[7]annulen-3-ol

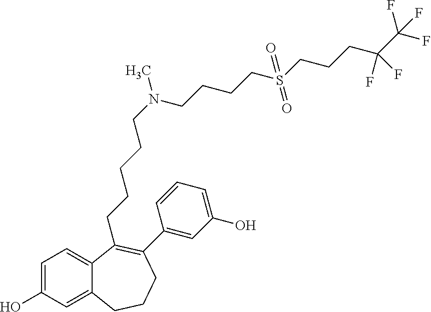 [SMILES: CN(CCCCCC1=C(c2cccc(O)c2)CCCc2cc(O)ccc21)CCCCS(=O)(=O)CCCC(F)(F)C(F)(F)F]

250 mg (0.62 mmol) of 9-(5-bromopentyl)-8-(3-hydroxyphenyl)-6,7-dihydro-5H-benzo[7]annulen-3-ol were reacted with 271 mg (0.87 mmol) of N-methyl-4-[(4,4,5,5,5-pentafluoropentyl)sulphonyl]butane-1-amine according to Procedure 11. The material was purified by HPLC method 1. This gave 339 mg (88% of theory) of product.

¹H NMR (400 MHz, chloroform-d₁): δ=1.19-1.37 (m, 4H), 1.51 (mc, 2H), 1.69-1.78 (m, 2H), 1.84-1.93 (m, 2H), 2.02-2.31 (m, 11H), 2.36-2.50 (m, 6H), 2.61 (t, 2H), 2.98-3.06 (m, 4H), 6.58-6.60 (m, 1H), 6.68-6.76 (m, 4H), 7.16 (d, 1H), 7.23 (t, 1H).

### Example 8

### 8-(3-Hydroxyphenyl)-9-[5-(methyl{5-[(RS)-(4,4,5,5,5-pentafluoropentyl)sulphinyl]pentyl}amino)pentyl]-6,7-dihydro-5H-benzo[7]annulen-3-ol

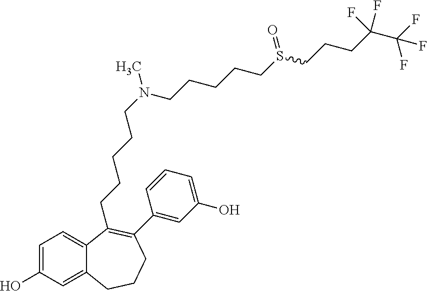 [SMILES: CN(CCCCCC1=C(c2cccc(O)c2)CCCc2cc(O)ccc21)CCCCCS(=O)CCCC(F)(F)C(F)(F)F]

250 mg (0.62 mmol) of 9-(5-bromopentyl)-8-(3-hydroxyphenyl)-6,7-dihydro-5H-benzo[7]annulen-3-ol were reacted with 270 mg (0.87 mmol) of N-methyl-5-[(RS)-(4,4,5,5,5-pentafluoropentyl)sulphinyl]pentane-1-amine according to Procedure 11. The material was purified by HPLC method 1. This gave 344 mg (88% of theory) of product.

¹H NMR (400 MHz, chloroform-d₁): δ=1.17-1.35 (m, 4H), 1.38-1.65 (mc, 6H), 1.83 (mc, 2H), 2.02-2.31 (m, 11H), 2.32-2.49 (m, 6H), 2.60 (t, 2H), 2.62-2.83 (m, 4H), 6.61-6.64 (m, 1H), 6.68-6.76 (m, 4H), 7.15 (d, 1H), 7.21 (t, 1H).

### Example 9

### 8-(3-Hydroxyphenyl)-9-[5-(methyl{5-[(4,4,5,5,5-pentafluoropentyl)sulphonyl]pentyl}amino)pentyl]-6,7-dihydro-5H-benzo[7]annulen-3-ol

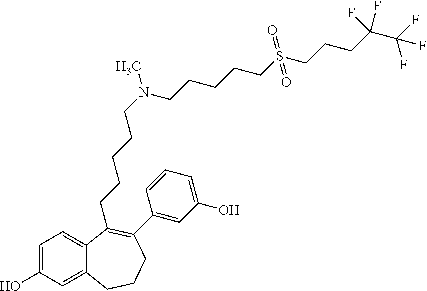 [SMILES: CN(CCCCCC1=C(c2cccc(O)c2)CCCc2cc(O)ccc21)CCCCCS(=O)(=O)CCCC(F)(F)C(F)(F)F]

250 mg (0.62 mmol) of 9-(5-bromopentyl)-8-(3-hydroxyphenyl)-6,7-dihydro-5H-benzo[7]annulen-3-ol were reacted with 284 mg (0.87 mmol) of N-methyl-5-[(4,4,5,5,5-pentafluoropentyl)sulphonyl]pentane-1-amine according to Procedure 11. The material was purified by HPLC method 1. This gave 313 mg (78% of theory) of product.

¹H NMR (400 MHz, chloroform-d₁): δ=1.20-1.37 (m, 4H), 1.42-1.57 (m, 4H), 1.59-1.69 (m, 2H), 1.89 (mc, 2H), 2.02-2.12 (m, 2H), 2.13-2.34 (m, 9H), 2.37-2.51 (m, 6H), 2.61 (t, 2H), 2.95-3.05 (m, 4H), 6.57-6.60 (m, 1H), 6.67-6.77 (m, 4H), 7.16 (d, 1H), 7.23 (t, 1H).

### Example 10

### 8-(3-Hydroxyphenyl)-9-[5-(methyl{6-[(RS)-(4,4,5,5,5-pentafluoropentyl)sulphinyl]hexyl}amino)pentyl]-6,7-dihydro-5H-benzo[7]annulen-3-ol

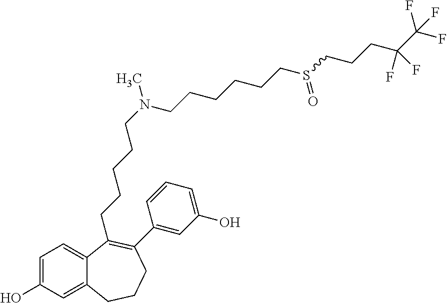 [SMILES: CN(CCCCCCS(=O)CCCC(F)(F)C(F)(F)F)CCCCCC1=C(c2cccc(O)c2)CCCc2cc(O)ccc21]

250 mg (0.62 mmol) of 9-(5-bromopentyl)-8-(3-hydroxyphenyl)-6,7-dihydro-5H-benzo[7]annulen-3-ol were reacted with 282 mg (0.87 mmol) of N-methyl-6-[(RS)-(4,4,5,5,5-pentafluoropentyl)sulphinyl]hexane-1-amine according to Procedure 11. The material was purified by HPLC method 1. This gave 355 mg (88% of theory) of product.

¹H NMR (400 MHz, chloroform-d₁): δ=1.17-1.39 (m, 6H), 1.41-1.62 (m, 6H), 1.79 (mc, 2H), 2.01-2.32 (m, 11H), 2.34-2.49 (m, 6H), 2.55-2.81 (m, 6H), 6.59-6.63 (m, 1H), 6.67-6.77 (m, 4H), 7.15 (d, 1H), 7.21 (t, 1H).

### Example 11

### 8-(3-Hydroxyphenyl)-9-[5-(methyl{6-[(4,4,5,5,5-pentafluoropentyl)sulphonyl]hexyl}amino)pentyl]-6,7-dihydro-5H-benzo[7]annulen-3-ol

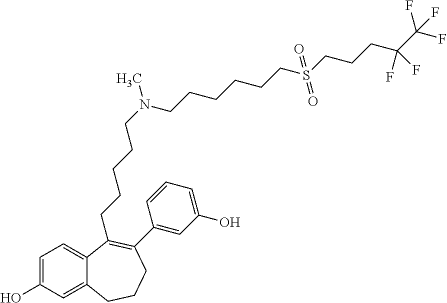 [SMILES: CN(CCCCCCS(=O)(=O)CCCC(F)(F)C(F)(F)F)CCCCCC1=C(c2cccc(O)c2)CCCc2cc(O)ccc21]

250 mg (0.62 mmol) of 9-(5-bromopentyl)-8-(3-hydroxyphenyl)-6,7-dihydro-5H-benzo[7]annulen-3-ol were reacted with 296 mg (0.87 mmol) of N-methyl-6-[(4,4,5,5,5-pentafluoropentyl)sulphonyl]hexane-1-amine according to Procedure 11. The material was purified by HPLC method 1. This gave 320 mg (78% of theory) of product.

¹H NMR (400 MHz, chloroform-d₁): δ=1.20-1.40 (m, 6H), 1.43-1.66 (m, 6H), 1.85 (mc, 2H), 2.02-2.11 (m, 2H), 2.13-2.36 (m, 9H), 2.38-2.51 (m, 6H), 2.60 (t, 2H), 2.92-3.05 (m, 4H), 6.57-6.60 (m, 1H), 6.66-6.77 (m, 4H), 7.17 (d, 1H), 7.23 (t, 1H).

### Example 12

### 8-(3-Hydroxyphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol

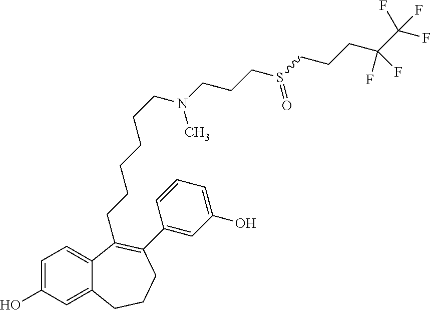 [SMILES: CN(CCCCCCC1=C(c2cccc(O)c2)CCCc2cc(O)ccc21)CCCS(=O)CCCC(F)(F)C(F)(F)F]

250 mg (0.60 mmol) of 9-(6-bromohexyl)-8-(3-hydroxyphenyl)-6,7-dihydro-5H-benzo[7]annulen-3-ol were reacted with 237 mg (0.84 mmol) of N-methyl-3-[(RS)(4,4,5,5,5-pentafluoropentyl)sulphinyl]propane-1-amine according to Procedure 11. The product was purified on silica gel 60 (mobile phase: dichloromethane, dichloromethane-methanol 95:5 and 90:10). This gave 290 mg (78% of theory) of product.

¹H NMR (400 MHz, DMSO-d₆): δ=0.97-1.17 (m, 6H), 1.19-1.31 (m, 2H), 1.75 (mc, 2H), 1.85-2.47 (m, 17H), 2.52-2.57 (m, 2H), 2.59-2.78 (m, 3H), 2.80-2.89 (m, 1H), 6.59-6.69 (m, 5H), 7.08-7.17 (m, 2H), 9.20-9.36 (m, 2H).

### Example 13

### 8-(3-Hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol

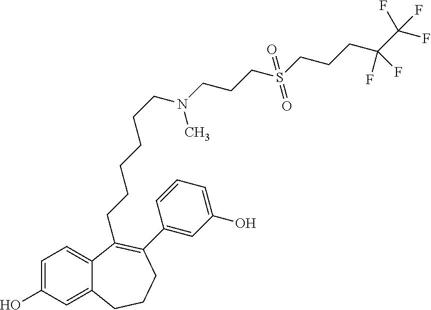 [SMILES: CN(CCCCCCC1=C(c2cccc(O)c2)CCCc2cc(O)ccc21)CCCS(=O)(=O)CCCC(F)(F)C(F)(F)F]

250 mg (0.60 mmol) of 9-(6-bromohexyl)-8-(3-hydroxyphenyl)-6,7-dihydro-5H-benzo[7]annulen-3-ol were reacted with 251 mg (0.84 mmol) of N-methyl-3-[(4,4,5,5,5-pentafluoropentyl)sulphonyl]propane-1-amine according to Procedure 11. The product was purified on silica gel 60 (mobile phase: dichloromethane, dichloromethane-methanol 95:5). This gave 300 mg (79% of theory) of product.

¹H NMR (400 MHz, DMSO-d₆): δ=0.98-1.17 (m, 6H), 1.19-1.38 (m, 2H), 1.78 (mc, 2H), 1.87-2.47 (m, 17H), 2.52-2.57 (m, 2H), 3.09 (t, 2H), 3.23 (t, 2H), 6.60-6.68 (m, 5H), 7.08-7.17 (m, 2H), 9.20-9.34 (m, 2H).

### Example 14

### 8-(3-Hydroxyphenyl)-9-[6-(methyl{4-[(RS)-(4,4,5,5,5-pentafluoropentyl)sulphinyl]butyl}amino)hexyl]-6,7-dihydro-5H-benzo[7]annulen-3-ol

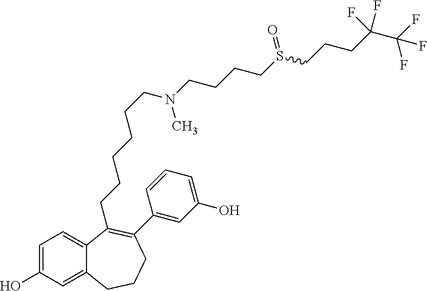 [SMILES: CN(CCCCCCC1=C(c2cccc(O)c2)CCCc2cc(O)ccc21)CCCCS(=O)CCCC(F)(F)C(F)(F)F]

250 mg (0.60 mmol) of 9-(6-bromohexyl)-8-(3-hydroxyphenyl)-6,7-dihydro-5H-benzo[7]annulen-3-ol were reacted with 249 mg (0.84 mmol) of N-methyl-4-[(RS)-(4,4,5,5,5-pentafluoropentyl)sulphinyl]butane-1-amine according to Procedure 11. The product was purified on silica gel 60 (mobile phase: dichloromethane, dichloromethane-methanol 95:5 and 90:10). This gave 290 mg (77% of theory) of product.

¹H NMR (400 MHz, chloroform-d₁): δ=1.05-1.28 (m, 6H), 1.40 (mc, 2H), 1.65-1.92 (m, 4H), 2.04-2.41 (m, 15H), 2.52 (mc, 2H), 2.59 (mc, 2H), 2.68-2.89 (m, 4H), 6.69-6.77 (m, 4H), 6.81 (s, 1H), 7.15 (d, 1H), 7.19 (t, 1H).

### Example 15

### 8-(3-Hydroxyphenyl)-9-[6-(methyl{4-[(4,4,5,5,5-pentafluoropentyl)sulphonyl]butyl}amino)hexyl]-6,7-dihydro-5H-benzo[7]annulen-3-ol

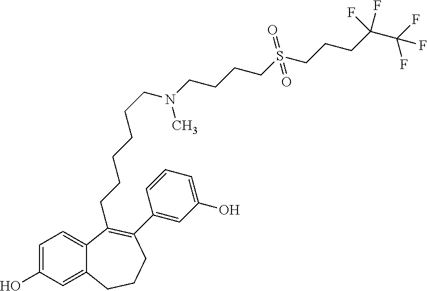 [SMILES: CN(CCCCCCC1=C(c2cccc(O)c2)CCCc2cc(O)ccc21)CCCCS(=O)(=O)CCCC(F)(F)C(F)(F)F]

250 mg (0.60 mmol) of 9-(6-bromohexyl)-8-(3-hydroxyphenyl)-6,7-dihydro-5H-benzo[7]annulen-3-ol were reacted with 262 mg (0.84 mmol) of N-methyl-4-[(4,4,5,5,5-pentafluoropentyl)sulphonyl]butane-1-amine according to Procedure 11. The product was purified on silica gel 60 (mobile phase: dichloromethane, dichloromethane-methanol 95:5 and 90:10). This gave 300 mg (77% of theory) of product.

¹H NMR (400 MHz, chloroform-d₁): δ=1.04-1.28 (m, 6H), 1.46 (mc, 2H), 1.79 (mc, 2H), 1.94 (mc, 2H), 2.05-2.39 (m, 15H), 2.52-2.63 (m, 4H), 3.03-3.11 (m, 4H), 6.69-6.77 (m, 4H), 6.82 (s, 1H), 7.16 (d, 1H), 7.21 (t, 1H).

### Example 16

### 8-(3-Hydroxyphenyl)-9-[6-(methyl{5-[(RS)-(4,4,5,5,5-pentafluoropentyl)sulphinyl]pentyl}amino)hexyl]-6,7-dihydro-5H-benzo[7]annulen-3-ol

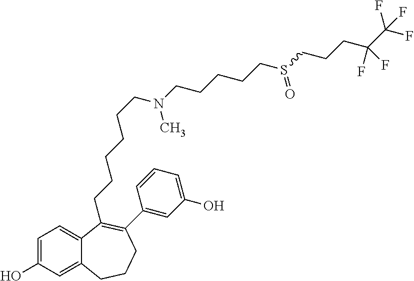 [SMILES: CN(CCCCCCC1=C(c2cccc(O)c2)CCCc2cc(O)ccc21)CCCCCS(=O)CCCC(F)(F)C(F)(F)F]

250 mg (0.60 mmol) of 9-(6-bromohexyl)-8-(3-hydroxyphenyl)-6,7-dihydro-5H-benzo[7]annulen-3-ol were reacted with 261 mg (0.84 mmol) of N-methyl-5-[(RS)-(4,4,5,5,5-pentafluoropentyl)sulphinyl]pentane-1-amine according to Procedure 11. The product was purified on silica gel 60 (mobile phase: dichloromethane, dichloromethane-methanol 95:5 and 90:10). This gave 300 mg (77% of theory) of product.

¹H NMR (400 MHz, chloroform-d₁): δ=1.04-1.31 (m, 6H), 1.40-1.74 (m, 6H), 1.87 (mc, 2H), 2.01-2.47 (m, 15H), 2.49-2.62 (m, 4H), 2.65-2.87 (m, 4H), 6.68-6.77 (m, 4H), 6.81 (s, 1H), 7.13 (d, 1H), 7.19 (t, 1H).

### Example 17

### 8-(3-Hydroxyphenyl)-9-[6-(methyl{5-[(4,4,5,5,5-pentafluoropentyl)sulphonyl]pentyl}amino)hexyl]-6,7-dihydro-5H-benzo[7]annulen-3-ol

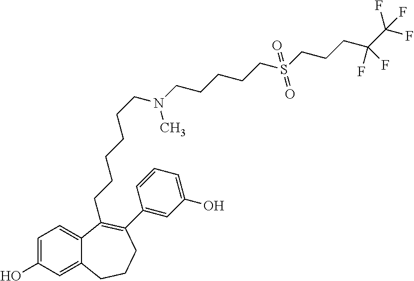 [SMILES: CN(CCCCCCC1=C(c2cccc(O)c2)CCCc2cc(O)ccc21)CCCCCS(=O)(=O)CCCC(F)(F)C(F)(F)F]

250 mg (0.60 mmol) of 9-(6-bromohexyl)-8-(3-hydroxyphenyl)-6,7-dihydro-5H-benzo[7]annulen-3-ol were reacted with 274 mg (0.84 mmol) of N-methyl-5-[(4,4,5,5,5-pentafluoropentyl)sulphonyl]pentane-1-amine according to Procedure 11. The product was purified on silica gel 60 (mobile phase: dichloromethane, dichloromethane-methanol 95:5 and 90:10). This gave 300 mg (76% of theory) of product.

¹H NMR (400 MHz, chloroform-d₁): δ=1.03-1.28 (m, 6H), 1.44-1.60 (m, 4H), 1.71 (mc, 2H), 1.92 (mc, 2H), 2.05-2.13 (m, 4H), 2.15-2.50 (m, 11H), 2.53-2.65 (m, 4H), 3.01-3.10 (m, 4H), 6.70-6.77 (m, 4H), 6.85 (s, 1H), 7.15 (d, 1H), 7.21 (t, 1H).

### Example 18

### 8-(3-Hydroxyphenyl)-9-[6-(methyl{6-[(RS)-(4,4,5,5,5-pentafluoropentyl)sulphinyl]hexyl}amino)hexyl]-6,7-dihydro-5H-benzo[7]annulen-3-ol

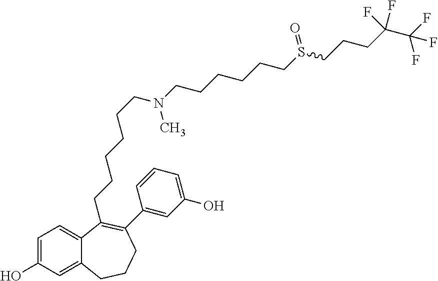 [SMILES: CN(CCCCCCC1=C(c2cccc(O)c2)CCCc2cc(O)ccc21)CCCCCCS(=O)CCCC(F)(F)C(F)(F)F]

250 mg (0.60 mmol) of 9-(6-bromohexyl)-8-(3-hydroxyphenyl)-6,7-dihydro-5H-benzo[7]annulen-3-ol were reacted with 272 mg (0.84 mmol) of N-methyl-6-[(RS)-(4,4,5,5,5-pentafluoropentyl)sulphinyl]hexane-1-amine according to Procedure 11. The product was purified on silica gel 60 (mobile phase: dichloromethane, dichloromethane-methanol 90:10 and 80:20). This gave 230 mg (58% of theory) of product.

¹H NMR (400 MHz, chloroform-d₁): δ=1.05-1.27 (m, 6H), 1.30-1.60 (m, 8H), 1.80 (mc, 2H), 2.06-2.10 (m, 4H), 2.11-2.44 (m, 13H), 2.56 (mc, 2H), 2.61-2.84 (m, 4H), 6.66-6.78 (m, 5H), 7.13 (d, 1H), 7.19 (t, 1H).

### Example 19

### 8-(3-Hydroxyphenyl)-9-[6-(methyl{6-[(4,4,5,5,5-pentafluoropentyl)sulphonyl]hexyl}amino)hexyl]-6,7-dihydro-5H-benzo[7]annulen-3-ol

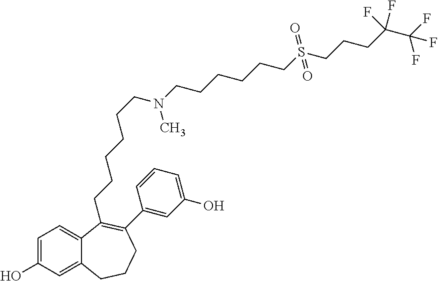 [SMILES: CN(CCCCCCC1=C(c2cccc(O)c2)CCCc2cc(O)ccc21)CCCCCCS(=O)(=O)CCCC(F)(F)C(F)(F)F]

250 mg (0.60 mmol) of 9-(6-bromohexyl)-8-(3-hydroxyphenyl)-6,7-dihydro-5H-benzo[7]annulen-3-ol were reacted with 286 mg (0.84 mmol) of N-methyl-6-[(4,4,5,5,5-pentafluoropentyl)sulphonyl]hexane-1-amine according to Procedure 11. The product was purified on silica gel 60 (mobile phase: dichloromethane, dichloromethane-methanol 95:5 and 90:10). This gave 300 mg (74% of theory) of product.

¹H NMR (400 MHz, chloroform-d₁): δ=1.03-1.29 (m, 6H), 1.44 (mc, 2H), 1.49-1.61 (m, 4H), 1.72 (mc, 2H), 1.89 (mc, 2H), 2.03-2.40 (m, 11H), 2.42-2.74 (m, 8H), 2.98-3.12 (m, 4H), 6.70-6.78 (m, 4H), 6.89 (s, 1H), 7.15 (d, 1H), 7.20 (t, 1H).

### Example 20

### 8-(4-Hydroxyphenyl)-9-[6-(methyl{3-[(4,4,5,5,5-pentafluoropentyl)sulphanyl]propyl}amino)hexyl]-6,7-dihydro-5H-benzo[7]annulen-3-ol

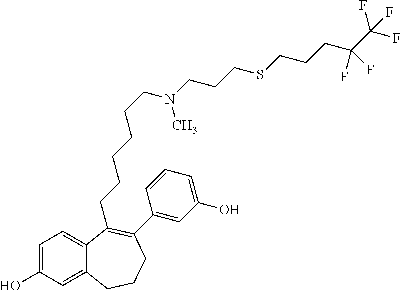 [SMILES: CN(CCCCCCC1=C(c2cccc(O)c2)CCCc2cc(O)ccc21)CCCSCCCC(F)(F)C(F)(F)F]

500 mg (1.20 mmol) of 9-(6-bromohexyl)-8-(4-hydroxyphenyl)-6,7-dihydro-5H-benzo[7]annulen-3-ol were reacted with 479 mg (1.81 mmol) of N-methyl-3-[(4,4,5,5,5-pentafluoropentyl)sulphanyl]propane-1-amine according to Procedure 11. The product was purified on silica gel 60 (mobile phase: dichloromethane, dichloromethane-methanol 95:5 and 90:10). The solid was digested with diisopropyl ether. This gave 348 mg (48% of theory) of product.

¹H NMR (300 MHz, methanol-d₄): δ=1.01-1.38 (m, 8H), 1.64-1.76 (m, 2H), 1.77-1.89 (m, 2H), 1.97-2.33 (m, 11H), 2.35-2.40 (m, 4H), 2.50 (t, 2H), 2.59 (t, 4H), 6.61-6.69 (m, 2H), 6.74 (dt, 2H), 7.03 (dt, 2H), 7.10 (d, 1H).

### Example 21

### 8-(3-Mesylphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol

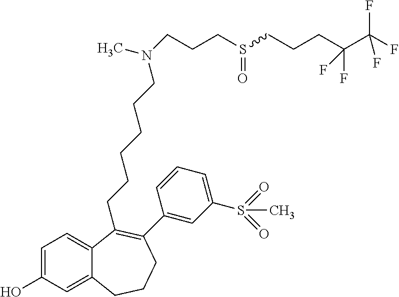 [SMILES: CN(CCCCCCC1=C(c2cccc(S(C)(=O)=O)c2)CCCc2cc(O)ccc21)CCCS(=O)CCCC(F)(F)C(F)(F)F]

A mixture of 163 mg of N-methyl-3-[(RS)-(4,4,5,5,5-pentafluoropentyl)sulphinyl]propane-1-amine and 198 mg of 9-(6-bromohexyl)-8-(3-mesylphenyl)-6,7-dihydro-5H-benzo[7]annulen-3-01, 44 mg of sodium carbonate and 34 mg of potassium iodide in 5 ml of DMF was stirred at 85° C. for 4 h. The mixture was then diluted with 50 ml of water and extracted three times with ethyl acetate, and the extracts were washed with brine, dried over sodium sulphate, concentrated and purified by preparative HPLC. This gave 161 mg of the title compound. C₃₃H₄₄F₅NO₄S₂ (677.84). 1H NMR (400 MHz, chloroform-d, selected signals): δ 1.0-1.4 (m, 8H), 3.1 (s), 6.8-6.9 (m, 2H), 7.2 (d, 1H), 7.5-7.7 (m, 2H), 7.8-7.9 (m, 2H). LC-MS (ESIpos) mass found: 677.00.

### Example 22

### 4-{3-Hydroxy-9-[6-(methyl{3-[(4,4,5,5,5-pentafluoropentyl)sulphonyl]propyl}amino)hexyl]-6,7-dihydro-5H-benzo[7]annulen-8-yl}benzonitrile

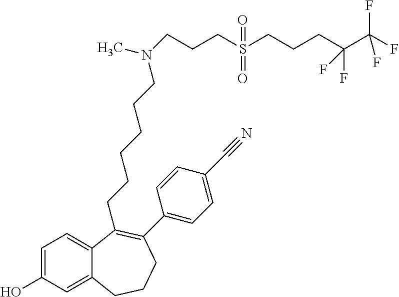 [SMILES: CN(CCCCCCC1=C(c2ccc(C#N)cc2)CCCc2cc(O)ccc21)CCCS(=O)(=O)CCCC(F)(F)C(F)(F)F]

100 mg of 4-[9-(6-bromohexyl)-3-hydroxy-6,7-dihydro-5H-benzo[7]annulen-8-yl]benzonitrile (crude product) were reacted analogously with 84 mg of N-methyl-3-[(4,4,5,5,5-pentafluoropentyl)sulphonyl]propane-1-amine. Purification by preparative HPLC gave 85 mg of the title compound.

C₃₃H₄₁F₆N₂O₃S (640.76). MS (ESIpos) mass found: 640.28. 1H NMR (selected signals, 300 MHz, DMSO-d₆): δ 1.66-1.80 (m, 2H), 2.03 (s), 2.10 (t, 2H), 2.99-3.10 (m, 2H), 3.19 (t, 2H), 6.59-6.67 (m, 2H), 7.11 (d, 1H), 7.40 (d, 2H), 7.79 (d, 2H).

### Example 23

### 8-(2-Fluoro-5-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol

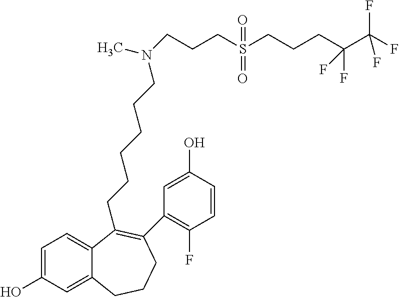 [SMILES: CN(CCCCCCC1=C(c2cc(O)ccc2F)CCCc2cc(O)ccc21)CCCS(=O)(=O)CCCC(F)(F)C(F)(F)F]

100 mg of 9-(6-bromohexyl)-8-(2-fluoro-5-hydroxyphenyl)-6,7-dihydro-5H-benzo[7]annulen-3-ol were reacted with 96 mg of N-methyl-3-[(4,4,5,5,5-pentafluoropentyl)sulphonyl]propane-1-amine analogously to General Procedure 11, followed by HPLC purification (column XBridge (from Waters, C18 5 μm 30×100 mm, acetonitrile/water+0.1% ammonia) and freeze drying to give 50 mg of the title compound. C₃₂H₄₁F₆NO₄S. MS (ESIpos) mass found: 649.27. 1H NMR (400 MHz, DMSO-d₆): δ 0.93-1.29 (m, 8H), 1.67-1.76 (m, 2H), 1.83-1.93 (m, 4H), 1.95-2.05 (5H), 2.09 (t, 2H), 2.19-2.44 (m), 3.01-3.07 (m, 2H), 3.19 (t, 2H), 6.53-6.57 (m, 1H), 6.59-6.65 (m, 3H), 6.95 (t, 1H), 7.09 (d, 1H), 9.29 (s).

### Example 24

### 4-Chloro-8-(4-fluoro-3-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol

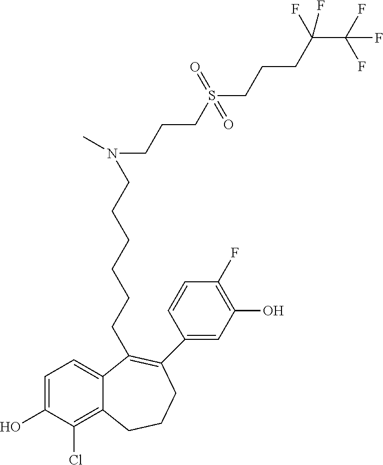 [SMILES: CN(CCCCCCC1=C(c2ccc(F)c(O)c2)CCCc2c1ccc(O)c2Cl)CCCS(=O)(=O)CCCC(F)(F)C(F)(F)F]

170 mg of 9-(6-bromohexyl)-4-chloro-8-(4-fluoro-3-hydroxyphenyl)-6,7-dihydro-5H-benzo[7]annulen-3-ol were reacted with 130 mg of N-methyl-3-[(4,4,5,5,5-pentafluoropentyl)sulphonyl]propane-1-amine analogously to General Procedure 11. Purification by preparative HPLC gave 30 mg of the title compound. C₃₂H₄₀ClF₆NO₄S (684.19). MS ESI (pos) mass found: 683.23. 1H NMR (300 MHz, DMSO-d₆): δ 0.94-1.26 (m, 8H), 1.66-1.78 (m, 2H), 1.81-2.01 (m), 2.02 (s), 2.11 (t, 2H), 2.24-2.41 (m), 2.99-3.08 (m, 2H), 3.19 (t, 2H), 6.55-6.63 (m, 1H), 6.77 (dd, 1H), 6.84 (d, 1H), 7.02-7.13 (m, 2H), 9.8-10.2 (broad s.).

### Example 25

### 8-(4-Fluoro-3-hydroxyphenyl)-9-[5-(methyl{4-[(4,4,5,5,5-pentafluoropentyl)sulphonyl]butyl}amino)pentyl]-6,7-dihydro-5H-benzo[7]annulen-3-ol

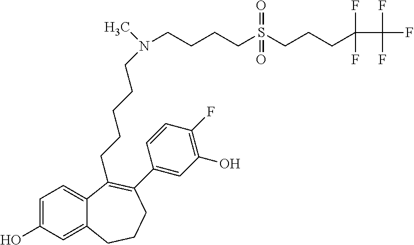 [SMILES: CN(CCCCCC1=C(c2ccc(F)c(O)c2)CCCc2cc(O)ccc21)CCCCS(=O)(=O)CCCC(F)(F)C(F)(F)F]

106 mg (0.25 mmol) of 9-(5-bromopentyl)-8-(4-fluoro-3-hydroxyphenyl)-6,7-dihydro-5H-benzo[7]annulen-3-ol were reacted with 95 mg (0.30 mmol) of N-methyl-4-[(4,4,5,5,5-pentafluoropentyl)sulphonyl]butane-1-amine analogously to General Procedure 11. Purification by preparative HPLC gave 72 mg (44% of theory) of the title compound. C₃₂H₄₁F₆NO₄S (649.74). MS ESI (pos) mass found: 649.27. 1H NMR (400 MHz, DMSO-d₆): d [ppm]=0.96-1.26 (m, 6H), 1.43 (quin, 2H), 1.51-1.68 (m, 2H), 1.81-2.06 (9H, comprises singulet at 2.02 ppm), 2.06-2.17 (m, 2H), 2.22 (t, 2H), 2.25-2.44 (m), 3.02-3.12 (m, 2H), 3.12-3.22 (m, 2H), 6.55-6.64 (m, 3H), 6.76 (dd, 1H), 6.95-7.18 (m, 2H), 8.17 (s).

### Example 26

### 8-(4-Hydroxyphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol

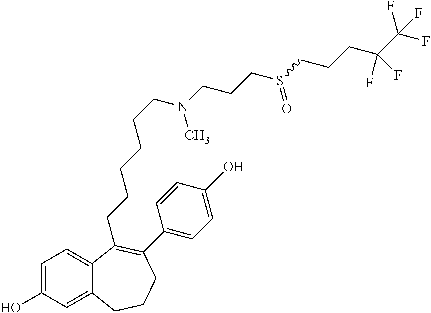 [SMILES: CN(CCCCCCC1=C(c2ccc(O)cc2)CCCc2cc(O)ccc21)CCCS(=O)CCCC(F)(F)C(F)(F)F]

500 mg (1.20 mmol) of 9-(6-bromohexyl)-8-(4-hydroxyphenyl)-6,7-dihydro-5H-benzo[7]annulen-3-ol were reacted with 508 mg (1.81 mmol) of N-methyl-3-[(RS)-(4,4,5,5,5-pentafluoropentyl)sulphinyl]propane-1-amine according to Procedure 11. The product was purified on silica gel 60 (mobile phase: dichloromethane, dichloromethane-methanol 95:5, 90:10 and 80:20). This gave 613 mg (83% of theory) of product.

¹H NMR (300 MHz, methanol-d₄): δ=1.01-1.40 (m, 8H), 1.85-1.97 (m, 2H), 1.98-2.12 (m, 6H), 2.20-2.43 (m, 9H), 2.47-2.63 (m, 4H), 2.70-2.96 (m, 4H), 6.61-6.68 (m, 2H), 6.74 (dt, 2H), 7.03 (dt, 2H), 7.10 (d, 1H).

### Example 27

### 8-(4-Hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol

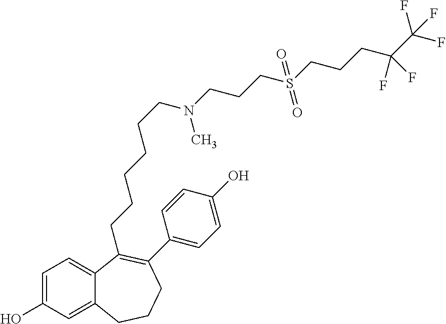 [SMILES: CN(CCCCCCC1=C(c2ccc(O)cc2)CCCc2cc(O)ccc21)CCCS(=O)(=O)CCCC(F)(F)C(F)(F)F]

500 mg (1.20 mmol) of 9-(6-bromohexyl)-8-(4-hydroxyphenyl)-6,7-dihydro-5H-benzo[7]annulen-3-ol were reacted with 537 mg (1.81 mmol) of N-methyl-3-[(4,4,5,5,5-pentafluoropentyl)sulphonyl]propane-1-amine according to Procedure 11. The product was purified on silica gel 60 (mobile phase: dichloromethane, dichloromethane-methanol 95:5, 90:10 and 80:20). This gave 489 mg (64% of theory) of product.

¹H NMR (400 MHz, methanol-d₄): δ=1.02-1.24 (m, 6H), 1.25-1.36 (m, 2H), 1.92 (mc, 2H), 1.99-2.13 (m, 6H), 2.16-2.43 (m, 9H), 2.46 (t, 2H), 2.59 (t, 2H), 3.10 (mc, 2H), 3.19 (t, 2H), 6.61-6.69 (m, 2H), 6.75 (dt, 2H), 7.03 (dt, 2H), 7.10 (d, 1H).

### Example 28

### 8-(4-Fluoro-3-hydroxyphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol

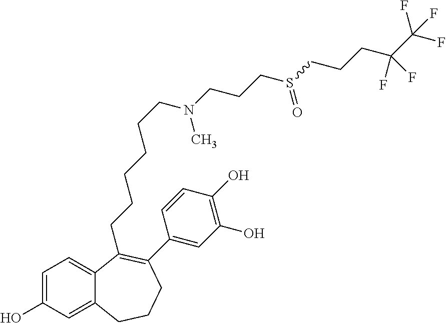 [SMILES: CN(CCCCCCC1=C(c2ccc(O)c(O)c2)CCCc2cc(O)ccc21)CCCS(=O)CCCC(F)(F)C(F)(F)F]

250 mg (0.58 mmol) of 9-(6-bromohexyl)-8-(4-fluoro-3-hydroxyphenyl)-6,7-dihydro-5H-benzo[7]annulen-3-ol were reacted with 227 mg (0.81 mmol) of N-methyl-3-[(RS)-(4,4,5,5,5-pentafluoropentyl)sulphinyl]propane-1-amine according to Procedure 11. The product was purified on silica gel 60 (mobile phase: dichloromethane, dichloromethane-methanol 95:5, 90:10 and 80:20). The material was then purified by HPLC method 3. This gave 184 mg (50% of theory) of product.

¹H NMR (400 MHz, chloroform-d₁): δ=1.01-1.36 (m, 8H), 2.00 (mc, 2H), 2.04-2.12 (m, 4H), 2.13-2.37 (m, 11H), 2.50 (mc, 2H), 2.59 (mc, 2H), 2.73-2.88 (m, 4H), 6.66 (ddd, 1H), 6.70. (d, 1H), 6.73 (dd, 1H), 6.90 (dd, 1H), 7.01 (dd, 1H), 7.14 (d, 1H).

### Example 29

### 8-(4-Fluoro-3-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol

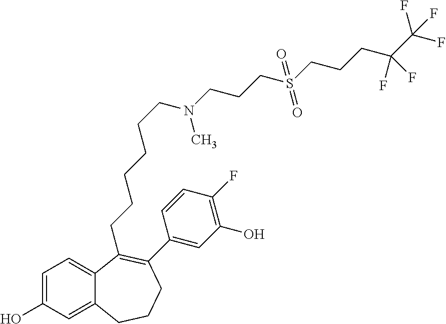 [SMILES: CN(CCCCCCC1=C(c2ccc(F)c(O)c2)CCCc2cc(O)ccc21)CCCS(=O)(=O)CCCC(F)(F)C(F)(F)F]

250 mg (0.58 mmol) of 9-(6-bromohexyl)-8-(4-fluoro-3-hydroxyphenyl)-6,7-dihydro-5H-benzo[7]annulen-3-ol were reacted with 240 mg (0.81 mmol) of N-methyl-3-[(4,4,5,5,5-pentafluoropentyl)sulphonyl]propane-1-amine according to Procedure 11. The product was purified on silica gel 60 (mobile phase: dichloromethane, dichloromethane-methanol 95:5 and 90:10). The material was then purified by HPLC method 3. This gave 170 mg (45% of theory) of product.

¹H NMR (400 MHz, DMSO-d₆): δ=1.02-1.11 (m, 2H), 1.13-1.28 (m, 4H), 1.30-1.40 (m, 2H), 2.02-2.13 (m, 6H), 2.15-2.35 (m, 11H), 2.51 (t, 2H), 2.60 (t, 2H), 3.06-3.13 (m, 4H), 6.66-6.75 (m, 3H), 6.92 (dd, 1H), 7.03 (dd, 1H), 7.16 (d, 1H).

### Example 30

### 8-(4-Fluoro-3-hydroxyphenyl)-9-[6-(methyl{4-[(RS)-(4,4,5,5,5-pentafluoropentyl)sulphinyl]butyl}amino)hexyl]-6,7-dihydro-5H-benzo[7]annulen-3-ol

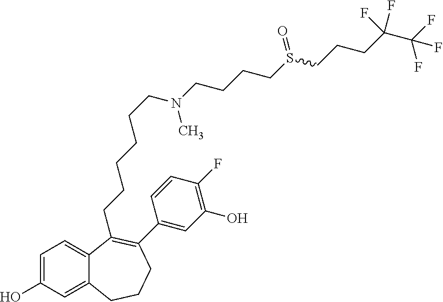 [SMILES: CN(CCCCCCC1=C(c2ccc(F)c(O)c2)CCCc2cc(O)ccc21)CCCCS(=O)CCCC(F)(F)C(F)(F)F]

250 mg (0.58 mmol) of 9-(6-bromohexyl)-8-(4-fluoro-3-hydroxyphenyl)-6,7-dihydro-5H-benzo[7]annulen-3-ol were reacted with 239 mg (0.81 mmol) of N-methyl-4-[(RS)-(4,4,5,5,5-pentafluoropentyl)sulphinyl]butane-1-amine according to Procedure 11. The product was purified on silica gel 60 (mobile phase: dichloromethane, dichloromethane-methanol 95:5, 90:10 and 80:20). The material was then purified by HPLC method 3. This gave 162 mg (43% of theory) of product.

¹H NMR (400 MHz, chloroform-d₁): δ=1.03-1.38 (m, 8H), 1.60-1.89 (m, 4H), 2.03-2.34 (m, 15H), 2.35-2.43 (m, 2H), 2.59 (mc, 2H), 2.67-2.88 (m, 4H), 6.66 (ddd, 1H), 6.70 (d, 1H), 6.73 (dd, 1H), 6.91 (dd, 1H), 7.01 (dd, 1H), 7.14 (d, 1H).

### Example 31

### 8-(4-Fluoro-3-hydroxyphenyl)-9-[6-(methyl{4-[(4,4,5,5,5-pentafluoropentyl)sulphonyl]butyl}amino)hexyl]-6,7-dihydro-5H-benzo[7]annulen-3-ol

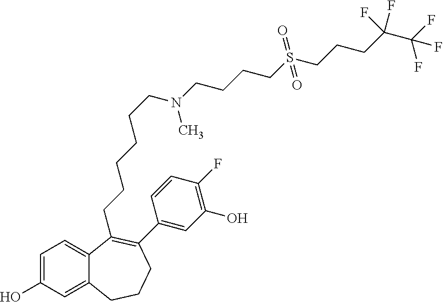 [SMILES: CN(CCCCCCC1=C(c2ccc(F)c(O)c2)CCCc2cc(O)ccc21)CCCCS(=O)(=O)CCCC(F)(F)C(F)(F)F]

250 mg (0.58 mmol) of 9-(6-bromohexyl)-8-(4-fluoro-3-hydroxyphenyl)-6,7-dihydro-5H-benzo[7]annulen-3-ol were reacted with 251 mg (0.81 mmol) of N-methyl-4-[(4,4,5,5,5-pentafluoropentyl)sulphonyl]butane-1-amine according to Procedure 11. The product was purified on silica gel 60 (mobile phase: dichloromethane, dichloromethane-methanol 95:5 and 90:10). The material was then purified by HPLC method 2. This gave 141 mg (57% of theory) of product.

¹H NMR (400 MHz, chloroform-d₁): δ=1.01-1.11 (m, 2H) 1.15-1.30 (m, 4H), 1.33-1.43 (m, 2H), 1.69-1.79 (m, 2H), 1.86-1.96 (m, 2H), 2.03-2.12 (m, 4H), 2.15-2.34 (m, 11H), 2.43 (mc, 2H), 2.59 (mc, 2H), 3.03-3.10 (m, 4H), 6.64-6.70 (m, 2H), 6.72 (dd, 1H), 6.92 (dd, 1H), 7.02 (dd, 1H), 7.16 (d, 1H).

### Example 32

### 8-(4-Fluoro-3-hydroxyphenyl)-9-[6-(methyl{5-[(RS)-(4,4,5,5,5-pentafluoropentyl)sulphinyl]pentyl}amino)hexyl]-6,7-dihydro-5H-benzo[7]annulen-3-ol

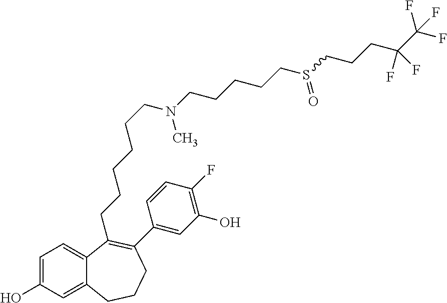 [SMILES: CN(CCCCCCC1=C(c2ccc(F)c(O)c2)CCCc2cc(O)ccc21)CCCCCS(=O)CCCC(F)(F)C(F)(F)F]

250 mg (0.58 mmol) of 9-(6-bromohexyl)-8-(4-fluoro-3-hydroxyphenyl)-6,7-dihydro-5H-benzo[7]annulen-3-ol were reacted with 250 mg (0.81 mmol) of N-methyl-5-[(RS)-(4,4,5,5,5-pentafluoropentyl)sulphinyl]pentane-1-amine according to Procedure 11. The material was purified by HPLC method 2. This gave 213 mg (56% of theory) of product.

¹H NMR (400 MHz, chloroform-d₁): δ=1.02-1.29 (m, 6H), 1.32-1.65 (m, 6H), 1.85 (mc, 2H), 2.03-2.11 (m, 4H), 2.12-2.33 (m, 11H), 2.39 (mc, 2H), 2.58 (mc, 2H), 2.64-2.84 (m, 4H), 6.64 (ddd, 1H), 6.69 (d, 1H), 6.72 (dd, 1H), 6.90 (dd, 1H), 7.00 (dd, 1H), 7.14 (d, 1H).

### Example 33

### 8-(4-Fluoro-3-hydroxyphenyl)-9-[6-(methyl{5-[(4,4,5,5,5-pentafluoropentyl)sulphonyl]pentyl}amino)hexyl]-6,7-dihydro-5H-benzo[7]annulen-3-ol

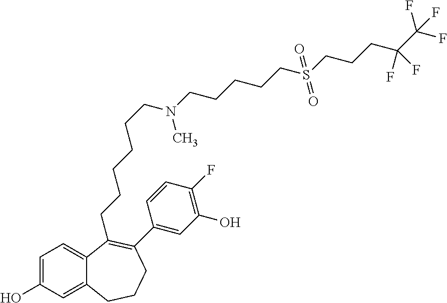 [SMILES: CN(CCCCCCC1=C(c2ccc(F)c(O)c2)CCCc2cc(O)ccc21)CCCCCS(=O)(=O)CCCC(F)(F)C(F)(F)F]

250 mg (0.58 mmol) of 9-(6-bromohexyl)-8-(4-fluoro-3-hydroxyphenyl)-6,7-dihydro-5H-benzo[7]annulen-3-ol were reacted with 263 mg (0.81 mmol) of N-methyl-5-[(4,4,5,5,5-pentafluoropentyl)sulphonyl]pentane-1-amine according to Procedure 11. The material was purified by HPLC method 2. This gave 201 mg (51% of theory) of product.

¹H NMR (400 MHz, chloroform-d₁): δ=1.01-1.11 (m, 2H), 1.15-1.28 (m, 4H), 1.36-1.56 (m, 4H), 1.60-1.70 (m, 2H), 1.91 (mc, 2H), 2.01-2.11 (m, 4H), 2.15-2.35 (m, 11H), 2.45 (mc, 2H), 2.57 (mc, 2H), 2.99-3.09 (m, 4H), 6.64 (ddd, 1H), 6.68 (d, 1H), 6.72 (dd, 1H), 6.91 (dd, 1H), 7.01 (dd, 1H), 7.15 d, 1H).

### Example 34

### 8-(4-Fluoro-3-hydroxyphenyl)-9-[6-(methyl{6-[(RS)-(4,4,5,5,5-pentafluoropentyl)sulphinyl]hexyl}amino)hexyl]-6,7-dihydro-5H-benzo[7]annulen-3-ol

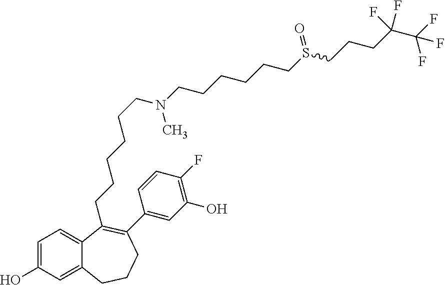 [SMILES: CN(CCCCCCC1=C(c2ccc(F)c(O)c2)CCCc2cc(O)ccc21)CCCCCCS(=O)CCCC(F)(F)C(F)(F)F]

250 mg (0.60 mmol) of 9-(6-bromohexyl)-8-(4-fluoro-3-hydroxyphenyl)-6,7-dihydro-5H-benzo[7]annulen-3-ol were reacted with 261 mg (0.81 mmol) of N-methyl-6-[(RS)-(4,4,5,5,5-pentafluoropentyl)sulphinyl]hexane-1-amine according to Procedure 11. The material was purified by HPLC method 2. This gave 232 mg (59% of theory) of product.

¹H NMR (400 MHz, chloroform-d₁): δ=1.01-1.26 (m, 6H), 1.31-1.64 (m, 8H), 1.81 (mc, 2H), 1.99-2.09 (m, 4H), 2.11-2.33 (m, 11H), 2.44 (mc, 2H), 2.55 (mc, 2H), 2.63-2.83 (m, 4H), 6.61 (ddd, 1H), 6.67 (d, 1H), 6.71 (dd, 1H), 6.89 (dd, 1H), 6.99 (dd, 1H), 7.12 (d, 1H).

### Example 35

### 8-(4-Fluoro-3-hydroxyphenyl)-9-[6-(methyl{6-[(4,4,5,5,5-pentafluoropentyl)sulphonyl]hexyl}amino)hexyl]-6,7-dihydro-5H-benzo[7]annulen-3-ol

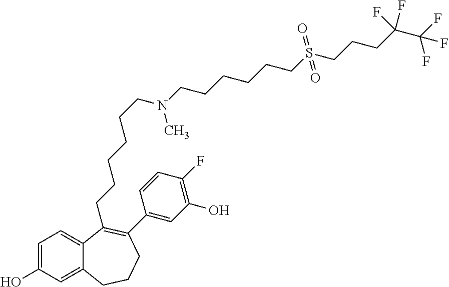 [SMILES: CN(CCCCCCC1=C(c2ccc(F)c(O)c2)CCCc2cc(O)ccc21)CCCCCCS(=O)(=O)CCCC(F)(F)C(F)(F)F]

250 mg (0.58 mmol) of 9-(6-bromohexyl)-8-(4-fluoro-3-hydroxyphenyl)-6,7-dihydro-5H-benzo[7]annulen-3-ol were reacted with 274 mg (0.81 mmol) of N-methyl-6-[(4,4,5,5,5-pentafluoropentyl)sulphonyl]hexane-1-amine according to Procedure 11. The material was purified by HPLC method 2. This gave 321 mg (80% of theory) of product.

¹H NMR (400 MHz, chloroform-d₁): δ=1.00-1.11 (m, 2H), 1.16-1.29 (m, 4H), 1.33-1.56 (m, 6H), 1.57-1.67 (m, 2H), 1.88 (mc, 2H), 2.03-2.12 (m, 4H), 2.14-2.34 (m, 11H), 2.40-2.47 (m, 2H), 2.58 (mc, 2H), 2.97-3.07 (m, 4H), 6.64 (ddd, 1H), 6.68 (d, 1H), 6.72 (dd, 1H), 6.92 (dd, 1H), 7.01 (dd, 1H), 7.15 (d, 1H).

### Example 36

### 8-(3-Fluoro-4-hydroxyphenyl)-9-[5-(methyl{3-[(4,4,5,5,5-pentafluoropentyl)sulphanyl]propyl}amino)pentyl]-6,7-dihydro-5H-benzo[7]annulen-3-ol

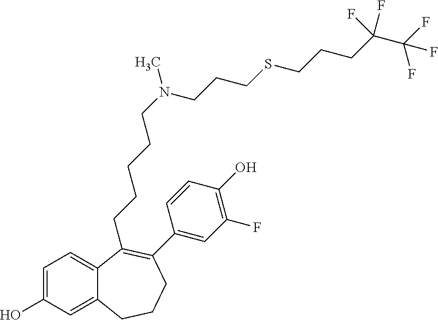 [SMILES: CN(CCCCCC1=C(c2ccc(O)c(F)c2)CCCc2cc(O)ccc21)CCCSCCCC(F)(F)C(F)(F)F]

500 mg (1.19 mmol) of 9-(5-bromopentyl)-8-(3-fluoro-4-hydroxyphenyl)-6,7-dihydro-5H-benzo[7]annulen-3-ol were reacted with 459 mg (1.73 mmol) of N-methyl-3-[(4,4,5,5,5-pentafluoropentyl)sulphanyl]propane-1-amine according to Procedure 11. The product was purified on silica gel 60 (mobile phase: dichloromethane, dichloromethane-methanol 95:5 and 90:10). The residue was triturated with diisopropyl ether. This gave 378 mg (53% of theory) of product.

¹H NMR (400 MHz, chloroform-d₁): δ=1.07-1.38 (m, 6H), 1.70-1.93 (m, 4H), 2.02-2.33 (m, 13H), 2.37 (mc, 2H), 2.52 (t, 2H), 2.56-2.65 (m, 4H), 6.70 (d, 1H), 6.73 (dd, 1H), 6.90 (dd, 1H), 6.94-7.02 (m, 2H), 7.16 (d, 1H).

### Example 37

### 8-(3-Fluoro-4-hydroxyphenyl)-9-[5-(methyl{3-[(RS)-(4,4,5,5,5-pentafluoropentyl)sulphinyl]propyl}amino)pentyl]-6,7-dihydro-5H-benzo[7]annulen-3-ol

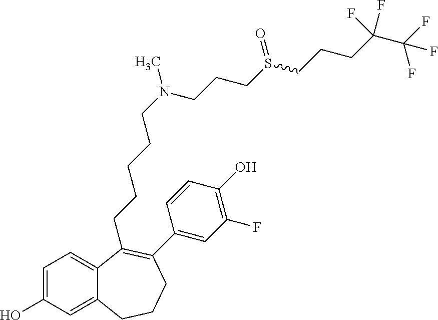 [SMILES: CN(CCCCCC1=C(c2ccc(O)c(F)c2)CCCc2cc(O)ccc21)CCCS(=O)CCCC(F)(F)C(F)(F)F]

270 mg (0.64 mmol) of 9-(5-bromopentyl)-8-(3-fluoro-4-hydroxyphenyl)-6,7-dihydro-5H-benzo[7]annulen-3-ol were reacted with 273 mg (0.97 mmol) of N-methyl-3-[(RS)-(4,4,5,5,5-pentafluoropentyl)sulphinyl]propane-1-amine according to Procedure 11. The product was purified on silica gel 60 (mobile phase: dichloromethane, dichloromethane-methanol 90:10). This gave 292 mg (73% of theory) of product.

¹H NMR (400 MHz, methanol-d₄): δ=1.08-1.35 (m, 6H), 1.85-1.97 (m, 2H), 1.99-2.14 (m, 6H), 2.18-2.43 (m, 9H), 2.46-2.53 (m, 2H), 2.59 (t, 2H), 2.71-2.94 (m, 4H), 6.63 (d, 1H), 6.66 (dd, 1H), 6.82-6.93 (m, 3H), 7.11 (d, 1H).

### Example 38

### 8-(3-Fluoro-4-hydroxyphenyl)-9-[5-(methyl{3-[(4,4,5,5,5-pentafluoropentyl)sulphonyl]propyl}amino)pentyl]-6,7-dihydro-5H-benzo[7]annulen-3-ol

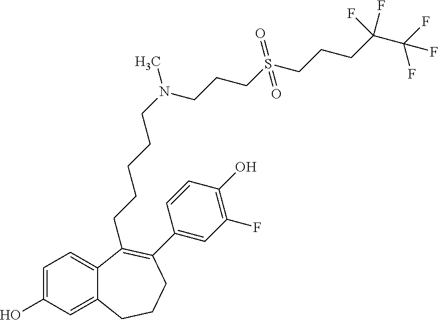 [SMILES: CN(CCCCCC1=C(c2ccc(O)c(F)c2)CCCc2cc(O)ccc21)CCCS(=O)(=O)CCCC(F)(F)C(F)(F)F]

500 mg (1.19 mmol) of 9-(5-bromopentyl)-8-(3-fluoro-4-hydroxyphenyl)-6,7-dihydro-5H-benzo[7]annulen-3-ol were reacted with 532 mg (1.79 mmol) of N-methyl-3-[(4,4,5,5,5-pentafluoropentyl)sulphonyl]propane-1-amine according to Procedure 11. The product was purified on silica gel 60 (mobile phase: dichloromethane, dichloromethane-methanol 95:5 and 90:10). This gave 477 mg (63% of theory) of product.

¹H NMR (400 MHz, chloroform-d₁): δ=1.05-1.14 (m, 2H), 1.16-1.30 (m, 4H), 1.95 (mc, 2H), 2.03-2.42 (m, 17H), 2.60 (t, 2H), 3.00-3.08 (m, 4H), 6.70 (d, 1H), 6.73 (dd, 1H), 6.90 (dd, 1H), 6.94-7.00 (m, 2H), 7.16 (d, 1H).

### Example 39

### 9-[5-(Methyl{3-[(4,4,5,5,5-pentafluoropentyl)sulphanyl]propyl}amino)pentyl]-8-(3-pyridyl)-6,7-dihydro-5H-benzo[7]annulen-3-ol

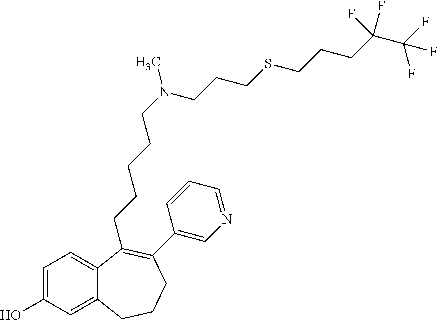 [SMILES: CN(CCCCCC1=C(c2cccnc2)CCCc2cc(O)ccc21)CCCSCCCC(F)(F)C(F)(F)F]

320 mg (0.83 mmol) of 9-(5-bromopentyl)-8-(3-pyridyl)-6,7-dihydro-5H-benzo[7]annulen-3-ol were reacted with 308 mg (1.16 mmol) of N-methyl-3-[(4,4,5,5,5-pentafluoropentyl)sulphanyl]propane-1-amine according to Procedure 11. The product was purified on silica gel 60 (mobile phase: dichloromethane, dichloromethane-methanol 95:5 and 92.5:7.5). This gave 310 mg (66% of theory) of product.

¹H NMR (600 MHz, chloroform-d₁): δ=1.08-1.15 (m, 2H), 1.19-1.27 (m, 2H), 1.34 (mc, 2H), 1.77 (mc, 2H), 1.84-1.90 (m, 2H), 2.07-2.32 (m, 11H), 2.36 (mc, 2H), 2.47 (mc, 2H), 2.51 (t, 2H), 2.58 (t, 2H), 2.62 (t, 2H), 6.72 (d, 1H), 6.74 (dd, 1H), 7.17 (d, 1H), 7.31 (dd, 1H), 7.56 (dt, 1H), 8.51 (dd, 1H), 8.53 (d, 1H).

### Example 40

### 9-[5-(Methyl{3-[(RS)-(4,4,5,5,5-pentafluoropentyl)sulphinyl]propyl}amino)pentyl]-8-(3-pyridyl)-6,7-dihydro-5H-benzo[7]annulen-3-ol

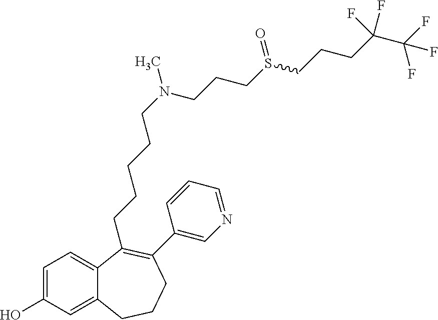 [SMILES: CN(CCCCCC1=C(c2cccnc2)CCCc2cc(O)ccc21)CCCS(=O)CCCC(F)(F)C(F)(F)F]

320 mg (0.83 mmol) of 9-(5-bromopentyl)-8-(3-pyridyl)-6,7-dihydro-5H-benzo[7]annulen-3-ol were reacted with 326 mg (1.16 mmol) of N-methyl-3-[(RS)-(4,4,5,5,5-pentafluoropentyl)sulphinyl]propane-1-amine according to Procedure 11. The product was purified on silica gel 60 (mobile phase: dichloromethane, dichloromethane-methanol 95:5 and 90:10). This gave 240 mg (49% of theory) of product.

¹H NMR (600 MHz, chloroform-d₁): δ=1.07 (mc, 2H), 1.19-1.31 (m, 4H), 1.81-1.92 (m, 2H), 2.05 (t, 2H), 2.09-2.31 (m, 11H), 2.32-2.41 (m, 4H), 2.60-2.69 (m, 3H), 2.72-2.84 (m, 3H), 6.75 (d, 1H), 6.79 (dd, 1H), 7.17 (d, 1H), 7.29 (dd, 1H), 7.56 (dt, 1H), 8.50 (dd, 1H), 8.54 (d, 1H).

### Example 41

### 9-[5-(Methyl{3-[(4,4,5,5,5-pentafluoropentyl)sulphonyl]propyl}amino)pentyl]-8-(3-pyridyl)-6,7-dihydro-5H-benzo[7]annulen-3-ol

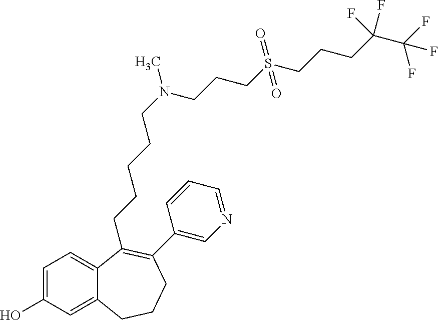 [SMILES: CN(CCCCCC1=C(c2cccnc2)CCCc2cc(O)ccc21)CCCS(=O)(=O)CCCC(F)(F)C(F)(F)F]

320 mg (0.83 mmol) of 9-(5-bromopentyl)-8-(3-pyridyl)-6,7-dihydro-5H-benzo[7]annulen-3-ol were reacted with 345 mg (1.16 mmol) of N-methyl-3-[(4,4,5,5,5-pentafluoropentyl)sulphonyl]propane-1-amine according to Procedure 11. The product was purified on silica gel 60 (mobile phase: dichloromethane, dichloromethane-methanol 95:5 and 92.5:7.5). This gave 310 mg (62% of theory) of product.

¹H NMR (600 MHz, chloroform-d₁): δ=1.06-1.13 (m, 2H), 1.19-1.30 (m, 4H), 1.96 (mc, 2H), 2.08-2.32 (m, 13H), 2.37 (t, 2H), 2.42 (t, 2H), 2.64 (t, 2H), 3.01-3.08 (m, 4H), 6.74 (d, 1H), 6.77 (dd, 1H), 7.18 (d, 1H), 7.31 (dd, 1H), 7.57 (dt, 1H), 8.51 (dd, 1H), 8.54 (d, 1H).

### Example 42

### 9-[6-(Methyl{3-[(4,4,5,5,5-pentafluoropentyl)sulphanyl]propyl}amino)hexyl]-8-(3-pyridyl)-6,7-dihydro-5H-benzo[7]annulen-3-ol

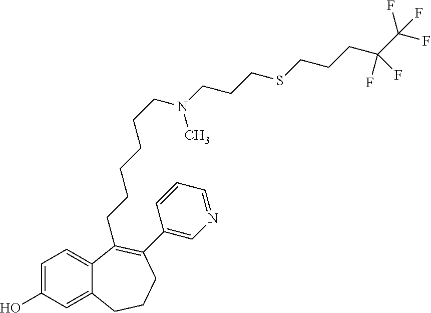 [SMILES: CN(CCCCCCC1=C(c2cccnc2)CCCc2cc(O)ccc21)CCCSCCCC(F)(F)C(F)(F)F]

331 mg (0.83 mmol) of 9-(6-bromohexyl)-8-(3-pyridyl)-6,7-dihydro-5H-benzo[7]annulen-3-ol were reacted with 308 mg (1.16 mmol) of N-methyl-3-[(4,4,5,5,5-pentafluoropentyl)sulphanyl]propane-1-amine according to Procedure 11. The product was purified on silica gel 60 (mobile phase: dichloromethane, dichloromethane-methanol 95:5 and 92.5:7.5). This gave 300 mg (62% of theory) of product.

¹H NMR (400 MHz, chloroform-d₁): δ=1.04-1.16 (m, 4H), 1.16-1.27 (m, 2H), 1.39 (mc, 2H), 1.78-1.92 (m, 4H), 2.06-2.23 (m, 6H), 2.31 (s, 3H), 2.33-2.41 (m, 4H), 2.51-2.66 (m, 8H), 6.74-6.79 (m, 2H), 7.16 (d, 1H), 7.32 (dd, 1H), 7.57 (dt, 1H), 8.51 (dd, 1H), 8.53 (d, 1H).

### Example 43

### 9-[6-(Methyl{3-[(RS)-(4,4,5,5,5-pentafluoropentyl)sulphinyl]propyl}amino)hexyl]-8-(3-pyridyl)-6,7-dihydro-5H-benzo[7]annulen-3-ol

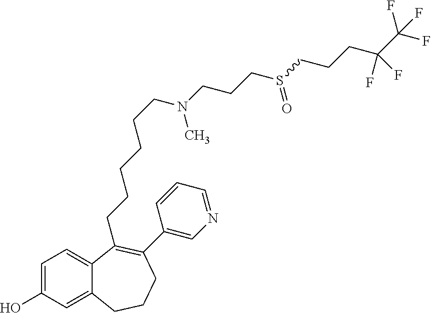 [SMILES: CN(CCCCCCC1=C(c2cccnc2)CCCc2cc(O)ccc21)CCCS(=O)CCCC(F)(F)C(F)(F)F]

331 mg (0.83 mmol) of 9-(6-bromohexyl)-8-(3-pyridyl)-6,7-dihydro-5H-benzo[7]annulen-3-ol were reacted with 326 mg (1.16 mmol) of N-methyl-3-[(RS)-(4,4,5,5,5-pentafluoropentyl)sulphinyl]propane-1-amine according to Procedure 11. The product was purified on silica gel 60 (mobile phase: dichloromethane, dichloromethane-methanol 95:5, 92.5:7.5 and 90:10). This gave 330 mg (66% of theory) of product.

¹H NMR (400 MHz, chloroform-d₁): δ=1.04-1.15 (m, 4H), 1.17-1.29 (m, 4H), 1.86-1.99 (m, 2H), 2.05-2.50 (m, 17H), 2.63 (mc, 2H), 2.76-2.86 (m, 4H), 6.74 (d, 1H), 6.78 dd, 1H), 7.16 (d, 1H), 7.30 (dd, 1H), 7.56 (dt, 1H), 8.50 (dd, 1H), 8.54 (d, 1H).

### Example 44

### 9-[6-(Methyl{3-[(4,4,5,5,5-pentafluoropentyl)sulphonyl]propyl}amino)hexyl]-8-(3-pyridyl)-6,7-dihydro-5H-benzo[7]annulen-3-ol

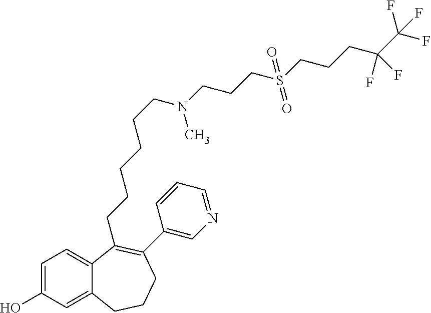 [SMILES: CN(CCCCCCC1=C(c2cccnc2)CCCc2cc(O)ccc21)CCCS(=O)(=O)CCCC(F)(F)C(F)(F)F]

331 mg (0.83 mmol) of 9-(6-bromohexyl)-8-(3-pyridyl)-6,7-dihydro-5H-benzo[7]annulen-3-ol were reacted with 345 mg (1.16 mmol) of N-methyl-3-[(4,4,5,5,5-pentafluoropentyl)sulphonyl]propane-1-amine according to Procedure 11. The product was purified on silica gel 60 (mobile phase: dichloromethane, dichloromethane-methanol 95:5 and 92.5:7.5). This gave 340 mg (67% of theory) of product.

¹H NMR (400 MHz, chloroform-d₁): δ=1.03-1.16 (m, 4H), 1.16-1.34 (m, 4H), 1.96-2.05 (m, 2H), 2.07-2.40 (m, 15H), 2.48 (t, 2H), 2.64 (t, 2H), 3.07 (mc, 4H), 6.74 (d, 1H), 6.77 dd, 1H), 7.18 (d, 1H), 7.31 (dd, 1H), 7.57 (dt, 1H), 8.51 (dd, 1H), 8.54 (d, 1H).

### Example 45

### 8-(4-Mesylphenyl)-9-[5-(methyl{3-[(4,4,5,5,5-pentafluoropentyl)sulphanyl]propyl}amino)pentyl]-6,7-dihydro-5H-benzo[7]annulen-3-ol

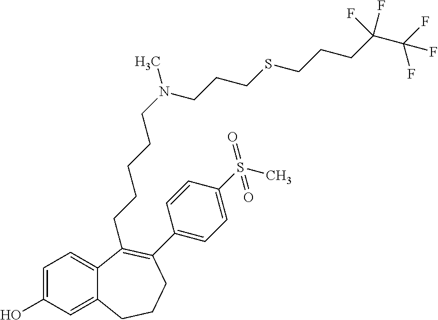 [SMILES: CN(CCCCCC1=C(c2ccc(S(C)(=O)=O)cc2)CCCc2cc(O)ccc21)CCCSCCCC(F)(F)C(F)(F)F]

300 mg (0.65 mmol) of 9-(5-bromopentyl)-8-(4-mesylphenyl)-6,7-dihydro-5H-benzo[7]annulen-3-ol were reacted with 258 mg (0.97 mmol) of N-methyl-3-[(4,4,5,5,5-pentafluoropentyl)sulphanyl]propane-1-amine according to Procedure 11. The product was purified on silica gel 60 (mobile phase: dichloromethane, dichloromethane-methanol 95:5 and 90:10). This gave 258 mg (60% of theory) of product.

¹H NMR (400 MHz, chloroform-d₁): δ=1.05-1.16 (m, 2H), 1.17-1.35 (m, 4H), 1.72 (mc, 2H), 1.81-1.92 (m, 2H), 2.07-2.26 (m, 11H), 2.30-2.44 (m, 4H), 2.50 (t, 2H), 2.57 (t, 2H), 2.62-2.67 (m, 2H), 3.10 (s, 3H), 6.69-6.74 (m, 2H), 7.16 (d, 1H), 7.44 (dt, 2H), 7.92 (dt, 2H).

### Example 46

### 8-(4-Mesylphenyl)-9-[5-(methyl{3-[(RS)-(4,4,5,5,5-pentafluoropentyl)sulphinyl]propyl}amino)pentyl]-6,7-dihydro-5H-benzo[7]annulen-3-ol

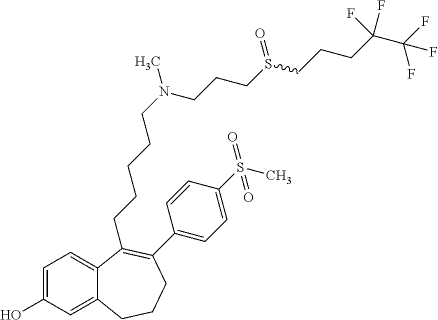 [SMILES: CN(CCCCCC1=C(c2ccc(S(C)(=O)=O)cc2)CCCc2cc(O)ccc21)CCCS(=O)CCCC(F)(F)C(F)(F)F]

300 mg (0.65 mmol) of 9-(5-bromopentyl)-8-(4-mesylphenyl)-6,7-dihydro-5H-benzo[7]annulen-3-ol were reacted with 273 mg (0.97 mmol) of N-methyl-3-[(RS)-(4,4,5,5,5-pentafluoropentyl)sulphinyl]propane-1-amine according to Procedure 11. The product was purified on silica gel 60 (mobile phase: dichloromethane, dichloromethane-methanol 90:10). This gave 346 mg (81% of theory) of product.

¹H NMR (600 MHz, chloroform-d₁): δ=1.04-1.11 (m, 2H), 1.18-1.33 (m, 4H), 1.84-1.94 (m, 2H), 2.06-2.31 (m, 13H), 2.35 (t, 2H), 2.41 (mc, 2H), 2.63 (t, 2H), 2.65-2.72 (m, 1H), 2.73-2.84 (m, 3H), 3.10 (s, 3H), 6.74 (d, 1H), 6.78 (dd, 1H), 7.15 (d, 1H), 7.44 (mc, 2H), 7.92 (mc, 2H).

### Example 47

### 8-(4-Mesylphenyl)-9-[5-(methyl{3-[(4,4,5,5,5-pentafluoropentyl)sulphonyl]propyl}amino)pentyl]-6,7-dihydro-5H-benzo[7]annulen-3-ol

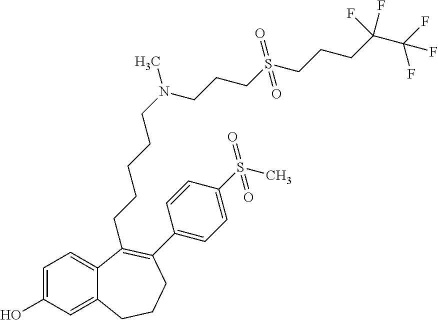 [SMILES: CN(CCCCCC1=C(c2ccc(S(C)(=O)=O)cc2)CCCc2cc(O)ccc21)CCCS(=O)(=O)CCCC(F)(F)C(F)(F)F]

500 mg (1.08 mmol) of 9-(5-bromopentyl)-8-(4-mesylphenyl)-6,7-dihydro-5H-benzo[7]annulen-3-ol were reacted with 481 mg (1.62 mmol) of N-methyl-3-[(4,4,5,5,5-pentafluoropentyl)sulphonyl]propane-1-amine according to Procedure 11. The product was purified on silica gel 60 (mobile phase: dichloromethane, dichloromethane-methanol 90:10). This gave 257 mg (35% of theory) of product.

¹H NMR (400 MHz, chloroform-d₁): δ=1.04-1.14 (m, 2H), 1.17-1.29 (m, 4H), 1.93 (mc, 2H), 2.07-2.41 (m, 17H), 2.65 (t, 2H), 2.98-3.09 (m, 4H), 3.10 (s, 3H), 6.73 (d, 1H), 6.77 (dd, 1H), 7.19 (d, 1H), 7.45 (dt, 2H), 7.93 (dt, 2H).

### Example 48

### 8-(4-Mesylphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol

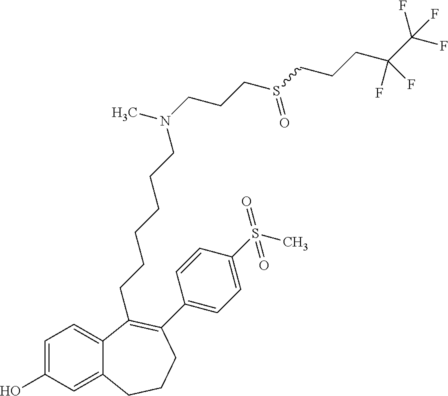 [SMILES: CN(CCCCCCC1=C(c2ccc(S(C)(=O)=O)cc2)CCCc2cc(O)ccc21)CCCS(=O)CCCC(F)(F)C(F)(F)F]

135 mg (0.28 mmol) of 9-(6-bromohexyl)-8-(4-mesylphenyl)-6,7-dihydro-5H-benzo[7]annulen-3-ol were reacted with 111.3 mg (0.40 mmol) of N-methyl-3-[(RS)-(4,4,5,5,5-pentafluoropentyl)sulphinyl]propane-1-amine according to Procedure 11. The product was purified on silica gel 60 (mobile phase: dichloromethane->dichloromethane-methanol 90:10). This gave 190 mg (99% of theory) of product.

¹H NMR (600 MHz, chloroform-d₁): δ=1.05-1.14 (m, 4H), 1.18-1.27 (m, 4H), 1.93 (mc, 2H), 2.08-2.31 (m, 13H), 2.35 (t, 2H), 2.39-2.50 (m, 2H), 2.63 (mc, 2H), 2.71-2.87 (m, 4H), 3.10 (s, 3H), 6.74 (d, 1H), 6.78 dd, 1H), 7.15 (d, 1H), 7.44 (dt, 2H), 7.92 (dt, 2H).

### Example 49

### 8-(4-Mesylphenyl)-9-[6-(methyl{3-[(4,4,5,5,5-pentafluoropentyl)sulphonyl]propyl}amino)hexyl]-6,7-dihydro-5H-benzo[7]annulen-3-ol

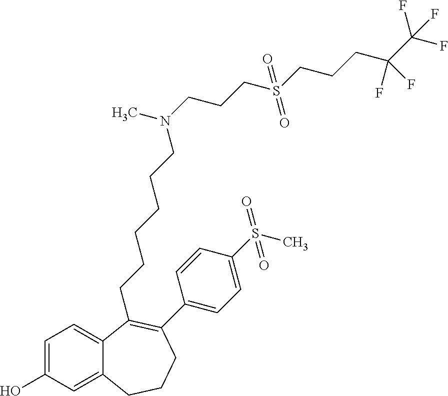 [SMILES: CN(CCCCCCC1=C(c2ccc(S(C)(=O)=O)cc2)CCCc2cc(O)ccc21)CCCS(=O)(=O)CCCC(F)(F)C(F)(F)F]

135 mg (0.28 mmol) of 9-(6-bromohexyl)-8-(4-mesylphenyl)-6,7-dihydro-5H-benzo[7]annulen-3-ol were reacted with 117.7 mg (0.40 mmol) of N-methyl-3-[(4,4,5,5,5-pentafluoropentyl)sulphonyl]propane-1-amine according to Procedure 11. The product was purified on silica gel 60 (mobile phase: dichloromethane->dichloromethane-methanol 95:5). This gave 100 mg (51% of theory) of product.

¹H NMR (500 MHz, chloroform-d₁): δ=1.04-1.14 (m, 4H), 1.16-1.30 (m, 4H), 1.97 (mc, 2H), 2.08-2.37 (m, 15H), 2.42 (t, 2H), 2.64 (t, 2H), 3.02-3.09 (m, 4H), 3.10 (s, 3H), 6.73 (d, 1H), 6.76 (dd, 1H), 7.18 (d, 1H), 7.44 (dt, 2H), 7.93 (dt, 2H).

### Example 50

### 9-[5-({3-[(RS)-(4,4,5,5,5-Pentafluoropentyl)sulphinyl]propyl}amino)pentyl]-8-phenyl-6,7-dihydro-5H-benzo[7]annulen-3-ol

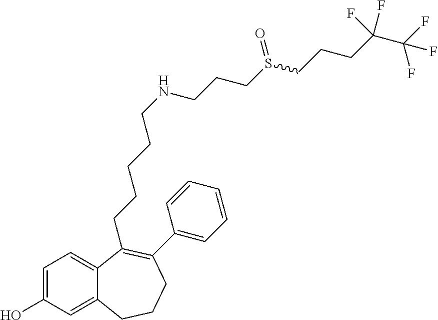 [SMILES: O=S(CCCNCCCCCC1=C(c2ccccc2)CCCc2cc(O)ccc21)CCCC(F)(F)C(F)(F)F]

300 mg (0.78 mmol) of 9-(5-bromopentyl)-8-phenyl-6,7-dihydro-5H-benzo[7]annulen-3-ol were reacted with 291 mg (1.09 mmol) of 3-[(RS)-(4,4,5,5,5-pentafluoropentyl)sulphinyl]propane-1-amine according to Procedure 11. The product was purified on silica gel 60 (mobile phase: dichloromethane, dichloromethane-methanol 95:5, 90:10 and 80:20). This gave 280 mg (63% of theory) of product.

¹H NMR (600 MHz, DMSO-d₆): δ=1.02-1.18 (m, 4H), 1.19-1.27 (m, 2H), 1.77 (mc, 2H), 1.91 (mc, 2H), 1.97-2.07 (m, 4H), 2.28-2.46 (m, 6H), 2.56 (t, 2H), 2.63 (t, 2H), 2.65-2.82 (m, 3H), 2.82-2.88 (m, 1H), 6.62-6.69 (m, 2H), 7.13 (d, 1H), 7.20-7.28 (m, 3H), 7.34-7.39 (m, 2H), 9.30 (s, 1H).

### Example 51

### 8-(3-Hydroxyphenyl)-9-[6-(methyl{3-[(4,4,4-trifluorobutyl)sulphonyl]propyl}amino)hexyl]-6,7-dihydro-5H-benzo[7]annulen-3-ol

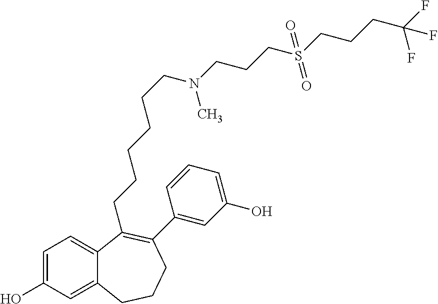 [SMILES: CN(CCCCCCC1=C(c2cccc(O)c2)CCCc2cc(O)ccc21)CCCS(=O)(=O)CCCC(F)(F)F]

200 mg (0.48 mmol) of 9-(6-bromohexyl)-8-(3-hydroxyphenyl)-6,7-dihydro-5H-benzo[7]annulen-3-ol were reacted with 167 mg (0.67 mmol) of N-methyl-3-[(4,4,4-trifluorobutyl)sulphonyl]propane-1-amine according to Procedure 11. The material was then purified by HPLC method 1. This gave 160 mg (57% of theory) of product.

¹H NMR (600 MHz, chloroform-d₁): δ=1.06-1.17 (m, 4H), 1.18-1.23 (m, 2H), 1.35 (mc, 2H), 2.01-2.11 (m, 6H), 2.12-2.18 (m, 2H), 2.23 (s, 3H), 2.26 (mc, 2H), 2.28-2.38 (m, 4H), 2.51 (t, 2H), 2.58 (t, 2H), 3.04-3.09 (m, 4H), 6.69 (d, 1H), 6.71-6.78 (m, 4H), 7.16 (d, 1H), 7.22 (t, 1H).

### Example 52

### 8-(3-Hydroxyphenyl)-9-[6-(methyl{4-[(4,4,4-trifluorobutyl)sulphonyl]butyl}amino)hexyl]-6,7-dihydro-5H-benzo[7]annulen-3-ol

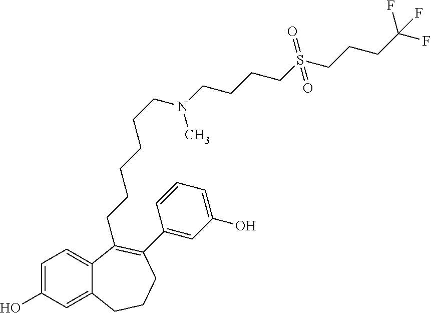 [SMILES: CN(CCCCCCC1=C(c2cccc(O)c2)CCCc2cc(O)ccc21)CCCCS(=O)(=O)CCCC(F)(F)F]

200 mg (0.48 mmol) of 9-(6-bromohexyl)-8-(3-hydroxyphenyl)-6,7-dihydro-5H-benzo[7]annulen-3-ol were reacted with 176.1 mg (0.67 mmol) of N-methyl-4-[(4,4,4-trifluorobutyl)sulphonyl]butane-1-amine according to Procedure 11. The product was then purified using HPLC Method 1 and Chiralpak IA (5μ, 250×20 mm, 20 ml/min, mobile phase: n-hexane with 0.1% diethylamine-ethanol 80:20). This gave 45 mg (16% of theory) of product.

¹H NMR (600 MHz, chloroform-d₁): δ=1.08-1.27 (m, 6H), 1.39 (mc, 2H), 1.70 (mc, 2H), 1.89 (mc, 2H), 2.04-2.11 (m, 4H), 2.12-2.19 (m, 2H), 2.23-2.37 (m, 9H), 2.43 (t, 2H), 2.58 (t, 2H), 3.02-3.07 (m, 4H), 6.68 (d, 1H), 6.72 (dd, 1H), 6.73-6.77 (m, 3H), 7.16 (d, 1H), 7.22 (t, 1H).

### Example 53

### 8-(3-Hydroxyphenyl)-9-[6-(methyl{3-[(RS)-(3,3,4,4,4-pentafluorobutyl)sulphinyl]propyl}amino)hexyl]-6,7-dihydro-5H-benzo[7]annulen-3-ol

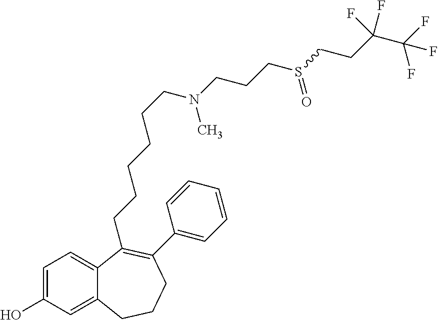 [SMILES: CN(CCCCCCC1=C(c2ccccc2)CCCc2cc(O)ccc21)CCCS(=O)CCC(F)(F)C(F)(F)F]

200 mg (0.48 mmol) of 9-(6-bromohexyl)-8-(3-hydroxyphenyl)-6,7-dihydro-5H-benzo[7]annulen-3-ol were reacted with 180 mg of N-methyl-3-[(RS)-(3,3,4,4,4-pentafluorobutyl)sulphinyl]propane-1-amine according to Procedure 11. The material was then purified by HPLC method 1. This gave 120 mg (41% of theory) of product.

¹H NMR (600 MHz, chloroform-d₁): δ=1.05-1.17 (m, 4H), 1.18-1.26 (m, 2H), 1.31 (mc, 2H), 1.92-2.03 (m, 2H), 2.05-2.13 (m, 4H), 2.17-2.27 (m, 5H), 2.38 (mc, 2H), 2.43-2.53 (m, 2H), 2.53-2.67 (m, 4H), 2.77-2.83 (m, 1H), 2.87-3.00 (m, 3H), 6.70 (d, 1H), 6.71-6.78 (m, 4H), 7.16 (d, 1H), 7.20 (t, 1H).

### Example 54

### 8-(3-Hydroxyphenyl)-9-[6-(methyl{3-[(3,3,4,4,4-pentafluorobutyl)sulphonyl]propyl}amino)hexyl]-6,7-dihydro-5H-benzo[7]annulen-3-ol

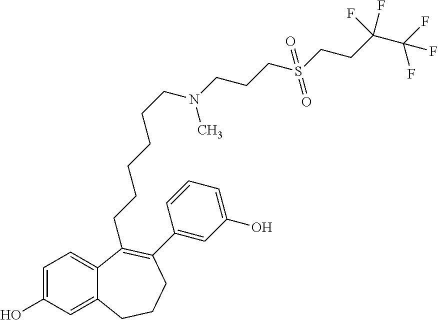 [SMILES: CN(CCCCCCC1=C(c2cccc(O)c2)CCCc2cc(O)ccc21)CCCS(=O)(=O)CCC(F)(F)C(F)(F)F]

200 mg (0.48 mmol) of 9-(6-bromohexyl)-8-(3-hydroxyphenyl)-6,7-dihydro-5H-benzo[7]annulen-3-ol were reacted with 191 mg (0.67 mmol) of N-methyl-3-[(3,3,4,4,4-pentafluorobutyl)sulphonyl]propane-1-amine according to Procedure 11. The product was then purified using HPLC Method 1 and Chiralpak IA (5μ, 250×20 mm, 20 ml/min, mobile phase: n-hexane with 0.1% diethylamine-ethanol 80:20). This gave 90 mg (30% of theory) of product.

¹H NMR (600 MHz, chloroform-d₁): δ=1.07-1.19 (m, 4H), 1.23 (mc, 2H), 1.34 (mc, 2H), 2.02-2.13 (m, 6H), 2.11 (s, 3H), 2.24 (t, 2H), 2.37 (t, 2H), 2.48 (t, 2H), 2.58-2.69 (m, 4H), 3.15 (mc, 2H), 3.23 (mc, 2H), 6.70 (d, 1H), 6.72-6.77 (m, 3H), 6.78 (d, 1H), 7.17 (d, 1H), 7.22 (t, 1H).

### Example 55

### 8-(3-Hydroxyphenyl)-9-[6-(methyl{3-[(RS)-(3,3,3-trifluoropropyl)sulphinyl]propyl}amino)hexyl]-6,7-dihydro-5H-benzo[7]annulen-3-ol

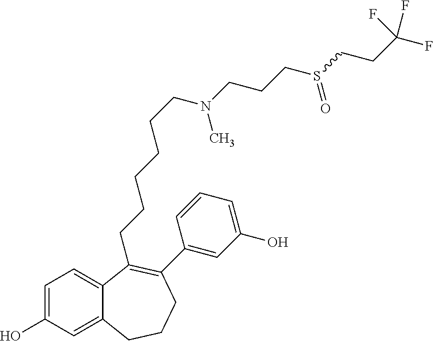 [SMILES: CN(CCCCCCC1=C(c2cccc(O)c2)CCCc2cc(O)ccc21)CCCS(=O)CCC(F)(F)F]

200 mg (0.48 mmol) of 9-(6-bromohexyl)-8-(3-hydroxyphenyl)-6,7-dihydro-5H-benzo[7]annulen-3-ol were reacted with 146 mg (0.67 mmol) of N-methyl-3-[(RS)-(3,3,3-trifluoropropyl)sulphinyl]propane-1-amine according to Procedure 11. The material was then purified by HPLC method 1. This gave 175 mg (66% of theory) of product.

¹H NMR (600 MHz, chloroform-d₁): δ=1.05-1.16 (m, 4H), 1.17-1.24 (m, 2H), 1.29-1.35 (m, 2H), 1.93-2.04 (m, 2H), 2.05-2.12 (m, 4H), 2.18-2.27 (m, 5H), 2.37 (mc, 2H), 2.44-2.54 (m, 2H), 2.56-2.68 (m, 4H), 2.76-2.82 (m, 1H), 2.85-2.96 (m, 3H), 6.69 (d, 1H), 6.71-6.77 (m, 4H), 7.15 (d, 1H), 7.20 (t, 1H).

### Example 56

### 8-(3-Hydroxyphenyl)-9-[6-(methyl{3-[(3,3,3-trifluoropropyl)sulphonyl]propyl}amino)hexyl]-6,7-dihydro-5H-benzo[7]annulen-3-ol

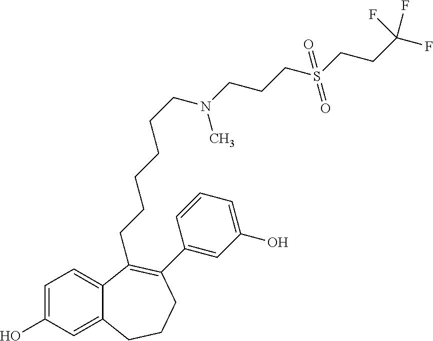 [SMILES: CN(CCCCCCC1=C(c2cccc(O)c2)CCCc2cc(O)ccc21)CCCS(=O)(=O)CCC(F)(F)F]

200 mg (0.48 mmol) of 9-(6-bromohexyl)-8-(3-hydroxyphenyl)-6,7-dihydro-5H-benzo[7]annulen-3-ol were reacted with 157 mg (0.67 mmol) of N-methyl-3-[(3,3,3-trifluoropropyl)sulphonyl]propane-1-amine according to Procedure 11. The material was then purified by HPLC method 1. This gave 120 mg (44% of theory) of product.

¹H NMR (600 MHz, chloroform-d₁): δ=1.07-1.18 (m, 4H), 1.19-1.29 (m, 2H), 1.31-1.37 (m, 2H), 2.01-2.13 (m, 6H), 2.21 (s, 3H), 2.24 (mc, 2H), 2.37 (mc, 2H), 2.48 (t, 2H), 2.60 (t, 2H), 2.64-2.73 (m, 2H), 3.13 (mc, 2H), 3.21 (mc, 2H), 6.70 (d, 1H), 6.72-6.77 (m, 3H), 6.78 (d, 1H), 7.17 (d, 1H), 7.22 (t, 1H).

### Example 57

### 9-[6-(Methyl{3-[(4,4,4-trifluorobutyl)sulphonyl]propyl}amino)hexyl]-8-phenyl-6,7-dihydro-5H-benzo[7]annulen-3-ol

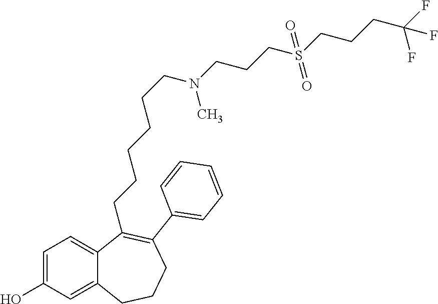 [SMILES: CN(CCCCCCC1=C(c2ccccc2)CCCc2cc(O)ccc21)CCCS(=O)(=O)CCCC(F)(F)F]

250 mg (0.63 mmol) of 9-(6-bromohexyl)-8-phenyl-6,7-dihydro-5H-benzo[7]annulen-3-ol were reacted with 185.8 mg (0.75 mmol) of N-methyl-3-[(4,4,4-trifluorobutyl)sulphonyl]propane-1-amine according to Procedure 11. The material was then purified by HPLC method 5 and HPLC method 6. This gave 77 mg (22% of theory) of product.

¹H NMR (600 MHz, chloroform-d₁): δ=1.04-1.14 (m, 4H), 1.17-1.28 (m, 4H), 1.94 (mc, 2H), 2.09-2.20 (m, 11H), 2.27-2.41 (m, 6H), 2.64 (mc, 2H), 3.00-3.05 (m, 4H), 6.72 (d, 1H), 6.74 (dd, 1H), 7.18 (d, 1H), 7.23-7.27 (m, 3H), 7.33-7.34 (m, 2H).

### Example 58

### 9-[6-(Methyl{4-[(4,4,4-trifluorobutyl)sulphonyl]butyl}amino)hexyl]-8-phenyl-6,7-dihydro-5H-benzo[7]annulen-3-ol

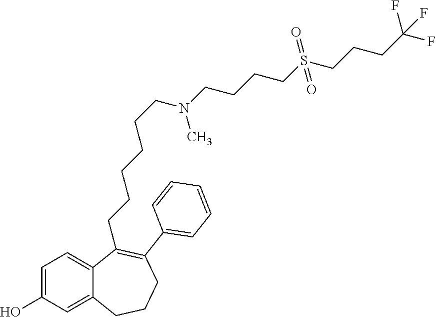 [SMILES: CN(CCCCCCC1=C(c2ccccc2)CCCc2cc(O)ccc21)CCCCS(=O)(=O)CCCC(F)(F)F]

250 mg (0.63 mmol) of 9-(6-bromohexyl)-8-phenyl-6,7-dihydro-5H-benzo[7]annulen-3-ol were reacted with 229 mg (0.88 mmol) of N-methyl-4-[(4,4,4-trifluorobutyl)sulphonyl]butane-1-amine according to Procedure 11. The material was then purified by HPLC method 5 and Chiralpak IB (5μ, 150×20 mm, 25 ml/min, mobile phase: hexane with 0.1% diethylamine-ethanol 90:10). This gave 67.8 mg (19% of theory) of product.

¹H NMR (600 MHz, chloroform-d₁): δ=1.07-1.14 (m, 4H), 1.17-1.23 (m, 2H), 1.24-1.31 (m, 2H), 1.61 (mc, 2H), 1.86 (mc, 2H), 2.09-2.24 (m, 11H), 2.28-2.40 (m, 6H), 2.63 (mc, 2H), 2.99-3.05 (m, 4H), 6.72 (d, 1H), 6.74 (dd, 1H), 7.17 (d, 1H), 7.23-7.26 (m, 3H), 7.33-7.37 (m, 2H).

### Example 59

### 9-[6-(Methyl{3-[(RS)-(3,3,3-trifluoropropyl)sulphinyl]propyl}amino)hexyl]-8-phenyl-6,7-dihydro-5H-benzo[7]annulen-3-ol

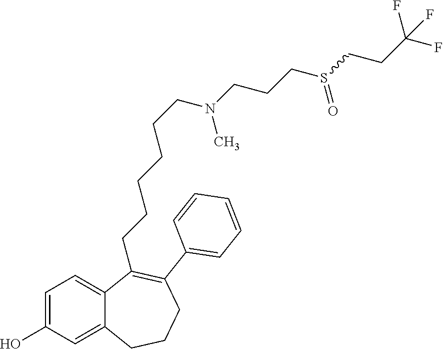 [SMILES: CN(CCCCCCC1=C(c2ccccc2)CCCc2cc(O)ccc21)CCCS(=O)CCC(F)(F)F]

250 mg (0.63 mmol) of 9-(6-bromohexyl)-8-phenyl-6,7-dihydro-5H-benzo[7]annulen-3-ol were reacted with 190.4 mg (0.88 mmol) of N-methyl-3-[(RS)-(3,3,3-trifluoropropyl)sulphinyl]propane-1-amine according to Procedure 11. The material was then purified by HPLC method 4 and HPLC method 9. This gave 148 mg (44% of theory) of product.

¹H NMR (600 MHz, chloroform-d₁): δ=1.06-1.13 (m, 4H), 1.17-1.26 (m, 4H), 1.83-1.96 (m, 2H), 2.07-2.18 (m, 9H), 2.33-2.43 (m, 4H), 2.58-2.69 (m, 4H), 2.72-2.81 (m, 2H), 2.84-2.93 (m, 2H), 6.72 (d, 1H), 6.75 (dd, 1H), 7.16 (d, 1H), 7.23-7.27 (m, 3H), 7.33-7.37 (m, 2H).

### Example 60

### 9-[6-(Methyl{3-[(3,3,3-trifluoropropyl)sulphonyl]propyl}amino)hexyl]-8-phenyl-6,7-dihydro-5H-benzo[7]annulen-3-ol

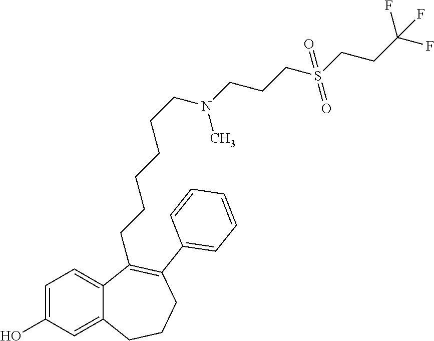 [SMILES: CN(CCCCCCC1=C(c2ccccc2)CCCc2cc(O)ccc21)CCCS(=O)(=O)CCC(F)(F)F]

250 mg (0.63 mmol) of 9-(6-bromohexyl)-8-phenyl-6,7-dihydro-5H-benzo[7]annulen-3-ol were reacted with 204 mg (0.88 mmol) of N-methyl-3-[(3,3,3-trifluoropropyl)sulphonyl]propane-1-amine according to Procedure 11. The material was then purified by HPLC method 4. This gave 160 mg (46% of theory) of product.

¹H NMR (600 MHz, chloroform-d₁): δ=1.05-1.14 (m, 4H), 1.16-1.23 (m, 2H), 1.24-1.31 (m, 2H), 2.00 (mc, 2H), 2.07-2.14 (m, 4H), 2.18 (s, 3H), 2.23 (t, 2H), 2.37 (t, 2H), 2.46 (t, 2H), 2.60-2.72 (m, 4H), 3.09 (mc, 2H), 3.18 (mc, 2H), 6.71 (d, 1H), 6.73 (dd, 1H), 7.17 (d, 1H), 7.23-7.27 (m, 3H), 7.35 (t, 2H).

### Example 61

### 9-[6-(Methyl{3-[(RS)-(3,3,4,4,4-pentafluorobutyl)sulphinyl]propyl}amino)hexyl]-8-phenyl-6,7-dihydro-5H-benzo[7]annulen-3-ol

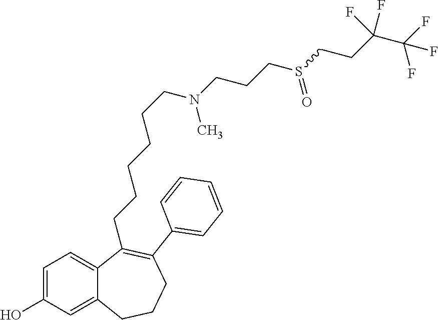 [SMILES: CN(CCCCCCC1=C(c2ccccc2)CCCc2cc(O)ccc21)CCCS(=O)CCC(F)(F)C(F)(F)F]

200 mg (0.50 mmol) of 9-(6-bromohexyl)-8-phenyl-6,7-dihydro-5H-benzo[7]annulen-3-ol were reacted with 187.4 mg (0.70 mmol) of N-methyl-3-[(RS)-(3,3,4,4,4-pentafluorobutyl)sulphinyl]propane-1-amine according to Procedure 11. The material was then purified by HPLC method 4. This gave 103 mg (35% of theory) of product.

¹H NMR (600 MHz, chloroform-d₁): δ=1.05 (mc, 2H), 1.14 (mc, 2H), 1.19-1.33 (m, 4H), 2.05-2.19 (m, 6H), 2.28-2.37 (m, 2H), 2.37-2.43 (m, 5H), 2.53-2.67 (m, 4H), 2.69-3.04 (m, 6H), 6.74 (d, 1H), 6.77 (dd, 1H), 7.16 (d, 1H), 7.22-7.28 (m, 3H), 7.35 (t, 2H).

### Example 62

9-[6-(Methyl{3-[(3,3,4,4,4-pentafluorobutyl)sulphonyl]propyl}amino)hexyl]-8-phenyl-6,7-dihydro-5H-benzo[7]annulen-3-ol:

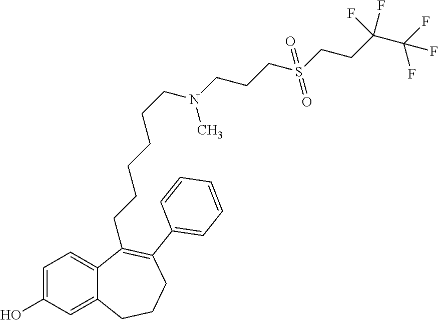 [SMILES: CN(CCCCCCC1=C(c2ccccc2)CCCc2cc(O)ccc21)CCCS(=O)(=O)CCC(F)(F)C(F)(F)F]

200 mg (0.50 mmol) of 9-(6-bromohexyl)-8-phenyl-6,7-dihydro-5H-benzo[7]annulen-3-ol were reacted with 199 mg (0.70 mmol) of N-methyl-3-[(3,3,4,4,4-pentafluorobutyl)sulphonyl]propane-1-amine according to Procedure 11. The material was then purified by HPLC method 4. This gave 85 mg (28% of theory) of product.

¹H NMR (600 MHz, chloroform-d₁): δ=1.04-1.14 (m, 4H), 1.21 (mc, 2H), 1.24-1.31 (m, 2H), 2.06-2.15 (m, 6H), 2.26-2.32 (m, 5H), 2.39 (t, 2H), 2.58-2.69 (m, 6H), 3.16 (t, 2H), 3.24 (mc, 2H), 6.73 (d, 1H), 6.76 (dd, 1H), 7.18 (d, 1H), 7.22-7.27 (m, 3H), 7.35 (t, 2H).

### Example 63

### 9-[6-(Methyl{3-[(4,4,5,5,5-pentafluoropentyl)sulphonyl]propyl}amino)hexyl]-8-phenyl-6,7-dihydro-5H-benzo[7]annulen-3-ol

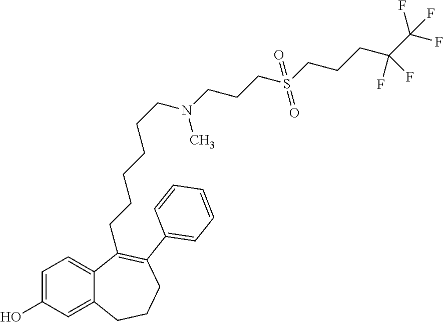 [SMILES: CN(CCCCCCC1=C(c2ccccc2)CCCc2cc(O)ccc21)CCCS(=O)(=O)CCCC(F)(F)C(F)(F)F]

240 mg (0.60 mmol) of 9-(6-bromohexyl)-8-phenyl-6,7-dihydro-5H-benzo[7]annulen-3-ol were reacted with 250 mg (0.84 mmol) of N-methyl-3-[(4,4,5,5,5-pentafluoropentyl)sulphonyl]propane-1-amine according to Procedure 11. The material was then purified by HPLC method 9. This gave 188 mg (51% of theory) of product.

¹H NMR (600 MHz, chloroform-d₁): δ=1.04-1.14 (m, 4H), 1.17-1.29 (m, 4H), 1.95 (mc, 2H), 2.08-2.15 (m, 7H), 2.15-2.32 (m, 6H), 2.35-2.41 (m, 4H), 2.60-2.67 (m, 2H), 3.00-3.06 (m, 4H), 6.71 (d, 1H), 6.74 (dd, 1H), 7.18 (d, 1H), 7.23-7.27 (m, 3H), 7.33-7.37 (m, 2H).

### Example 64

### 9-[6-(Methyl{3-[(RS)-(4,4,5,5,5-pentafluoropentyl)sulphinyl]propyl}amino)hexyl]-8-phenyl-6,7-dihydro-5H-benzo[7]annulen-3-ol

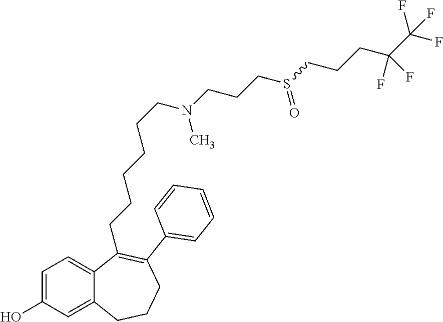 [SMILES: CN(CCCCCCC1=C(c2ccccc2)CCCc2cc(O)ccc21)CCCS(=O)CCCC(F)(F)C(F)(F)F]

250 mg (0.63 mmol) of 9-(6-bromohexyl)-8-phenyl-6,7-dihydro-5H-benzo[7]annulen-3-ol were reacted with 247 mg (0.88 mmol) of N-methyl-3-[(RS)-(4,4,5,5,5-pentafluoropentyl)sulphinyl]propane-1-amine according to Procedure 11. The material was then purified by HPLC method 6. This gave 193 mg (51% of theory) of product.

¹H NMR (600 MHz, chloroform-d₁): δ=1.04-1.14 (m, 4H), 1.15-1.26 (m, 4H), 1.82-1.95 (m, 2H), 2.06-2.30 (m, 13H), 2.32-2.43 (m, 4H), 2.63 (t, 2H), 2.69-2.85 (m, 4H), 6.73 (d, 1H), 6.76 (dd, 1H), 7.16 (d, 1H), 7.22-7.27 (m, 3H), 7.32-7.37 (m, 2H).

### Example 65

### 9-[6-(Ethyl{3-[(3,3,3-trifluoropropyl)sulphonyl]propyl}amino)hexyl]-8-(3-hydroxyphenyl)-6,7-dihydro-5H-benzo[7]annulen-3-ol

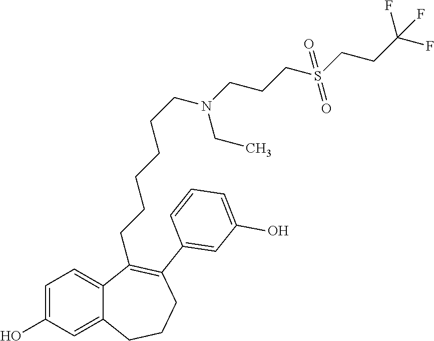 [SMILES: CCN(CCCCCCC1=C(c2cccc(O)c2)CCCc2cc(O)ccc21)CCCS(=O)(=O)CCC(F)(F)F]

200 mg (0.48 mmol) of 9-(6-bromohexyl)-8-(3-hydroxyphenyl)-6,7-dihydro-5H-benzo[7]annulen-3-ol were reacted with 166.7 mg (0.67 mmol) of N-ethyl-3-[(3,3,3-trifluoropropyl)sulphonyl]propane-1-amine according to Procedure 11. The material was then purified by HPLC method 10. This gave 100 mg (36% of theory) of product.

¹H NMR (600 MHz, chloroform-d₁): δ=1.04-1.17 (m, 7H), 1.21 (mc, 2H), 1.42 (mc, 2H), 2.04-2.16 (m, 6H), 2.37 (t, 2H), 2.50 (mc, 2H), 2.58 (t, 2H), 2.65-2.82 (m, 6H), 3.16 (t, 2H), 3.23 (mc, 2H) 6.70 (d, 1H), 6.71-6.76 (m, 3H), 6.79 (t, 1H), 7.16 (d, 1H), 7.21 (t, 1H).

### Example 66

### 9-[6-(Methyl{4-[(RS)-(4,4,5,5,5-pentafluoropentyl)sulphinyl]butyl}amino)hexyl]-8-phenyl-6,7-dihydro-5H-benzo[7]annulen-3-ol

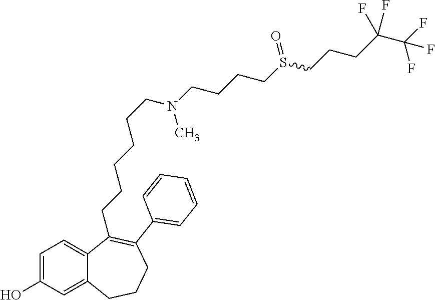 [SMILES: CN(CCCCCCC1=C(c2ccccc2)CCCc2cc(O)ccc21)CCCCS(=O)CCCC(F)(F)C(F)(F)F]

200 mg (0.50 mmol) of 9-(6-bromohexyl)-8-phenyl-6,7-dihydro-5H-benzo[7]annulen-3-ol were reacted with 207 mg (0.70 mmol) of N-methyl-4-[(RS)-(4,4,5,5,5-pentafluoropentyl)sulphinyl]butane-1-amine according to Procedure 11. The material was then purified by HPLC method 11. This gave 254 mg (78% of theory) of product.

¹H NMR (600 MHz, chloroform-d₁): δ=1.04-1.12 (m, 2H), 1.12-1.24 (m, 4H), 1.31-1.38 (m, 2H), 1.75-1.96 (m, 4H), 2.08-2.32 (m, 8H), 2.39 (t, 2H), 2.46-2.55 (m, 5H), 2.59-2.69 (m, 3H), 2.70-2.87 (m, 5H), 6.74 (d, 1H), 6.77 (dd, 1H), 7.14 (d, 1H), 7.22-7.27 (m, 3H), 7.32-7.37 (m, 2H).

### Example 67

### 9-[6-(Methyl{4-[(4,4,5,5,5-pentafluoropentyl)sulphonyl]butyl}amino)hexyl]-8-phenyl-6,7-dihydro-5H-benzo[7]annulen-3-ol

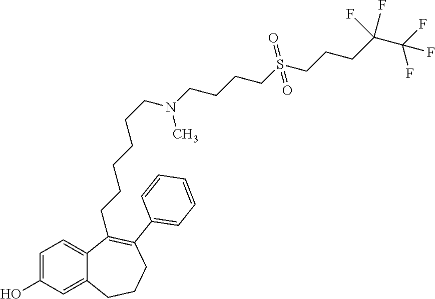 [SMILES: CN(CCCCCCC1=C(c2ccccc2)CCCc2cc(O)ccc21)CCCCS(=O)(=O)CCCC(F)(F)C(F)(F)F]

200 mg (0.50 mmol) of 9-(6-bromohexyl)-8-phenyl-6,7-dihydro-5H-benzo[7]annulen-3-ol were reacted with 218.3 mg (0.70 mmol) of N-methyl-4-[(4,4,5,5,5-pentafluoropentyl)sulphonyl]butane-1-amine according to Procedure 11. The material was then purified by HPLC method 11. This gave 231 mg (73% of theory) of product.

¹H NMR (600 MHz, chloroform-d₁): δ=1.05-1.14 (m, 4H), 1.16-1.24 (m, 2H), 1.30-1.36 (m, 2H), 1.73 (mc, 2H), 1.89 (mc, 2H), 2.06-2.14 (m, 4H), 2.15-2.32 (m, 4H), 2.33-2.41 (m, 7H), 2.55 (t, 2H), 2.62 (mc, 2H), 3.01-3.08 (m, 4H), 6.72 (d, 1H), 6.74 (dd, 1H), 7.15 (d, 1H), 7.22-7.27 (m, 3H), 7.33-7.37 (m, 2H).

### Example 68

### 9-[6-(Ethyl{3-[(RS)-(4,4,5,5,5-pentafluoropentyl)sulphinyl]propyl}amino)hexyl]-8-phenyl-6,7-dihydro-5H-benzo[7]annulen-3-ol

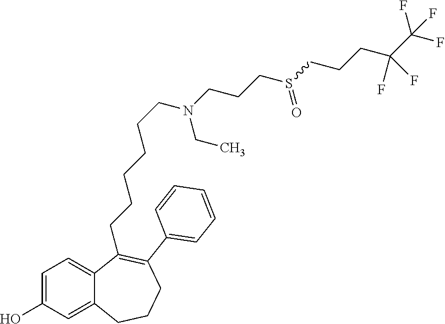 [SMILES: CCN(CCCCCCC1=C(c2ccccc2)CCCc2cc(O)ccc21)CCCS(=O)CCCC(F)(F)C(F)(F)F]

200 mg (0.50 mmol) of 9-(6-bromohexyl)-8-phenyl-6,7-dihydro-5H-benzo[7]annulen-3-ol were reacted with 207 mg (0.70 mmol) of N-ethyl-3-[(RS)-(4,4,5,5,5-pentafluoropentyl)sulphinyl]propane-1-amine according to Procedure 11. The material was then purified by HPLC method 11. This gave 121 mg (38% of theory) of product.

¹H NMR (600 MHz, chloroform-d₁): δ=1.01-1.07 (m, 2H), 1.10-1.17 (m, 5H), 1.21-1.30 (m, 4H), 2.07-2.32 (m, 10H), 2.37-2.45 (m, 4H), 2.60-2.67 (m, 2H), 2.76-2.93 (m, 8H), 6.75 (d, 1H), 6.78 (dd, 1H), 7.15 (d, 1H), 7.22-7.27 (m, 3H), 7.33-7.37 (m, 2H).

### Example 69

### 9-[6-(Ethyl{3-[(4,4,5,5,5-pentafluoropentyl)sulphonyl]propyl}amino)hexyl]-8-phenyl-6,7-dihydro-5H-benzo[7]annulen-3-ol

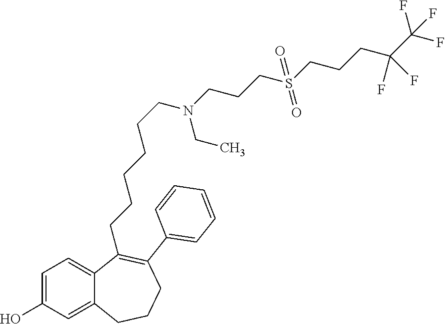 [SMILES: CCN(CCCCCCC1=C(c2ccccc2)CCCc2cc(O)ccc21)CCCS(=O)(=O)CCCC(F)(F)C(F)(F)F]

200 mg (0.50 mmol) of 9-(6-bromohexyl)-8-phenyl-6,7-dihydro-5H-benzo[7]annulen-3-ol were reacted with 218.3 mg (0.70 mmol) of N-ethyl-3-[(4,4,5,5,5-pentafluoropentyl)sulphonyl]propane-1-amine according to Procedure 11. The material was then purified by HPLC method 11. This gave 141 mg (45% of theory) of product.

¹H NMR (600 MHz, chloroform-d₁): δ=0.95 (t, 3H), 1.04-1.14 (m, 4H), 1.17-1.25 (m, 4H), 1.91 (mc, 2H), 2.08-2.32 (m, 10H), 2.37 (t, 2H), 2.42-2.49 (m, 4H), 2.64 (mc, 2H), 3.01-3.07 (m, 4H), 6.72 (d, 1H), 6.75 (dd, 1H), 7.18 (d, 1H), 7.23-7.28 (m, 3H), 7.33-7.37 (m, 2H).

### Example 70

### 9-{6-[(2-Hydroxyethyl){3-[(RS)-(4,4,5,5,5-pentafluoropentyl)sulphinyl]propyl}amino]hexyl}-8-phenyl-6,7-dihydro-5H-benzo[7]annulen-3-ol

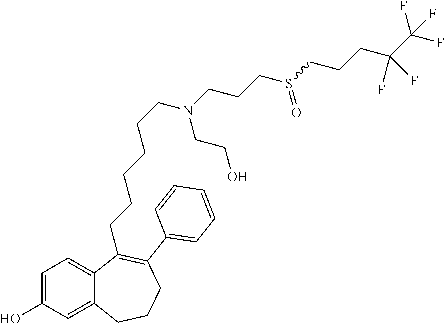 [SMILES: O=S(CCCN(CCO)CCCCCCC1=C(c2ccccc2)CCCc2cc(O)ccc21)CCCC(F)(F)C(F)(F)F]

200 mg (0.50 mmol) of 9-(6-bromohexyl)-8-phenyl-6,7-dihydro-5H-benzo[7]annulen-3-ol were reacted with 218.3 mg (0.70 mmol) of 2-({3-[(RS)-(4,4,5,5,5-pentafluoropentyl)sulphinyl]propyl}amino)ethanol according to Procedure 11. The material was then purified by HPLC method 11. This gave 90.6 mg (28% of theory) of product.

¹H NMR (600 MHz, chloroform-d₁): δ=1.02-1.08 (m, 2H), 1.09-1.16 (m, 2H), 1.19-1.26 (m, 4H), 2.03-2.32 (m, 10H), 2.36-2.42 (m, 4H), 2.61-2.67 (m, 2H), 2.69-2.82 (m, 7H), 2.84-2.90 (m, 1H), 3.66 (t, 2H), 6.74 (d, 1H), 6.78 (dd, 1H), 7.16 (d, 1H), 7.22-7.28 (m, 3H), 7.33-7.37 (m, 2H).

### Example 71

### 9-{6-[(2-Hydroxyethyl){3-[(4,4,5,5,5-pentafluoropentyl)sulphonyl]propyl}amino]hexyl}-8-phenyl-6,7-dihydro-5H-benzo[7]annulen-3-ol

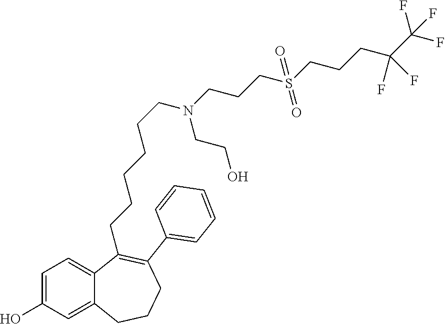 [SMILES: O=S(=O)(CCCN(CCO)CCCCCCC1=C(c2ccccc2)CCCc2cc(O)ccc21)CCCC(F)(F)C(F)(F)F]

200 mg (0.50 mmol) of 9-(6-bromohexyl)-8-phenyl-6,7-dihydro-5H-benzo[7]annulen-3-ol were reacted with 229.5 mg (0.70 mmol) of 2-({3-[(4,4,5,5,5-pentafluoropentyl)sulphonyl]propyl}amino)ethanol according to Procedure 11. The material was then purified by HPLC method 11. This gave 95.8 mg (29% of theory) of product.

¹H NMR (400 MHz, chloroform-d₁): δ=1.02-1.15 (m, 4H), 1.16-1.31 (m, 4H), 2.03-2.46 (m, 14H), 2.64 (mc, 2H), 2.69 (t, 2H), 2.74 (t, 2H), 3.03-3.12 (m, 4H), 3.65 (t, 2H), 6.74 (d, 1H), 6.77 (dd, 1H), 7.18 (d, 1H), 7.21-7.28 (m, 3H), 7.32-7.38 (m, 2H).

### Example 72

### 9-{6-[(3-Hydroxypropyl){3-[(RS)-(4,4,5,5,5-pentafluoropentyl)sulphinyl]propyl}amino]hexyl}-8-phenyl-6,7-dihydro-5H-benzo[7]annulen-3-ol

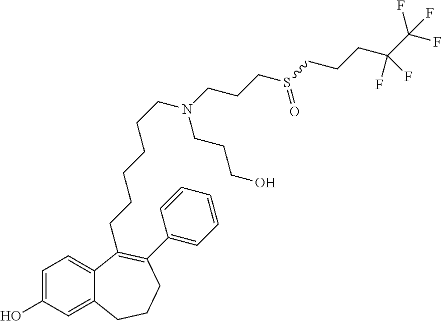 [SMILES: O=S(CCCN(CCCO)CCCCCCC1=C(c2ccccc2)CCCc2cc(O)ccc21)CCCC(F)(F)C(F)(F)F]

200 mg (0.50 mmol) of 9-(6-bromohexyl)-8-phenyl-6,7-dihydro-5H-benzo[7]annulen-3-ol were reacted with 218.3 mg (0.67 mmol) of 3-({3-[(RS)-(4,4,5,5,5-pentafluoropentyl)sulphinyl]propyl}amino)propan-1-ol according to Procedure 11. The material was then purified by HPLC method 11. This gave 147.5 mg (45% of theory) of product.

¹H NMR (600 MHz, chloroform-d₁): δ=1.03-1.18 (m, 4H), 1.19-1.28 (m, 4H), 1.77 (mc, 2H), 2.05-2.32 (m, 10H), 2.36-2.45 (m, 4H), 2.63 (mc, 2H), 2.72-2.90 (m, 8H), 3.74 (t, 2H), 6.74 (d, 1H), 6.77 (dd, 1H), 7.15 (d, 1H), 7.22-7.28 (m, 3H), 7.33-7.37 (m, 2H).

### Example 73

### 9-{6-[(3-Hydroxypropyl){3-[(4,4,5,5,5-pentafluoropentyl)sulphonyl]propyl}amino]hexyl}-8-phenyl-6,7-dihydro-5H-benzo[7]annulen-3-ol

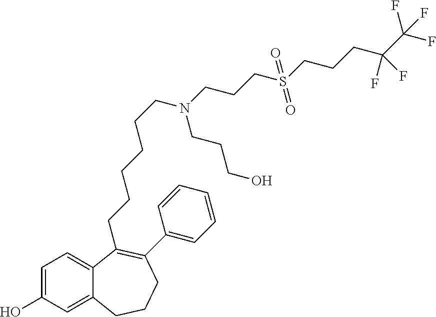 [SMILES: O=S(=O)(CCCN(CCCO)CCCCCCC1=C(c2ccccc2)CCCc2cc(O)ccc21)CCCC(F)(F)C(F)(F)F]

200 mg (0.50 mmol) of 9-(6-bromohexyl)-8-phenyl-6,7-dihydro-5H-benzo[7]annulen-3-ol were reacted with 239.3 mg (0.70 mmol) of 3-({3-[(4,4,5,5,5-pentafluoropentyl)sulphonyl]propyl}amino)propan-1-ol according to Procedure 11. The material was then purified by HPLC method 11. This gave 137.4 mg (42% of theory) of product.

¹H NMR (400 MHz, chloroform-d₁): δ=1.04-1.14 (m, 4H), 1.17-1.28 (m, 4H), 1.70 (mc, 2H), 2.01-2.42 (m, 14H), 2.58-2.71 (m, 6H), 3.04 (mc, 2H), 3.10 (t, 2H), 3.76 (t, 2H), 6.73 (d, 1H), 6.76 (dd, 1H), 7.18 (d, 1H), 7.22-7.28 (m, 3H), 7.32-7.38 (m, 2H).

### Example 74

### 9-{6-[(2-Hydroxy-2-methylpropyl){3-[(RS)-(4,4,5,5,5-pentafluoropentyl)sulphinyl]propyl}amino]hexyl}-8-phenyl-6,7-dihydro-5H-benzo[7]annulen-3-ol

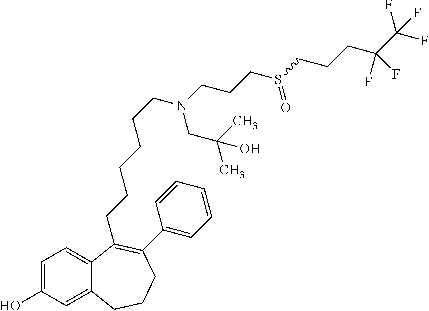 [SMILES: CC(C)(O)CN(CCCCCCC1=C(c2ccccc2)CCCc2cc(O)ccc21)CCCS(=O)CCCC(F)(F)C(F)(F)F]

200 mg (0.50 mmol) of 9-(6-bromohexyl)-8-phenyl-6,7-dihydro-5H-benzo[7]annulen-3-ol were reacted with 305.9 mg (0.90 mmol) of 2-methyl-1-({3-[(RS)-(4,4,5,5,5-pentafluoropentyl)sulphinyl]propyl}amino)propan-2-ol according to Procedure 11. The material was then purified by HPLC method 11. This gave 88.4 mg (27% of theory) of product.

¹H NMR (600 MHz, chloroform-d₁): δ=1.02-1.15 (m, 4H), 1.16-1.26 (m, 10H), 2.02 (mc, 2H), 2.08-2.32 (m, 8H), 2.35-2.45 (m, 4H), 2.46-2.51 (m, 2H), 2.59-2.68 (m, 2H), 2.70-2.82 (m, 5H), 2.84-2.90 (m, 1H), 6.74 (d, 1H), 6.78 (dd, 1H), 7.16 (d, 1H), 7.22-7.28 (m, 3H), 7.33-7.37 (m, 2H).

### Example 75

### 9-{6-[(2-Hydroxy-2-methylpropyl){3-[(4,4,5,5,5-pentafluoropentyl)sulphonyl]propyl}amino]hexyl}-8-phenyl-6,7-dihydro-5H-benzo[7]annulen-3-01

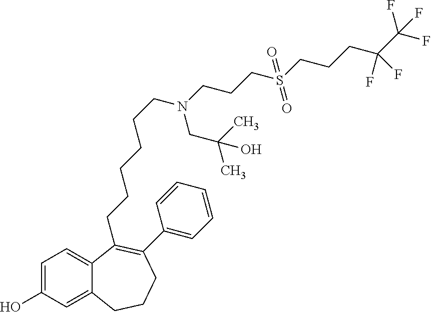 [SMILES: CC(C)(O)CN(CCCCCCC1=C(c2ccccc2)CCCc2cc(O)ccc21)CCCS(=O)(=O)CCCC(F)(F)C(F)(F)F]

200 mg (0.50 mmol) of 9-(6-bromohexyl)-8-phenyl-6,7-dihydro-5H-benzo[7]annulen-3-ol were reacted with 213.6 mg (0.60 mmol) of 2-methyl-1-({3-[(4,4,5,5,5-pentafluoropentyl)sulphonyl]propyl}amino)propan-2-ol according to Procedure 11. The material was then purified by HPLC method 11. This gave 42.6 mg (12% of theory) of product.

¹H NMR (400 MHz, chloroform-d₁): δ=1.01-1.30 (m, 14H), 1.96 (mc, 2H), 2.08-2.43 (m, 14H), 2.59-2.68 (m, 4H), 2.98-3.09 (m, 4H), 6.72 (d, 1H), 6.75 (dd, 1H), 7.18 (d, 1H), 7.22-7.28 (m, 3H), 7.32-7.38 (m, 2H).

### Example 76

### 9-[6-(Ethyl{3-[(RS)-(3,3,3-trifluoropropyl)sulphinyl]propyl}amino)hexyl]-8-phenyl-6,7-dihydro-5H-benzo[7]annulen-3-ol

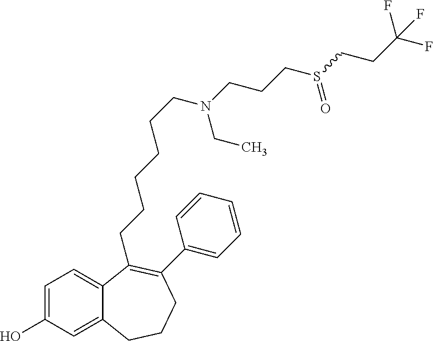 [SMILES: CCN(CCCCCCC1=C(c2ccccc2)CCCc2cc(O)ccc21)CCCS(=O)CCC(F)(F)F]

200 mg (0.50 mmol) of 9-(6-bromohexyl)-8-phenyl-6,7-dihydro-5H-benzo[7]annulen-3-ol were reacted with 162.1 mg (0.70 mmol) of N-ethyl-3-[(RS)-(3,3,3-trifluoropropyl)sulphinyl]propane-1-amine according to Procedure 11. The material was then purified by HPLC method 11. This gave 176 mg (63% of theory) of product.

¹H NMR (400 MHz, chloroform-d₁): δ=1.00-1.18 (m, 7H), 1.19-1.34 (m, 4H), 2.04-2.22 (m, 6H), 2.39 (t, 2H), 2.45 (mc, 2H), 2.57-2.70 (m, 4H), 2.76-3.01 (m, 8H), 6.74 (d, 1H), 6.77 (dd, 1H), 7.15 (d, 1H), 7.21-7.28 (m, 3H), 7.32-7.38 (m, 2H).

### Example 77

### 9-[6-(Ethyl{3-[(3,3,3-trifluoropropyl)sulphonyl]propyl}amino)hexyl]-8-phenyl-6,7-dihydro-5H-benzo[7]annulen-3-ol

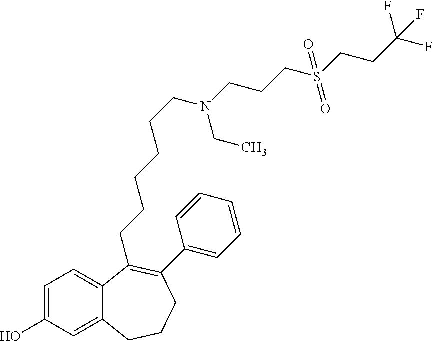 [SMILES: CCN(CCCCCCC1=C(c2ccccc2)CCCc2cc(O)ccc21)CCCS(=O)(=O)CCC(F)(F)F]

200 mg (0.50 mmol) of 9-(6-bromohexyl)-8-phenyl-6,7-dihydro-5H-benzo[7]annulen-3-ol were reacted with 173.4 mg (0.70 mmol) of N-ethyl-3-[(3,3,3-trifluoropropyl)sulphonyl]propane-1-amine according to Procedure 11. The material was then purified by HPLC method 11. This gave 185 mg (65% of theory) of product.

¹H NMR (400 MHz, chloroform-d₁): δ=1.00-1.36 (m, 11H), 2.06-2.17 (m, 4H), 2.22 (mc, 2H), 2.39 (t, 2H), 2.53 (mc, 2H), 2.60-2.76 (m, 4H), 2.88 (q, 2H), 2.93 (mc, 2H), 3.18-3.29 (m, 4H), 6.75 (d, 1H), 6.78 (dd, 1H), 7.16 (d, 1H), 7.21-7.28 (m, 3H), 7.31-7.38 (m, 2H).

### Example 78

### 9-{6-[(2-Hydroxyethyl){3-[(RS)-(3,3,3-trifluoropropyl)sulphinyl]propyl}amino]hexyl}-8-phenyl-6,7-dihydro-5H-benzo[7]annulen-3-ol

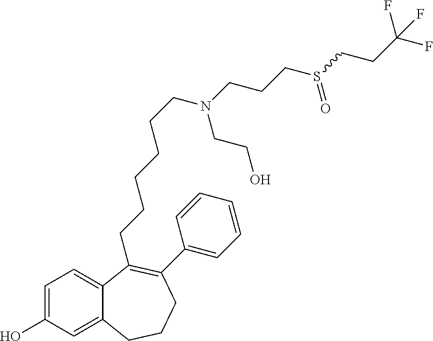 [SMILES: O=S(CCCN(CCO)CCCCCCC1=C(c2ccccc2)CCCc2cc(O)ccc21)CCC(F)(F)F]

200 mg (0.50 mmol) of 9-(6-bromohexyl)-8-phenyl-6,7-dihydro-5H-benzo[7]annulen-3-ol were reacted with 173.4 mg (0.70 mmol) of 2-({3-[(RS)-(3,3,3-trifluoropropyl)sulphinyl]propyl}amino)ethanol according to Procedure 11. The material was then purified by HPLC method 11. This gave 176 mg (61% of theory) of product.

¹H NMR (400 MHz, chloroform-d₁): δ=1.01-1.35 (m, 8H), 2.06-2.25 (m, 6H), 2.40 (t, 2H), 2.51 (mc, 2H), 2.59-2.71 (m, 4H), 2.78-3.01 (m, 8H), 3.76 (t, 2H), 6.74 (d, 1H), 6.77 (dd, 1H), 7.16 (d, 1H), 7.21-7.28 (m, 3H), 7.32-7.38 (m, 2H).

### Example 79

### 9-{6-[(2-Hydroxyethyl){3-[(3,3,3-trifluoropropyl)sulphonyl]propyl}amino]hexyl}-8-phenyl-6,7-dihydro-5H-benzo[7]annulen-3-ol

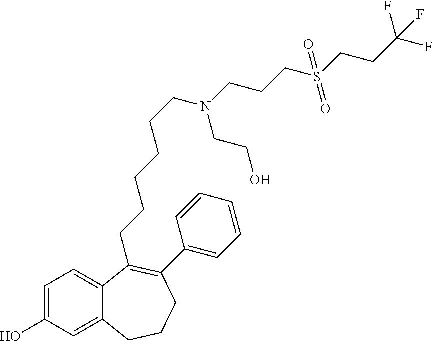 [SMILES: O=S(=O)(CCCN(CCO)CCCCCCC1=C(c2ccccc2)CCCc2cc(O)ccc21)CCC(F)(F)F]

200 mg (0.50 mmol) of 9-(6-bromohexyl)-8-phenyl-6,7-dihydro-5H-benzo[7]annulen-3-ol were reacted with 184.6 mg (0.70 mmol) of 2-({3-[(3,3,3-trifluoropropyl)sulphonyl]propyl}amino)ethanol according to Procedure 11. The material was then purified by HPLC method 11. This gave 136.5 mg (47% of theory) of product.

¹H NMR (400 MHz, chloroform-d₁): δ=1.02-1.35 (m, 8H), 2.06-2.19 (m, 6H), 2.39 (t, 2H), 2.51 (mc, 2H), 2.58-2.76 (m, 4H), 2.78 (t, 2H), 2.85 (t, 2H), 3.15 (t, 2H), 3.24 (mc, 2H), 3.71 (t, 2H), 6.74 (d, 1H), 6.77 (dd, 1H), 7.17 (d, 1H), 7.21-7.28 (m, 3H), 7.32-7.38 (m, 2H).

### Example 80

### 9-{6-[(3-Hydroxypropyl){3-[(RS)-(3,3,3-trifluoropropyl)sulphinyl]propyl}amino]hexyl}-8-phenyl-6,7-dihydro-5H-benzo[7]annulen-3-ol

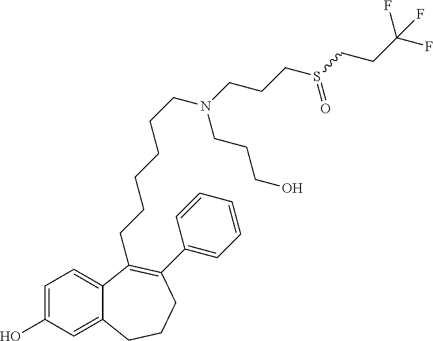 [SMILES: O=S(CCCN(CCCO)CCCCCCC1=C(c2ccccc2)CCCc2cc(O)ccc21)CCC(F)(F)F]

200 mg (0.50 mmol) of 9-(6-bromohexyl)-8-phenyl-6,7-dihydro-5H-benzo[7]annulen-3-ol were reacted with 183.2 mg (0.70 mmol) of 3-({3-[(RS)-(3,3,3-trifluoropropyl)sulphinyl]propyl}amino)propan-1-ol according to Procedure 11. The material was then purified by HPLC method 11. This gave 135.6 mg (46% of theory) of product.

¹H NMR (400 MHz, chloroform-d₁): δ=1.02-1.16 (m, 4H), 1.17-1.28 (m, 4H), 1.72 (mc, 2H), 1.98-2.16 (m, 6H), 2.30-2.42 (m, 4H), 2.56-2.83 (m, 10H), 2.87-2.99 (m, 2H), 3.75 (t, 2H), 6.73 (d, 1H), 6.76 (dd, 1H), 7.16 (d, 1H), 7.21-7.28 (m, 3H), 7.32-7.38 (m, 2H).

### Example 81

### 9-{6-[(3-Hydroxypropyl){3-[(3,3,3-trifluoropropyl)sulphonyl]propyl}amino]hexyl}-8-phenyl-6,7-dihydro-5H-benzo[7]annulen-3-ol

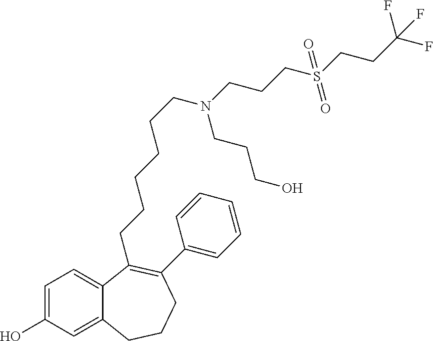 [SMILES: O=S(=O)(CCCN(CCCO)CCCCCCC1=C(c2ccccc2)CCCc2cc(O)ccc21)CCC(F)(F)F]

200 mg (0.50 mmol) of 9-(6-bromohexyl)-8-phenyl-6,7-dihydro-5H-benzo[7]annulen-3-ol were reacted with 194.4 mg (0.70 mmol) of 3-({3-[(3,3,3-trifluoropropyl)sulphonyl]propyl}amino)propan-1-ol according to Procedure 11. The material was then purified by HPLC method 11. This gave 115 mg (39% of theory) of product.

¹H NMR (400 MHz, chloroform-d₁): δ=1.02-1.17 (m, 4H), 1.18-1.32 (m, 4H), 1.77 (mc, 2H), 2.06-2.20 (m, 6H), 2.39 (t, 2H), 2.45 (mc, 2H), 2.58-2.86 (m, 8H), 3.15 (t, 2H), 3.25 (mc, 2H), 3.75 (t, 2H), 6.74-6.79 (m, 2H), 7.17 (d, 1H), 7.21-7.29 (m, 3H), 7.32-7.39 (m, 2H).

### Example 82

### 9-{6-[(2-Hydroxy-2-methylpropyl){3-[(RS)-(3,3,3-trifluoropropyl)sulphinyl]propyl}amino]hexyl}-8-phenyl-6,7-dihydro-5H-benzo[7]annulen-3-ol

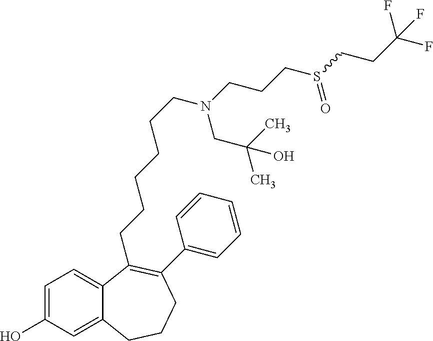 [SMILES: CC(C)(O)CN(CCCCCCC1=C(c2ccccc2)CCCc2cc(O)ccc21)CCCS(=O)CCC(F)(F)F]

250 mg (0.63 mmol) of 9-(6-bromohexyl)-8-phenyl-6,7-dihydro-5H-benzo[7]annulen-3-ol were reacted with 241.3 mg (0.88 mmol) of 2-methyl-1-({3-[(RS)-(3,3,3-trifluoropropyl)sulphinyl]propyl}amino)propan-2-ol according to Procedure 11. The material was then purified by HPLC method 11. This gave 43.8 mg (12% of theory) of product.

¹H NMR (400 MHz, chloroform-d₁): δ=1.02-1.13 (m, 4H), 1.15-1.29 (m, 4H), 1.99 (mc, 2H), 2.06-2.17 (m, 4H), 2.34-2.47 (m, 6H), 2.58-2.85 (m, 8H), 2.89-2.95 (m, 2H), 6.74 (d, 1H), 6.77 (dd, 1H), 7.16 (d, 1H), 7.21-7.28 (m, 3H), 7.32-7.38 (m, 2H).

### Example 83

### 9-{6-[(2-Hydroxy-2-methylpropyl){3-[(3,3,3-trifluoropropyl)sulphonyl]propyl}amino]hexyl}-8-phenyl-6,7-dihydro-5H-benzo[7]annulen-3-ol

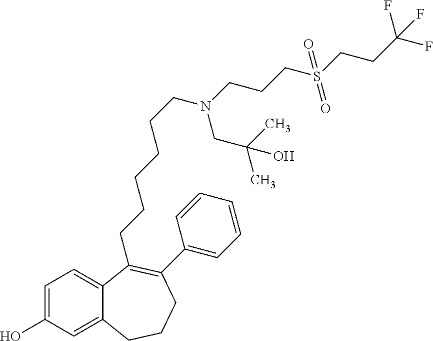 [SMILES: CC(C)(O)CN(CCCCCCC1=C(c2ccccc2)CCCc2cc(O)ccc21)CCCS(=O)(=O)CCC(F)(F)F]

250 mg (0.63 mmol) of 9-(6-bromohexyl)-8-phenyl-6,7-dihydro-5H-benzo[7]annulen-3-ol were reacted with 255.3 mg (0.88 mmol) of 2-methyl-1-({3-[(3,3,3-trifluoropropyl)sulphonyl]propyl}amino)propan-2-ol according to Procedure 11. The material was then purified by HPLC method 11. This gave 60.4 mg (16% of theory) of product.

¹H NMR (400 MHz, chloroform-d₁): δ=1.04-1.14 (m, 4H), 1.17-1.36 (m, 10H), 2.05-2.17 (m, 6H), 2.38 (t, 2H), 2.49-2.56 (m, 4H), 2.60-2.76 (m, 4H), 2.82 (t, 2H), 3.13 (t, 2H), 3.19-3.25 (m, 2H), 6.74 (d, 1H), 6.77 (dd, 1H), 7.17 (d, 1H), 7.22-7.28 (m, 3H), 7.32-7.39 (m, 2H).

### Example 84

### 9-[5-(Methyl{4-[(RS)-(4,4,5,5,5-pentafluoropentyl)sulphinyl]butyl}amino)pentyl]-8-phenyl-6,7-dihydro-5H-benzo[7]annulen-3-ol

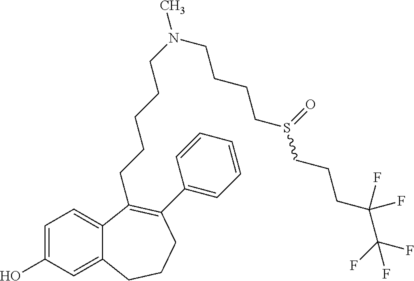 [SMILES: CN(CCCCCC1=C(c2ccccc2)CCCc2cc(O)ccc21)CCCCS(=O)CCCC(F)(F)C(F)(F)F]

210.8 mg (0.55 mmol) of 9-(5-bromopentyl)-8-phenyl-6,7-dihydro-5H-benzo[7]annulen-3-ol were reacted with 226.2 mg (0.77 mmol) of N-methyl-4-[(RS)-(4,4,5,5,5-pentafluoropentyl)sulphinyl]butane-1-amine according to Procedure 11. The material was then purified by HPLC method 11. This gave 287 mg (87% of theory) of product.

¹H NMR (600 MHz, chloroform-d₁): δ=1.07-1.15 (m, 2H), 1.20-1.29 (m, 2H), 1.36-1.45 (m, 2H), 1.69-1.92 (m, 4H), 2.07-2.32 (m, 8H), 2.37-2.51 (m, 7H), 2.59-2.88 (m, 8H), 6.73 (d, 1H), 6.77 (dd, 1H), 7.15 (d, 1H), 7.21-7.28 (m, 3H), 7.33-7.38 (m, 2H).

### Example 85

### 9-[5-(Methyl{4-[(4,4,5,5,5-pentafluoropentyl)sulphonyl]butyl}amino)pentyl]-8-phenyl-6,7-dihydro-5H-benzo[7]annulen-3-ol

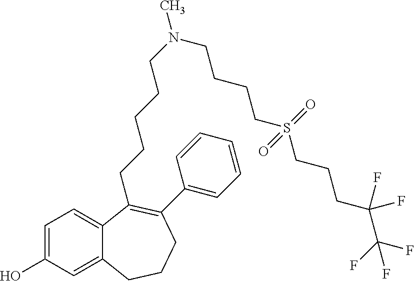 [SMILES: CN(CCCCCC1=C(c2ccccc2)CCCc2cc(O)ccc21)CCCCS(=O)(=O)CCCC(F)(F)C(F)(F)F]

200 mg (0.52 mmol) of 9-(5-bromopentyl)-8-phenyl-6,7-dihydro-5H-benzo[7]annulen-3-ol were reacted with 226.2 mg (0.73 mmol) of N-methyl-4-[(4,4,5,5,5-pentafluoropentyl)sulphonyl]butane-1-amine according to Procedure 11. The material was then purified by HPLC method 11. This gave 299.6 mg (94% of theory) of product.

¹H NMR (600 MHz, chloroform-d₁): δ=1.07-1.14 (m, 2H), 1.19-1.26 (m, 2H), 1.39-1.46 (m, 2H), 1.78-1.85 (m, 2H), 1.86-1.93 (m, 2H), 2.07-2.15 (m, 4H), 2.15-2.22 (m, 2H), 2.23-2.32 (m, 2H), 2.39 (t, 2H), 2.49 (s, 3H), 2.55 (mc, 2H), 2.61 (t, 2H), 2.76 (t, 2H), 3.03-3.10 (m, 4H), 6.72 (d, 1H), 6.75 (dd, 1H), 7.15 (d, 1H), 7.22-7.27 (m, 3H), 7.33-7.38 (m, 2H).

### Example 86

### 9-[6-(Ethyl{3-[(RS)-(4,4,5,5,5-pentafluoropentyl)sulphinyl]propyl}amino)hexyl]-8-(3-hydroxyphenyl)-6,7-dihydro-5H-benzo[7]annulen-3-ol

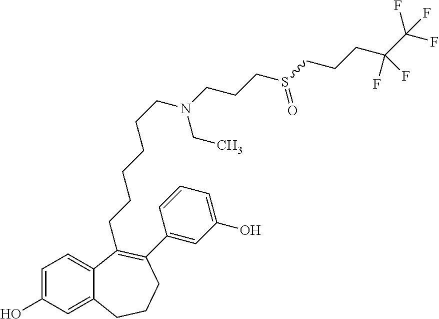 [SMILES: CCN(CCCCCCC1=C(c2cccc(O)c2)CCCc2cc(O)ccc21)CCCS(=O)CCCC(F)(F)C(F)(F)F]

200 mg (0.48 mmol) of 9-(6-bromohexyl)-8-(3-hydroxyphenyl)-6,7-dihydro-5H-benzo[7]annulen-3-ol were reacted with 199.1 mg (0.67 mmol) of N-ethyl-3-[(RS)-(4,4,5,5,5-pentafluoropentyl)sulphinyl]propane-1-amine according to Procedure 11. The material was then purified by HPLC method 12. This gave 164.2 mg (54% of theory) of product.

¹H NMR (400 MHz, chloroform-d₁): δ=1.01-1.26 (m, 9H), 1.44-1.53 (m, 2H), 2.05-2.34 (m, 10H), 2.40 (t, 2H), 2.57-2.68 (m, 4H), 2.75-3.05 (m, 8H), 6.67-6.76 (m, 4H), 6.82 (t-br., 1H), 7.14-7.22 (m, 2H).

### Example 87

### 9-{6-[(2-Hydroxyethyl){3-[(RS)-(4,4,5,5,5-pentafluoropentyl)sulphinyl]propyl}amino]hexyl}-8-(3-hydroxyphenyl)-6,7-dihydro-5H-benzo[7]annulen-3-ol

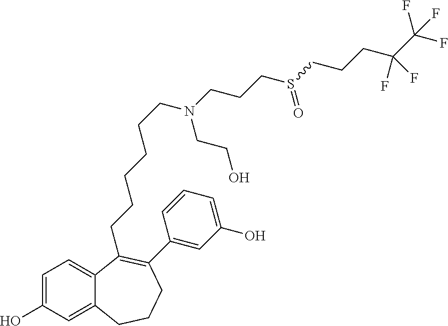 [SMILES: O=S(CCCN(CCO)CCCCCCC1=C(c2cccc(O)c2)CCCc2cc(O)ccc21)CCCC(F)(F)C(F)(F)F]

200 mg (0.48 mmol) of 9-(6-bromohexyl)-8-(3-hydroxyphenyl)-6,7-dihydro-5H-benzo[7]annulen-3-ol were reacted with 209.9 mg (0.67 mmol) of 2-({3-[(RS)-(4,4,5,5,5-pentafluoropentyl)sulphinyl]propyl}amino)ethanol according to Procedure 11. The material was then purified by HPLC method 12. This gave 165.2 mg (53% of theory) of product.

¹H NMR (400 MHz, chloroform-d₁): δ=1.01-1.17 (m, 4H), 1.18-1.30 (m, 4H), 1.39-1.49 (m, 2H), 2.05-2.34 (m, 10H), 2.41 (t, 2H), 2.59-2.66 (m, 4H), 2.75-3.00 (m, 8H), 3.76 (t, 2H), 6.69-6.76 (m, 4H), 6.78 (s-br., 1H), 7.14-7.22 (m, 2H).

### Example 88

### 8-(3-Hydroxyphenyl)-9-{6-[(3-hydroxypropyl){3-[(RS)-(4,4,5,5,5-pentafluoropentyl)sulphinyl]propyl}amino]hexyl}-6,7-dihydro-5H-benzo[7]annulen-3-ol

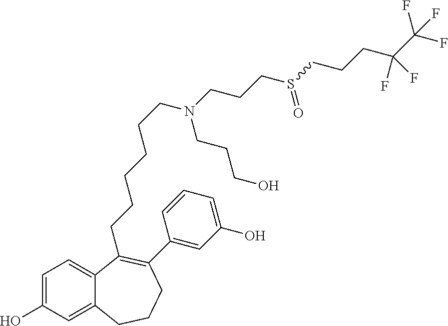 [SMILES: O=S(CCCN(CCCO)CCCCCCC1=C(c2cccc(O)c2)CCCc2cc(O)ccc21)CCCC(F)(F)C(F)(F)F]

200 mg (0.48 mmol) of 9-(6-bromohexyl)-8-(3-hydroxyphenyl)-6,7-dihydro-5H-benzo[7]annulen-3-ol were reacted with 209.9 mg (0.65 mmol) of 3-({3-[(RS)-(4,4,5,5,5-pentafluoropentyl)sulphinyl]propyl}amino)propan-1-ol according to Procedure 11. The material was then purified by HPLC method 12. This gave 169 mg (53% of theory) of product.

¹H NMR (400 MHz, chloroform-d₁): δ=1.01-1.29 (m, 6H), 1.42-1.52 (m, 2H), 1.86 (mc, 2H), 2.04-2.33 (m, 10H), 2.40 (t, 2H), 2.61 (t, 2H), 2.66 (mc, 2H), 2.74-2.90 (m, 4H), 2.92-3.07 (m, 4H), 3.79 (t, 2H), 6.67-6.77 (m, 4H), 6.80 (s-br., 1H), 7.13-7.22 (m, 2H).

### Example 89

### 9-{6-[(2-Hydroxy-2-methylpropyl){3-[(RS)-(4,4,5,5,5-pentafluoropentyl)sulphinyl]propyl}amino]hexyl}-8-(3-hydroxyphenyl)-6,7-dihydro-5H-benzo[7]annulen-3-ol

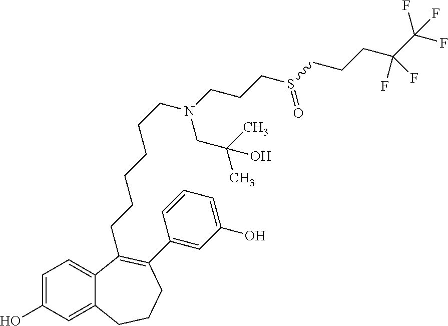 [SMILES: CC(C)(O)CN(CCCCCCC1=C(c2cccc(O)c2)CCCc2cc(O)ccc21)CCCS(=O)CCCC(F)(F)C(F)(F)F]

200 mg (0.48 mmol) of 9-(6-bromohexyl)-8-(3-hydroxyphenyl)-6,7-dihydro-5H-benzo[7]annulen-3-ol were reacted with 228.8 mg (0.67 mmol) of 2-methyl-1-({3-[(RS)-(4,4,5,5,5-pentafluoropentyl)sulphinyl]propyl}amino)propan-2-ol according to Procedure 11.

The material was then purified by HPLC method 12. This gave 54 mg (17% of theory) of product.

¹H NMR (600 MHz, chloroform-d₁): δ=1.01-1.07 (m, 2H), 1.12 (mc, 2H), 1.18-1.24 (m, 8H), 1.35 (mc, 2H), 2.02-2.32 (m, 10H), 2.41 (mc, 2H), 2.46-2.57 (m, 4H), 2.63 (t, 2H), 2.70-2.90 (m, 6H), 6.70-6.76 (m, 4H), 6.77 (t, 1H), 7.16-7.21 (m, 2H).

### Example 90

### 9-[6-(Ethyl{3-[(4,4,5,5,5-pentafluoropentyl)sulphonyl]propyl}amino)hexyl]-8-(3-hydroxyphenyl)-6,7-dihydro-5H-benzo[7]annulen-3-ol

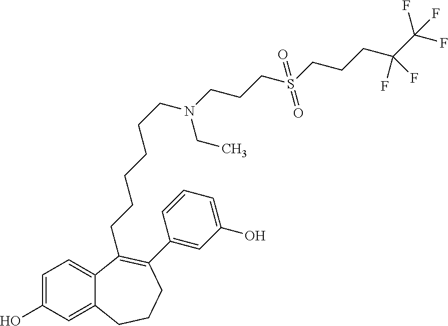 [SMILES: CCN(CCCCCCC1=C(c2cccc(O)c2)CCCc2cc(O)ccc21)CCCS(=O)(=O)CCCC(F)(F)C(F)(F)F]

200 mg (0.48 mmol) of 9-(6-bromohexyl)-8-(3-hydroxyphenyl)-6,7-dihydro-5H-benzo[7]annulen-3-ol were reacted with 209.9 mg (0.67 mmol) of N-ethyl-3-[(4,4,5,5,5-pentafluoropentyl)sulphonyl]propane-1-amine according to Procedure 11. The material was then purified by HPLC method 12. This gave 153 mg (49% of theory) of product.

¹H NMR (400 MHz, chloroform-d₁): δ=0.98-1.30 (m, 9H), 1.57 (mc, 2H), 2.02-2.14 (m, 4H), 2.15-2.41 (m, 8H), 2.57 (t, 2H), 2.77 (mc, 2H), 3.04-3.23 (m, 8H), 6.66-6.76 (m, 4H), 6.82 (t, 1H), 7.14 (d, 1H), 7.20 (dd, 1H).

### Example 91

### 9-{6-[(2-Hydroxyethyl){3-[(4,4,5,5,5-pentafluoropentyl)sulphonyl]propyl}amino]hexyl}-8-(3-hydroxyphenyl)-6,7-dihydro-5H-benzo[7]annulen-3-ol

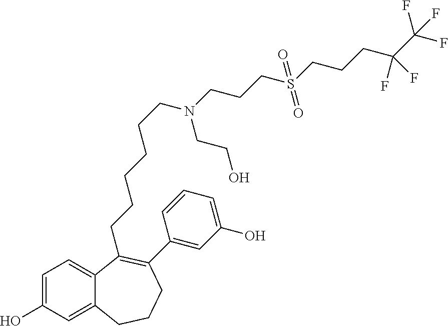 [SMILES: O=S(=O)(CCCN(CCO)CCCCCCC1=C(c2cccc(O)c2)CCCc2cc(O)ccc21)CCCC(F)(F)C(F)(F)F]

140 mg (0.34 mmol) of 9-(6-bromohexyl)-8-(3-hydroxyphenyl)-6,7-dihydro-5H-benzo[7]annulen-3-ol were reacted with 154.5 mg (0.47 mmol) of 2-({3-[(4,4,5,5,5-pentafluoropentyl)sulphonyl]propyl}amino)ethanol according to Procedure 11. The material was then purified by HPLC method 11. This gave 63.9 mg (29% of theory) of product.

¹H NMR (600 MHz, DMSO-d₆): δ=0.99-1.09 (m, 4H), 1.09-1.15 (m, 2H), 1.18-1.25 (m, 2H), 1.73 (mc, 2H), 1.88-1.98 (m, 4H), 1.99-2.05 (m, 2H), 2.27 (t, 2H), 2.33 (t, 2H), 2.36-2.43 (m, 4H), 2.45 (t, 2H), 2.51-2.55 (m, 2H), 3.09 (mc, 2H), 3.21 (t, 2H), 3.38 (t, 2H), 6.61-6.67 (m, 5H), 7.10-7.15 (m, 2H), 9.28 (s-br., 1H), 9.31 (s-br., 1H).

### Example 92

### 8-(3-Hydroxyphenyl)-9-{6-[(3-hydroxypropyl){3-[(4,4,5,5,5-m pentafluoropentyl)sulphonyl]propyl}amino]hexyl}-6,7-dihydro-5H-benzo[7]annulen-3-ol

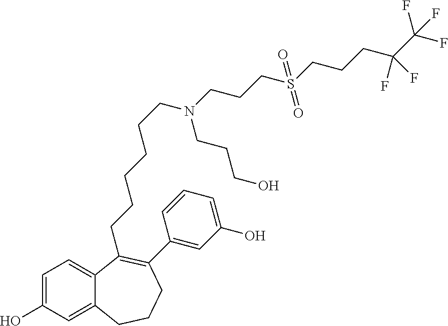 [SMILES: O=S(=O)(CCCN(CCCO)CCCCCCC1=C(c2cccc(O)c2)CCCc2cc(O)ccc21)CCCC(F)(F)C(F)(F)F]

140 mg (0.34 mmol) of 9-(6-bromohexyl)-8-(3-hydroxyphenyl)-6,7-dihydro-5H-benzo[7]annulen-3-ol were reacted with 161.1 mg (0.47 mmol) of 3-({3-[(4,4,5,5,5-pentafluoropentyl)sulphonyl]propyl}amino)propan-1-ol according to Procedure 11. The material was then purified by HPLC method 11. This gave 82.9 mg (36% of theory) of product.

¹H NMR (500 MHz, DMSO-d₆): δ=0.98-1.16 (m, 6H), 1.21 (mc, 2H), 1.48 (mc, 2H), 1.74 (mc, 2H), 1.88-1.98 (m, 4H), 1.99-2.06 (m, 2H), 2.24 (t, 2H), 2.30-2.46 (m, 8H), 2.53 (mc, 2H), 3.08 (mc, 2H), 3.22 (t, 2H), 3.39 (t, 2H), 6.60-6.67 (m, 5H), 7.07-7.16 (m, 2H), 9.30 (s-br., 2H).

### Example 93

### 9-{6-[(2-Hydroxy-2-methylpropyl){3-[(4,4,5,5,5-pentafluoropentyl)sulphonyl]propyl}amino]hexyl}-8-(3-hydroxyphenyl)-6,7-dihydro-5H-benzo[7]annulen-3-ol:

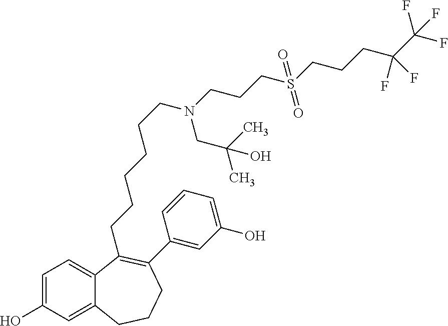 [SMILES: CC(C)(O)CN(CCCCCCC1=C(c2cccc(O)c2)CCCc2cc(O)ccc21)CCCS(=O)(=O)CCCC(F)(F)C(F)(F)F]

100 mg (0.24 mmol) of 9-(6-bromohexyl)-8-(3-hydroxyphenyl)-6,7-dihydro-5H-benzo[7]annulen-3-ol were reacted with 102.7 mg (0.29 mmol) of 2-methyl-1-({3-[(4,4,5,5,5-pentafluoropentyl)sulphonyl]propyl}amino)propan-2-ol according to Procedure 11. The material was then purified by HPLC method 12. This gave 14 mg (8% of theory) of product.

¹H NMR (400 MHz, chloroform-d₁): δ=1.01-1.31 (m, 12H), 1.42 (mc, 2H), 2.04-2.42 (m, 12H), 2.56-2.69 (m, 6H), 2.91 (t, 2H), 3.10 (mc, 4H), 6.70-6.78 (m, 5H), 7.16 (d, 1H), 7.20 (t, 1H).

### Example 94

### 9-[6-(Ethyl{3-[(RS)-(3,3,3-trifluoropropyl)sulphinyl]propyl}amino)hexyl]-8-(3-hydroxyphenyl)-6,7-dihydro-5H-benzo[7]annulen-3-ol

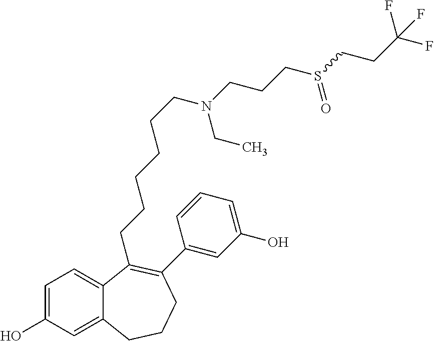 [SMILES: CCN(CCCCCCC1=C(c2cccc(O)c2)CCCc2cc(O)ccc21)CCCS(=O)CCC(F)(F)F]

200 mg (0.48 mmol) of 9-(6-bromohexyl)-8-(3-hydroxyphenyl)-6,7-dihydro-5H-benzo[7]annulen-3-ol were reacted with 155.9 mg (0.67 mmol) of N-ethyl-3-[(3,3,3-trifluoropropyl)sulphinyl]propane-1-amine according to Procedure 11. The material was then purified by HPLC method 8, HPLC method 7 and HPLC method 12. This gave 128 mg (47% of theory) of product.

¹H NMR (600 MHz, DMSO-d₆): δ=0.90 (t, 3H), 1.00-1.16 (m, 6H), 1.21 (mc, 2H), 1.70 (mc, 2H), 1.93-1.98 (m, 2H), 1.99-2.05 (m, 2H), 2.24 (t, 2H), 2.34 (t, 2H), 2.37-2.44 (m, 4H), 2.52-2.55 (m, 2H), 2.58-2.73 (m, 3H), 2.75-2.84 (m, 2H), 3.01 (ddd, 1H), 6.61-6.67 (m, 5H), 7.10-7.16 (m, 2H).

### Example 95

### 9-{6-[(2-Hydroxyethyl){3-[(RS)-(3,3,3-trifluoropropyl)sulphinyl]propyl}amino]hexyl}-8-(3-hydroxyphenyl)-6,7-dihydro-5H-benzo[7]annulen-3-ol

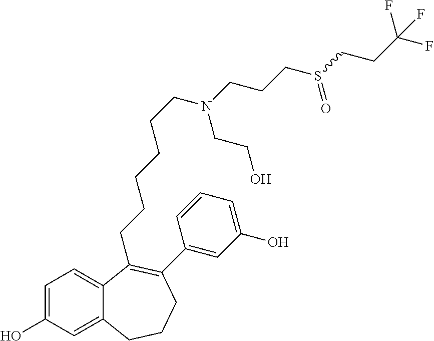 [SMILES: O=S(CCCN(CCO)CCCCCCC1=C(c2cccc(O)c2)CCCc2cc(O)ccc21)CCC(F)(F)F]

200 mg (0.48 mmol) of 9-(6-bromohexyl)-8-(3-hydroxyphenyl)-6,7-dihydro-5H-benzo[7]annulen-3-ol were reacted with 166.7 mg (0.67 mmol) of 2-({3-[(RS)-(3,3,3-trifluoropropyl)sulphinyl]propyl}amino)ethanol according to Procedure 11. The material was then purified by HPLC method 12. This gave 136 mg (49% of theory) of product.

¹H NMR (600 MHz, DMSO-d₆): δ=0.99-1.16 (m, 6H), 1.17-1.25 (m, 2H), 1.64-1.75 (m, 2H), 1.93-1.98 (m, 2H), 1.99-2.06 (m, 2H), 2.27 (t, 2H), 2.33 (t, 2H), 2.41 (t, 2H), 2.46 (t, 2H), 2.53 (t, 2H), 2.59-2.73 (m, 3H), 2.76-2.84 (m, 2H), 3.01 (ddd, 1H), 3.38 (t, 2H), 4.27 (s-br., 1H), 6.60-6.67 (m, 5H), 7.10-7.16 (m, 2H), 9.28 (s-br., 1H), 9.32 (s-br., 1H).

### Example 96

### 8-(3-Hydroxyphenyl)-9-{6-[(3-hydroxypropyl){3-[(RS)-(3,3,3-trifluoropropyl)sulphinyl]propyl}amino]hexyl}-6,7-dihydro-5H-benzo[7]annulen-3-ol

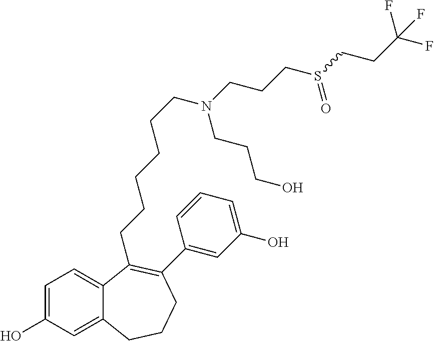 [SMILES: O=S(CCCN(CCCO)CCCCCCC1=C(c2cccc(O)c2)CCCc2cc(O)ccc21)CCC(F)(F)F]

200 mg (0.48 mmol) of 9-(6-bromohexyl)-8-(3-hydroxyphenyl)-6,7-dihydro-5H-benzo[7]annulen-3-ol were reacted with 176.1 mg (0.67 mmol) of 3-({3-[(RS)-(3,3,3-trifluoropropyl)sulphinyl]propyl}amino)propan-1-ol according to Procedure 11. The material was then purified by HPLC method 12. This gave 130 mg (45% of theory) of product.

¹H NMR (300 MHz, DMSO-d₆): δ=0.91-1.24 (m, 8H), 1.44 (mc, 2H), 1.67 (mc, 2H), 1.86-2.04 (m, 4H), 2.16-2.42 (m, 8H), 2.54-2.84 (m, 5H), 2.98 (ddd, 1H), 3.35 (t, 2H), 6.56-6.64 (m, 5H), 7.05-7.14 (m, 2H).

### Example 97

### 9-{6-[(2-Hydroxy-2-methylpropyl){3-[(RS)-(3,3,3-trifluoropropyl)sulphinyl]propyl}amino]hexyl}-8-(3-hydroxyphenyl)-6,7-dihydro-5H-benzo[7]annulen-3-ol

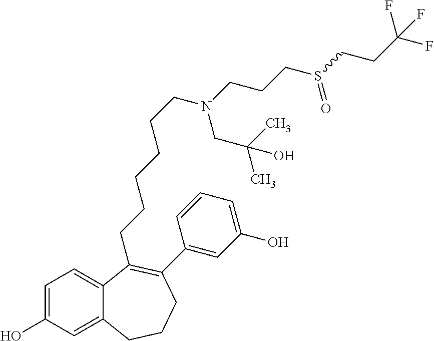 [SMILES: CC(C)(O)CN(CCCCCCC1=C(c2cccc(O)c2)CCCc2cc(O)ccc21)CCCS(=O)CCC(F)(F)F]

200 mg (0.48 mmol) of 9-(6-bromohexyl)-8-(3-hydroxyphenyl)-6,7-dihydro-5H-benzo[7]annulen-3-ol were reacted with 185.6 mg (0.67 mmol) of 2-methyl-1-({3-[(RS)-(3,3,3-trifluoropropyl)sulphinyl]propyl}amino)propan-2-ol according to Procedure 11.

The material was then purified by HPLC method 12. This gave 43 mg (15% of theory) of product.

¹H NMR (600 MHz, chloroform-d₁): δ=1.01-1.07 (m, 2H), 1.09-1.15 (m, 2H), 1.17-1.25 (m, 8H), 1.30-1.37 (m, 2H), 2.00-2.16 (m, 6H), 2.40 (t, 2H), 2.46-2.54 (m, 4H), 2.59-2.70 (m, 4H), 2.72-2.81 (m, 3H), 2.83-2.89 (m, 1H), 2.91-2.96 (m, 2H), 6.70-6.78 (m, 4H), 7.17 (d, 1H), 7.19 (t, 1H).

### Example 98

### 9-{6-[(2-Hydroxyethyl){3-[(3,3,3-trifluoropropyl)sulphonyl]propyl}amino]hexyl}-8-(3-hydroxyphenyl)-6,7-dihydro-5H-benzo[7]annulen-3-ol

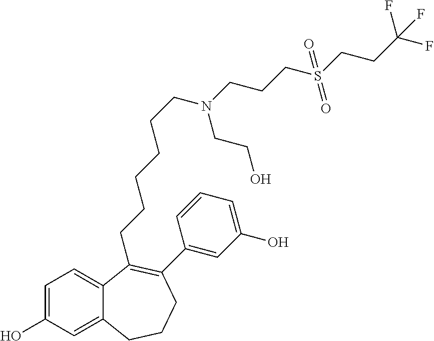 [SMILES: O=S(=O)(CCCN(CCO)CCCCCCC1=C(c2cccc(O)c2)CCCc2cc(O)ccc21)CCC(F)(F)F]

200 mg (0.48 mmol) of 9-(6-bromohexyl)-8-(3-hydroxyphenyl)-6,7-dihydro-5H-benzo[7]annulen-3-ol were reacted with 177.5 mg (0.67 mmol) of 2-({3-[(3,3,3-trifluoropropyl)sulphonyl]propyl}amino)ethanol according to Procedure 11. The material was then purified by HPLC method 12. This gave 136 mg (49% of theory) of product.

¹H NMR (600 MHz, DMSO-d₆): δ=0.98-1.16 (m, 6H), 1.18-1.25 (m, 2H), 1.64-1.75 (m, 2H), 1.93-1.98 (m, 2H), 1.99-2.05 (m, 2H), 2.27 (t, 2H), 2.33 (t, 2H), 2.41 (t, 2H), 2.45 (t, 2H), 2.53 (t, 2H), 2.58-2.73 (m, 3H), 2.76-2.84 (m, 2H), 3.00 (ddd, 1H), 3.38 (t, 2H), 4.27 (s-br., 1H), 6.60-6.67 (m, 5H), 7.09-7.16 (m, 2H), 9.28 (s-br., 1H), 9.32 (s-br., 1H).

### Example 99

### 8-(3-Hydroxyphenyl)-9-{6-[(3-hydroxypropyl){3-[(3,3,3-trifluoropropyl)sulphonyl]propyl}amino]hexyl}-6,7-dihydro-5H-benzo[7]annulen-3-ol

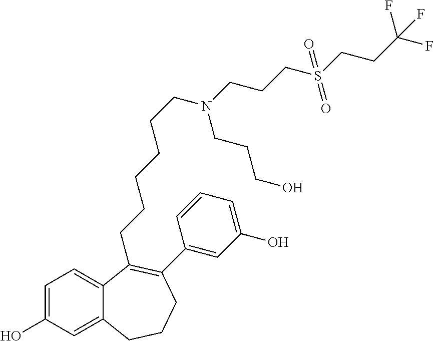 [SMILES: O=S(=O)(CCCN(CCCO)CCCCCCC1=C(c2cccc(O)c2)CCCc2cc(O)ccc21)CCC(F)(F)F]

200 mg (0.48 mmol) of 9-(6-bromohexyl)-8-(3-hydroxyphenyl)-6,7-dihydro-5H-benzo[7]annulen-3-ol were reacted with 186.9 mg (0.67 mmol) of 3-({3-[(3,3,3-trifluoropropyl)sulphonyl]propyl}amino)propan-1-ol according to Procedure 11. The material was then purified by HPLC method 12. This gave 134 mg (45% of theory) of product.

¹H NMR (300 MHz, DMSO-d₆): δ=0.92-1.25 (m, 8H), 1.38-1.51 (m, 2H), 1.66-1.79 (m, 2H), 1.85-2.05 (m, 4H), 2.16-2.43 (m, 8H), 2.59-2.78 (m, 2H), 3.13 (mc, 2H), 3.32-3.40 (m, 4H), 6.56-6.66 (m, 5H), 7.04-7.15 (m, 2H).

### Example 100

### 9-{6-[(2-Hydroxy-2-methylpropyl){3-[(3,3,3-trifluoropropyl)sulphonyl]propyl}amino]hexyl}-8-(3-hydroxyphenyl)-6,7-dihydro-5H-benzo[7]annulen-3-ol

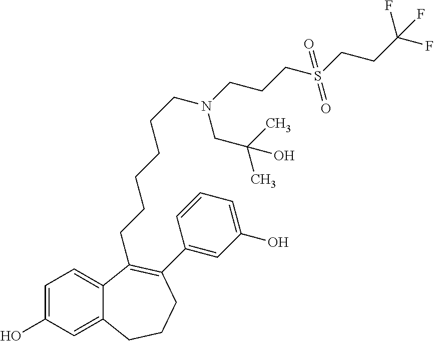 [SMILES: CC(C)(O)CN(CCCCCCC1=C(c2cccc(O)c2)CCCc2cc(O)ccc21)CCCS(=O)(=O)CCC(F)(F)F]

200 mg (0.48 mmol) of 9-(6-bromohexyl)-8-(3-hydroxyphenyl)-6,7-dihydro-5H-benzo[7]annulen-3-ol were reacted with 196.4 mg (0.67 mmol) of 2-methyl-1-({3-[(3,3,3-trifluoropropyl)sulphonyl]propyl}amino)propan-2-ol according to Procedure 11. The material was then purified by HPLC method 12. This gave 20.7 mg (7% of theory) of product.

¹H NMR (600 MHz, chloroform-d₁): δ=1.05-1.32 (m, 14H), 1.95-2.02 (m, 2H), 2.06-2.15 (m, 4H), 2.36-2.44 (m, 6H), 2.60-2.74 (m, 6H), 3.06-3.10 (m, 2H), 3.20 (mc, 2H), 6.70-6.77 (m, 5H), 7.18 (d, 1H), 7.20-7.23 (m, 1H).

### Example 101

### 2-Fluoro-8-(4-fluoro-3-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol

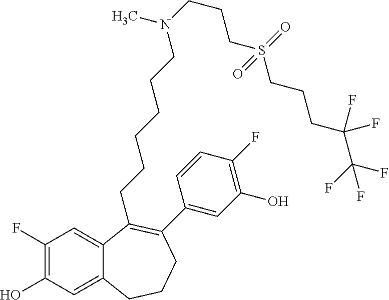 [SMILES: CN(CCCCCCC1=C(c2ccc(F)c(O)c2)CCCc2cc(O)c(F)cc21)CCCS(=O)(=O)CCCC(F)(F)C(F)(F)F]

130 mg (0.29 mmol) of 9-(6-bromohexyl)-2-fluoro-8-(4-fluoro-3-hydroxyphenyl)-6,7-dihydro-5H-benzo[7]annulen-3-ol were reacted with 119.9 mg (0.40 mmol) of N-methyl-3-[(4,4,5,5,5-pentafluoropentyl)sulphonyl]propane-1-amine according to Procedure 11. The material was purified by HPLC method 12. 20 mg (10% of theory) of product were isolated.

¹H NMR (400 MHz, chloroform-d₁): δ=1.00-1.26 (m, 6H), 1.45 (m, 2H), 2.01-2.14 (m, 4H), 2.15-2.37 (m, 8H), 2.40-2.58 (m, 7H), 2.77 (t, 2H), 3.10-3.21 (m, 4H), 6.63 (ddd, 1H), 6.81 (d, 1H), 6.89 (dd, 1H), 6.95-7.06 (m, 2H).

### Example 102

### 4-Chloro-9-[6-(methyl{3-[(4,4,5,5,5-pentafluoropentyl)sulphonyl]propyl}amino)hexyl]-8-phenyl-6,7-dihydro-5H-benzo[7]annulen-3-ol

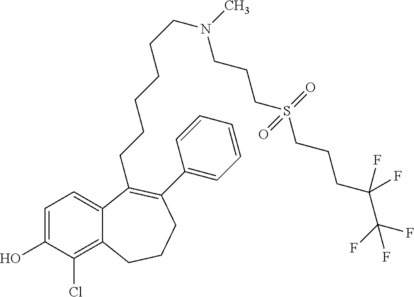 [SMILES: CN(CCCCCCC1=C(c2ccccc2)CCCc2c1ccc(O)c2Cl)CCCS(=O)(=O)CCCC(F)(F)C(F)(F)F]

340 mg (0.78 mmol) of 9-(6-bromohexyl)-4-chloro-8-phenyl-6,7-dihydro-5H-benzo[7]annulen-3-ol were reacted with 326.2 mg (1.10 mmol) of N-methyl-3-[(4,4,5,5,5-pentafluoropentyl)sulphonyl]propane-1-amine according to Procedure 11. The material was purified by HPLC method 12. This gave 271 mg (53% of theory) of product.

¹H NMR (300 MHz, DMSO-d₆): δ=0.92-1.25 (m, 8H), 1.66-1.79 (m, 2H), 1.81-2.06 (m, 9H), 2.11 (t, 2H), 2.25-2.40 (m, 6H), 2.79 (mc, 2H), 3.04 (mc, 2H), 3.19 (t, 2H), 6.85 (d, 1H), 7.10 (d, 1H), 7.16-7.27 (m, 3H), 7.29-7.38 (m, 2H).

### Example 103

### 4-Chloro-9-[6-(methyl{4-[(4,4,5,5,5-pentafluoropentyl)sulphonyl]butyl}amino)hexyl]-8-phenyl-6,7-dihydro-5H-benzo[7]annulen-3-ol

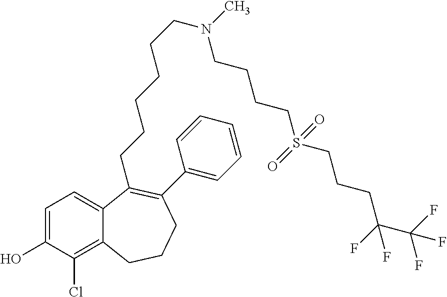 [SMILES: CN(CCCCCCC1=C(c2ccccc2)CCCc2c1ccc(O)c2Cl)CCCCS(=O)(=O)CCCC(F)(F)C(F)(F)F]

200 mg (0.46 mmol) of 9-(6-bromohexyl)-4-chloro-8-phenyl-6,7-dihydro-5H-benzo[7]annulen-3-ol were reacted with 200.9 mg (0.65 mmol) of N-methyl-4-[(4,4,5,5,5-pentafluoropentyl)sulphonyl]butane-1-amine according to Procedure 11. The material was purified by HPLC method 11. This gave 168.5 mg (55% of theory) of product.

¹H NMR (400 MHz, chloroform-d₁): δ=1.07-1.26 (m, 6H), 1.35-1.46 (m, 2H), 1.75-1.85 (m, 2H), 1.87-1.96 (m, 2H), 2.05-2.35 (m, 8H), 2.39 (t, 2H), 2.43 (s, 3H), 2.52 (mc, 2H), 2.68 (t, 2H), 2.89 (t, 2H), 3.02-3.10 (m, 4H), 6.95 (d, 1H), 7.13 (d, 1H), 7.21-7.29 (m, 3H), 7.33-7.38 (m, 2H).

### Example 104

### 4-Chloro-9-[6-(methyl{3-[(RS)-(4,4,5,5,5-pentafluoropentyl)sulphinyl]propyl}amino)hexyl]-8-phenyl-6,7-dihydro-5H-benzo[7]annulen-3-ol

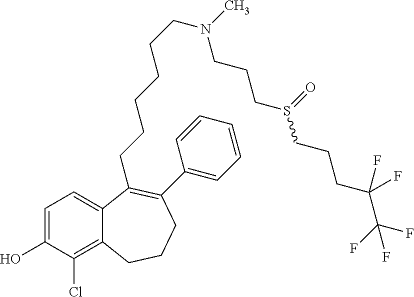 [SMILES: CN(CCCCCCC1=C(c2ccccc2)CCCc2c1ccc(O)c2Cl)CCCS(=O)CCCC(F)(F)C(F)(F)F]

200 mg (0.46 mmol) of 9-(6-bromohexyl)-4-chloro-8-phenyl-6,7-dihydro-5H-benzo[7]annulen-3-ol were reacted with 181.6 mg (0.65 mmol) of N-methyl-3-[(RS)-(4,4,5,5,5-pentafluoropentyl)sulphinyl]propane-1-amine according to Procedure 11. The material was purified by HPLC method 11. 242.7 mg (83% of theory) of product were isolated.

¹H NMR (400 MHz, chloroform-d₁): δ=1.05-1.29 (m, 6H), 1.31-1.43 (m, 2H), 2.02-2.33 (m, 10H), 2.34-2.42 (m, 5H), 2.46 (mc, 2H), 2.65-2.97 (m, 8H), 6.94 (d, 1H), 7.12 (d, 1H), 7.20-7.29 (m, 3H), 7.32-7.39 (m, 2H).

### Example 105

### 8-(4-Fluoro-3-hydroxyphenyl)-9-{6-[(2-methoxyethyl){3-[(4,4,5,5,5-pentafluoropentyl)sulphonyl]propyl}amino]hexyl}-6,7-dihydro-5H-benzo[7]annulen-3-ol

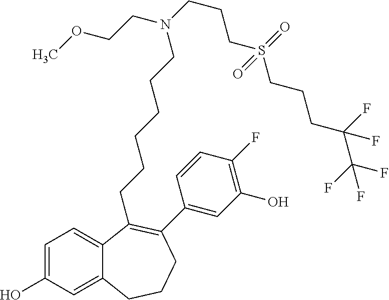 [SMILES: COCCN(CCCCCCC1=C(c2ccc(F)c(O)c2)CCCc2cc(O)ccc21)CCCS(=O)(=O)CCCC(F)(F)C(F)(F)F]

100 mg (0.231 mmol) of 9-(6-bromohexyl)-8-(4-fluoro-3-hydroxyphenyl)-6,7-dihydro-5H-benzo[7]annulen-3-ol were reacted with 94.5 mg (0.277 mmol) of N-(2-methoxyethyl)-3-[(4,4,5,5,5-pentafluoropentyl)sulphonyl]propane-1-amine according to Procedure 11. The material was purified by HPLC method 11. 13.4 mg (8% of theory) of product were isolated.

¹H NMR (400 MHz, chloroform-d₁): δ=0.99-1.23 (m, 6H), 1.38 (mc, 2H), 2.00-2.38 (m, 12H), 2.48-2.61 (m, 4H), 2.78-2.89 (m, 4H), 3.12 (mc, 4H), 3.34 (s, 3H), 3.54 (t, 2H), 6.65 (ddd, 1H), 6.68-6.76 (m, 2H), 6.90 (dd, 1H), 7.02 (dd, 1H), 7.14 (d, 1H).

### Example 106

### 8-(3-Hydroxyphenyl)-9-{6-[(2-methoxyethyl){3-[(4,4,5,5,5-pentafluoropentyl)sulphonyl]propyl}amino]hexyl}-6,7-dihydro-5H-benzo[7]annulen-3-ol

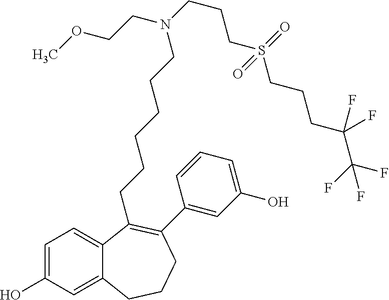 [SMILES: COCCN(CCCCCCC1=C(c2cccc(O)c2)CCCc2cc(O)ccc21)CCCS(=O)(=O)CCCC(F)(F)C(F)(F)F]

100 mg (0.241 mmol) of 9-(6-bromohexyl)-8-(3-hydroxyphenyl)-6,7-dihydro-5H-benzo[7]annulen-3-ol were reacted with 98.6 mg (0.289 mmol) of N-(2-methoxyethyl)-3-[(4,4,5,5,5-pentafluoropentyl)sulphonyl]propane-1-amine according to Procedure 11. The material was purified by HPLC method 12. 28 mg (17% of theory) of product were isolated.

¹H NMR (400 MHz, chloroform-d₁): δ=0.98-1.23 (m, 6H), 1.41 (mc, 2H), 2.01-2.40 (m, 12H), 2.51-2.65 (m, 4H), 2.92 (mc, 4H), 3.07-3.16 (m, 4H), 3.34 (s, 3H), 3.57 (t, 2H), 6.68-6.75 (m, 4H), 6.78 (s, 1H), 7.14 (d, 1H), 7.20 (t, 1H).

### Example 107

### 8-(3-Hydroxyphenyl)-9-{6-[(3-methoxypropyl){3-[(4,4,5,5,5-pentafluoropentyl)sulphonyl]propyl}amino]hexyl}-6,7-dihydro-5H-benzo[7]annulen-3-ol

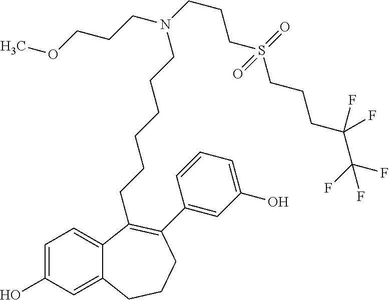 [SMILES: COCCCN(CCCCCCC1=C(c2cccc(O)c2)CCCc2cc(O)ccc21)CCCS(=O)(=O)CCCC(F)(F)C(F)(F)F]

100 mg (0.241 mmol) of 9-(6-bromohexyl)-8-(3-hydroxyphenyl)-6,7-dihydro-5H-benzo[7]annulen-3-ol were reacted with 102.7 mg (0.289 mmol) of 3-methoxy-N-{3-[(4,4,5,5,5-pentafluoropentyl)sulphonyl]propyl}propane-1-amine according to Procedure 11. The material was purified by HPLC method 12. 51 mg (30% of theory) of product were isolated.

¹H NMR (400 MHz, chloroform-d₁): δ=0.96-1.23 (m, 6H), 1.48 (mc, 2H), 1.86 (mc, 2H), 2.02-2.42 (m, 12H), 2.56 (mc, 2H), 2.66 (mc, 2H), 2.94 (mc, 2H), 3.01 (t, 2H), 3.10-3.18 (m, 4H), 3.34 (s, 3H), 3.44 (t, 2H), 6.68-6.75 (m, 4H), 6.80 (s, 1H), 7.14 (d, 1H), 7.20 (t, 1H).

### Example 108

### 8-(3-Hydroxyphenyl)-9-{6-[(3-methoxypropyl){3-[(RS)-(4,4,5,5,5-pentafluoropentyl)sulphinyl]propyl}amino]hexyl}-6,7-dihydro-5H-benzo[7]annulen-3-ol

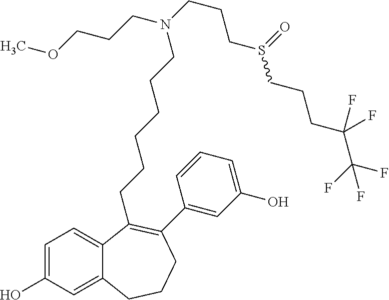 [SMILES: COCCCN(CCCCCCC1=C(c2cccc(O)c2)CCCc2cc(O)ccc21)CCCS(=O)CCCC(F)(F)C(F)(F)F]

150 mg (0.36 mmol) of 9-(6-bromohexyl)-8-(3-hydroxyphenyl)-6,7-dihydro-5H-benzo[7]annulen-3-ol were reacted with 147.1 mg (0.43 mmol) of 3-methoxy-N-{3-[(RS)-(4,4,5,5,5-pentafluoropentyl)sulphinyl]propyl}propane-1-amine according to Procedure 11. The material was purified by HPLC method 11. 50 mg (20% of theory) of product were isolated.

¹H NMR (300 MHz, chloroform-d₁): δ=0.96-1.35 (m, 8H), 1.71 (mc, 2H), 1.88-2.44 (m, 14H), 2.51-2.67 (m, 6H), 2.69-2.90 (m, 4H), 3.32 (s, 3H), 3.40 (t, 2H), 6.67-6.79 (m, 5H), 7.11-7.22 (m, 2H).

### Example 109

### 4-Chloro-9-[6-(methyl{4-[(3,3,3-trifluoropropyl)sulphonyl]butyl}amino)hexyl]-8-phenyl-6,7-dihydro-5H-benzo[7]annulen-3-ol

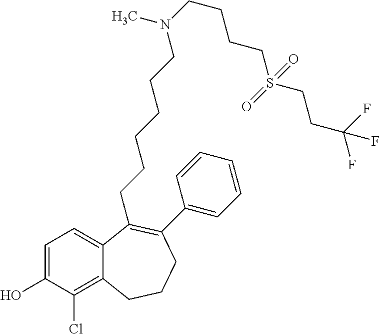 [SMILES: CN(CCCCCCC1=C(c2ccccc2)CCCc2c1ccc(O)c2Cl)CCCCS(=O)(=O)CCC(F)(F)F]

150 mg (0.35 mmol) of 9-(6-bromohexyl)-4-chloro-8-phenyl-6,7-dihydro-5H-benzo[7]annulen-3-ol were reacted with 102.6 mg (0.41 mmol) of N-methyl-4-[(3,3,3-trifluoropropyl)sulphonyl]butane-1-amine according to Procedure 11. The material was purified by HPLC method 12. 75 mg (36% of theory) of product were isolated.

¹H NMR (400 MHz, chloroform-d₁): δ=1.05-1.26 (m, 6H), 1.43 (mc, 2H), 1.81 (quin, 2H), 1.93 (quin, 2H), 2.03-2.17 (m, 4H), 2.38 (t, 2H), 2.43 (s, 3H), 2.52 (mc, 2H), 2.60-2.74 (m, 4H), 2.89 (mc, 2H), 3.11 (mc, 2H), 3.20 (mc, 2H), 6.94 (d, 1H), 7.11 (d, 1H), 7.20-7.29 (m, 3H), 7.33-7.39 (m, 2H).

### Example 110

### 8-(4-Fluoro-3-hydroxyphenyl)-9-[6-(methyl{4-[(3,3,3-trifluoropropyl)sulphonyl]butyl}amino)hexyl]-6,7-dihydro-5H-benzo[7]annulen-3-ol

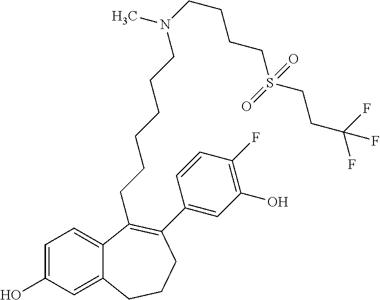 [SMILES: CN(CCCCCCC1=C(c2ccc(F)c(O)c2)CCCc2cc(O)ccc21)CCCCS(=O)(=O)CCC(F)(F)F]

130 mg (0.30 mmol) of 9-(6-bromohexyl)-8-(4-fluoro-3-hydroxyphenyl)-6,7-dihydro-5H-benzo[7]annulen-3-ol were reacted with 89.0 mg (0.36 mmol) of N-methyl-4-[(3,3,3-trifluoropropyl)sulphonyl]butane-1-amine according to Procedure 11. The material was purified by HPLC method 12. 82 mg (46% of theory) of product were isolated.

¹H NMR (400 MHz, methanol-d₄): δ=1.08-1.27 (m, 6H), 1.48 (mc, 2H), 1.74-1.91 (m, 4H), 1.98-2.13 (m, 4H), 2.39 (t, 2H), 2.55-2.81 (m, 9H), 2.91 (mc, 2H), 3.23 (t, 2H), 3.33-3.39 (m, 2H), 6.60-6.69 (m, 3H), 6.78 (dd, 1H), 6.99 (dd, 1H), 7.11 (d, 1H).

### Example 111

### 9-[5-(Methyl{4-[(3,3,3-trifluoropropyl)sulphonyl]butyl}amino)pentyl]-8-phenyl-6,7-dihydro-5H-benzo[7]annulen-3-ol

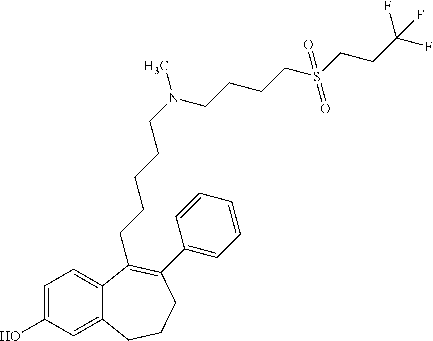 [SMILES: CN(CCCCCC1=C(c2ccccc2)CCCc2cc(O)ccc21)CCCCS(=O)(=O)CCC(F)(F)F]

130 mg (0.34 mmol) of 9-(5-bromopentyl)-8-phenyl-6,7-dihydro-5H-benzo[7]annulen-3-ol were reacted with 100.1 mg (0.40 mmol) of N-methyl-4-[(3,3,3-trifluoropropyl)sulphonyl]butane-1-amine according to Procedure 11. The material was purified by HPLC method 12. 115 mg (62% of theory) of product were isolated.

¹H NMR (400 MHz, chloroform-d₁): δ=1.10 (quin, 2H), 1.22 (quin, 2H), 1.39 (mc, 2H), 1.75 (quin, 2H), 1.88 (quin, 2H), 2.04-2.16 (m, 4H), 2.33-2.49 (m, 7H), 2.54-2.73 (m, 6H), 3.07 (mc, 2H), 3.15-3.22 (m, 2H), 6.69-6.76 (m, 2H), 7.13 (d, 1H), 7.20-7.28 (m, 3H), 7.35 (t, 2H).

### Example 112

### 8-(3-Hydroxyphenyl)-9-[5-(methyl{4-[(3,3,3-trifluoropropyl)sulphonyl]butyl}amino)pentyl]-6,7-dihydro-5H-benzo[7]annulen-3-ol

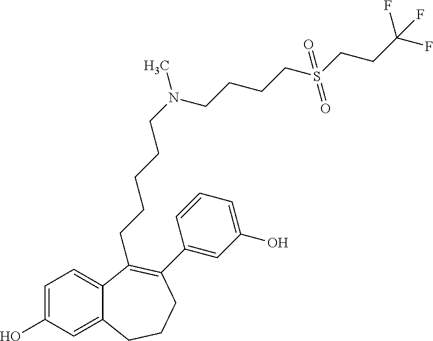 [SMILES: CN(CCCCCC1=C(c2cccc(O)c2)CCCc2cc(O)ccc21)CCCCS(=O)(=O)CCC(F)(F)F]

130 mg (0.32 mmol) of 9-(5-bromopentyl)-8-(3-hydroxyphenyl)-6,7-dihydro-5H-benzo[7]annulen-3-ol were reacted with 96.1 mg (0.39 mmol) of N-methyl-4-[(3,3,3-trifluoropropyl)sulphonyl]butane-1-amine according to Procedure 11. The material was purified by HPLC method 12. 100 mg (54% of theory) of product were isolated.

¹H NMR (400 MHz, methanol-d₄): δ=1.14-1.32 (m, 4H), 1.46 (mc, 2H), 1.74-1.90 (m, 4H), 2.00-2.15 (m, 4H), 2.43 (t, 2H), 2.61 (t, 2H), 2.63-2.84 (m, 7H), 2.92-2.98 (m, 2H), 3.22 (t, 2H), 3.33-3.39 (m, 2H), 6.63-6.71 (m, 5H), 7.11-7.18 (m, 2H).

### Example 113

### 8-(3-Hydroxyphenyl)-9-[6-(methyl{4-[(3,3,3-trifluoropropyl)sulphonyl]butyl}amino)hexyl]-6,7-dihydro-5H-benzo[7]annulen-3-ol

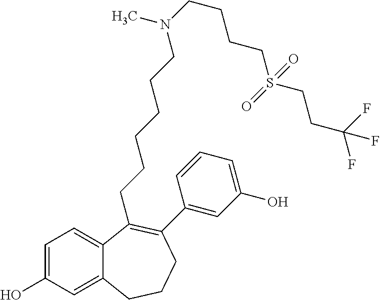 [SMILES: CN(CCCCCCC1=C(c2cccc(O)c2)CCCc2cc(O)ccc21)CCCCS(=O)(=O)CCC(F)(F)F]

130 mg (0.31 mmol) of 9-(6-bromohexyl)-8-(3-hydroxyphenyl)-6,7-dihydro-5H-benzo[7]annulen-3-ol were reacted with 92.9 mg (0.38 mmol) of N-methyl-4-[(3,3,3-trifluoropropyl)sulphonyl]butane-1-amine according to Procedure 11. The material was purified by HPLC method 12. 120 mg (66% of theory) of product were isolated.

¹H NMR (400 MHz, methanol-d₄): δ=1.09-1.28 (m, 6H), 1.49 (mc, 2H), 1.75-1.92 (m, 4H), 2.00-2.14 (m, 4H), 2.41 (t, 2H), 2.61 (t, 2H), 2.64-2.77 (m, 5H), 2.82 (mc, 2H), 2.96 (mc, 2H), 3.23 (t, 2H), 3.36 (mc, 2H), 6.63-6.69 (m, 5H), 7.10-7.17 (m, 2H).

### Example 114

### 4-Fluoro-8-(4-fluoro-3-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol

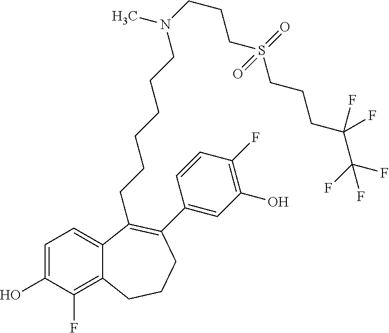 [SMILES: CN(CCCCCCC1=C(c2ccc(F)c(O)c2)CCCc2c1ccc(O)c2F)CCCS(=O)(=O)CCCC(F)(F)C(F)(F)F]

130 mg (0.29 mmol) of 9-(6-bromohexyl)-4-fluoro-8-(4-fluoro-3-hydroxyphenyl)-6,7-dihydro-5H-benzo[7]annulen-3-ol were reacted with 102.8 mg (0.35 mmol) of N-methyl-3-[(4,4,5,5,5-pentafluoropentyl)sulphonyl]propane-1-amine according to Procedure 11. The material was purified by HPLC method 12. 24.4 mg (13% of theory) of product were isolated.

¹H NMR (300 MHz, chloroform-d₁): δ=1.00-1.28 (m, 6H), 1.47 (m, 2H), 2.01-2.38 (m, 12H), 2.39-2.49 (m, 5H), 2.70 (m, 2H), 2.77 (t, 2H), 3.10-3.20 (m, 4H), 6.65 (ddd, 1H), 6.86 (t, 1H), 6.90-6.98 (m, 2H), 7.03 (dd, 1H).

### Example 115

### 4-Fluoro-8-(4-fluoro-3-hydroxyphenyl)-9-{6-[(2-hydroxyethyl){3-[(4,4,5,5,5-pentafluoropentyl)sulphonyl]propyl}amino]hexyl}-6,7-dihydro-5H-benzo[7]annulen-3-ol

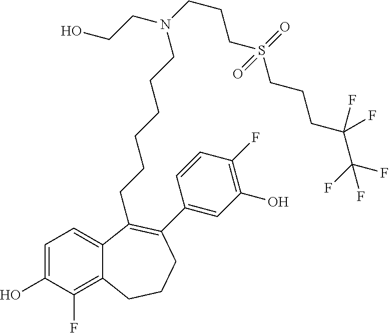 [SMILES: O=S(=O)(CCCN(CCO)CCCCCCC1=C(c2ccc(F)c(O)c2)CCCc2c1ccc(O)c2F)CCCC(F)(F)C(F)(F)F]

130 mg (0.29 mmol) of 9-(6-bromohexyl)-4-fluoro-8-(4-fluoro-3-hydroxyphenyl)-6,7-dihydro-5H-benzo[7]annulen-3-ol were reacted with 113.1 mg (0.35 mmol) of 2-({3-[(4,4,5,5,5-pentafluoropentyl)sulphonyl]propyl}amino)ethanol according to Procedure 11. The material was purified by HPLC method 12. 3.5 mg (2% of theory) of product were isolated.

¹H NMR (300 MHz, chloroform-d₁): δ=1.01-1.27 (m, 6H), 1.35 (m, 2H), 1.98-2.39 (m, 12H), 2.45 (m, 2H), 2.64-2.76 (m, 6H), 3.04-3.15 (m, 4H), 3.65 (t, 2H), 6.67 (ddd, 1H), 6.84-6.92 (m, 2H), 6.97 (d, 1H), 7.04 (dd, 1H).

### Example 116

### 4-Fluoro-8-(4-fluoro-3-hydroxyphenyl)-9-[6-(methyl{3-[(3,3,4,4,4-pentafluorobutyl)sulphonyl]propyl}amino)hexyl]-6,7-dihydro-5H-benzo[7]annulen-3-ol

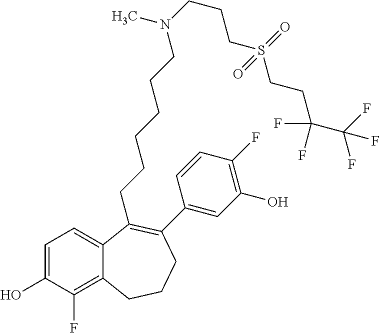 [SMILES: CN(CCCCCCC1=C(c2ccc(F)c(O)c2)CCCc2c1ccc(O)c2F)CCCS(=O)(=O)CCC(F)(F)C(F)(F)F]

130 mg (0.29 mmol) of 9-(6-bromohexyl)-4-fluoro-8-(4-fluoro-3-hydroxyphenyl)-6,7-dihydro-5H-benzo[7]annulen-3-ol were reacted with 97.9 mg (0.35 mmol) of N-methyl-3-[(3,3,4,4,4-pentafluorobutyl)sulphonyl]propane-1-amine according to Procedure 11. The material was purified by HPLC method 12. 25.2 mg (13% of theory) of product were isolated.

¹H NMR (300 MHz, chloroform-d₁): δ=1.00-1.28 (m, 6H), 1.50 (m, 2H), 2.01-2.15 (m, 4H), 2.20-2.36 (m, 4H), 2.45-2.56 (m, 5H), 2.57-2.76 (m, 4H), 2.86 (t, 2H), 3.21-3.34 (m, 4H), 6.64 (ddd, 1H), 6.87 (t, 1H), 6.91-6.99 (m, 2H), 7.03 (dd, 1H).

### Example 117

### 4-Fluoro-8-(4-fluoro-3-hydroxyphenyl)-9-[6-(methyl{4-[(4,4,5,5,5-pentafluoropentyl)sulphonyl]butyl}amino)hexyl]-6,7-dihydro-5H-benzo[7]annulen-3-ol

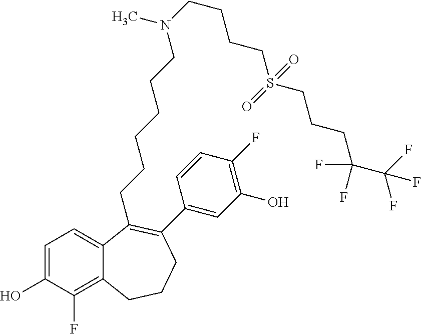 [SMILES: CN(CCCCCCC1=C(c2ccc(F)c(O)c2)CCCc2c1ccc(O)c2F)CCCCS(=O)(=O)CCCC(F)(F)C(F)(F)F]

130 mg (0.29 mmol) of 9-(6-bromohexyl)-4-fluoro-8-(4-fluoro-3-hydroxyphenyl)-6,7-dihydro-5H-benzo[7]annulen-3-ol were reacted with 107.6 mg (0.35 mmol) of N-methyl-4-[(4,4,5,5,5-pentafluoropentyl)sulphonyl]butane-1-amine according to Procedure 11. The material was purified by HPLC method 12. 25.3 mg (13% of theory) of product were isolated.

¹H NMR (300 MHz, chloroform-d₁): δ=0.99-1.28 (m, 6H), 1.52 (m, 2H), 1.79-2.12 (m, 8H), 2.14-2.38 (m, 6H), 2.43-2.56 (m, 5H), 2.63-2.77 (m, 4H), 3.08-3.16 (m, 4H), 6.62 (ddd, 1H), 6.85 (t, 1H), 6.91-6.98 (m, 2H), 7.02 (dd, 1H).

### Example 118

### 4-Fluoro-8-(4-fluoro-3-hydroxyphenyl)-9-[6-(methyl{4-[(4,4,4-trifluorobutyl)sulphonyl]butyl}amino)hexyl]-6,7-dihydro-5H-benzo[7]annulen-3-ol

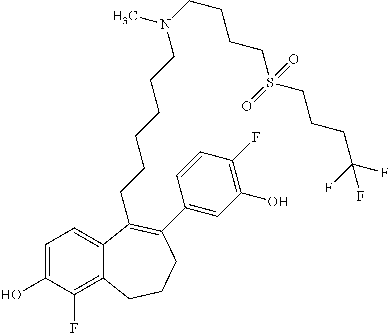 [SMILES: CN(CCCCCCC1=C(c2ccc(F)c(O)c2)CCCc2c1ccc(O)c2F)CCCCS(=O)(=O)CCCC(F)(F)F]

130 mg (0.29 mmol) of 9-(6-bromohexyl)-4-fluoro-8-(4-fluoro-3-hydroxyphenyl)-6,7-dihydro-5H-benzo[7]annulen-3-ol were reacted with 90.3 mg (0.35 mmol) of N-methyl-4-[(4,4,4-trifluorobutyl)sulphonyl]butane-1-amine according to Procedure 11. The material was purified by HPLC method 12. 41.9 mg (23% of theory) of product were isolated.

¹H NMR (400 MHz, chloroform-d₁): δ=1.06 (m, 2H), 1.13-1.26 (m, 4H), 1.55 (m, 2H), 1.85-2.01 (m, 4H), 2.02-2.11 (m, 4H), 2.17 (m, 2H), 2.27-2.40 (m, 4H), 2.54 (s, 3H), 2.59 (m, 2H), 2.67 (m, 2H), 2.80 (m, 2H), 3.12 (t, 4H), 6.60 (ddd, 1H), 6.85 (t, 1H), 6.92-6.97 (m, 2H), 7.02 (dd, 1H).

### Example 119

### 4-Fluoro-8-(4-fluoro-3-hydroxyphenyl)-9-[6-(methyl{4-[(3,3,3-trifluoropropyl)sulphonyl]butyl}amino)hexyl]-6,7-dihydro-5H-benzo[7]annulen-3-ol

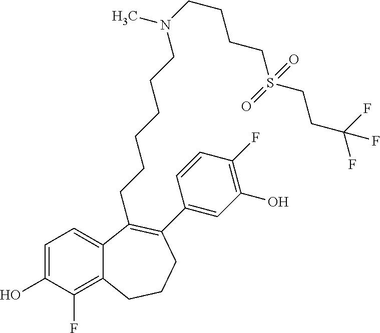 [SMILES: CN(CCCCCCC1=C(c2ccc(F)c(O)c2)CCCc2c1ccc(O)c2F)CCCCS(=O)(=O)CCC(F)(F)F]

130 mg (0.29 mmol) of 9-(6-bromohexyl)-4-fluoro-8-(4-fluoro-3-hydroxyphenyl)-6,7-dihydro-5H-benzo[7]annulen-3-ol were reacted with 85.5 mg (0.35 mmol) of N-methyl-4-[(3,3,3-trifluoropropyl)sulphonyl]butane-1-amine according to Procedure 11. The material was purified by HPLC method 12. 25.7 mg (14% of theory) of product were isolated.

¹H NMR (400 MHz, chloroform-d₁): δ=1.06 (m, 2H), 1.14-1.27 (m, 4H), 1.56 (m, 2H), 1.87-2.14 (m, 8H), 2.32 (t, 2H), 2.53 (s, 3H), 2.58 (m, 2H), 2.64-2.75 (m, 4H), 2.79 (m, 2H), 3.17 (t, 2H), 3.25 (m, 2H), 6.62 (ddd, 1H), 6.86 (t, 1H), 6.94-6.98 (m, 2H), 7.02 (dd, 1H).

### Example 120

### 4-Fluoro-8-(4-fluoro-3-hydroxyphenyl)-9-[6-(methyl{4-[(RS)-(3,3,3-trifluoropropyl)sulphinyl]butyl}amino)hexyl]-6,7-dihydro-5H-benzo[7]annulen-3-ol

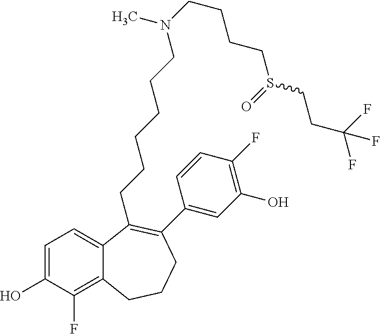 [SMILES: CN(CCCCCCC1=C(c2ccc(F)c(O)c2)CCCc2c1ccc(O)c2F)CCCCS(=O)CCC(F)(F)F]

130 mg (0.29 mmol) of 9-(6-bromohexyl)-4-fluoro-8-(4-fluoro-3-hydroxyphenyl)-6,7-dihydro-5H-benzo[7]annulen-3-ol were reacted with 79.9 mg (0.35 mmol) of N-methyl-4-[(RS)-(3,3,3-trifluoropropyl)sulphinyl]butane-1-amine according to Procedure 11. The material was purified by HPLC method 12. 59.8 mg (35% of theory) of product were isolated.

¹H NMR (400 MHz, chloroform-d₁): δ=1.06 (m, 2H), 1.12-1.26 (m, 4H), 1.51 (m, 2H), 1.80-1.96 (m, 4H), 2.01-2.13 (m, 4H), 2.32 (t, 2H), 2.49 (s, 3H), 2.53 (m, 2H), 2.58-2.99 (m, 10H), 6.60 (ddd, 1H), 6.85 (t, 1H), 6.91-6.97 (m, 2H), 7.01 (dd, 1H).

### Example 121

### 2-Fluoro-8-(4-fluoro-3-hydroxyphenyl)-9-[6-(methyl{4-[(4,4,5,5,5-pentafluoropentyl)sulphonyl]butyl}amino)hexyl]-6,7-dihydro-5H-benzo[7]annulen-3-ol

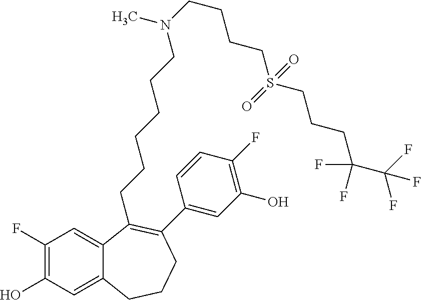 [SMILES: CN(CCCCCCC1=C(c2ccc(F)c(O)c2)CCCc2cc(O)c(F)cc21)CCCCS(=O)(=O)CCCC(F)(F)C(F)(F)F]

120 mg (0.27 mmol) of 9-(6-bromohexyl)-2-fluoro-8-(4-fluoro-3-hydroxyphenyl)-6,7-dihydro-5H-benzo[7]annulen-3-ol were reacted with 115.9 mg (0.37 mmol) of N-methyl-4-[(4,4,5,5,5-pentafluoropentyl)sulphonyl]butane-1-amine according to Procedure 11. The material was purified by HPLC method 12. 44.8 mg (25% of theory) of product were isolated.

¹H NMR (300 MHz, chloroform-d₁): δ=1.00-1.21 (m, 6H), 1.52 (m, 2H), 1.80-2.12 (m, 8H), 2.14-2.38 (m, 6H), 2.42-2.60 (m, 7H), 2.76 (m, 2H), 3.12 (t, 4H), 6.60 (ddd, 1H), 6.80 (d, 1H), 6.90 (dd, 1H), 6.95 (d, 1H), 7.01 (dd, 1H).

### Example 122

### 2-Fluoro-8-(4-fluoro-3-hydroxyphenyl)-9-[6-(methyl{3-[(RS)-(3,3,4,4,4-pentafluorobutyl)sulphinyl]propyl}amino)hexyl]-6,7-dihydro-5H-benzo[7]annulen-3-ol

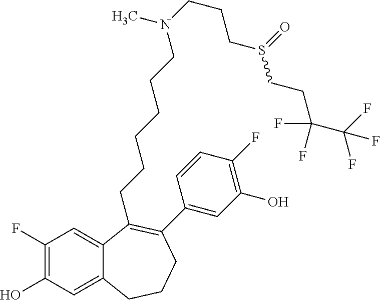 [SMILES: CN(CCCCCCC1=C(c2ccc(F)c(O)c2)CCCc2cc(O)c(F)cc21)CCCS(=O)CCC(F)(F)C(F)(F)F]

120 mg (0.27 mmol) of 9-(6-bromohexyl)-2-fluoro-8-(4-fluoro-3-hydroxyphenyl)-6,7-dihydro-5H-benzo[7]annulen-3-ol were reacted with 85.3 mg (0.32 mmol) of N-methyl-3-[(RS)-(3,3,4,4,4-pentafluorobutyl)sulphinyl]propane-1-amine according to Procedure 11. The material was purified by HPLC method 12. 20.3 mg (12% of theory) of product were isolated.

¹H NMR (300 MHz, chloroform-d₁): δ=0.99-1.26 (m, 6H), 1.41 (m, 2H), 1.98-2.17 (m, 6H), 2.27 (m, 2H), 2.33-2.44 (m, 5H), 2.48-2.75 (m, 6H), 2.82-3.07 (m, 4H), 6.62 (ddd, 1H), 6.81 (d, 1H), 6.89 (dd, 1H), 6.94-7.06 (m, 2H).

### Example 123

### 2-Fluoro-8-(4-fluoro-3-hydroxyphenyl)-9-[6-(methyl{3-[(3,3,4,4,4-pentafluorobutyl)sulphonyl]propyl}amino)hexyl]-6,7-dihydro-5H-benzo[7]annulen-3-ol

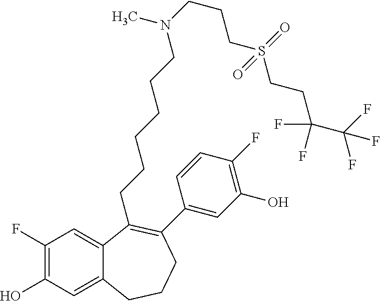 [SMILES: CN(CCCCCCC1=C(c2ccc(F)c(O)c2)CCCc2cc(O)c(F)cc21)CCCS(=O)(=O)CCC(F)(F)C(F)(F)F]

120 mg (0.27 mmol) of 9-(6-bromohexyl)-2-fluoro-8-(4-fluoro-3-hydroxyphenyl)-6,7-dihydro-5H-benzo[7]annulen-3-ol were reacted with 90.4 mg (0.32 mmol) of N-methyl-3-[(3,3,4,4,4-pentafluorobutyl)sulphonyl]propane-1-amine according to Procedure 11. The material was purified by HPLC method 12. 43.5 mg (25% of theory) of product were isolated.

¹H NMR (300 MHz, chloroform-d₁): δ=0.98-1.22 (m, 6H), 1.47 (m, 2H), 2.00-2.14 (m, 4H), 2.18-2.31 (m, 4H), 2.44-2.75 (m, 9H), 2.86 (t, 2H), 3.20-3.33 (m, 4H), 6.62 (ddd, 1H), 6.82 (d, 1H), 6.89 (dd, 1H), 6.93-7.06 (m, 2H).

### Example 124

### 2-Fluoro-8-(4-fluoro-3-hydroxyphenyl)-9-[6-(methyl{4-[(RS)-(4,4,5,5,5-pentafluoropentyl)sulphinyl]butyl}amino)hexyl]-6,7-dihydro-5H-benzo[7]annulen-3-ol

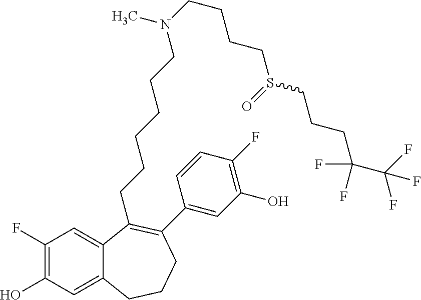 [SMILES: CN(CCCCCCC1=C(c2ccc(F)c(O)c2)CCCc2cc(O)c(F)cc21)CCCCS(=O)CCCC(F)(F)C(F)(F)F]

120 mg (0.27 mmol) of 9-(6-bromohexyl)-2-fluoro-8-(4-fluoro-3-hydroxyphenyl)-6,7-dihydro-5H-benzo[7]annulen-3-ol were reacted with 94.2 mg (0.32 mmol) of N-methyl-4-[(RS)-(4,4,5,5,5-pentafluoropentyl)sulphinyl]butane-1-amine according to Procedure 11. The material was purified by HPLC method 12. 50.3 mg (28% of theory) of product were isolated.

¹H NMR (300 MHz, chloroform-d₁): δ=0.99-1.23 (m, 6H), 1.49 (m, 2H), 1.74-1.96 (m, 4H), 1.98-2.10 (m, 4H), 2.11-2.35 (m, 6H), 2.41-2.58 (m, 7H), 2.61-2.90 (m, 6H), 6.59 (ddd, 1H), 6.80 (d, 1H), 6.89 (dd, 1H), 6.92-7.05 (m, 2H).

### Example 125

### 2-Fluoro-8-(4-fluoro-3-hydroxyphenyl)-9-[6-(methyl{3-[(RS)-(3,3,3-trifluoropropyl)sulphinyl]propyl}amino)hexyl]-6,7-dihydro-5H-benzo[7]annulen-3-ol

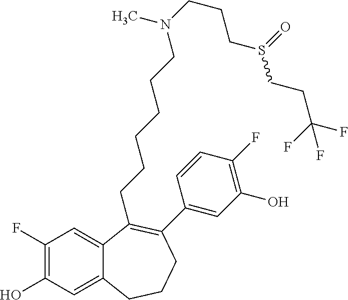 [SMILES: CN(CCCCCCC1=C(c2ccc(F)c(O)c2)CCCc2cc(O)c(F)cc21)CCCS(=O)CCC(F)(F)F]

130 mg (0.29 mmol) of 9-(6-bromohexyl)-2-fluoro-8-(4-fluoro-3-hydroxyphenyl)-6,7-dihydro-5H-benzo[7]annulen-3-ol were reacted with 75.1 mg (0.35 mmol) of N-methyl-3-[(RS)-(3,3,3-trifluoropropyl)sulphinyl]propane-1-amine according to Procedure 11. The material was purified by HPLC method 12. 25.7 mg (15% of theory) of product were isolated.

¹H NMR (300 MHz, chloroform-d₁): δ=1.00-1.29 (m, 6H), 1.40 (m, 2H), 2.01-2.14 (m, 6H), 2.23-2.41 (m, 7H), 2.52-2.72 (m, 6H), 2.82-2.98 (m, 4H), 6.63 (ddd, 1H), 6.84 (d, 1H), 6.91 (dd, 1H), 6.97-7.06 (m, 2H).

### Example 126

### 2-Fluoro-8-(4-fluoro-3-hydroxyphenyl)-9-[6-(methyl{3-[(3,3,3-trifluoropropyl)sulphonyl]propyl}amino)hexyl]-6,7-dihydro-5H-benzo[7]annulen-3-ol

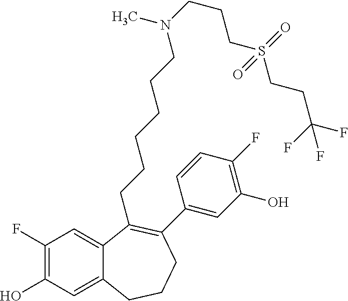 [SMILES: CN(CCCCCCC1=C(c2ccc(F)c(O)c2)CCCc2cc(O)c(F)cc21)CCCS(=O)(=O)CCC(F)(F)F]

130 mg (0.29 mmol) of 9-(6-bromohexyl)-2-fluoro-8-(4-fluoro-3-hydroxyphenyl)-6,7-dihydro-5H-benzo[7]annulen-3-ol were reacted with 80.6 mg (0.35 mmol) of N-methyl-3-[(3,3,3-trifluoropropyl)sulphonyl]propane-1-amine according to Procedure 11. The material was purified by HPLC method 12. 36.2 mg (21% of theory) of product were isolated.

¹H NMR (300 MHz, chloroform-d₁): δ=0.98-1.27 (m, 6H), 1.45 (m, 2H), 2.01-2.14 (m, 4H), 2.15-2.32 (m, 4H), 2.38-2.60 (m, 7H), 2.62-2.84 (m, 4H), 3.17-3.31 (m, 4H), 6.64 (ddd, 1H), 6.83 (d, 1H), 6.90 (dd, 1H), 6.96-7.06 (m, 2H).

### Example 127

### 2-Fluoro-8-(4-fluoro-3-hydroxyphenyl)-9-[6-(methyl{3-[(4,4,4-trifluorobutyl)sulphonyl]propyl}amino)hexyl]-6,7-dihydro-5H-benzo[7]annulen-3-ol

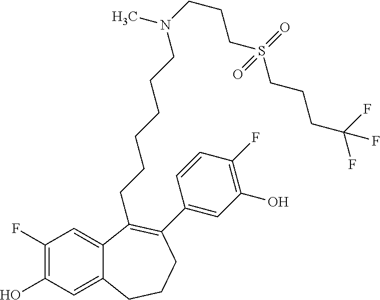 [SMILES: CN(CCCCCCC1=C(c2ccc(F)c(O)c2)CCCc2cc(O)c(F)cc21)CCCS(=O)(=O)CCCC(F)(F)F]

130 mg (0.29 mmol) of 9-(6-bromohexyl)-2-fluoro-8-(4-fluoro-3-hydroxyphenyl)-6,7-dihydro-5H-benzo[7]annulen-3-ol were reacted with 85.5 mg (0.35 mmol) of N-methyl-3-[(4,4,4-trifluorobutyl)sulphonyl]propane-1-amine according to Procedure 11. The material was purified by HPLC method 12. 31.8 mg (18% of theory) of product were isolated.

¹H NMR (300 MHz, chloroform-d₁): δ=0.98-1.26 (m, 6H), 1.46 (m, 2H), 1.98-2.41 (m, 12H), 2.43-2.58 (m, 7H), 2.80 (m, 2H), 3.07-3.21 (m, 4H), 6.62 (ddd, 1H), 6.81 (d, 1H), 6.89 (dd, 1H), 6.94-7.06 (m, 2H).

### Example 128

### 2-Fluoro-8-(4-fluoro-3-hydroxyphenyl)-9-[6-(methyl{4-[(4,4,4-trifluorobutyl)sulphonyl]butyl}amino)hexyl]-6,7-dihydro-5H-benzo[7]annulen-3-ol

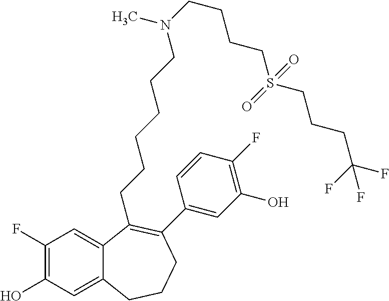 [SMILES: CN(CCCCCCC1=C(c2ccc(F)c(O)c2)CCCc2cc(O)c(F)cc21)CCCCS(=O)(=O)CCCC(F)(F)F]

140 mg (0.31 mmol) of 9-(6-bromohexyl)-2-fluoro-8-(4-fluoro-3-hydroxyphenyl)-6,7-dihydro-5H-benzo[7]annulen-3-ol were reacted with 97.3 mg (0.37 mmol) of N-methyl-4-[(4,4,4-trifluorobutyl)sulphonyl]butane-1-amine according to Procedure 11. The material was purified by HPLC (XBridge C18, 5μ, 100×30 mm, 50 ml/min, mobile phase: water with 0.1% formic acid-acetonitrile 90:10, 0-1 minute; 90:10->1:99, 1-7.5 minutes; 1:99, 7.5-10 minutes). The residue was dissolved in dichloromethane, washed once with saturated sodium bicarbonate solution and three times with water, dried over magnesium sulphate and concentrated. 12.4 mg (6% of theory) of product were isolated.

¹H NMR (300 MHz, chloroform-d₁): δ=1.00-1.35 (m, 6H), 1.43 (m, 2H), 1.70-1.99 (m, 4H), 2.00-2.41 (m, 15H), 2.45-2.58 (m, 4H), 3.02-3.12 (m, 4H), 6.63 (ddd, 1H), 6.80 (d, 1H), 6.90 (dd, 1H), 6.95-7.06 (m, 2H).

### Example 129

### 2-Fluoro-8-(4-fluoro-3-hydroxyphenyl)-9-{6-[(2-hydroxy-2-methylpropyl){3-[(RS)-(3,3,3-trifluoropropyl)sulphinyl]propyl}amino]hexyl}-6,7-dihydro-5H-benzo[7]annulen-3-ol

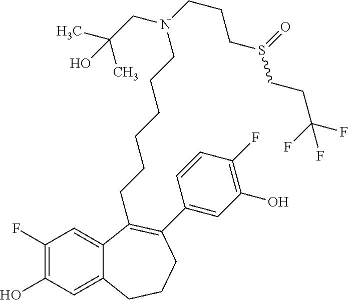 [SMILES: CC(C)(O)CN(CCCCCCC1=C(c2ccc(F)c(O)c2)CCCc2cc(O)c(F)cc21)CCCS(=O)CCC(F)(F)F]

150 mg (0.33 mmol) of 9-(6-bromohexyl)-2-fluoro-8-(4-fluoro-3-hydroxyphenyl)-6,7-dihydro-5H-benzo[7]annulen-3-ol were reacted with 128.1 mg (0.47 mmol) of 2-methyl-1-({3-[(RS)-(3,3,3-trifluoropropyl)sulphinyl]propyl}amino)propan-2-ol according to Procedure 11 over 40 hours. The material was purified by HPLC method 12. 4.6 mg (2% of theory) of product were isolated.

¹H NMR (300 MHz, chloroform-d₁): δ=1.00-1.37 (m, 14H), 1.90-2.16 (m, 6H), 2.29-2.47 (m, 6H), 2.53-2.97 (m, 10H), 6.63 (ddd, 1H), 6.81-6.90 (m, 2H), 6.97-7.06 (m, 2H).

### Example 130

### 2-Fluoro-8-(4-fluoro-3-hydroxyphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol

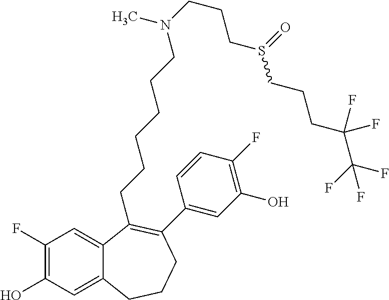 [SMILES: CN(CCCCCCC1=C(c2ccc(F)c(O)c2)CCCc2cc(O)c(F)cc21)CCCS(=O)CCCC(F)(F)C(F)(F)F]

130 mg (0.29 mmol) of 9-(6-bromohexyl)-2-fluoro-8-(4-fluoro-3-hydroxyphenyl)-6,7-dihydro-5H-benzo[7]annulen-3-ol were reacted with 111.0 mg (0.40 mmol) of N-methyl-3-[(RS)-(4,4,5,5,5-pentafluoropentyl)sulphinyl]propane-1-amine according to Procedure 11. The material was purified by HPLC method 12. 53 mg (28% of theory) of product were isolated.

¹H NMR (300 MHz, chloroform-d₁): δ=0.99-1.25 (m, 6H), 1.44 (m, 2H), 1.97-2.35 (m, 12H), 2.37-2.56 (m, 7H), 2.68-2.91 (m, 6H), 6.60 (ddd, 1H), 6.81 (d, 1H), 6.89 (dd, 1H), 6.93-7.05 (m, 2H).

### Example 131

### 2-Fluoro-8-(4-fluoro-3-hydroxyphenyl)-9-{6-[(2-methoxyethyl){3-[(RS)-(4,4,5,5,5-pentafluoropentyl)sulphinyl]propyl}amino]hexyl}-6,7-dihydro-5H-benzo[7]annulen-3-ol

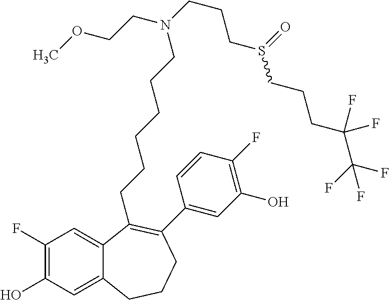 [SMILES: COCCN(CCCCCCC1=C(c2ccc(F)c(O)c2)CCCc2cc(O)c(F)cc21)CCCS(=O)CCCC(F)(F)C(F)(F)F]

150 mg (0.33 mmol) of 9-(6-bromohexyl)-2-fluoro-8-(4-fluoro-3-hydroxyphenyl)-6,7-dihydro-5H-benzo[7]annulen-3-ol were reacted with 129.7 mg (0.40 mmol) of N-(2-methoxyethyl)-3-[(RS)-(4,4,5,5,5-pentafluoropentyl)sulphinyl]propane-1-amine according to Procedure 11 over 40 hours. The material was purified by HPLC method 12. 3.7 mg (2% of theory) of product were isolated.

¹H NMR (300 MHz, chloroform-d₁): δ=0.98-1.27 (m, 6H), 1.35 (m, 2H), 1.92-2.37 (m, 12H), 2.47 (m, 2H), 2.55 (m, 2H), 2.68-2.92 (m, 8H), 3.33 (s, 3H), 3.50 (t, 2H), 6.61 (ddd, 1H), 6.83 (d, 1H), 6.89 (dd, 1H), 6.95-7.05 (m, 2H).

### Example 132

### 2-Fluoro-8-(4-fluoro-3-hydroxyphenyl)-9-{6-[(3-methoxypropyl){3-[(RS)-(4,4,5,5,5-pentafluoropentyl)sulphinyl]propyl}amino]hexyl}-6,7-dihydro-5H-benzo[7]annulen-3-ol

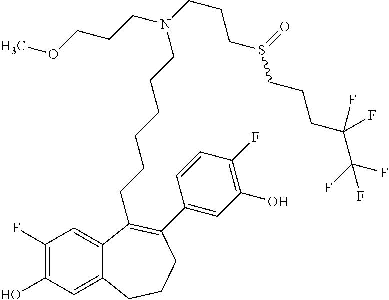 [SMILES: COCCCN(CCCCCCC1=C(c2ccc(F)c(O)c2)CCCc2cc(O)c(F)cc21)CCCS(=O)CCCC(F)(F)C(F)(F)F]

150 mg (0.33 mmol) of 9-(6-bromohexyl)-2-fluoro-8-(4-fluoro-3-hydroxyphenyl)-6,7-dihydro-5H-benzo[7]annulen-3-ol were reacted with 135.3 mg (0.40 mmol) of 3-methoxy-N-{3-[(RS)-(4,4,5,5,5-pentafluoropentyl)sulphinyl]propyl}propane-1-amine according to Procedure 11 over 40 hours. The material was purified by HPLC method 12. 15 mg (6% of theory) of product were isolated.

¹H NMR (300 MHz, chloroform-d₁): δ=0.98-1.25 (m, 6H), 1.39 (m, 2H), 1.77 (m, 2H), 1.96-2.38 (m, 12H), 2.43-2.59 (m, 4H), 2.67-2.91 (m, 8H), 3.32 (s, 3H), 3.41 (t, 2H), 6.60 (ddd, 1H), 6.82 (d, 1H), 6.89 (dd, 1H), 6.94-7.05 (m, 2H).

### Biological Examples

### Abbreviations and Acronyms

ER oestrogen receptor
E2 17β-estradiol
SERM selective oestrogen receptor modulator
d day
OVX ovariectomized animals

### Pharmacological In Vitro Examination of the Compounds According to the Invention

### Example 133

### Effect on Stability of the ERα Protein

In addition to inhibition of the transcriptional activity of the ER, anti-oestrogens influence the expression level of the ER in the target tissues through stimulation of the proteolytic degradation of the ER. In comparison with an ER-E2 complex, the ER bound in a complex with the pure anti-oestrogen fulvestrant has a substantially shorter half-life. Conversely, the ER stability is enhanced by the SERM tamoxifen, so that overall there is ER stabilization. All things considered, it can be assumed that the capacity of pure anti-oestrogens and certain SERMs for inducing ER degradation contributes significantly to the overall action of the compounds. Compounds that have a destabilizing property, but at the same time display the desired tissue-specific agonistic qualities, e.g. bone protection, should on the whole present a superior pharmacological profile, as they have a lower potential for side effects, such as stimulation of the endometrium.

The effect of the claimed pharmacological compounds on the stability of the ER was analysed in T47D breast cancer cells (see Table 1, column Standardized ER-destabilization [%]). These cells express the ER in functional form. The cells are incubated for 24 hours with the claimed compounds at a concentration of 1 μM. After lysis of the cells, the content of ER protein was determined by ELISA. Treatment with the complete destabilizer fulvestrant (0% ER), the stabilizer tamoxifen (100% ER) and the control medium (approx. 30% ER) was used as comparison. Compounds having an ER content below 30% are classified as destabilizing.

As described, the claimed pharmacological substances were investigated for their action on the stability of the ERα protein (see Table 1). Over most of the claimed structural range, the pharmacological substances display a destabilizing action on the ERα content (remaining relative ERα content of less than or equal to 30%).

TABLE 1

| | Standardized ER- |
|---|---|
| | destabilization |
| Ex. | [%] |
| 1 | 6 |
| 2 | 4 |
| 3 | 4 |
| 4 | 7 |
| 5 | 14 |
| 6 | 0 |
| 7 | −1 |
| 8 | 1 |
| 9 | 0 |
| 10 | |
| 11 | |
| 12 | 0 |
| 13 | −1 |
| 14 | 6 |
| 15 | 3 |
| 16 | 9 |
| 17 | 6 |
| 18 | 13 |
| 19 | 15 |
| 20 | 4 |
| 21 | 10 |
| 22 | 3 |
| 23 | 6 |
| 24 | 18 |
| 25 | −3 |
| 26 | 1 |
| 27 | 2 |
| 28 | 4 |
| 29 | 2 |
| 30 | 1 |
| 31 | 2 |
| 32 | 4 |
| 33 | 6 |
| 34 | 6 |
| 35 | 11 |
| 36 | 5 |
| 37 | 0 |
| 38 | 3 |
| 39 | 5 |
| 40 | 8 |
| 41 | 6 |
| 42 | 7 |
| 43 | 4 |
| 44 | 3 |
| 45 | 4 |
| 46 | 5 |
| 47 | 4 |
| 48 | 6 |
| 49 | 1 |
| 50 | 0 |
| 51 | 26 |
| 52 | 10 |
| 53 | 25 |
| 54 | 25 |
| 55 | 72 |
| 56 | 86 |
| 57 | 18 |
| 58 | 8 |
| 59 | 69 |
| 60 | 81 |
| 61 | 23 |
| 62 | 16 |
| 63 | 0 |
| 64 | 0 |
| 65 | 116 |
| 66 | |
| 67 | 4 |
| 68 | 2 |
| 69 | 4 |
| 70 | 1 |
| 71 | |
| 72 | 1 |
| 73 | |
| 74 | −2 |
| 75 | |
| 76 | 65 |
| 77 | |
| 78 | |
| 79 | |
| 80 | |
| 81 | |
| 82 | 13 |
| 83 | 13 |
| 84 | 2 |
| 85 | 1 |
| 86 | 6 |
| 87 | 0 |
| 88 | 2 |
| 89 | 0 |
| 90 | 6 |
| 91 | |
| 92 | 0 |
| 93 | |
| 94 | 62 |
| 95 | |
| 96 | 31 |
| 97 | 13 |
| 98 | 32 |
| 99 | |
| 100 | 30 |
| 101 | 2 |
| 102 | 10 |
| 103 | 0 |
| 104 | 5 |
| 105 | |
| 106 | |
| 107 | |
| 108 | 3 |
| 109 | 14 |
| 110 | 5 |
| 111 | 23 |
| 112 | 21 |
| 113 | 8 |
| 114 | 4 |
| 115 | |
| 116 | 10 |
| 117 | 3 |
| 118 | 2 |
| 119 | 9 |
| 120 | 8 |
| 121 | 4 |
| 122 | 16 |
| 123 | 16 |
| 124 | 5 |
| 125 | 59 |
| 126 | 24 |
| 127 | 20 |
| 128 | 6 |
| 129 | 20 |
| 130 | 6 |
| 131 | 0 |
| 132 | 5 |

### Example 134

### Anti-Oestrogenic Action in MVLN Cells

The anti-oestrogenic action of the claimed pharmacological compounds was investigated in so-called MVLN cells in vitro. MVLN cells are derivatives of the hormone-responsive MCF7 breast cancer cells known by a person skilled in the art. These MVLN cells express, along with the functional oestrogen receptor (ER), a reporter construct, which under ER-activation expresses luciferase. Determination of the activity of the induced luciferase permits a direct conclusion on the oestrogen properties of substances. To investigate the anti-oestrogenic properties of the pharmacological compounds, they were investigated in the presence of oestrogen for their potential for inhibiting the luciferase signal induced by estradiol.

The claimed pharmacological test substances were investigated in MVLN cells for their anti-oestrogenic potential, as described (see Table 2). Over the entire structural range, these compounds show high potency (IC₅₀ values below 0.3 μM) and primarily even double-digit or single-digit nanomolar IC₅₀ values for the inhibition of estradiol-induced luciferase activity.

TABLE 2

| | Anti-oestrogenic action in vitro: |
|---|---|
| | MVLN transactivation antagonism |
| Ex. | IC50 (nM) |
| 1 | 37 |
| 2 | 8 |
| 3 | 15 |
| 4 | 3 |
| 5 | 5 |
| 6 | 4 |
| 7 | 5 |
| 8 | 8 |
| 9 | 8 |
| 10 | 2 |
| 11 | 4 |
| 12 | 2 |
| 13 | 3 |
| 14 | 2 |
| 15 | 3 |
| 16 | 2 |
| 17 | 4 |
| 18 | 2 |
| 19 | 5 |
| 20 | 2 |
| 21 | 5 |
| 22 | 17 |
| 23 | 2 |
| 24 | 13 |
| 25 | 24 |
| 26 | 1 |
| 27 | 1 |
| 28 | 2 |
| 29 | 3 |
| 30 | 2 |
| 31 | 3 |
| 32 | 3 |
| 33 | 6 |
| 34 | 5 |
| 35 | 11 |
| 36 | 3 |
| 37 | 1 |
| 38 | 2 |
| 39 | 146 |
| 40 | 27 |
| 41 | 110 |
| 42 | 7 |
| 43 | 8 |
| 44 | 7 |
| 45 | 65 |
| 46 | 14 |
| 47 | 34 |
| 48 | |
| 49 | 3 |
| 50 | 7 |
| 51 | 2 |
| 52 | 1 |
| 53 | 2 |
| 54 | 2 |
| 55 | 2 |
| 56 | 3 |
| 57 | 5 |
| 58 | 3 |
| 59 | 10 |
| 60 | 26 |
| 61 | 9 |
| 62 | 12 |
| 63 | 9 |
| 64 | 5 |
| 65 | 6 |
| 66 | 5 |
| 67 | 3 |
| 68 | 5 |
| 69 | 6 |
| 70 | 2 |
| 71 | 13 |
| 72 | 4 |
| 73 | 17 |
| 74 | 3 |
| 75 | 14 |
| 76 | 23 |
| 77 | 61 |
| 78 | 11 |
| 79 | 9 |
| 80 | 9 |
| 81 | 39 |
| 82 | 9 |
| 83 | 7 |
| 84 | 31 |
| 85 | 40 |
| 86 | 1 |
| 87 | 1 |
| 88 | 1 |
| 89 | 2 |
| 90 | 2 |
| 91 | 3 |
| 92 | 1 |
| 93 | 1 |
| 94 | 9 |
| 95 | 7 |
| 96 | 5 |
| 97 | 2 |
| 98 | 3 |
| 99 | 8 |
| 100 | 3 |
| 101 | 3 |
| 102 | 32 |
| 103 | 26 |
| 104 | 25 |
| 105 | 2 |
| 106 | 1 |
| 107 | 3 |
| 108 | 2 |
| 109 | 87 |
| 110 | 5 |
| 111 | 250 |
| 112 | 45 |
| 113 | 2 |
| 114 | 20 |
| 115 | 12 |
| 116 | 34 |
| 117 | 11 |
| 118 | 9 |
| 119 | 7 |
| 120 | 5 |
| 121 | 7 |
| 122 | 6 |
| 123 | 11 |
| 124 | 6 |
| 125 | 16 |
| 126 | 32 |
| 127 | 6 |
| 128 | 5 |
| 129 | 4 |
| 130 | 5 |
| 131 | 2 |
| 132 | 5 |

The suitability of the compounds according to the invention for the treatment of endometriosis can be demonstrated in the following animal models. The influence of the compounds according to the invention on the uterus was investigated in the uterus growth test (oestrogenic action) and in the anti-uterus growth test (anti-oestrogenic action), both conducted in rats.

### Example 135

### Anti-Uterus Growth Test in the Adult Rat

The uterus of oestrogen-substituted rats can be used as a test model for detecting a direct action of substances with anti-oestrogenic properties. The parameter of oestrogenic action is estradiol-induced uterus growth in rats, which is inhibited by simultaneous administration of a substance with anti-oestrogenic action.

The experimental animals (n=5-6 animals/group) were ovariectomized before the start of the test, to rule out the influence of endogenous oestrogens. After a phase of 6 to 10 days, the test substances are administered s.c. on 3 consecutive days (d1-d3) in combination with a substitution dose of 1.5 μg/kg/day of 17β-estradiol. 17β-estradiol alone serves as positive control, and the excipients serve as negative control. On day 4 (d4) the animals are killed, and the uteri and vaginae are removed and weighed. The organ weights are converted to mg/100 g body weight, then the mean value and the standard deviation are calculated for each dosage. The inhibition of uterus or vaginal growth induced by 17β-estradiol is shown as percentage inhibition.

The compounds according to the invention largely show very pronounced inhibition of uterus growth induced by 17β-estradiol.

The compounds according to the invention are therefore superior in the sense of the present invention, with respect to their action on the uterus, to the compounds of the prior art, in that they have less, if any, oestrogenic action on this organ.

The selected claimed substances were investigated in adult female rats as described for their anti-oestrogenic, inhibitory action on the weight of the uterus. At the dosage used, the substances show a marked anti-oestrogenic action in vivo (Table 3 and Table 4).

TABLE 3

| | Anti-oestrogenic activity in vivo in % |
|---|---|
| Example | at a dose of 0.3 mg/kg (rat) |
| 2 | 51 |
| 6 | 22 |
| 7 | 31 |
| 13 | 79 |
| 19 | 38 |
| 21 | 37 |
| 23 | 77 |
| 24 | 14 |
| 27 | 80 |
| 29 | 61 |
| 37 | 59 |
| 38 | 57 |
| 44 | 32 |
| 49 | 72 |
| 51 | 62 |
| 56 | 33 |
| 58 | 88 |
| 64 | 85 |
| 102 | 24 |

TABLE 4

| | Anti-oestrogenic activity in vivo in % |
|---|---|
| Example | at a dose of 1 mg/kg (rat) |
| 1 | 65 |
| 2 | 94 |
| 3 | 64 |
| 6 | 64 |
| 46 | 31 |

### Example 136

### Stimulation of Ovarian Oestrogen Synthesis

The clinical use both of pure anti-oestrogens and of various SERMs for the treatment of premenopausal women is limited by their property of stimulating the ovaries through activation of the hypothalamic-pituitary-gonadal axis (HPG axis), which leads to the increase in peripheral estradiol levels (Palomba, S., Orio, F., Jr., Morelli, M., Russo, T., Pellicano, M., Zupi, E., Lombardi, G., Nappi, C., Panici, P. L., and Zullo, F. (2002). Raloxifene administration in premenopausal women with uterine leiomyomas: a pilot study. J Clin Endocrinol Metab 87, 3603-3608). This stimulation of the HPG axis is associated with penetration of the blood-brain barrier and penetration of the brain. To investigate the ovary-stimulating properties of the claimed pharmacological compounds, hormonally intact adult rats were treated with the substances daily for a period of 10 days. The study end point is the quotient of peripheral estradiol values after and before treatment.

In comparison with pure anti-oestrogens and the classical SERMs such as raloxifene or bazedoxifene, the selected claimed pharmacological compounds show markedly less stimulation of the HPG axis at equal dosage. They therefore display superior properties for clinical use in premenopausal women.

Selected claimed pharmacological substances were investigated as described for their stimulating action on the HPG axis or ovarian estradiol synthesis. The selected substances show markedly less stimulation of the ovaries than the control compound raloxifene at equal dosages (cf. Table 5).

TABLE 5

| | Stimulation of ovarian oestrogen synthesis: |
|---|---|
| Example | factor at a dose of 3 mg/kg rat |
| 2 | 1.8 |
| 38 | 1.7 |
| 27 | 2.4 |
| 29 | 1.1 |
| 7 | 1.8 |
| raloxifene | 3.1 |
| hydrochloride | |

### Example 137

### Bioavailability in the Rat

Determination of bioavailability after intragastric application of test substances was carried out in conscious female rats with a body weight from at least 0.2 kg to at most 0.25 kg. For this, the test substances were applied in dissolved form both for intravenous and for intragastric application, where compatible solubilizers such as PEG400 and/or ethanol were used in a compatible amount.

a) Intravenous Administration:

The test substances were administered at a dose of 0.5-1 mg/kg as quick infusion over a period of 15 minutes. At the time points 2 min, 8 min, 15 min (infusion) and 5 min, 15 min, 30 min, 45 min, 1 h, 2 h, 4 h, 6 h, 8 h, 12 h, 16 h, 20 h, 24 h after the end of infusion, about 150 μl blood samples were taken via a catheter from the jugular vein. Lithium-heparin was added as anticoagulant to the blood samples and they were stored in a refrigerator until required for further processing. After centrifugation of the samples at 3000 rpm for 15 min, an aliquot of 100 μl was taken from the supernatant (plasma) and was precipitated by adding 400 μl of cold acetonitrile or methanol (absolute). The precipitated samples were frozen-out overnight at −20° C., then centrifuged once again for 15 min at 3000 rpm, before taking 150 μl of the clear supernatant for determination of concentration. Analysis used an Agilent 1200 HPLC system coupled to LCMS/MS detection.

Calculation of the PK parameters (using PK calculation software, for example WinNonLin®): CLₚₗₐₛₘₐ total plasma clearance of the test substance (in l*kg/h); CL_{blood}: total blood clearance of the test substance (in l*kg/h), where (CL_{blood=CL}ₚₗₐₛₘₐ*Cₚ/C_{b}), Vₛₛ: apparent steady-state distribution volume (in l/kg); t_{1/2}: half-life within a specified interval (here: terminal t_{1/2}, in h); AUCₙₒᵣₘ: area under the plasma concentration time profile from time point zero extrapolated to infinity divided by the dose normalized for body weight (in kg*l/h); AUC₍₀₋ₜₙ₎ₙₒᵣₘ: integrated area under the plasma concentration time profile from time point zero until the last time point at which a plasma concentration was measurable, divided by the dose normalized for body weight (in kg*l/h); Cₘₐₓ: maximum concentration of the test substance in the plasma (in μg/I); C_{max,norm}: maximum concentration of the test substance in the plasma divided by the dose normalized for body weight (in kg/l); C_{b}/Cₚ: ratio of blood to plasma concentration distribution.

b) Intragastric Administration:

The test substances were administered to fasting female rats at a dose of 2-5 mg/kg intragastrically as a bolus using a feeding tube. At time points 8 min, 15 min, 30 min, 45 min, 1 h, 2 h, 4 h, 6 h, 8 h, 12 h, 16 h, 20 h, 24 h, approx. 150 μl blood samples were taken via a catheter from the jugular vein. Lithium-heparin was added as anticoagulant to the blood samples and they were stored in a refrigerator until required for further processing. After centrifuging the samples for 15 min at 3000 rpm, an aliquot of 100 μl was taken from the supernatant (plasma) and was precipitated by adding 400 μl of cold acetonitrile or methanol (absolute). The precipitated samples were frozen-out overnight at −20° C., then centrifuged for 15 min at 3000 rpm before 150 μl of the clear supernatant was drawn off for determination of concentration. Analysis was carried out using an Agilent 1200 HPLC system coupled to LCMS/MS detection.

Calculation of the PK parameters (using PK calculation software, for example WinNonLin®):

AUCₙₒᵣₘ: area under the plasma concentration time profile from time point zero extrapolated to infinity divided by the dose normalized for body weight (in kg*l/h); AUC₍₀₋ₜₙ₎ₙₒᵣₘ: integrated area under the plasma concentration time profile from time point zero until the last time point at which a plasma concentration was measurable, divided by the dose normalized for body weight (in kg*l/h); Cₘₐₓ: maximum concentration of the test substance in the plasma (in μg/l); C_{max,norm}: maximum concentration of the test substance in the plasma divided by the dose normalized for body weight (in kg/l); t_{1/2}: half-life within a specified interval (here: terminal t_{1/2}, in h); F_{obs %}: observed oral bioavailability, AUC₍₀₋ₜₙ₎ₙₒᵣₘ after i.g. administration divided by AUC₍₀₋ₜₙ₎ₙₒᵣₘ after i.v. administration. Tₘₐₓ: time point at which the maximum concentration of the test substance is measured in the plasma.

### Working Examples of Pharmaceutical Compositions

The compounds according to the invention can be transformed as follows into pharmaceutical preparations. The claimed compounds can be administered as a tablet. A possible composition for such a tablet can have the following appearance:

### Tablet:

### Composition:

100 mg of the compound of Example 1, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of convexity 12 mm.

### Production:

The mixture of compound according to the invention, lactose and starch is granulated with a 5% solution (w/w) of the PVP in water. After drying, the granules are mixed with the magnesium stearate for 5 minutes. This mixture is compacted with a customary tablet press (see above for tablet format). A compressing force of 15 kN is used as a guide value for compaction.

Recipe, ingredients, amount of substance and manner of preparation can deviate from this. The claimed compounds can also be administered as suspension for oral application. A possible composition for such a tablet can have the following appearance:

### Suspension for Oral Administration

### Composition:

1000 mg of the compound of example 1, 1000 mg of ethanol (96%), 400 mg of Rhodigel® (xanthan gum from the company FMC, Pennsylvania, USA) and 99 g of water.

An individual dose of 100 mg of the compound according to the invention is equivalent to 10 ml of oral suspension.

### Production:

The Rhodigel is suspended in ethanol, and the compound according to the invention is added to the suspension. The water is added with stirring. The mixture is stirred for about 6 h until the Rhodigel ceases to swell.

Recipe, ingredients, amount of substance and manner of preparation can deviate from this. The claimed compounds can also be administered as solution for oral application. A possible composition for such a tablet can have the following appearance:

### Solution for Oral Administration:

### Composition:

500 mg of the compound of example 1, 2.5 g of polysorbate and 97 g of polyethylene glycol 400. An individual dose of 100 mg of the compound according to the invention is equivalent to 20 g of oral solution.

### Production:

The compound according to the invention is suspended in the mixture of polyethylene glycol and polysorbate with stirring. Stirring is continued until the compound according to the invention has dissolved completely.

Recipe, ingredients, amount of substance and manner of preparation can deviate from this.

1. Compound of the general formula (I), 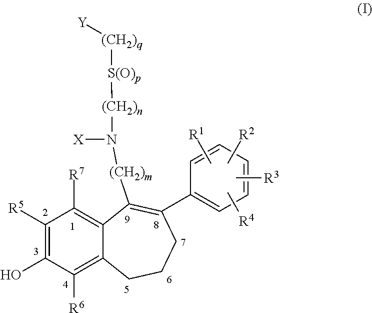
in which
R¹, R², R³ and R⁴ independently of one another represent hydrogen, hydroxy, alkoxy, nitrile, alkylsulphonyl or represent the replacement of a CH group in the aromatic ring by a nitrogen atom or
provided one or more of the other substituents represent hydroxy, alkoxy, nitrile or alkylsulphonyl—independently of one another represent fluorine
R⁵, R⁶ and R⁷ independently of one another represent hydrogen, fluorine, chlorine, bromine, methyl, ethyl, trifluoromethyl or nitrile,
X represents hydrogen or alkyl-, cycloalkyl-, alkoxyalkyl, C₃-C₆-alkenyl or C₃-C₆-alkynyl which are optionally mono- or polysubstituted by halogen, hydroxy, —CN or deuterium,
Y represents perfluorinated or partially fluorinated C₁-C₄-alkyl or perfluorinated or partially fluorinated C₃-C₈-cycloalkyl,
m represents 4, 5, 6 or 7,
n represents 2, 3, 4, 5, 6 or 7,
p represents 0, 1 or 2 and
q represents 1, 2, 3 or 4,
and salts, solvates or solvates of the salts thereof, including all crystal modifications.

2. Compound according to claim 1 in which
R¹, R², R³ and R⁴ independently of one another represent hydrogen, hydroxy, nitrile, methylsulphonyl or represent the replacement of a CH group in the aromatic ring by a nitrogen atom or
provided one or more of the other substituents represent hydroxy, nitrile, methylsulphonyl—independently of one another represent fluorine,
R⁵ and R⁶ independently of one another represent hydrogen, chlorine or fluorine,
R⁷ represents hydrogen,
X represents hydrogen or represents optionally hydroxy- or methoxy-substituted C₁-C₄-alkyl,
Y represents —CF₃, —C₂F₅, —CF₂CF₂CF₃ or —CF(CF₃)₂,
m represents 5 or 6,
n represents 3, 4, 5 or 6,
p represents 0, 1 or 2 and
q represents 2, 3 or 4,
and salts, solvates or solvates of the salts thereof, including all crystal modifications.

3. Compound according to claim 2 in which
R¹ and R² represent hydrogen and
either
R³ represents hydrogen and
R⁴ represents hydrogen, hydroxy, nitrile, methylsulphonyl or represents the replacement of a CH group in the aromatic ring by a nitrogen atom or
R³ represents fluorine and
R⁴ represents hydroxy, nitrile or methylsulphonyl,
R⁵ and R⁶ represent hydrogen, chlorine or fluorine, but do not both represent chlorine and do not both represent fluorine
X represents optionally hydroxy- or methoxy-substituted C₁-C₄-alkyl,
Y represents —CF₃ or —C₂F₅,
m represents 5 or 6,
n represents 3, 4, 5 or 6,
p represents 0, 1 or 2 and
q represents 2 or 3,
and salts, solvates or solvates of the salts thereof, including all crystal modifications.

4. Compound according to claim 3 of the formula (II) 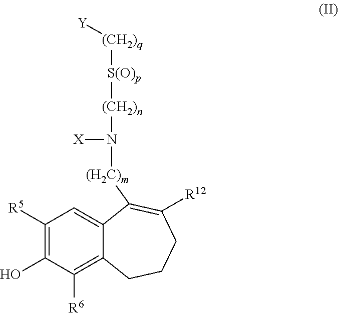
in which
R¹² represents phenyl, 3-hydroxyphenyl-, 4-hydroxyphenyl-, 3-fluoro-4-hydroxphenyl-, 4-fluoro-3-hydroxyphenyl, 2-fluoro-5-hydroxyphenyl, 4-methylsulphonylphenyl, 3-methylsulphonylphenyl, 4-cyanophenyl or 3-pyridyl,
R⁵ and R⁶ either represent hydrogen or fluorine, but do not both represent fluorine or—if R⁵ represents hydrogen—R⁶ represents chlorine,
X represents methyl, ethyl, methoxyethyl, methoxypropyl, hydroxyethyl, 3-hydroxypropyl or 2-hydroxy-2-methylpropyl,
Y represents —CF₃ or —C₂F₅,
m represents 5 or 6,
n represents 3, 4, 5 or 6,
p represents 0, 1 or 2,
q represents 2 or 3,
and salts, solvates or solvates of the salts thereof, including all crystal modifications.

5. Compounds according to claim 1 having the names
9-[5-(methyl{3-[(4,4,5,5,5-pentafluoropentyl)sulphanyl]propyl}amino)pentyl]-8-phenyl-6,7-dihydro-5H-benzo[7]annulen-3-ol
9-[5-(methyl{3-[(RS)-(4,4,5,5,5-pentafluoropentyl)sulphinyl]propyl}amino)pentyl]-8-phenyl-6,7-dihydro-5H-benzo[7]annulen-3-ol
9-[5-(methyl{3-[(4,4,5,5,5-pentafluoropentyl)sulphonyl]propyl}amino)pentyl]-8-phenyl-6,7-dihydro-5H-benzo[7]annulen-3-ol
8-(3-hydroxyphenyl)-9-[5-(methyl{3-[(RS)-(4,4,5,5,5-pentafluoropentyl)sulphinyl]propyl}amino)pentyl]-6,7-dihydro-5H-benzo[7]annulen-3-ol
8-(3-hydroxyphenyl)-9-[5-(methyl{3-[(4,4,5,5,5-pentafluoropentyl)sulphonyl]propyl}amino)pentyl]-6,7-dihydro-5H-benzo[7]annulen-3-ol
8-(3-hydroxyphenyl)-9-[5-(methyl{4-[(RS)-(4,4,5,5,5-pentafluoropentyl)sulphinyl]butyl}amino)pentyl]-6,7-dihydro-5H-benzo[7]annulen-3-ol
8-(3-hydroxyphenyl)-9-[5-(methyl{4-[(4,4,5,5,5-pentafluoropentyl)sulphonyl]butyl}amino)pentyl]-6,7-dihydro-5H-benzo[7]annulen-3-ol
8-(3-hydroxyphenyl)-9-[5-(methyl{5-[(RS)-(4,4,5,5,5-pentafluoropentyl)sulphinyl]pentyl}amino)pentyl]-6,7-dihydro-5H-benzo[7]annulen-3-ol
8-(3-hydroxyphenyl)-9-[5-(methyl{5-[(4,4,5,5,5-pentafluoropentyl)sulphonyl]pentyl}amino)pentyl]-6,7-dihydro-5H-benzo[7]annulen-3-ol
8-(3-hydroxyphenyl)-9-[5-(methyl{6-[(RS)-(4,4,5,5,5-pentafluoropentyl)sulphinyl]hexyl}amino)pentyl]-6,7-dihydro-5H-benzo[7]annulen-3-ol
8-(3-hydroxyphenyl)-9-[5-(methyl{6-[(4,4,5,5,5-pentafluoropentyl)sulphonyl]hexyl}amino)pentyl]-6,7-dihydro-5H-benzo[7]annulen-3-ol
8-(3-hydroxyphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol
8-(3-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol
8-(3-hydroxyphenyl)-9-[6-(methyl{4-[(RS)-(4,4,5,5,5-pentafluoropentyl)sulphinyl]butyl}amino)hexyl]-6,7-dihydro-5H-benzo[7]annulen-3-ol
8-(3-hydroxyphenyl)-9-[6-(methyl{4-[(4,4,5,5,5-pentafluoropentyl)sulphonyl]butyl}amino)hexyl]-6,7-dihydro-5H-benzo[7]annulen-3-ol
8-(3-hydroxyphenyl)-9-[6-(methyl{5-[(RS)-(4,4,5,5,5-pentafluoropentyl)sulphinyl]pentyl}amino)hexyl]-6,7-dihydro-5H-benzo[7]annulen-3-ol
8-(3-hydroxyphenyl)-9-[6-(methyl{5-[(4,4,5,5,5-pentafluoropentyl)sulphonyl]pentyl}amino)hexyl]-6,7-dihydro-5H-benzo[7]annulen-3-ol
8-(3-hydroxyphenyl)-9-[6-(methyl{6-[(RS)-(4,4,5,5,5-pentafluoropentyl)sulphinyl]hexyl}amino)hexyl]-6,7-dihydro-5H-benzo[7]annulen-3-ol
8-(3-hydroxyphenyl)-9-[6-(methyl{6-[(4,4,5,5,5-pentafluoropentyl)sulphonyl]hexyl}amino)hexyl]-6,7-dihydro-5H-benzo[7]annulen-3-ol
8-(4-hydroxyphenyl)-9-[6-(methyl{3-[(4,4,5,5,5-pentafluoropentyl)sulphanyl]propyl}amino)hexyl]-6,7-dihydro-5H-benzo[7]annulen-3-ol
8-(3-mesylphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol
4-{3-hydroxy-9-[6-(methyl{3-[(4,4,5,5,5-pentafluoropentyl)sulphonyl]propyl}amino)hexyl]-6,7-dihydro-5H-benzo[7]annulen-8-yl}benzonitrile
8-(2-fluoro-5-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol
4-chloro-8-(4-fluoro-3-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol
8-(4-fluoro-3-hydroxyphenyl)-9-[5-(methyl{4-[(4,4,5,5,5-pentafluoropentyl)sulphonyl]butyl}amino)pentyl]-6,7-dihydro-5H-benzo[7]annulen-3-ol
8-(4-hydroxyphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol
8-(4-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol
8-(4-fluoro-3-hydroxyphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol
8-(4-fluoro-3-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol
8-(4-fluoro-3-hydroxyphenyl)-9-[6-(methyl{4-[(RS)-(4,4,5,5,5-pentafluoropentyl)sulphinyl]butyl}amino)hexyl]-6,7-dihydro-5H-benzo[7]annulen-3-ol
8-(4-fluoro-3-hydroxyphenyl)-9-[6-(methyl{4-[(4,4,5,5,5-pentafluoropentyl)sulphonyl]butyl}amino)hexyl]-6,7-dihydro-5H-benzo[7]annulen-3-ol
8-(4-fluoro-3-hydroxyphenyl)-9-[6-(methyl{5-[(RS)-(4,4,5,5,5-pentafluoropentyl)sulphinyl]pentyl}amino)hexyl]-6,7-dihydro-5H-benzo[7]annulen-3-ol
8-(4-fluoro-3-hydroxyphenyl)-9-[6-(methyl{5-[(4,4,5,5,5-pentafluoropentyl)sulphonyl]pentyl}amino)hexyl]-6,7-dihydro-5H-benzo[7]annulen-3-ol
8-(4-fluoro-3-hydroxyphenyl)-9-[6-(methyl{6-[(RS)-(4,4,5,5,5-pentafluoropentyl)sulphinyl]hexyl}amino)hexyl]-6,7-dihydro-5H-benzo[7]annulen-3-ol
8-(4-fluoro-3-hydroxyphenyl)-9-[6-(methyl{6-[(4,4,5,5,5-pentafluoropentyl)sulphonyl]hexyl}amino)hexyl]-6,7-dihydro-5H-benzo[7]annulen-3-ol
8-(3-fluoro-4-hydroxyphenyl)-9-[5-(methyl{3-[(4,4,5,5,5-pentafluoropentyl)sulphanyl]propyl}amino)pentyl]-6,7-dihydro-5H-benzo[7]annulen-3-ol
8-(3-fluoro-4-hydroxyphenyl)-9-[5-(methyl{3-[(RS)-(4,4,5,5,5-pentafluoropentyl)sulphinyl]propyl}amino)pentyl]-6,7-dihydro-5H-benzo[7]annulen-3-ol
8-(3-fluoro-4-hydroxyphenyl)-9-[5-(methyl{3-[(4,4,5,5,5-pentafluoropentyl)sulphonyl]propyl}amino)pentyl]-6,7-dihydro-5H-benzo[7]annulen-3-ol
9-[5-(methyl{3-[(4,4,5,5,5-pentafluoropentyl)sulphanyl]propyl}amino)pentyl]-8-(3-pyridyl)-6,7-dihydro-5H-benzo[7]annulen-3-ol
9-[5-(methyl{3-[(RS)-(4,4,5,5,5-pentafluoropentyl)sulphinyl]propyl}amino)pentyl]-8-(3-pyridyl)-6,7-dihydro-5H-benzo[7]annulen-3-ol
9-[5-(methyl{3-[(4,4,5,5,5-pentafluoropentyl)sulphonyl]propyl}amino)pentyl]-8-(3-pyridyl)-6,7-dihydro-5H-benzo[7]annulen-3-ol
9-[6-(methyl{3-[(4,4,5,5,5-pentafluoropentyl)sulphanyl]propyl}amino)hexyl]-8-(3-pyridyl)-6,7-dihydro-5H-benzo[7]annulen-3-ol
9-[6-(methyl{3-[(RS)-(4,4,5,5,5-pentafluoropentyl)sulphinyl]propyl}amino)hexyl]-8-(3-pyridyl)-6,7-dihydro-5H-benzo[7]annulen-3-ol
9-[6-(methyl{3-[(4,4,5,5,5-pentafluoropentyl)sulphonyl]propyl}amino)hexyl]-8-(3-pyridyl)-6,7-dihydro-5H-benzo[7]annulen-3-ol
8-(4-mesylphenyl)-9-[5-(methyl{3-[(4,4,5,5,5-pentafluoropentyl)sulphanyl]propyl}amino)pentyl]-6,7-dihydro-5H-benzo[7]annulen-3-ol
8-(4-mesylphenyl)-9-[5-(methyl{3-[(RS)-(4,4,5,5,5-pentafluoropentyl)sulphinyl]propyl}amino)pentyl]-6,7-dihydro-5H-benzo[7]annulen-3-ol
8-(4-mesylphenyl)-9-[5-(methyl{3-[(4,4,5,5,5-pentafluoropentyl)sulphonyl]propyl}amino)pentyl]-6,7-dihydro-5H-benzo[7]annulen-3-ol
8-(4-mesylphenyl)-9-[6-(methyl{3-[(RS)-(4,4,5,5,5-pentafluoropentyl)sulphinyl]propyl}amino)hexyl]-6,7-dihydro-5H-benzo[7]annulen-3-ol
8-(4-mesylphenyl)-9-[6-(methyl{3-[(4,4,5,5,5-pentafluoropentyl)sulphonyl]propyl}amino)hexyl]-6,7-dihydro-5H-benzo[7]annulen-3-ol
9-[5-({3-[(RS)-(4,4,5,5,5-pentafluoropentyl)sulphinyl]propyl}amino)pentyl]-8-phenyl-6,7-dihydro-5H-benzo[7]annulen-3-ol
8-(3-hydroxyphenyl)-9-[6-(methyl{3-[(4,4,4-trifluorobutyl)sulphonyl]propyl}amino)hexyl]-6,7-dihydro-5H-benzo[7]annulen-3-ol
8-(3-hydroxyphenyl)-9-[6-(methyl{4-[(4,4,4-trifluorobutyl)sulphonyl]butyl}amino)hexyl]-6,7-dihydro-5H-benzo[7]annulen-3-ol
8-(3-hydroxyphenyl)-9-[6-(methyl{3-[(RS)-(3,3,4,4,4-pentafluorobutyl)sulphinyl]propyl}amino)hexyl]-6,7-dihydro-5H-benzo[7]annulen-3-ol
8-(3-hydroxyphenyl)-9-[6-(methyl{3-[(3,3,4,4,4-pentafluorobutyl)sulphonyl]propyl}amino)hexyl]-6,7-dihydro-5H-benzo[7]annulen-3-ol
8-(3-hydroxyphenyl)-9-[6-(methyl{3-[(RS)-(3,3,3-trifluoropropyl)sulphinyl]propyl}amino)hexyl]-6,7-dihydro-5H-benzo[7]annulen-3-ol
8-(3-hydroxyphenyl)-9-[6-(methyl{3-[(3,3,3-trifluoropropyl)sulphonyl]propyl}amino)hexyl]-6,7-dihydro-5H-benzo[7]annulen-3-ol
9-[6-(methyl{3-[(4,4,4-trifluorobutyl)sulphonyl]propyl}amino)hexyl]-8-phenyl-6,7-dihydro-5H-benzo[7]annulen-3-ol
9-[6-(methyl{4-[(4,4,4-trifluorobutyl)sulphonyl]butyl}amino)hexyl]-8-phenyl-6,7-dihydro-5H-benzo[7]annulen-3-ol
9-[6-(methyl{3-[(RS)-(3,3,3-trifluoropropyl)sulphinyl]propyl}amino)hexyl]-8-phenyl-6,7-dihydro-5H-benzo[7]annulen-3-ol
9-[6-(methyl{3-[(3,3,3-trifluoropropyl)sulphonyl]propyl}amino)hexyl]-8-phenyl-6,7-dihydro-5H-benzo[7]annulen-3-ol
9-[6-(methyl{3-[(RS)-(3,3,4,4,4-pentafluorobutyl)sulphinyl]propyl}amino)hexyl]-8-phenyl-6,7-dihydro-5H-benzo[7]annulen-3-ol
9-[6-(methyl{3-[(3,3,4,4,4-pentafluorobutyl)sulphonyl]propyl}amino)hexyl]-8-phenyl-6,7-dihydro-5H-benzo[7]annulen-3-ol
9-[6-(methyl{3-[(4,4,5,5,5-pentafluoropentyl)sulphonyl]propyl}amino)hexyl]-8-phenyl-6,7-dihydro-5H-benzo[7]annulen-3-ol
9-[6-(methyl{3-[(RS)-(4,4,5,5,5-pentafluoropentyl)sulphinyl]propyl}amino)hexyl]-8-phenyl-6,7-dihydro-5H-benzo[7]annulen-3-ol
9-[6-(ethyl{3-[(3,3,3-trifluoropropyl)sulphonyl]propyl}amino)hexyl]-8-(3-hydroxyphenyl)-6,7-dihydro-5H-benzo[7]annulen-3-ol
9-[6-(methyl{4-[(RS)-(4,4,5,5,5-pentafluoropentyl)sulphinyl]butyl}amino)hexyl]-8-phenyl-6,7-dihydro-5H-benzo[7]annulen-3-ol
9-[6-(methyl{4-[(4,4,5,5,5-pentafluoropentyl)sulphonyl]butyl}amino)hexyl]-8-phenyl-6,7-dihydro-5H-benzo[7]annulen-3-ol
9-[6-(ethyl{3-[(RS)-(4,4,5,5,5-pentafluoropentyl)sulphinyl]propyl}amino)hexyl]-8-phenyl-6,7-dihydro-5H-benzo[7]annulen-3-ol
9-[6-(ethyl{3-[(4,4,5,5,5-pentafluoropentyl)sulphonyl]propyl}amino)hexyl]-8-phenyl-6,7-dihydro-5H-benzo[7]annulen-3-ol
9-{6-[(2-hydroxyethyl){3-[(RS)-(4,4,5,5,5-pentafluoropentyl)sulphinyl]propyl}amino]hexyl}-8-phenyl-6,7-dihydro-5H-benzo[7]annulen-3-ol
9-{6-[(2-hydroxyethyl){3-[(4,4,5,5,5-pentafluoropentyl)sulphonyl]propyl}amino]hexyl}-8-phenyl-6,7-dihydro-5H-benzo[7]annulen-3-ol
9-{6-[(3-hydroxypropyl){3-[(RS)-(4,4,5,5,5-pentafluoropentyl)sulphinyl]propyl}amino]hexyl}-8-phenyl-6,7-dihydro-5H-benzo[7]annulen-3-ol
9-{6-[(3-hydroxypropyl){3-[(4,4,5,5,5-pentafluoropentyl)sulphonyl]propyl}amino]hexyl}-8-phenyl-6,7-dihydro-5H-benzo[7]annulen-3-ol
9-{6-[(2-hydroxy-2-methylpropyl){3-[(RS)-(4,4,5,5,5-pentafluoropentyl)sulphinyl]propyl}amino]hexyl}-8-phenyl-6,7-dihydro-5H-benzo[7]annulen-3-ol
9-{6-[(2-hydroxy-2-methylpropyl){3-[(4,4,5,5,5-pentafluoropentyl)sulphonyl]propyl}amino]hexyl}-8-phenyl-6,7-dihydro-5H-benzo[7]annulen-3-ol
9-[6-(ethyl{3-[(RS)-(3,3,3-trifluoropropyl)sulphinyl]propyl}amino)hexyl]-8-phenyl-6,7-dihydro-5H-benzo[7]annulen-3-ol
9-[6-(ethyl{3-[(3,3,3-trifluoropropyl)sulphonyl]propyl}amino)hexyl]-8-phenyl-6,7-dihydro-5H-benzo[7]annulen-3-ol
9-{6-[(2-hydroxyethyl){3-[(RS)-(3,3,3-trifluoropropyl)sulphinyl]propyl}amino]hexyl}-8-phenyl-6,7-dihydro-5H-benzo[7]annulen-3-ol
9-{6-[(2-hydroxyethyl){3-[(3,3,3-trifluoropropyl)sulphonyl]propyl}amino]hexyl}-8-phenyl-6,7-dihydro-5H-benzo[7]annulen-3-ol
9-{6-[(3-hydroxypropyl){3-[(RS)-(3,3,3-trifluoropropyl)sulphinyl]propyl}amino]hexyl}-8-phenyl-6,7-dihydro-5H-benzo[7]annulen-3-ol
9-{6-[(3-hydroxypropyl){3-[(3,3,3-trifluoropropyl)sulphonyl]propyl}amino]hexyl}-8-phenyl-6,7-dihydro-5H-benzo[7]annulen-3-ol
9-{6-[(2-hydroxy-2-methylpropyl){3-[(RS)-(3,3,3-trifluoropropyl)sulphinyl]propyl}amino]hexyl}-8-phenyl-6,7-dihydro-5H-benzo[7]annulen-3-ol
9-{6-[(2-hydroxy-2-methylpropyl){3-[(3,3,3-trifluoropropyl)sulphonyl]propyl}amino]hexyl}-8-phenyl-6,7-dihydro-5H-benzo[7]annulen-3-ol
9-[5-(methyl{4-[(RS)-(4,4,5,5,5-pentafluoropentyl)sulphinyl]butyl}amino)pentyl]-8-phenyl-6,7-dihydro-5H-benzo[7]annulen-3-ol
9-[5-(methyl{4-[(4,4,5,5,5-pentafluoropentyl)sulphonyl]butyl}amino)pentyl]-8-phenyl-6,7-dihydro-5H-benzo[7]annulen-3-ol
9-[6-(ethyl{3-[(RS)-(4,4,5,5,5-pentafluoropentyl)sulphinyl]propyl}amino)hexyl]-8-(3-hydroxyphenyl)-6,7-dihydro-5H-benzo[7]annulen-3-ol
9-{6-[(2-hydroxyethyl){3-[(RS)-(4,4,5,5,5-pentafluoropentyl)sulphinyl]propyl}amino]hexyl}-8-(3-hydroxyphenyl)-6,7-dihydro-5H-benzo[7]annulen-3-ol
8-(3-hydroxyphenyl)-9-{6[(3-hydroxypropyl){3-[(RS)-(4,4,5,5,5-pentafluoropentyl)sulphinyl]propyl}amino]hexyl}-6,7-dihydro-5H-benzo[7]annulen-3-ol
9-{6-[(2-hydroxy-2-methylpropyl){3-[(RS)-(4,4,5,5,5-pentafluoropentyl)sulphinyl]propyl}amino]hexyl}-8-(3-hydroxyphenyl)-6,7-dihydro-5H-benzo[7]annulen-3-ol
9-[6-(ethyl{3-[(4,4,5,5,5-pentafluoropentyl)sulphonyl]propyl}amino)hexyl]-8-(3-hydroxyphenyl)-6,7-dihydro-5H-benzo[7]annulen-3-ol
9-{6-[(2-hydroxyethyl){3-[(4,4,5,5,5-pentafluoropentyl)sulphonyl]propyl}amino]hexyl}-8-(3-hydroxyphenyl)-6,7-dihydro-5H-benzo[7]annulen-3-ol
8-(3-hydroxyphenyl)-9-{6[(3-hydroxypropyl){3-[(4,4,5,5,5-pentafluoropentyl)sulphonyl]propyl}amino]hexyl}-6,7-dihydro-5H-benzo[7]annulen-3-ol
9-{6-[(2-hydroxy-2-methylpropyl){3-[(4,4,5,5,5-pentafluoropentyl)sulphonyl]propyl}amino]hexyl}-8-(3-hydroxyphenyl)-6,7-dihydro-5H-benzo[7]annulen-3-ol
9-[6-(ethyl{3-[(RS)-(3,3,3-trifluoropropyl)sulphinyl]propyl}amino)hexyl]-8-(3-hydroxyphenyl)-6,7-dihydro-5H-benzo[7]annulen-3-ol
9-{6-[(2-hydroxyethyl){3-[(RS)-(3,3,3-trifluoropropyl)sulphinyl]propyl}amino]hexyl}-8-(3-hydroxyphenyl)-6,7-dihydro-5H-benzo[7]annulen-3-ol
8-(3-hydroxyphenyl)-9-{6-[(3-hydroxypropyl){3-[(RS)-(3,3,3-trifluoropropyl)sulphinyl]propyl}amino]hexyl}-6,7-dihydro-5H-benzo[7]annulen-3-ol
9-{6-[(2-hydroxy-2-methylpropyl){3-[(RS)-(3,3,3-trifluoropropyl)sulphinyl]propyl}amino]hexyl}-8-(3-hydroxyphenyl)-6,7-dihydro-5H-benzo[7]annulen-3-ol
9-{6-[(2-hydroxyethyl){3-[(3,3,3-trifluoropropyl)sulphonyl]propyl}amino]hexyl}-8-(3-hydroxyphenyl)-6,7-dihydro-5H-benzo[7]annulen-3-ol
8-(3-hydroxyphenyl)-9-{6[(3-hydroxypropyl){3-[(3,3,3-trifluoropropyl)sulphonyl]propyl}amino]hexyl}-6,7-dihydro-5H-benzo[7]annulen-3-ol
9-{6-[(2-hydroxy-2-methylpropyl){3-[(3,3, 3-trifluoropropyl)sulphonyl]propyl}amino]hexyl}-8-(3-hydroxyphenyl)-6,7-dihydro-5H-benzo[7]annulen-3-ol
2-fluoro-8-(4-fluoro-3-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol
4-chloro-9-[6-(methyl{3-[(4,4,5,5,5-pentafluoropentyl)sulphonyl]propyl}amino)hexyl]-8-phenyl-6,7-dihydro-5H-benzo[7]annulen-3-ol
4-chloro-9-[6-(methyl{4-[(4,4,5,5,5-pentafluoropentyl)sulphonyl]butyl}amino)hexyl]-8-phenyl-6,7-dihydro-5H-benzo[7]annulen-3-ol
4-chloro-9-[6-(methyl{3-[(RS)-(4,4,5,5,5-pentafluoropentyl)sulphinyl]propyl}amino)hexyl]-8-phenyl-6,7-dihydro-5H-benzo[7]annulen-3-ol
8-(4-fluoro-3-hydroxyphenyl)-9-{6-[(2-methoxyethyl){3-[(4,4,5,5,5-pentafluoropentyl)sulphonyl]propyl}amino]hexyl}-6,7-dihydro-5H-benzo[7]annulen-3-ol
8-(3-hydroxyphenyl)-9-{6-[(2-methoxyethyl){3-[(4,4,5,5,5-pentafluoropentyl)sulphonyl]propyl}amino]hexyl}-6,7-dihydro-5H-benzo[7]annulen-3-ol
8-(3-hydroxyphenyl)-9-{6-[(3-methoxypropyl){3-[(4,4,5,5,5-pentafluoropentyl)sulphonyl]propyl}amino]hexyl}-6,7-dihydro-5H-benzo[7]annulen-3-ol
8-(3-hydroxyphenyl)-9-{6-[(3-methoxypropyl){3-[(RS)-(4,4,5,5,5-pentafluoropentyl)sulphinyl]propyl}amino]hexyl}-6,7-dihydro-5H-benzo[7]annulen-3-ol
4-chloro-9-[6-(methyl{4-[(3,3,3-trifluoropropyl)sulphonyl]butyl}amino)hexyl]-8-phenyl-6,7-dihydro-5H-benzo[7]annulen-3-ol
8-(4-fluoro-3-hydroxyphenyl)-9-[6-(methyl{4-[(3, 3,3-trifluoropropyl)sulphonyl]butyl}amino)hexyl]-6,7-dihydro-5H-benzo[7]annulen-3-ol
9-[5-(methyl{4-[(3,3,3-trifluoropropyl)sulphonyl]butyl}amino)pentyl]-8-phenyl-6,7-dihydro-5H-benzo[7]annulen-3-ol
8-(3-hydroxyphenyl)-9-[5-(methyl{4-[(3,3,3-trifluoropropyl)sulphonyl]butyl}amino)pentyl]-6,7-dihydro-5H-benzo[7]annulen-3-ol
8-(3-hydroxyphenyl)-9-[6-(methyl{4-[(3,3,3-trifluoropropyl)sulphonyl]butyl}amino)hexyl]-6,7-dihydro-5H-benzo[7]annulen-3-ol
4-fluoro-8-(4-fluoro-3-hydroxyphenyl)-9-[6-(methyl{3-[(4,4,5,5,5-pentafluoropentyl)sulphonyl]propyl}amino)hexyl]-6,7-dihydro-5H-benzo[7]annulen-3-ol
4-fluoro-8-(4-fluoro-3-hydroxyphenyl)-9-{6-[(2-hydroxyethyl){3-[(4,4,5,5,5-pentafluoropentyl)sulphonyl]propyl}amino]hexyl}-6,7-dihydro-5H-benzo[7]annulen-3-ol
4-fluoro-8-(4-fluoro-3-hydroxyphenyl)-9-[6-(methyl{3-[(3,3,4,4,4-pentafluorobutyl)sulphonyl]propyl}amino)hexyl]-6,7-dihydro-5H-benzo[7]annulen-3-ol
4-fluoro-8-(4-fluoro-3-hydroxyphenyl)-9-[6-(methyl{4-[(4,4,5,5,5-pentafluoropentyl)sulphonyl]butyl}amino)hexyl]-6,7-dihydro-5H-benzo[7]annulen-3-ol
4-fluoro-8-(4-fluoro-3-hydroxyphenyl)-9-[6-(methyl{4-[(4,4,4-trifluorobutyl)sulphonyl]butyl}amino)hexyl]-6,7-dihydro-5H-benzo[7]annulen-3-ol
4-fluoro-8-(4-fluoro-3-hydroxyphenyl)-9-[6-(methyl{4-[(3,3,3-trifluoropropyl)sulphonyl]butyl}amino)hexyl]-6,7-dihydro-5H-benzo[7]annulen-3-ol
4-fluoro-8-(4-fluoro-3-hydroxyphenyl)-9-[6-(methyl{4-[(RS)-(3,3,3-trifluoropropyl)sulphinyl]butyl}amino)hexyl]-6,7-dihydro-5H-benzo[7]annulen-3-ol
2-fluoro-8-(4-fluoro-3-hydroxyphenyl)-9-[6-(methyl{4-[(4,4,5,5,5-pentafluoropentyl)sulphonyl]butyl}amino)hexyl]-6,7-dihydro-5H-benzo[7]annulen-3-ol
2-fluoro-8-(4-fluoro-3-hydroxyphenyl)-9-[6-(methyl{3-[(RS)-(3, 3,4,4,4-pentafluorobutyl)sulphinyl]propyl}amino)hexyl]-6,7-dihydro-5H-benzo[7]annulen-3-ol
2-fluoro-8-(4-fluoro-3-hydroxyphenyl)-9-[6-(methyl{3-[(3,3,4,4,4-pentafluorobutyl)sulphonyl]propyl}amino)hexyl]-6,7-dihydro-5H-benzo[7]annulen-3-ol
2-fluoro-8-(4-fluoro-3-hydroxyphenyl)-9-[6-(methyl{4-[(RS)-(4,4,5,5,5-pentafluoropentyl)sulphinyl]butyl}amino)hexyl]-6,7-dihydro-5H-benzo[7]annulen-3-ol
2-fluoro-8-(4-fluoro-3-hydroxyphenyl)-9-[6-(methyl{3-[(RS)-(3,3,3-trifluoropropyl)sulphinyl]propyl}amino)hexyl]-6,7-dihydro-5H-benzo[7]annulen-3-ol
2-fluoro-8-(4-fluoro-3-hydroxyphenyl)-9-[6-(methyl{3-[(3,3,3-trifluoropropyl)sulphonyl]propyl}amino)hexyl]-6,7-dihydro-5H-benzo[7]annulen-3-ol
2-fluoro-8-(4-fluoro-3-hydroxyphenyl)-9-[6-(methyl{3-[(4,4,4-trifluorobutyl)sulphonyl]propyl}amino)hexyl]-6,7-dihydro-5H-benzo[7]annulen-3-ol
2-fluoro-8-(4-fluoro-3-hydroxyphenyl)-9-[6-(methyl{4-[(4,4,4-trifluorobutyl)sulphonyl]butyl}amino)hexyl]-6,7-dihydro-5H-benzo[7]annulen-3-ol
2-fluoro-8-(4-fluoro-3-hydroxyphenyl)-9-{6-[(2-hydroxy-2-methylpropyl){3-[(RS)-(3,3,3-trifluoropropyl)sulphinyl]propyl}amino]hexyl}-6,7-dihydro-5H-benzo[7]annulen-3-ol
2-fluoro-8-(4-fluoro-3-hydroxyphenyl)-9-[6-(methyl{3[(RS)-(4,4,5,5,5-pentafluoropentyl)sulphinyl]propyl}amino)hexyl]-6,7-dihydro-5H-benzo[7]annulen-3-ol
2-fluoro-8-(4-fluoro-3-hydroxyphenyl)-9-{6-[(2-methoxyethyl){3-[(RS)-(4,4,5,5,5-pentafluoropentyl)sulphinyl]propyl}amino]hexyl}-6,7-dihydro-5H-benzo[7]annulen-3-ol
2-fluoro-8-(4-fluoro-3-hydroxyphenyl)-9-{6-[(3-methoxypropyl){3-[(RS)-(4,4,5,5,5-pentafluoropentyl)sulphinyl]propyl}amino]hexyl}-6,7-dihydro-5H-benzo[7]annulen-3-ol.

6. Compound as defined in claim 1 for the treatment and/or prophylaxis of diseases.

7. Use of a compound as defined in claim 1 for the production of a medicament for the treatment and/or prophylaxis of diseases.

8. Compound of the formula (I) as defined in claim 1 for use in a method of induction of ovulation, for inhibition of sperm maturation, for alleviating the symptoms of the andropause and menopause, i.e. for male and female hormone replacement therapy, for the prevention or prophylaxis and for the treatment of disorders accompanying dysmenorrhoea, of dysfunctional uterine bleeding, acne, cardiovascular diseases, hypercholesterolaemia and hyperlipidaemia, atherosclerosis, proliferation of arterial smooth muscle cells, respiratory distress syndrome of the newborn, primary pulmonary hypertension, osteoporosis, bone loss in postmenopausal women, in hysterectomized women or in women who have been treated with LHRH agonists or antagonists, rheumatoid arthritis, Alzheimer's disease, endometriosis, myomata, hormone-dependent tumours, for example breast or endometrial carcinoma, infertility, prostatic diseases, benign diseases of the breast, e.g. mastopathy, stroke, Alzheimer's and other diseases of the central nervous system that are associated with cellular death of neurons.

9. Use of a compound as defined in claim 1 for preparing a medicament for induction of ovulation, for inhibition of sperm maturation, for alleviating the symptoms of the andropause and menopause, i.e. for male and female hormone replacement therapy, for the prevention or prophylaxis and for the treatment of disorders accompanying dysmenorrhoea, of dysfunctional uterine bleeding, acne, cardiovascular diseases, hypercholesterolaemia and hyperlipidaemia, atherosclerosis, proliferation of arterial smooth muscle cells, respiratory distress syndrome of the newborn, primary pulmonary hypertension, osteoporosis, bone loss in postmenopausal women, in hysterectomized women or in women who have been treated with LHRH agonists or antagonists, rheumatoid arthritis, Alzheimer's disease, endometriosis, myomata, hormone-dependent tumours, for example breast or endometrial carcinoma, infertility, prostatic diseases, benign diseases of the breast, e.g. mastopathy, stroke, Alzheimer's and other diseases of the central nervous system that are associated with cellular death of neurons.

10. Medicament comprising a compound as defined in claim 1 in combination with one or more further active compounds, in particular with aromatase inhibitors, 17beta HSD1 inhibitors, steroid sulphatase (STS) inhibitors, LHRH analogues, LHRH antagonists, GnRH agonists and antagonists, kisspeptin receptor (KISSR) antagonists, selective androgen receptor modulators (SARMs), androgens, selective progesterone receptor modulators (SPRMs), gestagens, antigestagens (progesterone receptor antagonists), oral contraceptives, oestrogens, inhibitors of mitogen-activated protein (MAP) kinases and inhibitors of MAP kinases kinases (Mkk3/6, Mek1/2, Erk1/2) inhibitors of protein kinases B (PKBa/β/γ; Akt1/2/3), inhibitors of phosphoinositide 3-kinases (PI3K), inhibitors of cyclin-dependent kinase (CDK1/2), inhibitors of the hypoxia-induced signal pathway (HIF1alpha inhibitors, activators of prolyl hydroxylases), histone deacetylase (HDAC) inhibitors, prostaglandin F receptor (FP) (PTGFR) antagonists and non-steroidal antiinflammatory drugs (NSAIDs) for the treatment of endometriosis.

11. Medicament comprising a compound as defined in claim 1 in combination with an inert, non-toxic, pharmaceutically suitable excipient.

12. Medicament according to claim 10 for induction of ovulation, for inhibition of sperm maturation, for alleviating the symptoms of the andropause and menopause, i.e. for male and female hormone replacement therapy, for the prevention or prophylaxis and for the treatment of disorders accompanying dysmenorrhoea, of dysfunctional uterine bleeding, acne, cardiovascular diseases, hypercholesterolaemia and hyperlipidaemia, atherosclerosis, proliferation of arterial smooth muscle cells, respiratory distress syndrome of the newborn, primary pulmonary hypertension, osteoporosis, bone loss in postmenopausal women, in hysterectomized women or in women who have been treated with LHRH agonists or antagonists, rheumatoid arthritis, Alzheimer's disease, endometriosis, myomata, hormone-dependent tumours, for example breast or endometrial carcinoma, infertility, prostatic diseases, benign diseases of the breast, e.g. mastopathy, stroke, Alzheimer's and other diseases of the central nervous system that are associated with cellular death of neurons.
